SEQUENCE LISTING (8 sequences)

SEQ ID NO: 1 - DNA, 807 bp, Thermomyces lanuginosus
  source 1..807: mol_type = genomic DNA
gaggtctcgcaggatctgtttaaccagttcaatctctttgcacagtattctgcagccgcatactgcggaaaaaacaatgatgccccagctggtacaaacattacgtgcacgggaaatgcctgccccgaggtagagaaggcggatgcaacgtttctctactcgtttgaagactctggagtgggcgatgtcaccggcttccttgctctcgacaacacgaacaaattgatcgtcctctctttccgtggctctcgttccatagagaactggatcgggaatcttaacttcgacttgaaagaaataaatgacatttgctccggctgcaggggacatgacggcttcacttcgtcctggaggtctgtagccgatacgttaaggcagaaggtggaggatgctgtgagggagcatcccgactatcgcgtggtgtttaccggacatagcttgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcgaattcggttacagccattctagcccagagtactggatcaaatctggaacccttgtccccgtcacccgaaacgatatcgtgaagatagaaggcatcgatgccaccggcggcaataaccagcctaacattccggatatccctgcgcacctatggtacttcgggttaattgggacatgtctt

SEQ ID NO: 2 - protein, 269 aa, Thermomyces lanuginosus [sequence omitted]

SEQ ID NO: 3 - DNA, 807 bp, synthetic construct
  source 1..807: mol_type = other DNA
gaggtctcgcaggatctgtttaaccagttcaatctctttgcacagtattctgcagccgcatactgcggaaaaaacaatgatgccccagctggtacaaacattacgtgcacgggcaatgaatgccccgaggtagagaaggcggatgcaacgtttctctactcgttccgcaactctggagtgggcgatgtcaccggcttccttgctctcgacaacacgaacaaattgatcgtcctctctttccgtggctctcgttccatagagaactggatcaccaatcttaacttcgacttggaagaaataaatgacatttgctccggctgcaaaggacatgacggcttcacctcgtcctggttttccgtagccgatacgttaaggcagaaggtggaggatgctgtgagggagcatcccgactatcgcgtggtgtttaccggacatagcttgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcaaattcggttacagccattctagcccagaatactggatcaaatctggaacccttgtccccgtccggcgacgagacatcgtgaagatagaaggcatcgatgccgagggtggcaataaccagcctaacattccgtcgatccctgcgcacctatggtacttcggccttattgggacatgtctt

SEQ ID NO: 4 - protein, 269 aa, synthetic construct
EVSQDLFNQFNLFAQYSAAAYCGKNNDAPAGTNITCTGNECPEVEKADATFLYSFRNSGVGDVTGFLALDNTNKLIVLSFRGSRSIENWITNLNFDLEEINDICSGCKGHDGFTSSWFSVADTLRQKVEDAVREHPDYRVVFTGHSLGGALATVAGADLRGNGYDIDVFSYGAPRVGNRAFAEFLTVQTGGTLYRITHTNDIVPRLPPRKFGYSHSSPEYWIKSGTLVPVRRRDIVKIEGIDAEGGNNQPNIPSIPAHLWYFGLIGTCL

SEQ ID NO: 5 - DNA, 807 bp, synthetic construct
  source 1..807: mol_type = other DNA
gaggtctcgcaggatctgtttaaccagttcaatctctttgcacagtattctgcagccgcatactgctccaaaaacaataacgccccagctggtacaaacattacgtgcacgggcaatatctgccccgaggtagagaaggcggatgcaacgatcctctactcgtttagggactctggaaaaggcgatgtcaccggcttccttgctctcgacaacacgaacaaattgatcgtcctctctttccgtggctctcgttccatagagaactggatcgggaatcttaacttcgacttgaaagaaataaatgacatttgctccggctgcaggggacatgacggcttcacttcgtcctggttctctgtagccgatacgttaaggcagaaggtggaggatgctgtgagggagcatcccgactatcgcgtggtgtttaccggacatagcttgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcgaattcggttacagccattctagcccagagtactggatcaaatctggaacccttgtccccgtcaggcgaagggatatcgtgaagatagaaggcatcgatgccgaaggcggcaataaccagcctaacattccggatatcaccgcgcacctatggtacttcgggttaattgggacatgtctt

SEQ ID NO: 6 - protein, 269 aa, synthetic construct
EVSQDLFNQFNLFAQYSAAAYCSKNNNAPAGTNITCTGNICPEVEKADATILYSFRDSGKGDVTGFLALDNTNKLIVLSFRGSRSIENWIGNLNFDLKEINDICSGCRGHDGFTSSWFSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVRRRDIVKIEGIDAEGGNNQPNIPDITAHLWYFGLIGTCL

SEQ ID NO: 7 - DNA, 807 bp, synthetic construct
  source 1..807: mol_type = other DNA
gaggtctcgcaggatctgtttaaccagttcaatctctttgcacagtattctgcagccgcatactgcggaaaaaacaatgatgccccagctggtacaaacattacgtgcacgggaaatgcctgccccgaggtagagaaggcggatgcaacgtttctctactcgtttgaagactctggagtgggcgatgtcaccggcttccttgctctcgacaacacgaacaaattgatcgtcctctctttccgtggctctcgttccatagagaactggatcgggaatcttaacttcgacttgaaagaaataaatgacatttgctccggctgcaggggacatgacggcttcacttcgtcctggaggtctgtagccgatacgttaaggcagaaggtggaggatgctgtgagggagcatcccgactatcgcgtggtgtttaccggacatagcttgggtggtgcattggcaactgttgccggagcagacctgcgtggaaatgggtatgatatcgacgtgttttcatatggcgccccccgagtcggaaacagggcttttgcagaattcctgaccgtacagaccggcggaacactctaccgcattacccacaccaatgatattgtccctagactcccgccgcgcgaattcggttacagccattctagcccagaatactggatcaaatctggaacccttgtccccgtccggcgacgagacatcgtgaagatagaaggcatcgatgccaccggcggcaataaccagcctaacattccggatatccctgcgcacctatggtacttcgggttaattgggacatgtctt

SEQ ID NO: 8 - protein, 269 aa, synthetic construct
EVSQDLFNQFNLFAQYSAAAYCGKNNDAPAGTNITCTGNACPEVEKADATFLYSFEDSGVGDVTGFLALDNTNKLIVLSFRGSRSIENWIGNLNFDLKEINDICSGCRGHDGFTSSWRSVADTLRQKVEDAVREHPDYRVVFTGHSLGGALATVAGADLRGNGYDIDVFSYGAPRVGNRAFAEFLTVQTGGTLYRITHTNDIVPRLPPREFGYSHSSPEYWIKSGTLVPVRRRDIVKIEGIDATGGNNQPNIPDIPAHLWYFGLIGTCL

(19) United States
(12) Patent Application Publication
(10) Pub. No.: US 2025/0368924 A1
(43) Pub. Date: Dec. 4, 2025

(54) LIPASE VARIANTS AND COMPOSITIONS COMPRISING SUCH LIPASE VARIANTS

(71) Applicant: Novozymes A/S, Bagsvaerd (DK)

(72) Inventor: Jesper VIND, Bagsvaerd (DK)

(73) Assignee: Novozymes A/S, Bagsvaerd (DK)

(21) Appl. No.: 18/872,791

(22) PCT Filed: Jun. 22, 2023
(86) PCT No.: PCT/EP2023/066896

§ 371 Date: Dec. 6, 2024

(30) Foreign Application Priority Data
Jun. 24, 2022 (EP) 22180938.7

(51) Int. Cl. C11D 3/386; C12N 9/20

(52) U.S. Cl. CPC C11D 3/38627; C12N 9/20; C12Y 301/01003

(57) ABSTRACT

The present invention relates to lipase variants with reduced activity at pHs around neutral compared to the parent. The present invention also relates to compositions comprising a lipase variant of the invention; polynucleotides encoding lipase variants of the invention; nucleic acid constructs, vectors, and host cells comprising the polynucleotides; and methods of producing and using the variants for cleaning.

### REFERENCE TO A SEQUENCE LISTING

This application contains a Sequence Listing in computer readable form, which is incorporated herein by reference.

### BACKGROUND OF THE INVENTION

### Field of the Invention

The present invention relates to lipase variants, compositions comprising lipase variants of the invention, polynucleotides encoding variants of the invention, nucleic acid constructs comprising polynucleotides of the invention, expression vectors comprising polynucleotides or nucleic acid constructs of the invention, host cells comprising nucleic acid constructs or expression vectors of the invention. Finally, the invention relates methods for cleaning surfaces with variants or compositions of the invention, methods of hydrolyzing lipase substrates with lipase variants or compositions of the invention, method for washing laundry with variants or compositions of the invention, and methods of producing variants of the invention.

### Description of the Related Art

Lipases are important biocatalysts which have shown to be useful for various applications. Variants of the wild-type Thermomyces lanuginosus lipase (synonym Humicola lanuginosa) have been commercialized as active ingredient in detergent compositions for the removal of lipid stains by hydrolyzing triglycerides to generate fatty acids.

Detergent, cleaning and/or fabric care compositions comprise active ingredients which interfere with the ability of lipases to remove lipid stains. Many known Thermomyces lanuginosus lipase variants with good wash performance form odor-generating short-chain fatty acids during wash and/or have a short storage stability.

WO 2016/050661 (Novozymes) concerns Thermomyces lanuginosus lipase variants, with reduced odor generation, where the lipase variants comprise a substitution at positions corresponding to position 210 which is not a negatively charged amino acid, and position 255 which is not I, and wherein position 256 is not K.

WO 2017/001673 (Novozymes) discloses Thermomyces lanuginosus lipase variants with reduced odor generation, wherein the lipase variants comprise one or more substitutions selected from F7H/K/R, F51A/I/L/VN/Y, T143A/G/S/V, A150G/V, H198A/D/E/F/G/I/L/N/Q/S/T/Y, N200H/K/Q/R, 1202G/L/V, S224C/F/H/I/L/P/Y, L227D/E/K/R, V228P, P229H/K/R, V230H/K/L/R, 1255A/G/N/P/S/T/V/Y, P256A/K/N/Q/R/S/T/W, A257F/H/I/L/V/Y, L259F/Y, and W260D/E/F/H/I/L/N/Q/S/T/Y using SEQ ID NO:10 for position numbering or selected from H198A/D/E/F/G/I/L/N/Q/S/T/V/Y, F7H/K/R, F51A/I/L/V/Y, T143A/G/SN, A150G/V, N200H/K/Q/R, 1202G/L/V, S224C/F/H/I/L/P/Y, L227D/E/K/R, V228P, P229H/K/R, V230H/K/L/R, 1255A/G/N/P/S/T/V/Y, T256A/K/N/Q/R/S/P/W, A257F/H/I/L/V/Y, L259F/Y, and W260D/E/F/H/I/L/N/Q/S/T/Y.

Dispite progress there is still a need and desire for lipases with improved properties.

### SUMMARY OF THE INVENTION

An important goal of the present invention is to provide lipase variants with reduced lipase activity at pHs around neutral, i.e., around pH 6-8, in particular around pH 7. Reduced activity results in reduced odor-generated by the lipase variant which hydrolyzes short chained lipid substrates. At higher pHs, i.e., around pH 8-11, the lipase variant inflicted odor-generation is higher than at pHs around neutral. Such lipase variants can advantageous be used, e.g., for cleaning laundry. During laundry washing, the pH of the wash solution is high, e.g., pH 8-11, while the pH of the rinse water during the subsequent rinse cycle is around neutral, i.e., pH 6-8. Thus, lipase variants of the invention mitigate the odor-generation problem occurring during the washing cycle done at high pHs by reducing odor generation during the rinse cycle where the pH is lower, i.e., around neutral.

The present invention relates to isolated lipase variants, selected from one or more of groups (i), (ii) and (iii) comprising
(i) a substitution at one or more positions corresponding to positions 202, 252, and 269 of the polypeptide of SEQ ID NO: 8;
(ii) a substitution at one or more positions corresponding to positions 40, 56, 57, 91, 98, 108, 118, 210, 244, and 254 of the polypeptide of SEQ ID NO: 8; and
(iii) a substitution at one or more positions corresponding to positions 23, 27, 40, 51, 56, 60, 118, 244 and 256 of the polypeptide of SEQ ID NO: 8;
wherein the variant has lipase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 8, wherein the variant optionally comprises an extension of one or more amino acids at the N-terminal and/or C-terminal ends or a truncation of one or more amino acids at the N-terminal and/or C-terminal ends and wherein the variant has lipase activity.

In a preferred embodiment, the lipase variant has one or more substitutions, in particular all substitutions, from group (i). In a preferred embodiment, the lipase variant has one or more substitutions, in particular all substitutions, from group (ii). In a preferred embodiment, the lipase variant has one or more substitutions, in particular all substitutions, from groups (i) and one or more substitutions, in particular all substitutions, from group (ii). In another preferred embodiment, the lipase variant has one or more substitutions, in particular all substitutions, from groups (i) and one or more substitutions, in particular all substitutions, from group (iii).

In an aspect, the invention relates to granules, which comprise:
(a) a core comprising the variant of the invention, and, optionally
(b) a coating consisting of one or more layer(s) surrounding the core.

In another aspect, the invention relates to liquid compositions comprising the variant of the invention and an enzyme stabilizer, e.g., a polyol such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

The invention also relates to compositions comprising the variant of the invention, a granule of the invention, or the liquid compositions of the invention. In a preferred embodiment, the composition comprises one or more surfactants.

The present invention also relates to a polynucleotide encoding a variant of the invention.

In an aspect, the invention relates to a nucleic acid construct or expression vector comprising the polynucleotide of the invention. The invention also relates to a recombinant host cell transformed with the polynucleotide of the invention. In an aspect the invention relates to methods of producing a lipase variant of the invention, comprising:
a. cultivating the recombinant host cell of the invention under conditions suitable for expression of the variant; and
b. recovering the variant.

The invention also relates to methods for hydrolyzing a lipase substrate comprising mixing the substrate with a lipase variant of the invention or the composition of the invention at conditions conductive for the lipase variant hydrolyzing the substrate.

In another aspect, the invention relates to methods for removing lipid stain material from a surface comprising contacting the lipid stain material with a lipase variant of the invention or the composition of the invention at conditions conductive for the lipase variant hydrolyzing the lipid stain material.

The invention also relates to methods for lipid stain removal from a surface comprising: contacting said stain with a lipase variant of the invention or a composition of the invention, followed by rinsing the surface, and optionally drying.

The invention also relates to methods for lipid stain removal from a surface comprising: contacting said stain with a lipase variant of the invention, or a composition of the invention, followed by rinsing, and optionally drying, in which method, the odor generation is reduced when compared to the method wherein the parent lipase, in particular one of SEQ ID NOs: 2, 4, 6 or 8, respectively, is contacted to the stain.

Finally, the invention also relates to the use of a lipase variant of the invention or a composition of the invention for cleaning a surface comprising applying the lipase variant to the surface to be cleaned, followed by rinsing, and optionally drying.

### BRIEF DESCRIPTION OF THE FIGURES

FIG. 1 is an alignment of the parent lipases of SEQ ID NO: 2 (wild-type Thermomyces lanuginosus lipase), SEQ ID NO: 4, SEQ ID NO: 6, and SEQ ID NO: 8 using the Clustal Omega (1.2.4) multiple sequence alignment software available on EMBL's European Bioinformatics Institute webpage (www.ebi.ac.uk).

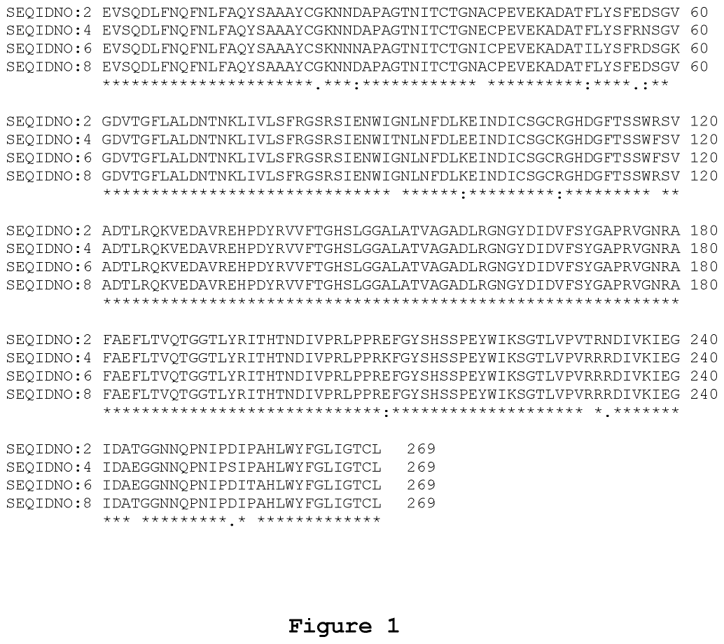

### DEFINITIONS

In accordance with this detailed description, the following definitions apply. Note that the singular forms “a,” “an,” and “the” include plural references unless the context clearly dictates otherwise.

Unless defined otherwise or clearly indicated by context, all technical and scientific terms used herein have the same meaning as commonly understood by one of ordinary skill in the art to which this invention belongs.

Lipase: The term “lipase”, “lipase enzyme”, “lipolytic enzyme”, “lipid esterase”, “lipolytic polypeptide”, and “lipolytic protein” refers to an enzyme in class EC 3.1.1 as defined by Enzyme Nomenclature. It may have lipase activity (triacylglycerol lipase, EC 3.1.1.3), cutinase activity (EC 3.1.1.74), sterol esterase activity (EC 3.1.1.13) and/or wax-ester hydrolase activity (EC 3.1.1.50).

Lipase Activity: For purposes of the present invention lipase activity (i.e. the hydrolytic activity of the lipase) may be determined with a pNP assay using substrates with various chain length as described in the Examples.

In one aspect, the variants of the present invention have at least 20%, e.g., at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the lipase activity of the parent lipase. In one aspect, the parent lipase is the polypeptide of SEQ ID NO: 8, or a fragment thereof with lipase activity. SEQ ID NO: 8 is the same as the wild-type Thermomyces lanuginosus lipase shown in SEQ ID NO: 2 with T231R+N233R substitutions.

Benefit Risk factor: The Benefit Risk factor (BRF) describes the wash performance (Benefit) compared to the odor release (Risk) and is defined as RP (wash)/RP (odor). If the Benefit Risk factor of a lipase variant is higher than 1.0, the lipase has better wash performance relative to the released odor compared to the reference lipase, in particular parent lipase in SEQ ID NO: 2, 4, 6, or 8, respectively.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences involved in regulation of expression of a polynucleotide in a specific organism or in vitro. Each control sequence may be native (i.e., from the same gene) or heterologous (i.e., from a different gene) to the polynucleotide encoding the variant, and native or heterologous to each other. Such control sequences include, but are not limited to leader, polyadenylation, prepropeptide, propeptide, signal peptide, promoter, terminator, enhancer, and transcription or translation initiator and terminator sequences. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Expression: The term “expression” includes any step involved in the production of a variant including, but not limited to, transcription, post-transcriptional modification, translation, post-translational modification, and secretion.

Expression vector: An “expression vector” refers to a linear or circular DNA construct comprising a DNA sequence encoding a variant, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

Extension: The term “extension” means an addition of one or more amino acids to the amino and/or carboxyl terminus of a variant, wherein the “extended” variant has lipase activity.

Fragment: The term “fragment” means a variant having one or more amino acids absent from the amino and/or carboxyl terminus of the variant; wherein the fragment has lipase activity.

Fusion polypeptide: The term “fusion polypeptide” is a polypeptide in which one polypeptide is fused at the N-terminus and/or the C-terminus of a variant of the present invention. A fusion polypeptide is produced by fusing a polynucleotide encoding another polypeptide to a polynucleotide of the present invention, or by fusing two or more polynucleotides of the present invention together. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, of a polypeptide or nucleic acid is not naturally associated with the polypeptide or nucleic acid, i.e., the control sequence is from a gene other than the gene encoding the mature polypeptide.

Host Strain or Host Cell: A “host strain” or “host cell” is an organism into which an expression vector, phage, virus, or other DNA construct, including a polynucleotide encoding a variant has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

Improved property: The term “improved property” means a characteristic associated with a variant that is improved compared to the parent. Such improved properties include but are not limited to: reduced lipase activity and/or reduced odor generation at pHs around neutral, i.e., around pH 6-8, preferably around pH 7 and/or increased benefit risk factor (BRF) compared to the parent lipase, in particular SEQ ID NOs: 2, 4, 6 or 8, respectively.

Introduced: The term “introduced” in the context of inserting a nucleic acid sequence into a cell, means “transfection”, “transformation” or “transduction,” as known in the art.

Isolated: The term “isolated” means a variant, nucleic acid, cell, or other specified material or component that is separated from at least one other material or component, including but not limited to, other proteins, nucleic acids, cells, etc. An isolated polypeptide, nucleic acid, cell or other material is thus in a form that does not occur in nature. An isolated polypeptide includes, but is not limited to, a culture broth containing the secreted variant expressed in a host cell.

Mature polypeptide: The term “mature polypeptide” means a polypeptide in its mature form following N-terminal processing and/or C-terminal processing (e.g., removal of signal peptide).

Mature polypeptide coding sequence: The term “mature polypeptide coding sequence” means a polynucleotide that encodes a mature polypeptide having lipase activity.

Mutant: The term “mutant” means a polynucleotide encoding a variant.

Native: The term “native” means a nucleic acid or polypeptide naturally occurring in a host cell.

Nucleic acid: The term “nucleic acid” encompasses DNA, RNA, heteroduplexes, and synthetic molecules capable of encoding a variant. Nucleic acids may be single stranded or double stranded, and may be chemical modified. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, and which comprises one or more control sequences operably linked to the nucleic acid sequence.

Operably linked: The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequence.

Parent or parent lipase: The term “parent” or “parent lipase” means a lipase to which an alteration is made to produce the enzyme variants of the present invention.

Purified: The term “purified” means a nucleic acid, variant or cell that is substantially free from other components as determined by analytical techniques well known in the art (e.g., a purified variant or nucleic acid may form a discrete band in an electrophoretic gel, chromatographic eluate, and/or a media subjected to density gradient centrifugation). A purified nucleic acid or variant is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight or on a molar basis). In a related sense, a composition is enriched for a molecule when there is a substantial increase in the concentration of the molecule after application of a purification or enrichment technique. The term “enriched” refers to a compound, variant, cell, nucleic acid, amino acid, or other specified material or component that is present in a composition at a relative or absolute concentration that is higher than a starting composition.

In one aspect, the term “purified” as used herein refers to the variant or cell being essentially free from components (especially insoluble components) from the production organism. In other aspects. the term “purified” refers to the variant being essentially free of insoluble components (especially insoluble components) from the native organism from which it is obtained. In one aspect, the variant is separated from some of the soluble components of the organism and culture medium from which it is recovered. The variant may be purified (i.e., separated) by one or more of the unit operations filtration, precipitation, or chromatography.

Accordingly, the variant may be purified such that only minor amounts of other proteins, in particular, other polypeptides, are present. The term “purified” as used herein may refer to removal of other components, particularly other proteins and most particularly other enzymes present in the cell of origin of the polypeptide. The variant may be “substantially pure”, i.e., free from other components from the organism in which it is produced, e.g., a host organism for recombinantly produced variant. In one aspect, the polypeptide is at least 40% pure by weight of the total polypeptide material present in the preparation. In one aspect, the polypeptide is at least 50%, 60%, 70%, 80% or 90% pure by weight of the total polypeptide material present in the preparation. As used herein. a “substantially pure polypeptide” may denote a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which the polypeptide is natively or recombinantly associated.

It is, therefore, preferred that the substantially pure variant is at least 92% pure, preferably at least 94% pure, more preferably at least 95% pure, more preferably at least 96% pure, more preferably at least 97% pure, more preferably at least 98% pure, even more preferably at least 99% pure, most preferably at least 99.5% pure by weight of the total polypeptide material present in the preparation. The variant of the present invention is preferably in a substantially pure form (i.e., the preparation is essentially free of other polypeptide material with which it is natively or recombinantly associated). This can be accomplished, for example by preparing the variant by well-known recombinant methods or by classical purification methods.

Recombinant: The term “recombinant” is used in its conventional meaning to refer to the manipulation, e.g., cutting and rejoining, of nucleic acid sequences to form constellations different from those found in nature. The term recombinant refers to a cell, nucleic acid, variant or vector that has been modified from its native state. Thus, for example, recombinant cells express genes that are not found within the native (non-recombinant) form of the cell, or express native genes at different levels or under different conditions than found in nature. The term “recombinant” is synonymous with “genetically modified” and “transgenic”.

Recover: The terms “recover” or “recovery” means the removal of a polypeptide from at least one fermentation broth component selected from the list of a cell, a nucleic acid, or other specified material, e.g., recovery of the polypeptide from the whole fermentation broth, or from the cell-free fermentation broth, by polypeptide crystal harvest, by filtration, e.g., depth filtration (by use of filter aids or packed filter medias, cloth filtration in chamber filters, rotary-drum filtration, drum filtration, rotary vacuum-drum filters, candle filters, horizontal leaf filters or similar, using sheed or pad filtration in framed or modular setups) or membrane filtration (using sheet filtration, module filtration, candle filtration, microfiltration, ultrafiltration in either cross flow, dynamic cross flow or dead end operation), or by centrifugation (using decanter centrifuges, disc stack centrifuges, hyrdo cyclones or similar), or by precipitating the polypeptide and using relevant solid-liquid separation methods to harvest the polypeptide from the broth media by use of classification separation by particle sizes. Recovery encompasses isolation and/or purification of the polypeptide.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, the-nobrief option must be specified in the command line. The output of Needle labeled “longest identity” is calculated as follows:

(Identical Residues×100)/(Length of Alignment−Total Number of Gaps in Alignment)

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

(Identical Deoxyribonucleotides×100)/(Length of Alignment−Total Number of Gaps in Alignment)

Signal Peptide: A “signal peptide” is a sequence of amino acids attached to the N-terminal portion of a protein, which facilitates the secretion of the protein outside the cell. The mature form of an extracellular protein lacks the signal peptide, which is cleaved off during the secretion process.

Subsequence: The term “subsequence” means a polynucleotide having one or more nucleotides absent from the 5′ and/or 3′ end of a mature polypeptide coding sequence; wherein the subsequence encodes a fragment having lipase activity.

Variant: The term “variant” means a polypeptide having lipase activity comprising a substitution, an insertion (including extension), and/or a deletion (e.g., truncation), at one or more positions. A substitution means replacement of the amino acid occupying a position with a different amino acid; a deletion means removal of the amino acid occupying a position; and an insertion means adding 1-5 amino acids (e.g., 1-3 amino acids, in particular, 1 amino acid) adjacent to and immediately following the amino acid occupying a position.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### Conventions for Designation of Variants

For purposes of the present invention, the mature polypeptide disclosed in SEQ ID NO: 4 is used to determine the corresponding amino acid positions in another lipase. The amino acid sequence of another lipase is aligned with the polypeptide disclosed in SEQ ID NO: 4, and based on the alignment, the amino acid position number corresponding to any amino acid residue in the polypeptide disclosed in SEQ ID NO: 4 is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix.

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine (S) with phenylalanine (F), respectively.

Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions. For an amino acid insertion, the following nomenclature is used: Original amino acid, position, original amino acid, inserted amino acid. Accordingly, the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different alterations. Where different alterations can be introduced at a position, the different alterations are separated by a comma, e.g., “Arg170Tyr,Glu” represents a substitution of arginine at position 170 with tyrosine or glutamic acid. Thus, “Tyr167Gly,Ala+Arg170Gly,Ala” designates the following variants:

### “Tyr167Gly+Arg170Gly”, “Tyr167Gly+Arg170Ala”, “Tyr167Ala+Arg170Gly”, and “Tyr167Ala+Arg170Ala”.

### DETAILED DESCRIPTION OF THE INVENTION

### Variant of the Invention

The present invention relates to lipase variants, selected from one or more of groups (i), (ii) and (iii) comprising
(i) a substitution at one or more positions corresponding to positions 202, 252, and 269 of the polypeptide of SEQ ID NO: 8;
(ii) a substitution at one or more positions corresponding to positions 40, 56, 57, 91, 98, 108, 118, 210, 244, and 254 of the polypeptide of SEQ ID NO: 8; and
(iii) a substitution at one or more positions corresponding to positions 23, 27, 40, 51, 56, 60, 118 244 and 256 of the polypeptide of SEQ ID NO: 8;
wherein the variant has lipase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 8.

In a preferred embodiment, the lipase variant has one or more substitutions, in particular all substitutions, from group (i). In a preferred embodiment, the lipase variant has one or more substitutions, in particular all substitutions, from group (ii). In a preferred embodiment, the lipase variant has one or more substitutions, in particular all substitutions, from groups (i) and one or more substitutions, in particular all substitutions, from group (ii). In another preferred embodiment, the lipase variant has one or more substitutions, in particular all substitutions, from groups (i) and one or more substitutions, in particular all substitutions, from group (iii).

The variants may further comprise an extension (i.e., peptide addition) of one or more amino acids at the N-terminal and/or C-terminal ends. In one preferred embodiment, the extension is a SPIRR-peptide (or one or more amino acids thereof) located at the N-terminal of the lipase. Suitable lipase extensions are disclosed in WO 1997/004079 (hereby incorporated by reference). Examples of C-terminal extensions are disclosed in WO 2000/060063 (hereby incorporated by reference). Alternatively, the variants may further comprise a truncation of one or more amino acids at the N-terminal and/or C-terminal ends.

In an embodiment, the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of the parent lipase.

In another embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the polypeptide of SEQ ID NO: 2.

In another embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the polypeptide of SEQ ID NO: 4.

In another embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the polypeptide of SEQ ID NO: 6.

In another embodiment, the variant has at least 60%, e.g., at least 65%, at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the polypeptide of SEQ ID NO: 8.

In one aspect, the number of alterations, in particular substitutions, in the variants of the present invention is 1-20, e.g., 1-10 and 1-5, such as 1, 2, 3, 4, 5, 6, 7, 8, 9 or 10 alterations, in particular substitutions.

In an embodiment, the variant of the invention comprises or consists of one or more substitutions, in particular all substitutions, corresponding to the positions in SEQ ID NO: 8, selected from the group consisting of:
a substitution of the amino acid residue at position 202 with H;
a substitution of the amino acid residue at position 252 with H; and
a substitution of the amino acid residue at position 269 with H.

In an embodiment, the variant of the invention comprises or consists of one of the following set of substitutions corresponding to: 202H+252H; 202H+269H; 252H+269H; or 202H+252H+269H (using SEQ ID NO: 8 for numbering).

In a preferred embodiment, the variant of the invention comprises or consists of one or more substitutions, in particular all substitutions, corresponding to the positions in SEQ ID NO: 8, selected from the group consisting of:
a substitution of the amino acid residue at position 40 with E;
a substitution of the amino acid residue at position 56 with R;
a substitution of the amino acid residue at position 57 with N;
a substitution of the amino acid residue at position 91 with T;
a substitution of the amino acid residue at position 98 with E;
a substitution of the amino acid residue at position 108 with K;
a substitution of the amino acid residue at position 118 with F;
a substitution of the amino acid residue at position 210 with K;
a substitution of the amino acid residue at position 244 with E; and
a substitution of the amino acid residue at position 254 with S.

In another preferred embodiment, the variant of the invention comprises or consists of one or more substitutions, in particular all substitutions, corresponding to the positions in SEQ ID NO: 8, selected from the group consisting of:
a substitution of the amino acid residue at position 23 with S;
a substitution of the amino acid residue at position 27 with N;
a substitution of the amino acid residue at position 40 with I;
a substitution of the amino acid residue at position 51 with I;
a substitution of the amino acid residue at position 56 with R;
a substitution of the amino acid residue at position 60 with K;
a substitution of the amino acid residue at position 118 with F;
a substitution of the amino acid residue at position 244 with E; and a substitution of the amino acid residue at position 256 with T.

In an embodiment, a variant of the invention has a substitution corresponding to I202H of SEQ ID NO: 8 and further comprises one of the following substitutions or set of substitutions corresponding to: A40E, E56R, D57N, G91T, K98E, R108K, R118F, E210K, T244E, A40E+E56R, A40E+D57N, A40E+G91T, A40E+K98E, A40E+R108K, A40E+R118F, A40E+E210K, A40E+T244E, A40E+D254S, E56R+D57N, E56R+G91T, E56R+K98E, E56R+R108K, E56R+R118F, E56R+E210K, E56R+T244E, E56R+D254S, D57N+G91T, D57N+K98E, D57N+R108K, D57N+R118F, D57N+E210K, D57N+T244E, D57N+D254S, G91T+K98E, G91T+R108K, G91T+R118F, G91T+E210K, G91T+T244E, G91T+D254S, K98E+R108K, K98E+R118F, K98E+E210K, K98E+T244E, K98E+D254S, R108K+R118F, R108K+E210K, R108K+T244E, R108K+D254S, R118F+E210K, R118F+T244E, R118F+D254S, E210K+T244E, E210K+D254S, T244E+D254S, A40E+E56R+D57N, A40E+E56R+G91T, A40E+E56R+K98E, A40E+E56R+R108K, A40E+E56R+R118F, A40E+E56R+E210K, A40E+E56R+T244E, A40E+E56R+D254S, A40E+D57N+G91T, A40E+D57N+K98E, A40E+D57N+R108K, A40E+D57N+R118F, A40E+D57N+E210K, A40E+D57N+T244E, A40E+D57N+D254S, A40E+G91T+K98E, A40E+G91T+R108K, A40E+G91T+R118F, A40E+G91T+E210K, A40E+G91T+T244E, A40E+G91T+D254S, A40E+K98E+R108K, A40E+K98E+R118F, A40E+K98E+E210K, A40E+K98E+T244E, A40E+K98E+D254S, A40E+R108K+R118F, A40E+R108K+E210K, A40E+R108K+T244E, A40E+R108K+D254S, A40E+R118F+E210K, A40E+R118F+T244E, A40E+R118F+D254S, A40E+E210K+T244E, A40E+E210K+D254S, A40E+T244E+D254S, E56R+D57N+G91T, E56R+D57N+K98E, E56R+D57N+R108K, E56R+D57N+R118F, E56R+D57N+E210K, E56R+D57N+T244E, E56R+D57N+D254S, E56R+G91T+K98E, E56R+G91T+R108K, E56R+G91T+R118F, E56R+G91T+E210K, E56R+G91T+T244E, E56R+G91T+D254S, E56R+K98E+R108K, E56R+K98E+R118F, E56R+K98E+E210K, E56R+K98E+T244E, E56R+K98E+D254S, E56R+R108K+R118F, E56R+R108K+E210K, E56R+R108K+T244E, E56R+R108K+D254S, E56R+R118F+E210K, E56R+R118F+T244E, E56R+R118F+D254S, E56R+E210K+T244E, E56R+E210K+D254S, E56R+T244E+D254S, D57N+G91T+K98E, D57N+G91T+R108K, D57N+G91T+R118F, D57N+G91T+E210K, D57N+G91T+T244E, D57N+G91T+D254S, D57N+K98E+R108K, D57N+K98E+R118F, D57N+K98E+E210K, D57N+K98E+T244E, D57N+K98E+D254S, D57N+R108K+R118F, D57N+R108K+E210K, D57N+R108K+T244E, D57N+R108K+D254S, D57N+R118F+E210K, D57N+R118F+T244E, D57N+R118F+D254S, D57N+E210K+T244E, D57N+E210K+D254S, D57N+T244E+D254S, G91T+K98E+R108K, G91T+K98E+R118F, G91T+K98E+E210K, G91T+K98E+T244E, G91T+K98E+D254S, G91T+R108K+R118F, G91T+R108K+E210K, G91T+R108K+T244E, G91T+R108K+D254S, G91T+R118F+E210K, G91T+R118F+T244E, G91T+R118F+D254S, G91T+E210K+T244E, G91T+E210K+D254S, G91T+T244E+D254S, K98E+R108K+R118F, K98E+R108K+E210K, K98E+R108K+T244E, K98E+R108K+D254S, K98E+R118F+E210K, K98E+R118F+T244E, K98E+R118F+D254S, K98E+E210K+T244E, K98E+E210K+D254S, K98E+T244E+D254S, R108K+R118F+E210K, R108K+R118F+T244E, R108K+R118F+D254S, R108K+E210K+T244E, R108K+E210K+D254S, R108K+T244E+D254S, R118F+E210K+T244E, R118F+E210K+D254S, R118F+T244E+D254S, E210K+T244E+D254S, A40E+E56R+D57N+G91T, A40E+E56R+D57N+K98E, A40E+E56R+D57N+R108K, A40E+E56R+D57N+R118F, A40E+E56R+D57N+E210K, A40E+E56R+D57N+T244E, A40E+E56R+D57N+D254S, A40E+E56R+G91T+K98E, A40E+E56R+G91T+R108K, A40E+E56R+G91T+R118F, A40E+E56R+G91T+E210K, A40E+E56R+G91T+T244E, A40E+E56R+G91T+D254S, A40E+E56R+K98E+R108K, A40E+E56R+K98E+R118F, A40E+E56R+K98E+E210K, A40E+E56R+K98E+T244E, A40E+E56R+K98E+D254S, A40E+E56R+R108K+R118F, A40E+E56R+R108K+E210K, A40E+E56R+R108K+T244E, A40E+E56R+R108K+D254S, A40E+E56R+R118F+E210K, A40E+E56R+R118F+T244E, A40E+E56R+R118F+D254S, A40E+E56R+E210K+T244E, A40E+E56R+E210K+D254S, A40E+E56R+T244E+D254S, A40E+D57N+G91T+K98E, A40E+D57N+G91T+R108K, A40E+D57N+G91T+R118F, A40E+D57N+G91T+E210K, A40E+D57N+G91T+T244E, A40E+D57N+G91T+D254S, A40E+D57N+K98E+R108K, A40E+D57N+K98E+R118F, A40E+D57N+K98E+E210K, A40E+D57N+K98E+T244E, A40E+D57N+K98E+D254S, A40E+D57N+R108K+R118F, A40E+D57N+R108K+E210K, A40E+D57N+R108K+T244E, A40E+D57N+R108K+D254S, A40E+D57N+R118F+E210K, A40E+D57N+R118F+T244E, A40E+D57N+R118F+D254S, A40E+D57N+E210K+T244E, A40E+D57N+E210K+D254S, A40E+D57N+T244E+D254S, A40E+G91T+K98E+R108K, A40E+G91T+K98E+R118F, A40E+G91T+K98E+E210K, A40E+G91T+K98E+T244E, A40E+G91T+K98E+D254S, A40E+G91T+R108K+R118F, A40E+G91T+R108K+E210K, A40E+G91T+R108K+T244E, A40E+G91T+R108K+D254S, A40E+G91T+R118F+E210K, A40E+G91T+R118F+T244E, A40E+G91T+R118F+D254S, A40E+G91T+E210K+T244E, A40E+G91T+E210K+D254S, A40E+G91T+T244E+D254S, A40E+K98E+R108K+R118F, A40E+K98E+R108K+E210K, A40E+K98E+R108K+T244E, A40E+K98E+R108K+D254S, A40E+K98E+R118F+E210K, A40E+K98E+R118F+T244E, A40E+K98E+R118F+D254S, A40E+K98E+E210K+T244E, A40E+K98E+E210K+D254S, A40E+K98E+T244E+D254S, A40E+R108K+R118F+E210K, A40E+R108K+R118F+T244E, A40E+R108K+R118F+D254S, A40E+R108K+E210K+T244E, A40E+R108K+E210K+D254S, A40E+R108K+T244E+D254S, A40E+R118F+E210K+T244E, A40E+R118F+E210K+D254S, A40E+R118F+T244E+D254S, A40E+E210K+T244E+D254S, E56R+D57N+G91T+K98E, E56R+D57N+G91T+R108K, E56R+D57N+G91T+R118F, E56R+D57N+G91T+E210K, E56R+D57N+G91T+T244E, E56R+D57N+G91T+D254S, E56R+D57N+K98E+R108K, E56R+D57N+K98E+R118F, E56R+D57N+K98E+E210K, E56R+D57N+K98E+T244E, E56R+D57N+K98E+D254S, E56R+D57N+R108K+R118F, E56R+D57N+R108K+E210K, E56R+D57N+R108K+T244E, E56R+D57N+R108K+D254S, E56R+D57N+R118F+E210K, E56R+D57N+R118F+T244E, E56R+D57N+R118F+D254S, E56R+D57N+E210K+T244E, E56R+D57N+E210K+D254S, E56R+D57N+T244E+D254S, E56R+G91T+K98E+R108K, E56R+G91T+K98E+R118F, E56R+G91T+K98E+E210K, E56R+G91T+K98E+T244E, E56R+G91T+K98E+D254S, E56R+G91T+R108K+R118F, E56R+G91T+R108K+E210K, E56R+G91T+R108K+T244E, E56R+G91T+R108K+D254S, E56R+G91T+R118F+E210K, E56R+G91T+R118F+T244E, E56R+G91T+R118F+D254S, E56R+G91T+E210K+T244E, E56R+G91T+E210K+D254S, E56R+G91T+T244E+D254S, E56R+K98E+R108K+R118F, E56R+K98E+R108K+E210K, E56R+K98E+R108K+T244E, E56R+K98E+R108K+D254S, E56R+K98E+R118F+E210K, E56R+K98E+R118F+T244E, E56R+K98E+R118F+D254S, E56R+K98E+E210K+T244E, E56R+K98E+E210K+D254S, E56R+K98E+T244E+D254S, E56R+R108K+R118F+E210K, E56R+R108K+R118F+T244E, E56R+R108K+R118F+D254S, E56R+R108K+E210K+T244E, E56R+R108K+E210K+D254S, E56R+R108K+T244E+D254S, E56R+R118F+E210K+T244E, E56R+R118F+E210K+D254S, E56R+R118F+T244E+D254S, E56R+E210K+T244E+D254S, D57N+G91T+K98E+R108K, D57N+G91T+K98E+R118F, D57N+G91T+K98E+E210K, D57N+G91T+K98E+T244E, D57N+G91T+K98E+D254S, D57N+G91T+R108K+R118F, D57N+G91T+R108K+E210K, D57N+G91T+R108K+T244E, D57N+G91T+R108K+D254S, D57N+G91T+R118F+E210K, D57N+G91T+R118F+T244E, D57N+G91T+R118F+D254S, D57N+G91T+E210K+T244E, D57N+G91T+E210K+D254S, D57N+G91T+T244E+D254S, D57N+K98E+R108K+R118F, D57N+K98E+R108K+E210K, D57N+K98E+R108K+T244E, D57N+K98E+R108K+D254S, D57N+K98E+R118F+E210K, D57N+K98E+R118F+T244E, D57N+K98E+R118F+D254S, D57N+K98E+E210K+T244E, D57N+K98E+E210K+D254S, D57N+K98E+T244E+D254S, D57N+R108K+R118F+E210K, D57N+R108K+R118F+T244E, D57N+R108K+R118F+D254S, D57N+R108K+E210K+T244E, D57N+R108K+E210K+D254S, D57N+R108K+T244E+D254S, D57N+R118F+E210K+T244E, D57N+R118F+E210K+D254S, D57N+R118F+T244E+D254S, D57N+E210K+T244E+D254S, G91T+K98E+R108K+R118F, G91T+K98E+R108K+E210K, G91T+K98E+R108K+T244E, G91T+K98E+R108K+D254S, G91T+K98E+R118F+E210K, G91T+K98E+R118F+T244E, G91T+K98E+R118F+D254S, G91T+K98E+E210K+T244E, G91T+K98E+E210K+D254S, G91T+K98E+T244E+D254S, G91T+R108K+R118F+E210K, G91T+R108K+R118F+T244E, G91T+R108K+R118F+D254S, G91T+R108K+E210K+T244E, G91T+R108K+E210K+D254S, G91T+R108K+T244E+D254S, G91T+R118F+E210K+T244E, G91T+R118F+E210K+D254S, G91T+R118F+T244E+D254S, G91T+E210K+T244E+D254S, K98E+R108K+R118F+E210K, K98E+R108K+R118F+T244E, K98E+R108K+R118F+D254S, K98E+R108K+E210K+T244E, K98E+R108K+E210K+D254S, K98E+R108K+T244E+D254S, K98E+R118F+E210K+T244E, K98E+R118F+E210K+D254S, K98E+R118F+T244E+D254S, K98E+E210K+T244E+D254S, R108K+R118F+E210K+T244E, R108K+R118F+E210K+D254S, R108K+R118F+T244E+D254S, R108K+E210K+T244E+D254S, R118F+E210K+T244E+D254S, A40E+E56R+D57N+G91T+K98E, A40E+E56R+D57N+G91T+R108K, A40E+E56R+D57N+G91T+R118F, A40E+E56R+D57N+G91T+E210K, A40E+E56R+D57N+G91T+T244E, A40E+E56R+D57N+G91T+D254S, A40E+E56R+D57N+K98E+R108K, A40E+E56R+D57N+K98E+R118F, A40E+E56R+D57N+K98E+E210K, A40E+E56R+D57N+K98E+T244E, A40E+E56R+D57N+K98E+D254S, A40E+E56R+D57N+R108K+R118F, A40E+E56R+D57N+R108K+E210K, A40E+E56R+D57N+R108K+T244E, A40E+E56R+D57N+R108K+D254S, A40E+E56R+D57N+R118F+E210K, A40E+E56R+D57N+R118F+T244E, A40E+E56R+D57N+R118F+D254S, A40E+E56R+D57N+E210K+T244E, A40E+E56R+D57N+E210K+D254S, A40E+E56R+D57N+T244E+D254S, A40E+E56R+G91T+K98E+R108K, A40E+E56R+G91T+K98E+R118F, A40E+E56R+G91T+K98E+E210K, A40E+E56R+G91T+K98E+T244E, A40E+E56R+G91T+K98E+D254S, A40E+E56R+G91T+R108K+R118F, A40E+E56R+G91T+R108K+E210K, A40E+E56R+G91T+R108K+T244E, A40E+E56R+G91T+R108K+D254S, A40E+E56R+G91T+R118F+E210K, A40E+E56R+G91T+R118F+T244E, A40E+E56R+G91T+R118F+D254S, A40E+E56R+G91T+E210K+T244E, A40E+E56R+G91T+E210K+D254S, A40E+E56R+G91T+T244E+D254S, A40E+E56R+K98E+R108K+R118F, A40E+E56R+K98E+R108K+E210K, A40E+E56R+K98E+R108K+T244E, A40E+E56R+K98E+R108K+D254S, A40E+E56R+K98E+R118F+E210K, A40E+E56R+K98E+R118F+T244E, A40E+E56R+K98E+R118F+D254S, A40E+E56R+K98E+E210K+T244E, A40E+E56R+K98E+E210K+D254S, A40E+E56R+K98E+T244E+D254S, A40E+E56R+R108K+R118F+E210K, A40E+E56R+R108K+R118F+T244E, A40E+E56R+R108K+R118F+D254S, A40E+E56R+R108K+E210K+T244E, A40E+E56R+R108K+E210K+D254S, A40E+E56R+R108K+T244E+D254S, A40E+E56R+R118F+E210K+T244E, A40E+E56R+R118F+E210K+D254S, A40E+E56R+R118F+T244E+D254S, A40E+E56R+E210K+T244E+D254S, A40E+D57N+G91T+K98E+R108K, A40E+D57N+G91T+K98E+R118F, A40E+D57N+G91T+K98E+E210K, A40E+D57N+G91T+K98E+T244E, A40E+D57N+G91T+K98E+D254S, A40E+D57N+G91T+R108K+R118F, A40E+D57N+G91T+R108K+E210K, A40E+D57N+G91T+R108K+T244E, A40E+D57N+G91T+R108K+D254S, A40E+D57N+G91T+R118F+E210K, A40E+D57N+G91T+R118F+T244E, A40E+D57N+G91T+R118F+D254S, A40E+D57N+G91T+E210K+T244E, A40E+D57N+G91T+E210K+D254S, A40E+D57N+G91T+T244E+D254S, A40E+D57N+K98E+R108K+R118F, A40E+D57N+K98E+R108K+E210K, A40E+D57N+K98E+R108K+T244E, A40E+D57N+K98E+R108K+D254S, A40E+D57N+K98E+R118F+E210K, A40E+D57N+K98E+R118F+T244E, A40E+D57N+K98E+R118F+D254S, A40E+D57N+K98E+E210K+T244E, A40E+D57N+K98E+E210K+D254S, A40E+D57N+K98E+T244E+D254S, A40E+D57N+R108K+R118F+E210K, A40E+D57N+R108K+R118F+T244E, A40E+D57N+R108K+R118F+D254S, A40E+D57N+R108K+E210K+T244E, A40E+D57N+R108K+E210K+D254S, A40E+D57N+R108K+T244E+D254S, A40E+D57N+R118F+E210K+T244E, A40E+D57N+R118F+E210K+D254S, A40E+D57N+R118F+T244E+D254S, A40E+D57N+E210K+T244E+D254S, A40E+G91T+K98E+R108K+R118F, A40E+G91T+K98E+R108K+E210K, A40E+G91T+K98E+R108K+T244E, A40E+G91T+K98E+R108K+D254S, A40E+G91T+K98E+R118F+E210K, A40E+G91T+K98E+R118F+T244E, A40E+G91T+K98E+R118F+D254S, A40E+G91T+K98E+E210K+T244E, A40E+G91T+K98E+E210K+D254S, A40E+G91T+K98E+T244E+D254S, A40E+G91T+R108K+R118F+E210K, A40E+G91T+R108K+R118F+T244E, A40E+G91T+R108K+R118F+D254S, A40E+G91T+R108K+E210K+T244E, A40E+G91T+R108K+E210K+D254S, A40E+G91T+R108K+T244E+D254S, A40E+G91T+R118F+E210K+T244E, A40E+G91T+R118F+E210K+D254S, A40E+G91T+R118F+T244E+D254S, A40E+G91T+E210K+T244E+D254S, A40E+K98E+R108K+R118F+E210K, A40E+K98E+R108K+R118F+T244E, A40E+K98E+R108K+R118F+D254S, A40E+K98E+R108K+E210K+T244E, A40E+K98E+R108K+E210K+D254S, A40E+K98E+R108K+T244E+D254S, A40E+K98E+R118F+E210K+T244E, A40E+K98E+R118F+E210K+D254S, A40E+K98E+R118F+T244E+D254S, A40E+K98E+E210K+T244E+D254S, A40E+R108K+R118F+E210K+T244E, A40E+R108K+R118F+E210K+D254S, A40E+R108K+R118F+T244E+D254S, A40E+R108K+E210K+T244E+D254S, A40E+R118F+E210K+T244E+D254S, E56R+D57N+G91T+K98E+R108K, E56R+D57N+G91T+K98E+R118F, E56R+D57N+G91T+K98E+E210K, E56R+D57N+G91T+K98E+T244E, E56R+D57N+G91T+K98E+D254S, E56R+D57N+G91T+R108K+R118F, E56R+D57N+G91T+R108K+E210K, E56R+D57N+G91T+R108K+T244E, E56R+D57N+G91T+R108K+D254S, E56R+D57N+G91T+R118F+E210K, E56R+D57N+G91T+R118F+T244E, E56R+D57N+G91T+R118F+D254S, E56R+D57N+G91T+E210K+T244E, E56R+D57N+G91T+E210K+D254S, E56R+D57N+G91T+T244E+D254S, E56R+D57N+K98E+R108K+R118F, E56R+D57N+K98E+R108K+E210K, E56R+D57N+K98E+R108K+T244E, E56R+D57N+K98E+R108K+D254S, E56R+D57N+K98E+R118F+E210K, E56R+D57N+K98E+R118F+T244E, E56R+D57N+K98E+R118F+D254S, E56R+D57N+K98E+E210K+T244E, E56R+D57N+K98E+E210K+D254S, E56R+D57N+K98E+T244E+D254S, E56R+D57N+R108K+R118F+E210K, E56R+D57N+R108K+R118F+T244E, E56R+D57N+R108K+R118F+D254S, E56R+D57N+R108K+E210K+T244E, E56R+D57N+R108K+E210K+D254S, E56R+D57N+R108K+T244E+D254S, E56R+D57N+R118F+E210K+T244E, E56R+D57N+R118F+E210K+D254S, E56R+D57N+R118F+T244E+D254S, E56R+D57N+E210K+T244E+D254S, E56R+G91T+K98E+R108K+R118F, E56R+G91T+K98E+R108K+E210K, E56R+G91T+K98E+R108K+T244E, E56R+G91T+K98E+R108K+D254S, E56R+G91T+K98E+R118F+E210K, E56R+G91T+K98E+R118F+T244E, E56R+G91T+K98E+R118F+D254S, E56R+G91T+K98E+E210K+T244E, E56R+G91T+K98E+E210K+D254S, E56R+G91T+K98E+T244E+D254S, E56R+G91T+R108K+R118F+E210K, E56R+G91T+R108K+R118F+T244E, E56R+G91T+R108K+R118F+D254S, E56R+G91T+R108K+E210K+T244E, E56R+G91T+R108K+E210K+D254S, E56R+G91T+R108K+T244E+D254S, E56R+G91T+R118F+E210K+T244E, E56R+G91T+R118F+E210K+D254S, E56R+G91T+R118F+T244E+D254S, E56R+G91T+E210K+T244E+D254S, E56R+K98E+R108K+R118F+E210K, E56R+K98E+R108K+R118F+T244E, E56R+K98E+R108K+R118F+D254S, E56R+K98E+R108K+E210K+T244E, E56R+K98E+R108K+E210K+D254S, E56R+K98E+R108K+T244E+D254S, E56R+K98E+R118F+E210K+T244E, E56R+K98E+R118F+E210K+D254S, E56R+K98E+R118F+T244E+D254S, E56R+K98E+E210K+T244E+D254S, E56R+R108K+R118F+E210K+T244E, E56R+R108K+R118F+E210K+D254S, E56R+R108K+R118F+T244E+D254S, E56R+R108K+E210K+T244E+D254S, E56R+R118F+E210K+T244E+D254S, D57N+G91T+K98E+R108K+R118F, D57N+G91T+K98E+R108K+E210K, D57N+G91T+K98E+R108K+T244E, D57N+G91T+K98E+R108K+D254S, D57N+G91T+K98E+R118F+E210K, D57N+G91T+K98E+R118F+T244E, D57N+G91T+K98E+R118F+D254S, D57N+G91T+K98E+E210K+T244E, D57N+G91T+K98E+E210K+D254S, D57N+G91T+K98E+T244E+D254S, D57N+G91T+R108K+R118F+E210K, D57N+G91T+R108K+R118F+T244E, D57N+G91T+R108K+R118F+D254S, D57N+G91T+R108K+E210K+T244E, D57N+G91T+R108K+E210K+D254S, D57N+G91T+R108K+T244E+D254S, D57N+G91T+R118F+E210K+T244E, D57N+G91T+R118F+E210K+D254S, D57N+G91T+R118F+T244E+D254S, D57N+G91T+E210K+T244E+D254S, D57N+K98E+R108K+R118F+E210K, D57N+K98E+R108K+R118F+T244E, D57N+K98E+R108K+R118F+D254S, D57N+K98E+R108K+E210K+T244E, D57N+K98E+R108K+E210K+D254S, D57N+K98E+R108K+T244E+D254S, D57N+K98E+R118F+E210K+T244E, D57N+K98E+R118F+E210K+D254S, D57N+K98E+R118F+T244E+D254S, D57N+K98E+E210K+T244E+D254S, D57N+R108K+R118F+E210K+T244E, D57N+R108K+R118F+E210K+D254S, D57N+R108K+R118F+T244E+D254S, D57N+R108K+E210K+T244E+D254S, D57N+R118F+E210K+T244E+D254S, G91T+K98E+R108K+R118F+E210K, G91T+K98E+R108K+R118F+T244E, G91T+K98E+R108K+R118F+D254S, G91T+K98E+R108K+E210K+T244E, G91T+K98E+R108K+E210K+D254S, G91T+K98E+R108K+T244E+D254S, G91T+K98E+R118F+E210K+T244E, G91T+K98E+R118F+E210K+D254S, G91T+K98E+R118F+T244E+D254S, G91T+K98E+E210K+T244E+D254S, G91T+R108K+R118F+E210K+T244E, G91T+R108K+R118F+E210K+D254S, G91T+R108K+R118F+T244E+D254S, G91T+R108K+E210K+T244E+D254S, G91T+R118F+E210K+T244E+D254S, K98E+R108K+R118F+E210K+T244E, K98E+R108K+R118F+E210K+D254S, K98E+R108K+R118F+T244E+D254S, K98E+R108K+E210K+T244E+D254S, K98E+R118F+E210K+T244E+D254S, R108K+R118F+E210K+T244E+D254S, A40E+E56R+D57N+G91T+K98E+R108K, A40E+E56R+D57N+G91T+K98E+R118F, A40E+E56R+D57N+G91T+K98E+E210K, A40E+E56R+D57N+G91T+K98E+T244E, A40E+E56R+D57N+G91T+K98E+D254S, A40E+E56R+D57N+G91T+R108K+R118F, A40E+E56R+D57N+G91T+R108K+E210K, A40E+E56R+D57N+G91T+R108K+T244E, A40E+E56R+D57N+G91T+R108K+D254S, A40E+E56R+D57N+G91T+R118F+E210K, A40E+E56R+D57N+G91T+R118F+T244E, A40E+E56R+D57N+G91T+R118F+D254S, A40E+E56R+D57N+G91T+E210K+T244E, A40E+E56R+D57N+G91T+E210K+D254S, A40E+E56R+D57N+G91T+T244E+D254S, A40E+E56R+D57N+K98E+R108K+R118F, A40E+E56R+D57N+K98E+R108K+E210K, A40E+E56R+D57N+K98E+R108K+T244E, A40E+E56R+D57N+K98E+R108K+D254S, A40E+E56R+D57N+K98E+R118F+E210K, A40E+E56R+D57N+K98E+R118F+T244E, A40E+E56R+D57N+K98E+R118F+D254S, A40E+E56R+D57N+K98E+E210K+T244E, A40E+E56R+D57N+K98E+E210K+D254S, A40E+E56R+D57N+K98E+T244E+D254S, A40E+E56R+D57N+R108K+R118F+E210K, A40E+E56R+D57N+R108K+R118F+T244E, A40E+E56R+D57N+R108K+R118F+D254S, A40E+E56R+D57N+R108K+E210K+T244E, A40E+E56R+D57N+R108K+E210K+D254S, A40E+E56R+D57N+R108K+T244E+D254S, A40E+E56R+D57N+R118F+E210K+T244E, A40E+E56R+D57N+R118F+E210K+D254S, A40E+E56R+D57N+R118F+T244E+D254S, A40E+E56R+D57N+E210K+T244E+D254S, A40E+E56R+G91T+K98E+R108K+R118F, A40E+E56R+G91T+K98E+R108K+E210K, A40E+E56R+G91T+K98E+R108K+T244E, A40E+E56R+G91T+K98E+R108K+D254S, A40E+E56R+G91T+K98E+R118F+E210K, A40E+E56R+G91T+K98E+R118F+T244E, A40E+E56R+G91T+K98E+R118F+D254S, A40E+E56R+G91T+K98E+E210K+T244E, A40E+E56R+G91T+K98E+E210K+D254S, A40E+E56R+G91T+K98E+T244E+D254S, A40E+E56R+G91T+R108K+R118F+E210K, A40E+E56R+G91T+R108K+R118F+T244E, A40E+E56R+G91T+R108K+R118F+D254S, A40E+E56R+G91T+R108K+E210K+T244E, A40E+E56R+G91T+R108K+E210K+D254S, A40E+E56R+G91T+R108K+T244E+D254S, A40E+E56R+G91T+R118F+E210K+T244E, A40E+E56R+G91T+R118F+E210K+D254S, A40E+E56R+G91T+R118F+T244E+D254S, A40E+E56R+G91T+E210K+T244E+D254S, A40E+E56R+K98E+R108K+R118F+E210K, A40E+E56R+K98E+R108K+R118F+T244E, A40E+E56R+K98E+R108K+R118F+D254S, A40E+E56R+K98E+R108K+E210K+T244E, A40E+E56R+K98E+R108K+E210K+D254S, A40E+E56R+K98E+R108K+T244E+D254S, A40E+E56R+K98E+R118F+E210K+T244E, A40E+E56R+K98E+R118F+E210K+D254S, A40E+E56R+K98E+R118F+T244E+D254S, A40E+E56R+K98E+E210K+T244E+D254S, A40E+E56R+R108K+R118F+E210K+T244E, A40E+E56R+R108K+R118F+E210K+D254S, A40E+E56R+R108K+R118F+T244E+D254S, A40E+E56R+R108K+E210K+T244E+D254S, A40E+E56R+R118F+E210K+T244E+D254S, A40E+D57N+G91T+K98E+R108K+R118F, A40E+D57N+G91T+K98E+R108K+E210K, A40E+D57N+G91T+K98E+R108K+T244E, A40E+D57N+G91T+K98E+R108K+D254S, A40E+D57N+G91T+K98E+R118F+E210K, A40E+D57N+G91T+K98E+R118F+T244E, A40E+D57N+G91T+K98E+R118F+D254S, A40E+D57N+G91T+K98E+E210K+T244E, A40E+D57N+G91T+K98E+E210K+D254S, A40E+D57N+G91T+K98E+T244E+D254S, A40E+D57N+G91T+R108K+R118F+E210K, A40E+D57N+G91T+R108K+R118F+T244E, A40E+D57N+G91T+R108K+R118F+D254S, A40E+D57N+G91T+R108K+E210K+T244E, A40E+D57N+G91T+R108K+E210K+D254S, A40E+D57N+G91T+R108K+T244E+D254S, A40E+D57N+G91T+R118F+E210K+T244E, A40E+D57N+G91T+R118F+E210K+D254S, A40E+D57N+G91T+R118F+T244E+D254S, A40E+D57N+G91T+E210K+T244E+D254S, A40E+D57N+K98E+R108K+R118F+E210K, A40E+D57N+K98E+R108K+R118F+T244E, A40E+D57N+K98E+R108K+R118F+D254S, A40E+D57N+K98E+R108K+E210K+T244E, A40E+D57N+K98E+R108K+E210K+D254S, A40E+D57N+K98E+R108K+T244E+D254S, A40E+D57N+K98E+R118F+E210K+T244E, A40E+D57N+K98E+R118F+E210K+D254S, A40E+D57N+K98E+R118F+T244E+D254S, A40E+D57N+K98E+E210K+T244E+D254S, A40E+D57N+R108K+R118F+E210K+T244E, A40E+D57N+R108K+R118F+E210K+D254S, A40E+D57N+R108K+R118F+T244E+D254S, A40E+D57N+R108K+E210K+T244E+D254S, A40E+D57N+R118F+E210K+T244E+D254S, A40E+G91T+K98E+R108K+R118F+E210K, A40E+G91T+K98E+R108K+R118F+T244E, A40E+G91T+K98E+R108K+R118F+D254S, A40E+G91T+K98E+R108K+E210K+T244E, A40E+G91T+K98E+R108K+E210K+D254S, A40E+G91T+K98E+R108K+T244E+D254S, A40E+G91T+K98E+R118F+E210K+T244E, A40E+G91T+K98E+R118F+E210K+D254S, A40E+G91T+K98E+R118F+T244E+D254S, A40E+G91T+K98E+E210K+T244E+D254S, A40E+G91T+R108K+R118F+E210K+T244E, A40E+G91T+R108K+R118F+E210K+D254S, A40E+G91T+R108K+R118F+T244E+D254S, A40E+G91T+R108K+E210K+T244E+D254S, A40E+G91T+R118F+E210K+T244E+D254S, A40E+K98E+R108K+R118F+E210K+T244E, A40E+K98E+R108K+R118F+E210K+D254S, A40E+K98E+R108K+R118F+T244E+D254S, A40E+K98E+R108K+E210K+T244E+D254S, A40E+K98E+R118F+E210K+T244E+D254S, A40E+R108K+R118F+E210K+T244E+D254S, E56R+D57N+G91T+K98E+R108K+R118F, E56R+D57N+G91T+K98E+R108K+E210K, E56R+D57N+G91T+K98E+R108K+T244E, E56R+D57N+G91T+K98E+R108K+D254S, E56R+D57N+G91T+K98E+R118F+E210K, E56R+D57N+G91T+K98E+R118F+T244E, E56R+D57N+G91T+K98E+R118F+D254S, E56R+D57N+G91T+K98E+E210K+T244E, E56R+D57N+G91T+K98E+E210K+D254S, E56R+D57N+G91T+K98E+T244E+D254S, E56R+D57N+G91T+R108K+R118F+E210K, E56R+D57N+G91T+R108K+R118F+T244E, E56R+D57N+G91T+R108K+R118F+D254S, E56R+D57N+G91T+R108K+E210K+T244E, E56R+D57N+G91T+R108K+E210K+D254S, E56R+D57N+G91T+R108K+T244E+D254S, E56R+D57N+G91T+R118F+E210K+T244E, E56R+D57N+G91T+R118F+E210K+D254S, E56R+D57N+G91T+R118F+T244E+D254S, E56R+D57N+G91T+E210K+T244E+D254S, E56R+D57N+K98E+R108K+R118F+E210K, E56R+D57N+K98E+R108K+R118F+T244E, E56R+D57N+K98E+R108K+R118F+D254S, E56R+D57N+K98E+R108K+E210K+T244E, E56R+D57N+K98E+R108K+E210K+D254S, E56R+D57N+K98E+R108K+T244E+D254S, E56R+D57N+K98E+R118F+E210K+T244E, E56R+D57N+K98E+R118F+E210K+D254S, E56R+D57N+K98E+R118F+T244E+D254S, E56R+D57N+K98E+E210K+T244E+D254S, E56R+D57N+R108K+R118F+E210K+T244E, E56R+D57N+R108K+R118F+E210K+D254S, E56R+D57N+R108K+R118F+T244E+D254S, E56R+D57N+R108K+E210K+T244E+D254S, E56R+D57N+R118F+E210K+T244E+D254S, E56R+G91T+K98E+R108K+R118F+E210K, E56R+G91T+K98E+R108K+R118F+T244E, E56R+G91T+K98E+R108K+R118F+D254S, E56R+G91T+K98E+R108K+E210K+T244E, E56R+G91T+K98E+R108K+E210K+D254S, E56R+G91T+K98E+R108K+T244E+D254S, E56R+G91T+K98E+R118F+E210K+T244E, E56R+G91T+K98E+R118F+E210K+D254S, E56R+G91T+K98E+R118F+T244E+D254S, E56R+G91T+K98E+E210K+T244E+D254S, E56R+G91T+R108K+R118F+E210K+T244E, E56R+G91T+R108K+R118F+E210K+D254S, E56R+G91T+R108K+R118F+T244E+D254S, E56R+G91T+R108K+E210K+T244E+D254S, E56R+G91T+R118F+E210K+T244E+D254S, E56R+K98E+R108K+R118F+E210K+T244E, E56R+K98E+R108K+R118F+E210K+D254S, E56R+K98E+R108K+R118F+T244E+D254S, E56R+K98E+R108K+E210K+T244E+D254S, E56R+K98E+R118F+E210K+T244E+D254S, E56R+R108K+R118F+E210K+T244E+D254S, D57N+G91T+K98E+R108K+R118F+E210K, D57N+G91T+K98E+R108K+R118F+T244E, D57N+G91T+K98E+R108K+R118F+D254S, D57N+G91T+K98E+R108K+E210K+T244E, D57N+G91T+K98E+R108K+E210K+D254S, D57N+G91T+K98E+R108K+T244E+D254S, D57N+G91T+K98E+R118F+E210K+T244E, D57N+G91T+K98E+R118F+E210K+D254S, D57N+G91T+K98E+R118F+T244E+D254S, D57N+G91T+K98E+E210K+T244E+D254S, D57N+G91T+R108K+R118F+E210K+T244E, D57N+G91T+R108K+R118F+E210K+D254S, D57N+G91T+R108K+R118F+T244E+D254S, D57N+G91T+R108K+E210K+T244E+D254S, D57N+G91T+R118F+E210K+T244E+D254S, D57N+K98E+R108K+R118F+E210K+T244E, D57N+K98E+R108K+R118F+E210K+D254S, D57N+K98E+R108K+R118F+T244E+D254S, D57N+K98E+R108K+E210K+T244E+D254S, D57N+K98E+R118F+E210K+T244E+D254S, D57N+R108K+R118F+E210K+T244E+D254S, G91T+K98E+R108K+R118F+E210K+T244E, G91T+K98E+R108K+R118F+E210K+D254S, G91T+K98E+R108K+R118F+T244E+D254S, G91T+K98E+R108K+E210K+T244E+D254S, G91T+K98E+R118F+E210K+T244E+D254S, G91T+R108K+R118F+E210K+T244E+D254S, K98E+R108K+R118F+E210K+T244E+D254S,
A40E+E56R+D57N+G91T+K98E+R108K+R118F,
A40E+E56R+D57N+G91T+K98E+R108K+E210K,
A40E+E56R+D57N+G91T+K98E+R108K+T244E,
A40E+E56R+D57N+G91T+K98E+R108K+D254S,
A40E+E56R+D57N+G91T+K98E+R118F+E210K,
A40E+E56R+D57N+G91T+K98E+R118F+T244E,
A40E+E56R+D57N+G91T+K98E+R118F+D254S,
A40E+E56R+D57N+G91T+K98E+E210K+T244E,
A40E+E56R+D57N+G91T+K98E+E210K+D254S,
A40E+E56R+D57N+G91T+K98E+T244E+D254S,
A40E+E56R+D57N+G91T+R108K+R118F+E210K,
A40E+E56R+D57N+G91T+R108K+R118F+T244E,
A40E+E56R+D57N+G91T+R108K+R118F+D254S,
A40E+E56R+D57N+G91T+R108K+E210K+T244E,
A40E+E56R+D57N+G91T+R108K+E210K+D254S,
A40E+E56R+D57N+G91T+R108K+T244E+D254S,
A40E+E56R+D57N+G91T+R118F+E210K+T244E,
A40E+E56R+D57N+G91T+R118F+E210K+D254S,
A40E+E56R+D57N+G91T+R118F+T244E+D254S,
A40E+E56R+D57N+G91T+E210K+T244E+D254S,
A40E+E56R+D57N+K98E+R108K+R118F+E210K,
A40E+E56R+D57N+K98E+R108K+R118F+T244E,
A40E+E56R+D57N+K98E+R108K+R118F+D254S,
A40E+E56R+D57N+K98E+R108K+E210K+T244E,
A40E+E56R+D57N+K98E+R108K+E210K+D254S,
A40E+E56R+D57N+K98E+R108K+T244E+D254S,
A40E+E56R+D57N+K98E+R118F+E210K+T244E,
A40E+E56R+D57N+K98E+R118F+E210K+D254S,
A40E+E56R+D57N+K98E+R118F+T244E+D254S,
A40E+E56R+D57N+K98E+E210K+T244E+D254S,
A40E+E56R+D57N+R108K+R118F+E210K+T244E,
A40E+E56R+D57N+R108K+R118F+E210K+D254S,
A40E+E56R+D57N+R108K+R118F+T244E+D254S,
A40E+E56R+D57N+R108K+E210K+T244E+D254S,
A40E+E56R+D57N+R118F+E210K+T244E+D254S,
A40E+E56R+G91T+K98E+R108K+R118F+E210K,
A40E+E56R+G91T+K98E+R108K+R118F+T244E,
A40E+E56R+G91T+K98E+R108K+R118F+D254S,
A40E+E56R+G91T+K98E+R108K+E210K+T244E,
A40E+E56R+G91T+K98E+R108K+E210K+D254S,
A40E+E56R+G91T+K98E+R108K+T244E+D254S,
A40E+E56R+G91T+K98E+R118F+E210K+T244E,
A40E+E56R+G91T+K98E+R118F+E210K+D254S,
A40E+E56R+G91T+K98E+R118F+T244E+D254S,
A40E+E56R+G91T+K98E+E210K+T244E+D254S,
A40E+E56R+G91T+R108K+R118F+E210K+T244E,
A40E+E56R+G91T+R108K+R118F+E210K+D254S,
A40E+E56R+G91T+R108K+R118F+T244E+D254S,
A40E+E56R+G91T+R108K+E210K+T244E+D254S,
A40E+E56R+G91T+R118F+E210K+T244E+D254S,
A40E+E56R+K98E+R108K+R118F+E210K+T244E,
A40E+E56R+K98E+R108K+R118F+E210K+D254S,
A40E+E56R+K98E+R108K+R118F+T244E+D254S,
A40E+E56R+K98E+R108K+E210K+T244E+D254S,
A40E+E56R+K98E+R118F+E210K+T244E+D254S,
A40E+E56R+R108K+R118F+E210K+T244E+D254S,
A40E+D57N+G91T+K98E+R108K+R118F+E210K,
A40E+D57N+G91T+K98E+R108K+R118F+T244E,
A40E+D57N+G91T+K98E+R108K+R118F+D254S,
A40E+D57N+G91T+K98E+R108K+E210K+T244E,
A40E+D57N+G91T+K98E+R108K+E210K+D254S,
A40E+D57N+G91T+K98E+R108K+T244E+D254S,
A40E+D57N+G91T+K98E+R118F+E210K+T244E,
A40E+D57N+G91T+K98E+R118F+E210K+D254S,
A40E+D57N+G91T+K98E+R118F+T244E+D254S,
A40E+D57N+G91T+K98E+E210K+T244E+D254S,
A40E+D57N+G91T+R108K+R118F+E210K+T244E,
A40E+D57N+G91T+R108K+R118F+E210K+D254S,
A40E+D57N+G91T+R108K+R118F+T244E+D254S,
A40E+D57N+G91T+R108K+E210K+T244E+D254S,
A40E+D57N+G91T+R118F+E210K+T244E+D254S,
A40E+D57N+K98E+R108K+R118F+E210K+T244E,
A40E+D57N+K98E+R108K+R118F+E210K+D254S,
A40E+D57N+K98E+R108K+R118F+T244E+D254S,
A40E+D57N+K98E+R108K+E210K+T244E+D254S,
A40E+D57N+K98E+R118F+E210K+T244E+D254S,
A40E+D57N+R108K+R118F+E210K+T244E+D254S,
A40E+G91T+K98E+R108K+R118F+E210K+T244E,
A40E+G91T+K98E+R108K+R118F+E210K+D254S,
A40E+G91T+K98E+R108K+R118F+T244E+D254S,
A40E+G91T+K98E+R108K+E210K+T244E+D254S,
A40E+G91T+K98E+R118F+E210K+T244E+D254S,
A40E+G91T+R108K+R118F+E210K+T244E+D254S,
A40E+K98E+R108K+R118F+E210K+T244E+D254S,
E56R+D57N+G91T+K98E+R108K+R118F+E210K,
E56R+D57N+G91T+K98E+R108K+R118F+T244E,
E56R+D57N+G91T+K98E+R108K+R118F+D254S,
E56R+D57N+G91T+K98E+R108K+E210K+T244E,
E56R+D57N+G91T+K98E+R108K+E210K+D254S,
E56R+D57N+G91T+K98E+R108K+T244E+D254S,
E56R+D57N+G91T+K98E+R118F+E210K+T244E,
E56R+D57N+G91T+K98E+R118F+E210K+D254S,
E56R+D57N+G91T+K98E+R118F+T244E+D254S,
E56R+D57N+G91T+K98E+E210K+T244E+D254S,
E56R+D57N+G91T+R108K+R118F+E210K+T244E,
E56R+D57N+G91T+R108K+R118F+E210K+D254S,
E56R+D57N+G91T+R108K+R118F+T244E+D254S,
E56R+D57N+G91T+R108K+E210K+T244E+D254S,
E56R+D57N+G91T+R118F+E210K+T244E+D254S,
E56R+D57N+K98E+R108K+R118F+E210K+T244E,
E56R+D57N+K98E+R108K+R118F+E210K+D254S,
E56R+D57N+K98E+R108K+R118F+T244E+D254S,
E56R+D57N+K98E+R108K+E210K+T244E+D254S,
E56R+D57N+K98E+R118F+E210K+T244E+D254S,
E56R+D57N+R108K+R118F+E210K+T244E+D254S,
E56R+G91T+K98E+R108K+R118F+E210K+T244E,
E56R+G91T+K98E+R108K+R118F+E210K+D254S,
E56R+G91T+K98E+R108K+R118F+T244E+D254S,
E56R+G91T+K98E+R108K+E210K+T244E+D254S,
E56R+G91T+K98E+R118F+E210K+T244E+D254S,
E56R+G91T+R108K+R118F+E210K+T244E+D254S,
E56R+K98E+R108K+R118F+E210K+T244E+D254S,
D57N+G91T+K98E+R108K+R118F+E210K+T244E,
D57N+G91T+K98E+R108K+R118F+E210K+D254S,
D57N+G91T+K98E+R108K+R118F+T244E+D254S,
D57N+G91T+K98E+R108K+E210K+T244E+D254S,
D57N+G91T+K98E+R118F+E210K+T244E+D254S,
D57N+G91T+R108K+R118F+E210K+T244E+D254S,
D57N+K98E+R108K+R118F+E210K+T244E+D254S,
G91T+K98E+R108K+R118F+E210K+T244E+D254S,
A40E+E56R+D57N+G91T+K98E+R108K+R118F+E210K,
A40E+E56R+D57N+G91T+K98E+R108K+R118F+T244E,
A40E+E56R+D57N+G91T+K98E+R108K+R118F+D254S,
A40E+E56R+D57N+G91T+K98E+R108K+E210K+T244E,
A40E+E56R+D57N+G91T+K98E+R108K+E210K+D254S,
A40E+E56R+D57N+G91T+K98E+R108K+T244E+D254S,
A40E+E56R+D57N+G91T+K98E+R118F+E210K+T244E,
A40E+E56R+D57N+G91T+K98E+R118F+E210K+D254S,
A40E+E56R+D57N+G91T+K98E+R118F+T244E+D254S,
A40E+E56R+D57N+G91T+K98E+E210K+T244E+D254S,
A40E+E56R+D57N+G91T+R108K+R118F+E210K+T244E,
A40E+E56R+D57N+G91T+R108K+R118F+E210K+D254S,
A40E+E56R+D57N+G91T+R108K+R118F+T244E+D254S,
A40E+E56R+D57N+G91T+R108K+E210K+T244E+D254S,
A40E+E56R+D57N+G91T+R118F+E210K+T244E+D254S,
A40E+E56R+D57N+K98E+R108K+R118F+E210K+T244E,
A40E+E56R+D57N+K98E+R108K+R118F+E210K+D254S,
A40E+E56R+D57N+K98E+R108K+R118F+T244E+D254S,
A40E+E56R+D57N+K98E+R108K+E210K+T244E+D254S,
A40E+E56R+D57N+K98E+R118F+E210K+T244E+D254S,
A40E+E56R+D57N+R108K+R118F+E210K+T244E+D254S,
A40E+E56R+G91T+K98E+R108K+R118F+E210K+T244E,
A40E+E56R+G91T+K98E+R108K+R118F+E210K+D254S,
A40E+E56R+G91T+K98E+R108K+R118F+T244E+D254S,
A40E+E56R+G91T+K98E+R108K+E210K+T244E+D254S,
A40E+E56R+G91T+K98E+R118F+E210K+T244E+D254S,
A40E+E56R+G91T+R108K+R118F+E210K+T244E+D254S,
A40E+E56R+K98E+R108K+R118F+E210K+T244E+D254S,
A40E+D57N+G91T+K98E+R108K+R118F+E210K+T244E,
A40E+D57N+G91T+K98E+R108K+R118F+E210K+D254S,
A40E+D57N+G91T+K98E+R108K+R118F+T244E+D254S,
A40E+D57N+G91T+K98E+R108K+E210K+T244E+D254S,
A40E+D57N+G91T+K98E+R118F+E210K+T244E+D254S,
A40E+D57N+G91T+R108K+R118F+E210K+T244E+D254S,
A40E+D57N+K98E+R108K+R118F+E210K+T244E+D254S,
A40E+G91T+K98E+R108K+R118F+E210K+T244E+D254S,
E56R+D57N+G91T+K98E+R108K+R118F+E210K+T244E,
E56R+D57N+G91T+K98E+R108K+R118F+E210K+D254S,
E56R+D57N+G91T+K98E+R108K+R118F+T244E+D254S,
E56R+D57N+G91T+K98E+R108K+E210K+T244E+D254S,
E56R+D57N+G91T+K98E+R118F+E210K+T244E+D254S,
E56R+D57N+G91T+R108K+R118F+E210K+T244E+D254S,
E56R+D57N+K98E+R108K+R118F+E210K+T244E+D254S,
E56R+G91T+K98E+R108K+R118F+E210K+T244E+D254S,
D57N+G91T+K98E+R108K+R118F+E210K+T244E+D254S,
A40E+E56R+D57N+G91T+K98E+R108K+R118F+E210K+T244E,
A40E+E56R+D57N+G91T+K98E+R108K+R118F+E210K+D254S,
A40E+E56R+D57N+G91T+K98E+R108K+R118F+T244E+D254S,
A40E+E56R+D57N+G91T+K98E+R108K+E210K+T244E+D254S,
A40E+E56R+D57N+G91T+K98E+R118F+E210K+T244E+D254S,
A40E+E56R+D57N+G91T+R108K+R118F+E210K+T244E+D254S,
A40E+E56R+D57N+K98E+R108K+R118F+E210K+T244E+D254S,
A40E+E56R+G91T+K98E+R108K+R118F+E210K+T244E+D254S,
A40E+D57N+G91T+K98E+R108K+R118F+E210K+T244E+D254S,
E56R+D57N+G91T+K98E+R108K+R118F+E210K+T244E+D254S,
A40E+E56R+D57N+G91T+K98E+R108K+R118F+E210K+T244E+D254S.

In an embodiment, a variant of the invention has a substitution corresponding to I252H of SEQ ID NO: 8 and further comprises one of the following substitutions or set of substitutions corresponding to: A40E, E56R, D57N, G91T, K98E, R108K, R118F, E210K, T244E, A40E+E56R, A40E+D57N, A40E+G91T, A40E+K98E, A40E+R108K, A40E+R118F, A40E+E210K, A40E+T244E, A40E+D254S, E56R+D57N, E56R+G91T, E56R+K98E, E56R+R108K, E56R+R118F, E56R+E210K, E56R+T244E, E56R+D254S, D57N+G91T, D57N+K98E, D57N+R108K, D57N+R118F, D57N+E210K, D57N+T244E, D57N+D254S, G91T+K98E, G91T+R108K, G91T+R118F, G91T+E210K, G91T+T244E, G91T+D254S, K98E+R108K, K98E+R118F, K98E+E210K, K98E+T244E, K98E+D254S, R108K+R118F, R108K+E210K, R108K+T244E, R108K+D254S, R118F+E210K, R118F+T244E, R118F+D254S, E210K+T244E, E210K+D254S, T244E+D254S, A40E+E56R+D57N, A40E+E56R+G91T, A40E+E56R+K98E, A40E+E56R+R108K, A40E+E56R+R118F, A40E+E56R+E210K, A40E+E56R+T244E, A40E+E56R+D254S, A40E+D57N+G91T, A40E+D57N+K98E, A40E+D57N+R108K, A40E+D57N+R118F, A40E+D57N+E210K, A40E+D57N+T244E, A40E+D57N+D254S, A40E+G91T+K98E, A40E+G91T+R108K, A40E+G91T+R118F, A40E+G91T+E210K, A40E+G91T+T244E, A40E+G91T+D254S, A40E+K98E+R108K, A40E+K98E+R118F, A40E+K98E+E210K, A40E+K98E+T244E, A40E+K98E+D254S, A40E+R108K+R118F, A40E+R108K+E210K, A40E+R108K+T244E, A40E+R108K+D254S, A40E+R118F+E210K, A40E+R118F+T244E, A40E+R118F+D254S, A40E+E210K+T244E, A40E+E210K+D254S, A40E+T244E+D254S, E56R+D57N+G91T, E56R+D57N+K98E, E56R+D57N+R108K, E56R+D57N+R118F, E56R+D57N+E210K, E56R+D57N+T244E, E56R+D57N+D254S, E56R+G91T+K98E, E56R+G91T+R108K, E56R+G91T+R118F, E56R+G91T+E210K, E56R+G91T+T244E, E56R+G91T+D254S, E56R+K98E+R108K, E56R+K98E+R118F, E56R+K98E+E210K, E56R+K98E+T244E, E56R+K98E+D254S, E56R+R108K+R118F, E56R+R108K+E210K, E56R+R108K+T244E, E56R+R108K+D254S, E56R+R118F+E210K, E56R+R118F+T244E, E56R+R118F+D254S, E56R+E210K+T244E, E56R+E210K+D254S, E56R+T244E+D254S, D57N+G91T+K98E, D57N+G91T+R108K, D57N+G91T+R118F, D57N+G91T+E210K, D57N+G91T+T244E, D57N+G91T+D254S, D57N+K98E+R108K, D57N+K98E+R118F, D57N+K98E+E210K, D57N+K98E+T244E, D57N+K98E+D254S, D57N+R108K+R118F, D57N+R108K+E210K, D57N+R108K+T244E, D57N+R108K+D254S, D57N+R118F+E210K, D57N+R118F+T244E, D57N+R118F+D254S, D57N+E210K+T244E, D57N+E210K+D254S, D57N+T244E+D254S, G91T+K98E+R108K, G91T+K98E+R118F, G91T+K98E+E210K, G91T+K98E+T244E, G91T+K98E+D254S, G91T+R108K+R118F, G91T+R108K+E210K, G91T+R108K+T244E, G91T+R108K+D254S, G91T+R118F+E210K, G91T+R118F+T244E, G91T+R118F+D254S, G91T+E210K+T244E, G91T+E210K+D254S, G91T+T244E+D254S, K98E+R108K+R118F, K98E+R108K+E210K, K98E+R108K+T244E, K98E+R108K+D254S, K98E+R118F+E210K, K98E+R118F+T244E, K98E+R118F+D254S, K98E+E210K+T244E, K98E+E210K+D254S, K98E+T244E+D254S, R108K+R118F+E210K, R108K+R118F+T244E, R108K+R118F+D254S, R108K+E210K+T244E, R108K+E210K+D254S, R108K+T244E+D254S, R118F+E210K+T244E, R118F+E210K+D254S, R118F+T244E+D254S, E210K+T244E+D254S, A40E+E56R+D57N+G91T, A40E+E56R+D57N+K98E, A40E+E56R+D57N+R108K, A40E+E56R+D57N+R118F, A40E+E56R+D57N+E210K, A40E+E56R+D57N+T244E, A40E+E56R+D57N+D254S, A40E+E56R+G91T+K98E, A40E+E56R+G91T+R108K, A40E+E56R+G91T+R118F, A40E+E56R+G91T+E210K, A40E+E56R+G91T+T244E, A40E+E56R+G91T+D254S, A40E+E56R+K98E+R108K, A40E+E56R+K98E+R118F, A40E+E56R+K98E+E210K, A40E+E56R+K98E+T244E, A40E+E56R+K98E+D254S, A40E+E56R+R108K+R118F, A40E+E56R+R108K+E210K, A40E+E56R+R108K+T244E, A40E+E56R+R108K+D254S, A40E+E56R+R118F+E210K, A40E+E56R+R118F+T244E, A40E+E56R+R118F+D254S, A40E+E56R+E210K+T244E, A40E+E56R+E210K+D254S, A40E+E56R+T244E+D254S, A40E+D57N+G91T+K98E, A40E+D57N+G91T+R108K, A40E+D57N+G91T+R118F, A40E+D57N+G91T+E210K, A40E+D57N+G91T+T244E, A40E+D57N+G91T+D254S, A40E+D57N+K98E+R108K, A40E+D57N+K98E+R118F, A40E+D57N+K98E+E210K, A40E+D57N+K98E+T244E, A40E+D57N+K98E+D254S, A40E+D57N+R108K+R118F, A40E+D57N+R108K+E210K, A40E+D57N+R108K+T244E, A40E+D57N+R108K+D254S, A40E+D57N+R118F+E210K, A40E+D57N+R118F+T244E, A40E+D57N+R118F+D254S, A40E+D57N+E210K+T244E, A40E+D57N+E210K+D254S, A40E+D57N+T244E+D254S, A40E+G91T+K98E+R108K, A40E+G91T+K98E+R118F, A40E+G91T+K98E+E210K, A40E+G91T+K98E+T244E, A40E+G91T+K98E+D254S, A40E+G91T+R108K+R118F, A40E+G91T+R108K+E210K, A40E+G91T+R108K+T244E, A40E+G91T+R108K+D254S, A40E+G91T+R118F+E210K, A40E+G91T+R118F+T244E, A40E+G91T+R118F+D254S, A40E+G91T+E210K+T244E, A40E+G91T+E210K+D254S, A40E+G91T+T244E+D254S, A40E+K98E+R108K+R118F, A40E+K98E+R108K+E210K, A40E+K98E+R108K+T244E, A40E+K98E+R108K+D254S, A40E+K98E+R118F+E210K, A40E+K98E+R118F+T244E, A40E+K98E+R118F+D254S, A40E+K98E+E210K+T244E, A40E+K98E+E210K+D254S, A40E+K98E+T244E+D254S, A40E+R108K+R118F+E210K, A40E+R108K+R118F+T244E, A40E+R108K+R118F+D254S, A40E+R108K+E210K+T244E, A40E+R108K+E210K+D254S, A40E+R108K+T244E+D254S, A40E+R118F+E210K+T244E, A40E+R118F+E210K+D254S, A40E+R118F+T244E+D254S, A40E+E210K+T244E+D254S, E56R+D57N+G91T+K98E, E56R+D57N+G91T+R108K, E56R+D57N+G91T+R118F, E56R+D57N+G91T+E210K, E56R+D57N+G91T+T244E, E56R+D57N+G91T+D254S, E56R+D57N+K98E+R108K, E56R+D57N+K98E+R118F, E56R+D57N+K98E+E210K, E56R+D57N+K98E+T244E, E56R+D57N+K98E+D254S, E56R+D57N+R108K+R118F, E56R+D57N+R108K+E210K, E56R+D57N+R108K+T244E, E56R+D57N+R108K+D254S, E56R+D57N+R118F+E210K, E56R+D57N+R118F+T244E, E56R+D57N+R118F+D254S, E56R+D57N+E210K+T244E, E56R+D57N+E210K+D254S, E56R+D57N+T244E+D254S, E56R+G91T+K98E+R108K, E56R+G91T+K98E+R118F, E56R+G91T+K98E+E210K, E56R+G91T+K98E+T244E, E56R+G91T+K98E+D254S, E56R+G91T+R108K+R118F, E56R+G91T+R108K+E210K, E56R+G91T+R108K+T244E, E56R+G91T+R108K+D254S, E56R+G91T+R118F+E210K, E56R+G91T+R118F+T244E, E56R+G91T+R118F+D254S, E56R+G91T+E210K+T244E, E56R+G91T+E210K+D254S, E56R+G91T+T244E+D254S, E56R+K98E+R108K+R118F, E56R+K98E+R108K+E210K, E56R+K98E+R108K+T244E, E56R+K98E+R108K+D254S, E56R+K98E+R118F+E210K, E56R+K98E+R118F+T244E, E56R+K98E+R118F+D254S, E56R+K98E+E210K+T244E, E56R+K98E+E210K+D254S, E56R+K98E+T244E+D254S, E56R+R108K+R118F+E210K, E56R+R108K+R118F+T244E, E56R+R108K+R118F+D254S, E56R+R108K+E210K+T244E, E56R+R108K+E210K+D254S, E56R+R108K+T244E+D254S, E56R+R118F+E210K+T244E, E56R+R118F+E210K+D254S, E56R+R118F+T244E+D254S, E56R+E210K+T244E+D254S, D57N+G91T+K98E+R108K, D57N+G91T+K98E+R118F, D57N+G91T+K98E+E210K, D57N+G91T+K98E+T244E, D57N+G91T+K98E+D254S, D57N+G91T+R108K+R118F, D57N+G91T+R108K+E210K, D57N+G91T+R108K+T244E, D57N+G91T+R108K+D254S, D57N+G91T+R118F+E210K, D57N+G91T+R118F+T244E, D57N+G91T+R118F+D254S, D57N+G91T+E210K+T244E, D57N+G91T+E210K+D254S, D57N+G91T+T244E+D254S, D57N+K98E+R108K+R118F, D57N+K98E+R108K+E210K, D57N+K98E+R108K+T244E, D57N+K98E+R108K+D254S, D57N+K98E+R118F+E210K, D57N+K98E+R118F+T244E, D57N+K98E+R118F+D254S, D57N+K98E+E210K+T244E, D57N+K98E+E210K+D254S, D57N+K98E+T244E+D254S, D57N+R108K+R118F+E210K, D57N+R108K+R118F+T244E, D57N+R108K+R118F+D254S, D57N+R108K+E210K+T244E, D57N+R108K+E210K+D254S, D57N+R108K+T244E+D254S, D57N+R118F+E210K+T244E, D57N+R118F+E210K+D254S, D57N+R118F+T244E+D254S, D57N+E210K+T244E+D254S, G91T+K98E+R108K+R118F, G91T+K98E+R108K+E210K, G91T+K98E+R108K+T244E, G91T+K98E+R108K+D254S, G91T+K98E+R118F+E210K, G91T+K98E+R118F+T244E, G91T+K98E+R118F+D254S, G91T+K98E+E210K+T244E, G91T+K98E+E210K+D254S, G91T+K98E+T244E+D254S, G91T+R108K+R118F+E210K, G91T+R108K+R118F+T244E, G91T+R108K+R118F+D254S, G91T+R108K+E210K+T244E, G91T+R108K+E210K+D254S, G91T+R108K+T244E+D254S, G91T+R118F+E210K+T244E, G91T+R118F+E210K+D254S, G91T+R118F+T244E+D254S, G91T+E210K+T244E+D254S, K98E+R108K+R118F+E210K, K98E+R108K+R118F+T244E, K98E+R108K+R118F+D254S, K98E+R108K+E210K+T244E, K98E+R108K+E210K+D254S, K98E+R108K+T244E+D254S, K98E+R118F+E210K+T244E, K98E+R118F+E210K+D254S, K98E+R118F+T244E+D254S, K98E+E210K+T244E+D254S, R108K+R118F+E210K+T244E, R108K+R118F+E210K+D254S, R108K+R118F+T244E+D254S, R108K+E210K+T244E+D254S, R118F+E210K+T244E+D254S, A40E+E56R+D57N+G91T+K98E, A40E+E56R+D57N+G91T+R108K, A40E+E56R+D57N+G91T+R118F, A40E+E56R+D57N+G91T+E210K, A40E+E56R+D57N+G91T+T244E, A40E+E56R+D57N+G91T+D254S, A40E+E56R+D57N+K98E+R108K, A40E+E56R+D57N+K98E+R118F, A40E+E56R+D57N+K98E+E210K, A40E+E56R+D57N+K98E+T244E, A40E+E56R+D57N+K98E+D254S, A40E+E56R+D57N+R108K+R118F, A40E+E56R+D57N+R108K+E210K, A40E+E56R+D57N+R108K+T244E, A40E+E56R+D57N+R108K+D254S, A40E+E56R+D57N+R118F+E210K, A40E+E56R+D57N+R118F+T244E, A40E+E56R+D57N+R118F+D254S, A40E+E56R+D57N+E210K+T244E, A40E+E56R+D57N+E210K+D254S, A40E+E56R+D57N+T244E+D254S, A40E+E56R+G91T+K98E+R108K, A40E+E56R+G91T+K98E+R118F, A40E+E56R+G91T+K98E+E210K, A40E+E56R+G91T+K98E+T244E, A40E+E56R+G91T+K98E+D254S, A40E+E56R+G91T+R108K+R118F, A40E+E56R+G91T+R108K+E210K, A40E+E56R+G91T+R108K+T244E, A40E+E56R+G91T+R108K+D254S, A40E+E56R+G91T+R118F+E210K, A40E+E56R+G91T+R118F+T244E, A40E+E56R+G91T+R118F+D254S, A40E+E56R+G91T+E210K+T244E, A40E+E56R+G91T+E210K+D254S, A40E+E56R+G91T+T244E+D254S, A40E+E56R+K98E+R108K+R118F, A40E+E56R+K98E+R108K+E210K, A40E+E56R+K98E+R108K+T244E, A40E+E56R+K98E+R108K+D254S, A40E+E56R+K98E+R118F+E210K, A40E+E56R+K98E+R118F+T244E, A40E+E56R+K98E+R118F+D254S, A40E+E56R+K98E+E210K+T244E, A40E+E56R+K98E+E210K+D254S, A40E+E56R+K98E+T244E+D254S, A40E+E56R+R108K+R118F+E210K, A40E+E56R+R108K+R118F+T244E, A40E+E56R+R108K+R118F+D254S, A40E+E56R+R108K+E210K+T244E, A40E+E56R+R108K+E210K+D254S, A40E+E56R+R108K+T244E+D254S, A40E+E56R+R118F+E210K+T244E, A40E+E56R+R118F+E210K+D254S, A40E+E56R+R118F+T244E+D254S, A40E+E56R+E210K+T244E+D254S, A40E+D57N+G91T+K98E+R108K, A40E+D57N+G91T+K98E+R118F, A40E+D57N+G91T+K98E+E210K, A40E+D57N+G91T+K98E+T244E, A40E+D57N+G91T+K98E+D254S, A40E+D57N+G91T+R108K+R118F, A40E+D57N+G91T+R108K+E210K, A40E+D57N+G91T+R108K+T244E, A40E+D57N+G91T+R108K+D254S, A40E+D57N+G91T+R118F+E210K, A40E+D57N+G91T+R118F+T244E, A40E+D57N+G91T+R118F+D254S, A40E+D57N+G91T+E210K+T244E, A40E+D57N+G91T+E210K+D254S, A40E+D57N+G91T+T244E+D254S, A40E+D57N+K98E+R108K+R118F, A40E+D57N+K98E+R108K+E210K, A40E+D57N+K98E+R108K+T244E, A40E+D57N+K98E+R108K+D254S, A40E+D57N+K98E+R118F+E210K, A40E+D57N+K98E+R118F+T244E, A40E+D57N+K98E+R118F+D254S, A40E+D57N+K98E+E210K+T244E, A40E+D57N+K98E+E210K+D254S, A40E+D57N+K98E+T244E+D254S, A40E+D57N+R108K+R118F+E210K, A40E+D57N+R108K+R118F+T244E, A40E+D57N+R108K+R118F+D254S, A40E+D57N+R108K+E210K+T244E, A40E+D57N+R108K+E210K+D254S, A40E+D57N+R108K+T244E+D254S, A40E+D57N+R118F+E210K+T244E, A40E+D57N+R118F+E210K+D254S, A40E+D57N+R118F+T244E+D254S, A40E+D57N+E210K+T244E+D254S, A40E+G91T+K98E+R108K+R118F, A40E+G91T+K98E+R108K+E210K, A40E+G91T+K98E+R108K+T244E, A40E+G91T+K98E+R108K+D254S, A40E+G91T+K98E+R118F+E210K, A40E+G91T+K98E+R118F+T244E, A40E+G91T+K98E+R118F+D254S, A40E+G91T+K98E+E210K+T244E, A40E+G91T+K98E+E210K+D254S, A40E+G91T+K98E+T244E+D254S, A40E+G91T+R108K+R118F+E210K, A40E+G91T+R108K+R118F+T244E, A40E+G91T+R108K+R118F+D254S, A40E+G91T+R108K+E210K+T244E, A40E+G91T+R108K+E210K+D254S, A40E+G91T+R108K+T244E+D254S, A40E+G91T+R118F+E210K+T244E, A40E+G91T+R118F+E210K+D254S, A40E+G91T+R118F+T244E+D254S, A40E+G91T+E210K+T244E+D254S, A40E+K98E+R108K+R118F+E210K, A40E+K98E+R108K+R118F+T244E, A40E+K98E+R108K+R118F+D254S, A40E+K98E+R108K+E210K+T244E, A40E+K98E+R108K+E210K+D254S, A40E+K98E+R108K+T244E+D254S, A40E+K98E+R118F+E210K+T244E, A40E+K98E+R118F+E210K+D254S, A40E+K98E+R118F+T244E+D254S, A40E+K98E+E210K+T244E+D254S, A40E+R108K+R118F+E210K+T244E, A40E+R108K+R118F+E210K+D254S, A40E+R108K+R118F+T244E+D254S, A40E+R108K+E210K+T244E+D254S, A40E+R118F+E210K+T244E+D254S, E56R+D57N+G91T+K98E+R108K, E56R+D57N+G91T+K98E+R118F, E56R+D57N+G91T+K98E+E210K, E56R+D57N+G91T+K98E+T244E, E56R+D57N+G91T+K98E+D254S, E56R+D57N+G91T+R108K+R118F, E56R+D57N+G91T+R108K+E210K, E56R+D57N+G91T+R108K+T244E, E56R+D57N+G91T+R108K+D254S, E56R+D57N+G91T+R118F+E210K, E56R+D57N+G91T+R118F+T244E, E56R+D57N+G91T+R118F+D254S, E56R+D57N+G91T+E210K+T244E, E56R+D57N+G91T+E210K+D254S, E56R+D57N+G91T+T244E+D254S, E56R+D57N+K98E+R108K+R118F, E56R+D57N+K98E+R108K+E210K, E56R+D57N+K98E+R108K+T244E, E56R+D57N+K98E+R108K+D254S, E56R+D57N+K98E+R118F+E210K, E56R+D57N+K98E+R118F+T244E, E56R+D57N+K98E+R118F+D254S, E56R+D57N+K98E+E210K+T244E, E56R+D57N+K98E+E210K+D254S, E56R+D57N+K98E+T244E+D254S, E56R+D57N+R108K+R118F+E210K, E56R+D57N+R108K+R118F+T244E, E56R+D57N+R108K+R118F+D254S, E56R+D57N+R108K+E210K+T244E, E56R+D57N+R108K+E210K+D254S, E56R+D57N+R108K+T244E+D254S, E56R+D57N+R118F+E210K+T244E, E56R+D57N+R118F+E210K+D254S, E56R+D57N+R118F+T244E+D254S, E56R+D57N+E210K+T244E+D254S, E56R+G91T+K98E+R108K+R118F, E56R+G91T+K98E+R108K+E210K, E56R+G91T+K98E+R108K+T244E, E56R+G91T+K98E+R108K+D254S, E56R+G91T+K98E+R118F+E210K, E56R+G91T+K98E+R118F+T244E, E56R+G91T+K98E+R118F+D254S, E56R+G91T+K98E+E210K+T244E, E56R+G91T+K98E+E210K+D254S, E56R+G91T+K98E+T244E+D254S, E56R+G91T+R108K+R118F+E210K, E56R+G91T+R108K+R118F+T244E, E56R+G91T+R108K+R118F+D254S, E56R+G91T+R108K+E210K+T244E, E56R+G91T+R108K+E210K+D254S, E56R+G91T+R108K+T244E+D254S, E56R+G91T+R118F+E210K+T244E, E56R+G91T+R118F+E210K+D254S, E56R+G91T+R118F+T244E+D254S, E56R+G91T+E210K+T244E+D254S, E56R+K98E+R108K+R118F+E210K, E56R+K98E+R108K+R118F+T244E, E56R+K98E+R108K+R118F+D254S, E56R+K98E+R108K+E210K+T244E, E56R+K98E+R108K+E210K+D254S, E56R+K98E+R108K+T244E+D254S, E56R+K98E+R118F+E210K+T244E, E56R+K98E+R118F+E210K+D254S, E56R+K98E+R118F+T244E+D254S, E56R+K98E+E210K+T244E+D254S, E56R+R108K+R118F+E210K+T244E, E56R+R108K+R118F+E210K+D254S, E56R+R108K+R118F+T244E+D254S, E56R+R108K+E210K+T244E+D254S, E56R+R118F+E210K+T244E+D254S, D57N+G91T+K98E+R108K+R118F, D57N+G91T+K98E+R108K+E210K, D57N+G91T+K98E+R108K+T244E, D57N+G91T+K98E+R108K+D254S, D57N+G91T+K98E+R118F+E210K, D57N+G91T+K98E+R118F+T244E, D57N+G91T+K98E+R118F+D254S, D57N+G91T+K98E+E210K+T244E, D57N+G91T+K98E+E210K+D254S, D57N+G91T+K98E+T244E+D254S, D57N+G91T+R108K+R118F+E210K, D57N+G91T+R108K+R118F+T244E, D57N+G91T+R108K+R118F+D254S, D57N+G91T+R108K+E210K+T244E, D57N+G91T+R108K+E210K+D254S, D57N+G91T+R108K+T244E+D254S, D57N+G91T+R118F+E210K+T244E, D57N+G91T+R118F+E210K+D254S, D57N+G91T+R118F+T244E+D254S, D57N+G91T+E210K+T244E+D254S, D57N+K98E+R108K+R118F+E210K, D57N+K98E+R108K+R118F+T244E, D57N+K98E+R108K+R118F+D254S, D57N+K98E+R108K+E210K+T244E, D57N+K98E+R108K+E210K+D254S, D57N+K98E+R108K+T244E+D254S, D57N+K98E+R118F+E210K+T244E, D57N+K98E+R118F+E210K+D254S, D57N+K98E+R118F+T244E+D254S, D57N+K98E+E210K+T244E+D254S, D57N+R108K+R118F+E210K+T244E, D57N+R108K+R118F+E210K+D254S, D57N+R108K+R118F+T244E+D254S, D57N+R108K+E210K+T244E+D254S, D57N+R118F+E210K+T244E+D254S, G91T+K98E+R108K+R118F+E210K, G91T+K98E+R108K+R118F+T244E, G91T+K98E+R108K+R118F+D254S, G91T+K98E+R108K+E210K+T244E, G91T+K98E+R108K+E210K+D254S, G91T+K98E+R108K+T244E+D254S, G91T+K98E+R118F+E210K+T244E, G91T+K98E+R118F+E210K+D254S, G91T+K98E+R118F+T244E+D254S, G91T+K98E+E210K+T244E+D254S, G91T+R108K+R118F+E210K+T244E, G91T+R108K+R118F+E210K+D254S, G91T+R108K+R118F+T244E+D254S, G91T+R108K+E210K+T244E+D254S, G91T+R118F+E210K+T244E+D254S, K98E+R108K+R118F+E210K+T244E, K98E+R108K+R118F+E210K+D254S, K98E+R108K+R118F+T244E+D254S, K98E+R108K+E210K+T244E+D254S, K98E+R118F+E210K+T244E+D254S, R108K+R118F+E210K+T244E+D254S, A40E+E56R+D57N+G91T+K98E+R108K, A40E+E56R+D57N+G91T+K98E+R118F, A40E+E56R+D57N+G91T+K98E+E210K, A40E+E56R+D57N+G91T+K98E+T244E, A40E+E56R+D57N+G91T+K98E+D254S, A40E+E56R+D57N+G91T+R108K+R118F, A40E+E56R+D57N+G91T+R108K+E210K, A40E+E56R+D57N+G91T+R108K+T244E, A40E+E56R+D57N+G91T+R108K+D254S, A40E+E56R+D57N+G91T+R118F+E210K, A40E+E56R+D57N+G91T+R118F+T244E, A40E+E56R+D57N+G91T+R118F+D254S, A40E+E56R+D57N+G91T+E210K+T244E, A40E+E56R+D57N+G91T+E210K+D254S, A40E+E56R+D57N+G91T+T244E+D254S, A40E+E56R+D57N+K98E+R108K+R118F, A40E+E56R+D57N+K98E+R108K+E210K, A40E+E56R+D57N+K98E+R108K+T244E, A40E+E56R+D57N+K98E+R108K+D254S, A40E+E56R+D57N+K98E+R118F+E210K, A40E+E56R+D57N+K98E+R118F+T244E, A40E+E56R+D57N+K98E+R118F+D254S, A40E+E56R+D57N+K98E+E210K+T244E, A40E+E56R+D57N+K98E+E210K+D254S, A40E+E56R+D57N+K98E+T244E+D254S, A40E+E56R+D57N+R108K+R118F+E210K, A40E+E56R+D57N+R108K+R118F+T244E, A40E+E56R+D57N+R108K+R118F+D254S, A40E+E56R+D57N+R108K+E210K+T244E, A40E+E56R+D57N+R108K+E210K+D254S, A40E+E56R+D57N+R108K+T244E+D254S, A40E+E56R+D57N+R118F+E210K+T244E, A40E+E56R+D57N+R118F+E210K+D254S, A40E+E56R+D57N+R118F+T244E+D254S, A40E+E56R+D57N+E210K+T244E+D254S, A40E+E56R+G91T+K98E+R108K+R118F, A40E+E56R+G91T+K98E+R108K+E210K, A40E+E56R+G91T+K98E+R108K+T244E, A40E+E56R+G91T+K98E+R108K+D254S, A40E+E56R+G91T+K98E+R118F+E210K, A40E+E56R+G91T+K98E+R118F+T244E, A40E+E56R+G91T+K98E+R118F+D254S, A40E+E56R+G91T+K98E+E210K+T244E, A40E+E56R+G91T+K98E+E210K+D254S, A40E+E56R+G91T+K98E+T244E+D254S, A40E+E56R+G91T+R108K+R118F+E210K, A40E+E56R+G91T+R108K+R118F+T244E, A40E+E56R+G91T+R108K+R118F+D254S, A40E+E56R+G91T+R108K+E210K+T244E, A40E+E56R+G91T+R108K+E210K+D254S, A40E+E56R+G91T+R108K+T244E+D254S, A40E+E56R+G91T+R118F+E210K+T244E, A40E+E56R+G91T+R118F+E210K+D254S, A40E+E56R+G91T+R118F+T244E+D254S, A40E+E56R+G91T+E210K+T244E+D254S, A40E+E56R+K98E+R108K+R118F+E210K, A40E+E56R+K98E+R108K+R118F+T244E, A40E+E56R+K98E+R108K+R118F+D254S, A40E+E56R+K98E+R108K+E210K+T244E, A40E+E56R+K98E+R108K+E210K+D254S, A40E+E56R+K98E+R108K+T244E+D254S, A40E+E56R+K98E+R118F+E210K+T244E, A40E+E56R+K98E+R118F+E210K+D254S, A40E+E56R+K98E+R118F+T244E+D254S, A40E+E56R+K98E+E210K+T244E+D254S, A40E+E56R+R108K+R118F+E210K+T244E, A40E+E56R+R108K+R118F+E210K+D254S, A40E+E56R+R108K+R118F+T244E+D254S, A40E+E56R+R108K+E210K+T244E+D254S, A40E+E56R+R118F+E210K+T244E+D254S, A40E+D57N+G91T+K98E+R108K+R118F, A40E+D57N+G91T+K98E+R108K+E210K, A40E+D57N+G91T+K98E+R108K+T244E, A40E+D57N+G91T+K98E+R108K+D254S, A40E+D57N+G91T+K98E+R118F+E210K, A40E+D57N+G91T+K98E+R118F+T244E, A40E+D57N+G91T+K98E+R118F+D254S, A40E+D57N+G91T+K98E+E210K+T244E, A40E+D57N+G91T+K98E+E210K+D254S, A40E+D57N+G91T+K98E+T244E+D254S, A40E+D57N+G91T+R108K+R118F+E210K, A40E+D57N+G91T+R108K+R118F+T244E, A40E+D57N+G91T+R108K+R118F+D254S, A40E+D57N+G91T+R108K+E210K+T244E, A40E+D57N+G91T+R108K+E210K+D254S, A40E+D57N+G91T+R108K+T244E+D254S, A40E+D57N+G91T+R118F+E210K+T244E, A40E+D57N+G91T+R118F+E210K+D254S, A40E+D57N+G91T+R118F+T244E+D254S, A40E+D57N+G91T+E210K+T244E+D254S, A40E+D57N+K98E+R108K+R118F+E210K, A40E+D57N+K98E+R108K+R118F+T244E, A40E+D57N+K98E+R108K+R118F+D254S, A40E+D57N+K98E+R108K+E210K+T244E, A40E+D57N+K98E+R108K+E210K+D254S, A40E+D57N+K98E+R108K+T244E+D254S, A40E+D57N+K98E+R118F+E210K+T244E, A40E+D57N+K98E+R118F+E210K+D254S, A40E+D57N+K98E+R118F+T244E+D254S, A40E+D57N+K98E+E210K+T244E+D254S, A40E+D57N+R108K+R118F+E210K+T244E, A40E+D57N+R108K+R118F+E210K+D254S, A40E+D57N+R108K+R118F+T244E+D254S, A40E+D57N+R108K+E210K+T244E+D254S, A40E+D57N+R118F+E210K+T244E+D254S, A40E+G91T+K98E+R108K+R118F+E210K, A40E+G91T+K98E+R108K+R118F+T244E, A40E+G91T+K98E+R108K+R118F+D254S, A40E+G91T+K98E+R108K+E210K+T244E, A40E+G91T+K98E+R108K+E210K+D254S, A40E+G91T+K98E+R108K+T244E+D254S, A40E+G91T+K98E+R118F+E210K+T244E, A40E+G91T+K98E+R118F+E210K+D254S, A40E+G91T+K98E+R118F+T244E+D254S, A40E+G91T+K98E+E210K+T244E+D254S, A40E+G91T+R108K+R118F+E210K+T244E, A40E+G91T+R108K+R118F+E210K+D254S, A40E+G91T+R108K+R118F+T244E+D254S, A40E+G91T+R108K+E210K+T244E+D254S, A40E+G91T+R118F+E210K+T244E+D254S, A40E+K98E+R108K+R118F+E210K+T244E, A40E+K98E+R108K+R118F+E210K+D254S, A40E+K98E+R108K+R118F+T244E+D254S, A40E+K98E+R108K+E210K+T244E+D254S, A40E+K98E+R118F+E210K+T244E+D254S, A40E+R108K+R118F+E210K+T244E+D254S, E56R+D57N+G91T+K98E+R108K+R118F, E56R+D57N+G91T+K98E+R108K+E210K, E56R+D57N+G91T+K98E+R108K+T244E, E56R+D57N+G91T+K98E+R108K+D254S, E56R+D57N+G91T+K98E+R118F+E210K, E56R+D57N+G91T+K98E+R118F+T244E, E56R+D57N+G91T+K98E+R118F+D254S, E56R+D57N+G91T+K98E+E210K+T244E, E56R+D57N+G91T+K98E+E210K+D254S, E56R+D57N+G91T+K98E+T244E+D254S, E56R+D57N+G91T+R108K+R118F+E210K, E56R+D57N+G91T+R108K+R118F+T244E, E56R+D57N+G91T+R108K+R118F+D254S, E56R+D57N+G91T+R108K+E210K+T244E, E56R+D57N+G91T+R108K+E210K+D254S, E56R+D57N+G91T+R108K+T244E+D254S, E56R+D57N+G91T+R118F+E210K+T244E, E56R+D57N+G91T+R118F+E210K+D254S, E56R+D57N+G91T+R118F+T244E+D254S, E56R+D57N+G91T+E210K+T244E+D254S, E56R+D57N+K98E+R108K+R118F+E210K, E56R+D57N+K98E+R108K+R118F+T244E, E56R+D57N+K98E+R108K+R118F+D254S, E56R+D57N+K98E+R108K+E210K+T244E, E56R+D57N+K98E+R108K+E210K+D254S, E56R+D57N+K98E+R108K+T244E+D254S, E56R+D57N+K98E+R118F+E210K+T244E, E56R+D57N+K98E+R118F+E210K+D254S, E56R+D57N+K98E+R118F+T244E+D254S, E56R+D57N+K98E+E210K+T244E+D254S, E56R+D57N+R108K+R118F+E210K+T244E, E56R+D57N+R108K+R118F+E210K+D254S, E56R+D57N+R108K+R118F+T244E+D254S, E56R+D57N+R108K+E210K+T244E+D254S, E56R+D57N+R118F+E210K+T244E+D254S, E56R+G91T+K98E+R108K+R118F+E210K, E56R+G91T+K98E+R108K+R118F+T244E, E56R+G91T+K98E+R108K+R118F+D254S, E56R+G91T+K98E+R108K+E210K+T244E, E56R+G91T+K98E+R108K+E210K+D254S, E56R+G91T+K98E+R108K+T244E+D254S, E56R+G91T+K98E+R118F+E210K+T244E, E56R+G91T+K98E+R118F+E210K+D254S, E56R+G91T+K98E+R118F+T244E+D254S, E56R+G91T+K98E+E210K+T244E+D254S, E56R+G91T+R108K+R118F+E210K+T244E, E56R+G91T+R108K+R118F+E210K+D254S, E56R+G91T+R108K+R118F+T244E+D254S, E56R+G91T+R108K+E210K+T244E+D254S, E56R+G91T+R118F+E210K+T244E+D254S, E56R+K98E+R108K+R118F+E210K+T244E, E56R+K98E+R108K+R118F+E210K+D254S, E56R+K98E+R108K+R118F+T244E+D254S, E56R+K98E+R108K+E210K+T244E+D254S, E56R+K98E+R118F+E210K+T244E+D254S, E56R+R108K+R118F+E210K+T244E+D254S, D57N+G91T+K98E+R108K+R118F+E210K, D57N+G91T+K98E+R108K+R118F+T244E, D57N+G91T+K98E+R108K+R118F+D254S, D57N+G91T+K98E+R108K+E210K+T244E, D57N+G91T+K98E+R108K+E210K+D254S, D57N+G91T+K98E+R108K+T244E+D254S, D57N+G91T+K98E+R118F+E210K+T244E, D57N+G91T+K98E+R118F+E210K+D254S, D57N+G91T+K98E+R118F+T244E+D254S, D57N+G91T+K98E+E210K+T244E+D254S, D57N+G91T+R108K+R118F+E210K+T244E, D57N+G91T+R108K+R118F+E210K+D254S, D57N+G91T+R108K+R118F+T244E+D254S, D57N+G91T+R108K+E210K+T244E+D254S, D57N+G91T+R118F+E210K+T244E+D254S, D57N+K98E+R108K+R118F+E210K+T244E, D57N+K98E+R108K+R118F+E210K+D254S, D57N+K98E+R108K+R118F+T244E+D254S, D57N+K98E+R108K+E210K+T244E+D254S, D57N+K98E+R118F+E210K+T244E+D254S, D57N+R108K+R118F+E210K+T244E+D254S, G91T+K98E+R108K+R118F+E210K+T244E, G91T+K98E+R108K+R118F+E210K+D254S, G91T+K98E+R108K+R118F+T244E+D254S, G91T+K98E+R108K+E210K+T244E+D254S, G91T+K98E+R118F+E210K+T244E+D254S, G91T+R108K+R118F+E210K+T244E+D254S,
K98E+R108K+R118F+E210K+T244E+D254S,
A40E+E56R+D57N+G91T+K98E+R108K+R118F,
A40E+E56R+D57N+G91T+K98E+R108K+E210K,
A40E+E56R+D57N+G91T+K98E+R108K+T244E,
A40E+E56R+D57N+G91T+K98E+R108K+D254S,
A40E+E56R+D57N+G91T+K98E+R118F+E210K,
A40E+E56R+D57N+G91T+K98E+R118F+T244E,
A40E+E56R+D57N+G91T+K98E+R118F+D254S,
A40E+E56R+D57N+G91T+K98E+E210K+T244E,
A40E+E56R+D57N+G91T+K98E+E210K+D254S,
A40E+E56R+D57N+G91T+K98E+T244E+D254S,
A40E+E56R+D57N+G91T+R108K+R118F+E210K,
A40E+E56R+D57N+G91T+R108K+R118F+T244E,
A40E+E56R+D57N+G91T+R108K+R118F+D254S,
A40E+E56R+D57N+G91T+R108K+E210K+T244E,
A40E+E56R+D57N+G91T+R108K+E210K+D254S,
A40E+E56R+D57N+G91T+R108K+T244E+D254S,
A40E+E56R+D57N+G91T+R118F+E210K+T244E,
A40E+E56R+D57N+G91T+R118F+E210K+D254S,
A40E+E56R+D57N+G91T+R118F+T244E+D254S,
A40E+E56R+D57N+G91T+E210K+T244E+D254S,
A40E+E56R+D57N+K98E+R108K+R118F+E210K,
A40E+E56R+D57N+K98E+R108K+R118F+T244E,
A40E+E56R+D57N+K98E+R108K+R118F+D254S,
A40E+E56R+D57N+K98E+R108K+E210K+T244E,
A40E+E56R+D57N+K98E+R108K+E210K+D254S,
A40E+E56R+D57N+K98E+R108K+T244E+D254S,
A40E+E56R+D57N+K98E+R118F+E210K+T244E,
A40E+E56R+D57N+K98E+R118F+E210K+D254S,
A40E+E56R+D57N+K98E+R118F+T244E+D254S,
A40E+E56R+D57N+K98E+E210K+T244E+D254S,
A40E+E56R+D57N+R108K+R118F+E210K+T244E,
A40E+E56R+D57N+R108K+R118F+E210K+D254S,
A40E+E56R+D57N+R108K+R118F+T244E+D254S,
A40E+E56R+D57N+R108K+E210K+T244E+D254S,
A40E+E56R+D57N+R118F+E210K+T244E+D254S,
A40E+E56R+G91T+K98E+R108K+R118F+E210K,
A40E+E56R+G91T+K98E+R108K+R118F+T244E,
A40E+E56R+G91T+K98E+R108K+R118F+D254S,
A40E+E56R+G91T+K98E+R108K+E210K+T244E,
A40E+E56R+G91T+K98E+R108K+E210K+D254S,
A40E+E56R+G91T+K98E+R108K+T244E+D254S,
A40E+E56R+G91T+K98E+R118F+E210K+T244E,
A40E+E56R+G91T+K98E+R118F+E210K+D254S,
A40E+E56R+G91T+K98E+R118F+T244E+D254S,
A40E+E56R+G91T+K98E+E210K+T244E+D254S,
A40E+E56R+G91T+R108K+R118F+E210K+T244E,
A40E+E56R+G91T+R108K+R118F+E210K+D254S,
A40E+E56R+G91T+R108K+R118F+T244E+D254S,
A40E+E56R+G91T+R108K+E210K+T244E+D254S,
A40E+E56R+G91T+R118F+E210K+T244E+D254S,
A40E+E56R+K98E+R108K+R118F+E210K+T244E,
A40E+E56R+K98E+R108K+R118F+E210K+D254S,
A40E+E56R+K98E+R108K+R118F+T244E+D254S,
A40E+E56R+K98E+R108K+E210K+T244E+D254S,
A40E+E56R+K98E+R118F+E210K+T244E+D254S,
A40E+E56R+R108K+R118F+E210K+T244E+D254S,
A40E+D57N+G91T+K98E+R108K+R118F+E210K,
A40E+D57N+G91T+K98E+R108K+R118F+T244E,
A40E+D57N+G91T+K98E+R108K+R118F+D254S,
A40E+D57N+G91T+K98E+R108K+E210K+T244E,
A40E+D57N+G91T+K98E+R108K+E210K+D254S,
A40E+D57N+G91T+K98E+R108K+T244E+D254S,
A40E+D57N+G91T+K98E+R118F+E210K+T244E,
A40E+D57N+G91T+K98E+R118F+E210K+D254S,
A40E+D57N+G91T+K98E+R118F+T244E+D254S,
A40E+D57N+G91T+K98E+E210K+T244E+D254S,
A40E+D57N+G91T+R108K+R118F+E210K+T244E,
A40E+D57N+G91T+R108K+R118F+E210K+D254S,
A40E+D57N+G91T+R108K+R118F+T244E+D254S,
A40E+D57N+G91T+R108K+E210K+T244E+D254S,
A40E+D57N+G91T+R118F+E210K+T244E+D254S,
A40E+D57N+K98E+R108K+R118F+E210K+T244E,
A40E+D57N+K98E+R108K+R118F+E210K+D254S,
A40E+D57N+K98E+R108K+R118F+T244E+D254S,
A40E+D57N+K98E+R108K+E210K+T244E+D254S,
A40E+D57N+K98E+R118F+E210K+T244E+D254S,
A40E+D57N+R108K+R118F+E210K+T244E+D254S,
A40E+G91T+K98E+R108K+R118F+E210K+T244E,
A40E+G91T+K98E+R108K+R118F+E210K+D254S,
A40E+G91T+K98E+R108K+R118F+T244E+D254S,
A40E+G91T+K98E+R108K+E210K+T244E+D254S,
A40E+G91T+K98E+R118F+E210K+T244E+D254S,
A40E+G91T+R108K+R118F+E210K+T244E+D254S,
A40E+K98E+R108K+R118F+E210K+T244E+D254S,
E56R+D57N+G91T+K98E+R108K+R118F+E210K,
E56R+D57N+G91T+K98E+R108K+R118F+T244E,
E56R+D57N+G91T+K98E+R108K+R118F+D254S,
E56R+D57N+G91T+K98E+R108K+E210K+T244E,
E56R+D57N+G91T+K98E+R108K+E210K+D254S,
E56R+D57N+G91T+K98E+R108K+T244E+D254S,
E56R+D57N+G91T+K98E+R118F+E210K+T244E,
E56R+D57N+G91T+K98E+R118F+E210K+D254S,
E56R+D57N+G91T+K98E+R118F+T244E+D254S,
E56R+D57N+G91T+K98E+E210K+T244E+D254S,
E56R+D57N+G91T+R108K+R118F+E210K+T244E,
E56R+D57N+G91T+R108K+R118F+E210K+D254S,
E56R+D57N+G91T+R108K+R118F+T244E+D254S,
E56R+D57N+G91T+R108K+E210K+T244E+D254S,
E56R+D57N+G91T+R118F+E210K+T244E+D254S,
E56R+D57N+K98E+R108K+R118F+E210K+T244E,
E56R+D57N+K98E+R108K+R118F+E210K+D254S,
E56R+D57N+K98E+R108K+R118F+T244E+D254S,
E56R+D57N+K98E+R108K+E210K+T244E+D254S,
E56R+D57N+K98E+R118F+E210K+T244E+D254S,
E56R+D57N+R108K+R118F+E210K+T244E+D254S,
E56R+G91T+K98E+R108K+R118F+E210K+T244E,
E56R+G91T+K98E+R108K+R118F+E210K+D254S,
E56R+G91T+K98E+R108K+R118F+T244E+D254S,
E56R+G91T+K98E+R108K+E210K+T244E+D254S,
E56R+G91T+K98E+R118F+E210K+T244E+D254S,
E56R+G91T+R108K+R118F+E210K+T244E+D254S,
E56R+K98E+R108K+R118F+E210K+T244E+D254S,
D57N+G91T+K98E+R108K+R118F+E210K+T244E,
D57N+G91T+K98E+R108K+R118F+E210K+D254S,
D57N+G91T+K98E+R108K+R118F+T244E+D254S,
D57N+G91T+K98E+R108K+E210K+T244E+D254S,
D57N+G91T+K98E+R118F+E210K+T244E+D254S,
D57N+G91T+R108K+R118F+E210K+T244E+D254S,
D57N+K98E+R108K+R118F+E210K+T244E+D254S,
G91T+K98E+R108K+R118F+E210K+T244E+D254S,
A40E+E56R+D57N+G91T+K98E+R108K+R118F+E210K,
A40E+E56R+D57N+G91T+K98E+R108K+R118F+T244E,
A40E+E56R+D57N+G91T+K98E+R108K+R118F+D254S,
A40E+E56R+D57N+G91T+K98E+R108K+E210K+T244E,
A40E+E56R+D57N+G91T+K98E+R108K+E210K+D254S,
A40E+E56R+D57N+G91T+K98E+R108K+T244E+D254S,
A40E+E56R+D57N+G91T+K98E+R118F+E210K+T244E,
A40E+E56R+D57N+G91T+K98E+R118F+E210K+D254S,
A40E+E56R+D57N+G91T+K98E+R118F+T244E+D254S,
A40E+E56R+D57N+G91T+K98E+E210K+T244E+D254S,
A40E+E56R+D57N+G91T+R108K+R118F+E210K+T244E,
A40E+E56R+D57N+G91T+R108K+R118F+E210K+D254S,
A40E+E56R+D57N+G91T+R108K+R118F+T244E+D254S,
A40E+E56R+D57N+G91T+R108K+E210K+T244E+D254S,
A40E+E56R+D57N+G91T+R118F+E210K+T244E+D254S,
A40E+E56R+D57N+K98E+R108K+R118F+E210K+T244E,
A40E+E56R+D57N+K98E+R108K+R118F+E210K+D254S,
A40E+E56R+D57N+K98E+R108K+R118F+T244E+D254S,
A40E+E56R+D57N+K98E+R108K+E210K+T244E+D254S,
A40E+E56R+D57N+K98E+R118F+E210K+T244E+D254S,
A40E+E56R+D57N+R108K+R118F+E210K+T244E+D254S,
A40E+E56R+G91T+K98E+R108K+R118F+E210K+T244E,
A40E+E56R+G91T+K98E+R108K+R118F+E210K+D254S,
A40E+E56R+G91T+K98E+R108K+R118F+T244E+D254S,
A40E+E56R+G91T+K98E+R108K+E210K+T244E+D254S,
A40E+E56R+G91T+K98E+R118F+E210K+T244E+D254S,
A40E+E56R+G91T+R108K+R118F+E210K+T244E+D254S,
A40E+E56R+K98E+R108K+R118F+E210K+T244E+D254S,
A40E+D57N+G91T+K98E+R108K+R118F+E210K+T244E,
A40E+D57N+G91T+K98E+R108K+R118F+E210K+D254S,
A40E+D57N+G91T+K98E+R108K+R118F+T244E+D254S,
A40E+D57N+G91T+K98E+R108K+E210K+T244E+D254S,
A40E+D57N+G91T+K98E+R118F+E210K+T244E+D254S,
A40E+D57N+G91T+R108K+R118F+E210K+T244E+D254S,
A40E+D57N+K98E+R108K+R118F+E210K+T244E+D254S,
A40E+G91T+K98E+R108K+R118F+E210K+T244E+D254S,
E56R+D57N+G91T+K98E+R108K+R118F+E210K+T244E,
E56R+D57N+G91T+K98E+R108K+R118F+E210K+D254S,
E56R+D57N+G91T+K98E+R108K+R118F+T244E+D254S,
E56R+D57N+G91T+K98E+R108K+E210K+T244E+D254S,
E56R+D57N+G91T+K98E+R118F+E210K+T244E+D254S,
E56R+D57N+G91T+R108K+R118F+E210K+T244E+D254S,
E56R+D57N+K98E+R108K+R118F+E210K+T244E+D254S,
E56R+G91T+K98E+R108K+R118F+E210K+T244E+D254S,
D57N+G91T+K98E+R108K+R118F+E210K+T244E+D254S,
A40E+E56R+D57N+G91T+K98E+R108K+R118F+E210K+T244E,
A40E+E56R+D57N+G91T+K98E+R108K+R118F+E210K+D254S,
A40E+E56R+D57N+G91T+K98E+R108K+R118F+T244E+D254S,
A40E+E56R+D57N+G91T+K98E+R108K+E210K+T244E+D254S,
A40E+E56R+D57N+G91T+K98E+R118F+E210K+T244E+D254S,
A40E+E56R+D57N+G91T+R108K+R118F+E210K+T244E+D254S,
A40E+E56R+D57N+K98E+R108K+R118F+E210K+T244E+D254S,
A40E+E56R+G91T+K98E+R108K+R118F+E210K+T244E+D254S,
A40E+D57N+G91T+K98E+R108K+R118F+E210K+T244E+D254S,
E56R+D57N+G91T+K98E+R108K+R118F+E210K+T244E+D254S,
A40E+E56R+D57N+G91T+K98E+R108K+R118F+E210K+T244E+D254S.

In an embodiment, a variant of the invention has a substitution corresponding to L269H of SEQ ID NO: 8 and further comprises one of the following substitutions or set of substitutions corresponding to: A40E, E56R, D57N, G91T, K98E, R108K, R118F, E210K, T244E, A40E+E56R, A40E+D57N, A40E+G91T, A40E+K98E, A40E+R108K, A40E+R118F, A40E+E210K, A40E+T244E, A40E+D254S, E56R+D57N, E56R+G91T, E56R+K98E, E56R+R108K, E56R+R118F, E56R+E210K, E56R+T244E, E56R+D254S, D57N+G91T, D57N+K98E, D57N+R108K, D57N+R118F, D57N+E210K, D57N+T244E, D57N+D254S, G91T+K98E, G91T+R108K, G91T+R118F, G91T+E210K, G91T+T244E, G91T+D254S, K98E+R108K, K98E+R118F, K98E+E210K, K98E+T244E, K98E+D254S, R108K+R118F, R108K+E210K, R108K+T244E, R108K+D254S, R118F+E210K, R118F+T244E, R118F+D254S, E210K+T244E, E210K+D254S, T244E+D254S, A40E+E56R+D57N, A40E+E56R+G91T, A40E+E56R+K98E, A40E+E56R+R108K, A40E+E56R+R118F, A40E+E56R+E210K, A40E+E56R+T244E, A40E+E56R+D254S, A40E+D57N+G91T, A40E+D57N+K98E, A40E+D57N+R108K, A40E+D57N+R118F, A40E+D57N+E210K, A40E+D57N+T244E, A40E+D57N+D254S, A40E+G91T+K98E, A40E+G91T+R108K, A40E+G91T+R118F, A40E+G91T+E210K, A40E+G91T+T244E, A40E+G91T+D254S, A40E+K98E+R108K, A40E+K98E+R118F, A40E+K98E+E210K, A40E+K98E+T244E, A40E+K98E+D254S, A40E+R108K+R118F, A40E+R108K+E210K, A40E+R108K+T244E, A40E+R108K+D254S, A40E+R118F+E210K, A40E+R118F+T244E, A40E+R118F+D254S, A40E+E210K+T244E, A40E+E210K+D254S, A40E+T244E+D254S, E56R+D57N+G91T, E56R+D57N+K98E, E56R+D57N+R108K, E56R+D57N+R118F, E56R+D57N+E210K, E56R+D57N+T244E, E56R+D57N+D254S, E56R+G91T+K98E, E56R+G91T+R108K, E56R+G91T+R118F, E56R+G91T+E210K, E56R+G91T+T244E, E56R+G91T+D254S, E56R+K98E+R108K, E56R+K98E+R118F, E56R+K98E+E210K, E56R+K98E+T244E, E56R+K98E+D254S, E56R+R108K+R118F, E56R+R108K+E210K, E56R+R108K+T244E, E56R+R108K+D254S, E56R+R118F+E210K, E56R+R118F+T244E, E56R+R118F+D254S, E56R+E210K+T244E, E56R+E210K+D254S, E56R+T244E+D254S, D57N+G91T+K98E, D57N+G91T+R108K, D57N+G91T+R118F, D57N+G91T+E210K, D57N+G91T+T244E, D57N+G91T+D254S, D57N+K98E+R108K, D57N+K98E+R118F, D57N+K98E+E210K, D57N+K98E+T244E, D57N+K98E+D254S, D57N+R108K+R118F, D57N+R108K+E210K, D57N+R108K+T244E, D57N+R108K+D254S, D57N+R118F+E210K, D57N+R118F+T244E, D57N+R118F+D254S, D57N+E210K+T244E, D57N+E210K+D254S, D57N+T244E+D254S, G91T+K98E+R108K, G91T+K98E+R118F, G91T+K98E+E210K, G91T+K98E+T244E, G91T+K98E+D254S, G91T+R108K+R118F, G91T+R108K+E210K, G91T+R108K+T244E, G91T+R108K+D254S, G91T+R118F+E210K, G91T+R118F+T244E, G91T+R118F+D254S, G91T+E210K+T244E, G91T+E210K+D254S, G91T+T244E+D254S, K98E+R108K+R118F, K98E+R108K+E210K, K98E+R108K+T244E, K98E+R108K+D254S, K98E+R118F+E210K, K98E+R118F+T244E, K98E+R118F+D254S, K98E+E210K+T244E, K98E+E210K+D254S, K98E+T244E+D254S, R108K+R118F+E210K, R108K+R118F+T244E, R108K+R118F+D254S, R108K+E210K+T244E, R108K+E210K+D254S, R108K+T244E+D254S, R118F+E210K+T244E, R118F+E210K+D254S, R118F+T244E+D254S, E210K+T244E+D254S, A40E+E56R+D57N+G91T, A40E+E56R+D57N+K98E, A40E+E56R+D57N+R108K, A40E+E56R+D57N+R118F, A40E+E56R+D57N+E210K, A40E+E56R+D57N+T244E, A40E+E56R+D57N+D254S, A40E+E56R+G91T+K98E, A40E+E56R+G91T+R108K, A40E+E56R+G91T+R118F, A40E+E56R+G91T+E210K, A40E+E56R+G91T+T244E, A40E+E56R+G91T+D254S, A40E+E56R+K98E+R108K, A40E+E56R+K98E+R118F, A40E+E56R+K98E+E210K, A40E+E56R+K98E+T244E, A40E+E56R+K98E+D254S, A40E+E56R+R108K+R118F, A40E+E56R+R108K+E210K, A40E+E56R+R108K+T244E, A40E+E56R+R108K+D254S, A40E+E56R+R118F+E210K, A40E+E56R+R118F+T244E, A40E+E56R+R118F+D254S, A40E+E56R+E210K+T244E, A40E+E56R+E210K+D254S, A40E+E56R+T244E+D254S, A40E+D57N+G91T+K98E, A40E+D57N+G91T+R108K, A40E+D57N+G91T+R118F, A40E+D57N+G91T+E210K, A40E+D57N+G91T+T244E, A40E+D57N+G91T+D254S, A40E+D57N+K98E+R108K, A40E+D57N+K98E+R118F, A40E+D57N+K98E+E210K, A40E+D57N+K98E+T244E, A40E+D57N+K98E+D254S, A40E+D57N+R108K+R118F, A40E+D57N+R108K+E210K, A40E+D57N+R108K+T244E, A40E+D57N+R108K+D254S, A40E+D57N+R118F+E210K, A40E+D57N+R118F+T244E, A40E+D57N+R118F+D254S, A40E+D57N+E210K+T244E, A40E+D57N+E210K+D254S, A40E+D57N+T244E+D254S, A40E+G91T+K98E+R108K, A40E+G91T+K98E+R118F, A40E+G91T+K98E+E210K, A40E+G91T+K98E+T244E, A40E+G91T+K98E+D254S, A40E+G91T+R108K+R118F, A40E+G91T+R108K+E210K, A40E+G91T+R108K+T244E, A40E+G91T+R108K+D254S, A40E+G91T+R118F+E210K, A40E+G91T+R118F+T244E, A40E+G91T+R118F+D254S, A40E+G91T+E210K+T244E, A40E+G91T+E210K+D254S, A40E+G91T+T244E+D254S, A40E+K98E+R108K+R118F, A40E+K98E+R108K+E210K, A40E+K98E+R108K+T244E, A40E+K98E+R108K+D254S, A40E+K98E+R118F+E210K, A40E+K98E+R118F+T244E, A40E+K98E+R118F+D254S, A40E+K98E+E210K+T244E, A40E+K98E+E210K+D254S, A40E+K98E+T244E+D254S, A40E+R108K+R118F+E210K, A40E+R108K+R118F+T244E, A40E+R108K+R118F+D254S, A40E+R108K+E210K+T244E, A40E+R108K+E210K+D254S, A40E+R108K+T244E+D254S, A40E+R118F+E210K+T244E, A40E+R118F+E210K+D254S, A40E+R118F+T244E+D254S, A40E+E210K+T244E+D254S, E56R+D57N+G91T+K98E, E56R+D57N+G91T+R108K, E56R+D57N+G91T+R118F, E56R+D57N+G91T+E210K, E56R+D57N+G91T+T244E, E56R+D57N+G91T+D254S, E56R+D57N+K98E+R108K, E56R+D57N+K98E+R118F, E56R+D57N+K98E+E210K, E56R+D57N+K98E+T244E, E56R+D57N+K98E+D254S, E56R+D57N+R108K+R118F, E56R+D57N+R108K+E210K, E56R+D57N+R108K+T244E, E56R+D57N+R108K+D254S, E56R+D57N+R118F+E210K, E56R+D57N+R118F+T244E, E56R+D57N+R118F+D254S, E56R+D57N+E210K+T244E, E56R+D57N+E210K+D254S, E56R+D57N+T244E+D254S, E56R+G91T+K98E+R108K, E56R+G91T+K98E+R118F, E56R+G91T+K98E+E210K, E56R+G91T+K98E+T244E, E56R+G91T+K98E+D254S, E56R+G91T+R108K+R118F, E56R+G91T+R108K+E210K, E56R+G91T+R108K+T244E, E56R+G91T+R108K+D254S, E56R+G91T+R118F+E210K, E56R+G91T+R118F+T244E, E56R+G91T+R118F+D254S, E56R+G91T+E210K+T244E, E56R+G91T+E210K+D254S, E56R+G91T+T244E+D254S, E56R+K98E+R108K+R118F, E56R+K98E+R108K+E210K, E56R+K98E+R108K+T244E, E56R+K98E+R108K+D254S, E56R+K98E+R118F+E210K, E56R+K98E+R118F+T244E, E56R+K98E+R118F+D254S, E56R+K98E+E210K+T244E, E56R+K98E+E210K+D254S, E56R+K98E+T244E+D254S, E56R+R108K+R118F+E210K, E56R+R108K+R118F+T244E, E56R+R108K+R118F+D254S, E56R+R108K+E210K+T244E, E56R+R108K+E210K+D254S, E56R+R108K+T244E+D254S, E56R+R118F+E210K+T244E, E56R+R118F+E210K+D254S, E56R+R118F+T244E+D254S, E56R+E210K+T244E+D254S, D57N+G91T+K98E+R108K, D57N+G91T+K98E+R118F, D57N+G91T+K98E+E210K, D57N+G91T+K98E+T244E, D57N+G91T+K98E+D254S, D57N+G91T+R108K+R118F, D57N+G91T+R108K+E210K, D57N+G91T+R108K+T244E, D57N+G91T+R108K+D254S, D57N+G91T+R118F+E210K, D57N+G91T+R118F+T244E, D57N+G91T+R118F+D254S, D57N+G91T+E210K+T244E, D57N+G91T+E210K+D254S, D57N+G91T+T244E+D254S, D57N+K98E+R108K+R118F, D57N+K98E+R108K+E210K, D57N+K98E+R108K+T244E, D57N+K98E+R108K+D254S, D57N+K98E+R118F+E210K, D57N+K98E+R118F+T244E, D57N+K98E+R118F+D254S, D57N+K98E+E210K+T244E, D57N+K98E+E210K+D254S, D57N+K98E+T244E+D254S, D57N+R108K+R118F+E210K, D57N+R108K+R118F+T244E, D57N+R108K+R118F+D254S, D57N+R108K+E210K+T244E, D57N+R108K+E210K+D254S, D57N+R108K+T244E+D254S, D57N+R118F+E210K+T244E, D57N+R118F+E210K+D254S, D57N+R118F+T244E+D254S, D57N+E210K+T244E+D254S, G91T+K98E+R108K+R118F, G91T+K98E+R108K+E210K, G91T+K98E+R108K+T244E, G91T+K98E+R108K+D254S, G91T+K98E+R118F+E210K, G91T+K98E+R118F+T244E, G91T+K98E+R118F+D254S, G91T+K98E+E210K+T244E, G91T+K98E+E210K+D254S, G91T+K98E+T244E+D254S, G91T+R108K+R118F+E210K, G91T+R108K+R118F+T244E, G91T+R108K+R118F+D254S, G91T+R108K+E210K+T244E, G91T+R108K+E210K+D254S, G91T+R108K+T244E+D254S, G91T+R118F+E210K+T244E, G91T+R118F+E210K+D254S, G91T+R118F+T244E+D254S, G91T+E210K+T244E+D254S, K98E+R108K+R118F+E210K, K98E+R108K+R118F+T244E, K98E+R108K+R118F+D254S, K98E+R108K+E210K+T244E, K98E+R108K+E210K+D254S, K98E+R108K+T244E+D254S, K98E+R118F+E210K+T244E, K98E+R118F+E210K+D254S, K98E+R118F+T244E+D254S, K98E+E210K+T244E+D254S, R108K+R118F+E210K+T244E, R108K+R118F+E210K+D254S, R108K+R118F+T244E+D254S, R108K+E210K+T244E+D254S, R118F+E210K+T244E+D254S, A40E+E56R+D57N+G91T+K98E, A40E+E56R+D57N+G91T+R108K, A40E+E56R+D57N+G91T+R118F, A40E+E56R+D57N+G91T+E210K, A40E+E56R+D57N+G91T+T244E, A40E+E56R+D57N+G91T+D254S, A40E+E56R+D57N+K98E+R108K, A40E+E56R+D57N+K98E+R118F, A40E+E56R+D57N+K98E+E210K, A40E+E56R+D57N+K98E+T244E, A40E+E56R+D57N+K98E+D254S, A40E+E56R+D57N+R108K+R118F, A40E+E56R+D57N+R108K+E210K, A40E+E56R+D57N+R108K+T244E, A40E+E56R+D57N+R108K+D254S, A40E+E56R+D57N+R118F+E210K, A40E+E56R+D57N+R118F+T244E, A40E+E56R+D57N+R118F+D254S, A40E+E56R+D57N+E210K+T244E, A40E+E56R+D57N+E210K+D254S, A40E+E56R+D57N+T244E+D254S, A40E+E56R+G91T+K98E+R108K, A40E+E56R+G91T+K98E+R118F, A40E+E56R+G91T+K98E+E210K, A40E+E56R+G91T+K98E+T244E, A40E+E56R+G91T+K98E+D254S, A40E+E56R+G91T+R108K+R118F, A40E+E56R+G91T+R108K+E210K, A40E+E56R+G91T+R108K+T244E, A40E+E56R+G91T+R108K+D254S, A40E+E56R+G91T+R118F+E210K, A40E+E56R+G91T+R118F+T244E, A40E+E56R+G91T+R118F+D254S, A40E+E56R+G91T+E210K+T244E, A40E+E56R+G91T+E210K+D254S, A40E+E56R+G91T+T244E+D254S, A40E+E56R+K98E+R108K+R118F, A40E+E56R+K98E+R108K+E210K, A40E+E56R+K98E+R108K+T244E, A40E+E56R+K98E+R108K+D254S, A40E+E56R+K98E+R118F+E210K, A40E+E56R+K98E+R118F+T244E, A40E+E56R+K98E+R118F+D254S, A40E+E56R+K98E+E210K+T244E, A40E+E56R+K98E+E210K+D254S, A40E+E56R+K98E+T244E+D254S, A40E+E56R+R108K+R118F+E210K, A40E+E56R+R108K+R118F+T244E, A40E+E56R+R108K+R118F+D254S, A40E+E56R+R108K+E210K+T244E, A40E+E56R+R108K+E210K+D254S, A40E+E56R+R108K+T244E+D254S, A40E+E56R+R118F+E210K+T244E, A40E+E56R+R118F+E210K+D254S, A40E+E56R+R118F+T244E+D254S, A40E+E56R+E210K+T244E+D254S, A40E+D57N+G91T+K98E+R108K, A40E+D57N+G91T+K98E+R118F, A40E+D57N+G91T+K98E+E210K, A40E+D57N+G91T+K98E+T244E, A40E+D57N+G91T+K98E+D254S, A40E+D57N+G91T+R108K+R118F, A40E+D57N+G91T+R108K+E210K, A40E+D57N+G91T+R108K+T244E, A40E+D57N+G91T+R108K+D254S, A40E+D57N+G91T+R118F+E210K, A40E+D57N+G91T+R118F+T244E, A40E+D57N+G91T+R118F+D254S, A40E+D57N+G91T+E210K+T244E, A40E+D57N+G91T+E210K+D254S, A40E+D57N+G91T+T244E+D254S, A40E+D57N+K98E+R108K+R118F, A40E+D57N+K98E+R108K+E210K, A40E+D57N+K98E+R108K+T244E, A40E+D57N+K98E+R108K+D254S, A40E+D57N+K98E+R118F+E210K, A40E+D57N+K98E+R118F+T244E, A40E+D57N+K98E+R118F+D254S, A40E+D57N+K98E+E210K+T244E, A40E+D57N+K98E+E210K+D254S, A40E+D57N+K98E+T244E+D254S, A40E+D57N+R108K+R118F+E210K, A40E+D57N+R108K+R118F+T244E, A40E+D57N+R108K+R118F+D254S, A40E+D57N+R108K+E210K+T244E, A40E+D57N+R108K+E210K+D254S, A40E+D57N+R108K+T244E+D254S, A40E+D57N+R118F+E210K+T244E, A40E+D57N+R118F+E210K+D254S, A40E+D57N+R118F+T244E+D254S, A40E+D57N+E210K+T244E+D254S, A40E+G91T+K98E+R108K+R118F, A40E+G91T+K98E+R108K+E210K, A40E+G91T+K98E+R108K+T244E, A40E+G91T+K98E+R108K+D254S, A40E+G91T+K98E+R118F+E210K, A40E+G91T+K98E+R118F+T244E, A40E+G91T+K98E+R118F+D254S, A40E+G91T+K98E+E210K+T244E, A40E+G91T+K98E+E210K+D254S, A40E+G91T+K98E+T244E+D254S, A40E+G91T+R108K+R118F+E210K, A40E+G91T+R108K+R118F+T244E, A40E+G91T+R108K+R118F+D254S, A40E+G91T+R108K+E210K+T244E, A40E+G91T+R108K+E210K+D254S, A40E+G91T+R108K+T244E+D254S, A40E+G91T+R118F+E210K+T244E, A40E+G91T+R118F+E210K+D254S, A40E+G91T+R118F+T244E+D254S, A40E+G91T+E210K+T244E+D254S, A40E+K98E+R108K+R118F+E210K, A40E+K98E+R108K+R118F+T244E, A40E+K98E+R108K+R118F+D254S, A40E+K98E+R108K+E210K+T244E, A40E+K98E+R108K+E210K+D254S, A40E+K98E+R108K+T244E+D254S, A40E+K98E+R118F+E210K+T244E, A40E+K98E+R118F+E210K+D254S, A40E+K98E+R118F+T244E+D254S, A40E+K98E+E210K+T244E+D254S, A40E+R108K+R118F+E210K+T244E, A40E+R108K+R118F+E210K+D254S, A40E+R108K+R118F+T244E+D254S, A40E+R108K+E210K+T244E+D254S, A40E+R118F+E210K+T244E+D254S, E56R+D57N+G91T+K98E+R108K, E56R+D57N+G91T+K98E+R118F, E56R+D57N+G91T+K98E+E210K, E56R+D57N+G91T+K98E+T244E, E56R+D57N+G91T+K98E+D254S, E56R+D57N+G91T+R108K+R118F, E56R+D57N+G91T+R108K+E210K, E56R+D57N+G91T+R108K+T244E, E56R+D57N+G91T+R108K+D254S, E56R+D57N+G91T+R118F+E210K, E56R+D57N+G91T+R118F+T244E, E56R+D57N+G91T+R118F+D254S, E56R+D57N+G91T+E210K+T244E, E56R+D57N+G91T+E210K+D254S, E56R+D57N+G91T+T244E+D254S, E56R+D57N+K98E+R108K+R118F, E56R+D57N+K98E+R108K+E210K, E56R+D57N+K98E+R108K+T244E, E56R+D57N+K98E+R108K+D254S, E56R+D57N+K98E+R118F+E210K, E56R+D57N+K98E+R118F+T244E, E56R+D57N+K98E+R118F+D254S, E56R+D57N+K98E+E210K+T244E, E56R+D57N+K98E+E210K+D254S, E56R+D57N+K98E+T244E+D254S, E56R+D57N+R108K+R118F+E210K, E56R+D57N+R108K+R118F+T244E, E56R+D57N+R108K+R118F+D254S, E56R+D57N+R108K+E210K+T244E, E56R+D57N+R108K+E210K+D254S, E56R+D57N+R108K+T244E+D254S, E56R+D57N+R118F+E210K+T244E, E56R+D57N+R118F+E210K+D254S, E56R+D57N+R118F+T244E+D254S, E56R+D57N+E210K+T244E+D254S, E56R+G91T+K98E+R108K+R118F, E56R+G91T+K98E+R108K+E210K, E56R+G91T+K98E+R108K+T244E, E56R+G91T+K98E+R108K+D254S, E56R+G91T+K98E+R118F+E210K, E56R+G91T+K98E+R118F+T244E, E56R+G91T+K98E+R118F+D254S, E56R+G91T+K98E+E210K+T244E, E56R+G91T+K98E+E210K+D254S, E56R+G91T+K98E+T244E+D254S, E56R+G91T+R108K+R118F+E210K, E56R+G91T+R108K+R118F+T244E, E56R+G91T+R108K+R118F+D254S, E56R+G91T+R108K+E210K+T244E, E56R+G91T+R108K+E210K+D254S, E56R+G91T+R108K+T244E+D254S, E56R+G91T+R118F+E210K+T244E, E56R+G91T+R118F+E210K+D254S, E56R+G91T+R118F+T244E+D254S, E56R+G91T+E210K+T244E+D254S, E56R+K98E+R108K+R118F+E210K, E56R+K98E+R108K+R118F+T244E, E56R+K98E+R108K+R118F+D254S, E56R+K98E+R108K+E210K+T244E, E56R+K98E+R108K+E210K+D254S, E56R+K98E+R108K+T244E+D254S, E56R+K98E+R118F+E210K+T244E, E56R+K98E+R118F+E210K+D254S, E56R+K98E+R118F+T244E+D254S, E56R+K98E+E210K+T244E+D254S, E56R+R108K+R118F+E210K+T244E, E56R+R108K+R118F+E210K+D254S, E56R+R108K+R118F+T244E+D254S, E56R+R108K+E210K+T244E+D254S, E56R+R118F+E210K+T244E+D254S, D57N+G91T+K98E+R108K+R118F, D57N+G91T+K98E+R108K+E210K, D57N+G91T+K98E+R108K+T244E, D57N+G91T+K98E+R108K+D254S, D57N+G91T+K98E+R118F+E210K, D57N+G91T+K98E+R118F+T244E, D57N+G91T+K98E+R118F+D254S, D57N+G91T+K98E+E210K+T244E, D57N+G91T+K98E+E210K+D254S, D57N+G91T+K98E+T244E+D254S, D57N+G91T+R108K+R118F+E210K, D57N+G91T+R108K+R118F+T244E, D57N+G91T+R108K+R118F+D254S, D57N+G91T+R108K+E210K+T244E, D57N+G91T+R108K+E210K+D254S, D57N+G91T+R108K+T244E+D254S, D57N+G91T+R118F+E210K+T244E, D57N+G91T+R118F+E210K+D254S, D57N+G91T+R118F+T244E+D254S, D57N+G91T+E210K+T244E+D254S, D57N+K98E+R108K+R118F+E210K, D57N+K98E+R108K+R118F+T244E, D57N+K98E+R108K+R118F+D254S, D57N+K98E+R108K+E210K+T244E, D57N+K98E+R108K+E210K+D254S, D57N+K98E+R108K+T244E+D254S, D57N+K98E+R118F+E210K+T244E, D57N+K98E+R118F+E210K+D254S, D57N+K98E+R118F+T244E+D254S, D57N+K98E+E210K+T244E+D254S, D57N+R108K+R118F+E210K+T244E, D57N+R108K+R118F+E210K+D254S, D57N+R108K+R118F+T244E+D254S, D57N+R108K+E210K+T244E+D254S, D57N+R118F+E210K+T244E+D254S, G91T+K98E+R108K+R118F+E210K, G91T+K98E+R108K+R118F+T244E, G91T+K98E+R108K+R118F+D254S, G91T+K98E+R108K+E210K+T244E, G91T+K98E+R108K+E210K+D254S, G91T+K98E+R108K+T244E+D254S, G91T+K98E+R118F+E210K+T244E, G91T+K98E+R118F+E210K+D254S, G91T+K98E+R118F+T244E+D254S, G91T+K98E+E210K+T244E+D254S, G91T+R108K+R118F+E210K+T244E, G91T+R108K+R118F+E210K+D254S, G91T+R108K+R118F+T244E+D254S, G91T+R108K+E210K+T244E+D254S, G91T+R118F+E210K+T244E+D254S, K98E+R108K+R118F+E210K+T244E, K98E+R108K+R118F+E210K+D254S, K98E+R108K+R118F+T244E+D254S, K98E+R108K+E210K+T244E+D254S, K98E+R118F+E210K+T244E+D254S, R108K+R118F+E210K+T244E+D254S, A40E+E56R+D57N+G91T+K98E+R108K, A40E+E56R+D57N+G91T+K98E+R118F, A40E+E56R+D57N+G91T+K98E+E210K, A40E+E56R+D57N+G91T+K98E+T244E, A40E+E56R+D57N+G91T+K98E+D254S, A40E+E56R+D57N+G91T+R108K+R118F, A40E+E56R+D57N+G91T+R108K+E210K, A40E+E56R+D57N+G91T+R108K+T244E, A40E+E56R+D57N+G91T+R108K+D254S, A40E+E56R+D57N+G91T+R118F+E210K, A40E+E56R+D57N+G91T+R118F+T244E, A40E+E56R+D57N+G91T+R118F+D254S, A40E+E56R+D57N+G91T+E210K+T244E, A40E+E56R+D57N+G91T+E210K+D254S, A40E+E56R+D57N+G91T+T244E+D254S, A40E+E56R+D57N+K98E+R108K+R118F, A40E+E56R+D57N+K98E+R108K+E210K, A40E+E56R+D57N+K98E+R108K+T244E, A40E+E56R+D57N+K98E+R108K+D254S, A40E+E56R+D57N+K98E+R118F+E210K, A40E+E56R+D57N+K98E+R118F+T244E, A40E+E56R+D57N+K98E+R118F+D254S, A40E+E56R+D57N+K98E+E210K+T244E, A40E+E56R+D57N+K98E+E210K+D254S, A40E+E56R+D57N+K98E+T244E+D254S, A40E+E56R+D57N+R108K+R118F+E210K, A40E+E56R+D57N+R108K+R118F+T244E, A40E+E56R+D57N+R108K+R118F+D254S, A40E+E56R+D57N+R108K+E210K+T244E, A40E+E56R+D57N+R108K+E210K+D254S, A40E+E56R+D57N+R108K+T244E+D254S, A40E+E56R+D57N+R118F+E210K+T244E, A40E+E56R+D57N+R118F+E210K+D254S, A40E+E56R+D57N+R118F+T244E+D254S, A40E+E56R+D57N+E210K+T244E+D254S, A40E+E56R+G91T+K98E+R108K+R118F, A40E+E56R+G91T+K98E+R108K+E210K, A40E+E56R+G91T+K98E+R108K+T244E, A40E+E56R+G91T+K98E+R108K+D254S, A40E+E56R+G91T+K98E+R118F+E210K, A40E+E56R+G91T+K98E+R118F+T244E, A40E+E56R+G91T+K98E+R118F+D254S, A40E+E56R+G91T+K98E+E210K+T244E, A40E+E56R+G91T+K98E+E210K+D254S, A40E+E56R+G91T+K98E+T244E+D254S, A40E+E56R+G91T+R108K+R118F+E210K, A40E+E56R+G91T+R108K+R118F+T244E, A40E+E56R+G91T+R108K+R118F+D254S, A40E+E56R+G91T+R108K+E210K+T244E, A40E+E56R+G91T+R108K+E210K+D254S, A40E+E56R+G91T+R108K+T244E+D254S, A40E+E56R+G91T+R118F+E210K+T244E, A40E+E56R+G91T+R118F+E210K+D254S, A40E+E56R+G91T+R118F+T244E+D254S, A40E+E56R+G91T+E210K+T244E+D254S, A40E+E56R+K98E+R108K+R118F+E210K, A40E+E56R+K98E+R108K+R118F+T244E, A40E+E56R+K98E+R108K+R118F+D254S, A40E+E56R+K98E+R108K+E210K+T244E, A40E+E56R+K98E+R108K+E210K+D254S, A40E+E56R+K98E+R108K+T244E+D254S, A40E+E56R+K98E+R118F+E210K+T244E, A40E+E56R+K98E+R118F+E210K+D254S, A40E+E56R+K98E+R118F+T244E+D254S, A40E+E56R+K98E+E210K+T244E+D254S, A40E+E56R+R108K+R118F+E210K+T244E, A40E+E56R+R108K+R118F+E210K+D254S, A40E+E56R+R108K+R118F+T244E+D254S, A40E+E56R+R108K+E210K+T244E+D254S, A40E+E56R+R118F+E210K+T244E+D254S, A40E+D57N+G91T+K98E+R108K+R118F, A40E+D57N+G91T+K98E+R108K+E210K, A40E+D57N+G91T+K98E+R108K+T244E, A40E+D57N+G91T+K98E+R108K+D254S, A40E+D57N+G91T+K98E+R118F+E210K, A40E+D57N+G91T+K98E+R118F+T244E, A40E+D57N+G91T+K98E+R118F+D254S, A40E+D57N+G91T+K98E+E210K+T244E, A40E+D57N+G91T+K98E+E210K+D254S, A40E+D57N+G91T+K98E+T244E+D254S, A40E+D57N+G91T+R108K+R118F+E210K, A40E+D57N+G91T+R108K+R118F+T244E, A40E+D57N+G91T+R108K+R118F+D254S, A40E+D57N+G91T+R108K+E210K+T244E, A40E+D57N+G91T+R108K+E210K+D254S, A40E+D57N+G91T+R108K+T244E+D254S, A40E+D57N+G91T+R118F+E210K+T244E, A40E+D57N+G91T+R118F+E210K+D254S, A40E+D57N+G91T+R118F+T244E+D254S, A40E+D57N+G91T+E210K+T244E+D254S, A40E+D57N+K98E+R108K+R118F+E210K, A40E+D57N+K98E+R108K+R118F+T244E, A40E+D57N+K98E+R108K+R118F+D254S, A40E+D57N+K98E+R108K+E210K+T244E, A40E+D57N+K98E+R108K+E210K+D254S, A40E+D57N+K98E+R108K+T244E+D254S, A40E+D57N+K98E+R118F+E210K+T244E, A40E+D57N+K98E+R118F+E210K+D254S, A40E+D57N+K98E+R118F+T244E+D254S, A40E+D57N+K98E+E210K+T244E+D254S, A40E+D57N+R108K+R118F+E210K+T244E, A40E+D57N+R108K+R118F+E210K+D254S, A40E+D57N+R108K+R118F+T244E+D254S, A40E+D57N+R108K+E210K+T244E+D254S, A40E+D57N+R118F+E210K+T244E+D254S, A40E+G91T+K98E+R108K+R118F+E210K, A40E+G91T+K98E+R108K+R118F+T244E, A40E+G91T+K98E+R108K+R118F+D254S, A40E+G91T+K98E+R108K+E210K+T244E, A40E+G91T+K98E+R108K+E210K+D254S, A40E+G91T+K98E+R108K+T244E+D254S, A40E+G91T+K98E+R118F+E210K+T244E, A40E+G91T+K98E+R118F+E210K+D254S, A40E+G91T+K98E+R118F+T244E+D254S, A40E+G91T+K98E+E210K+T244E+D254S, A40E+G91T+R108K+R118F+E210K+T244E, A40E+G91T+R108K+R118F+E210K+D254S, A40E+G91T+R108K+R118F+T244E+D254S, A40E+G91T+R108K+E210K+T244E+D254S, A40E+G91T+R118F+E210K+T244E+D254S, A40E+K98E+R108K+R118F+E210K+T244E, A40E+K98E+R108K+R118F+E210K+D254S, A40E+K98E+R108K+R118F+T244E+D254S, A40E+K98E+R108K+E210K+T244E+D254S, A40E+K98E+R118F+E210K+T244E+D254S, A40E+R108K+R118F+E210K+T244E+D254S, E56R+D57N+G91T+K98E+R108K+R118F, E56R+D57N+G91T+K98E+R108K+E210K, E56R+D57N+G91T+K98E+R108K+T244E, E56R+D57N+G91T+K98E+R108K+D254S, E56R+D57N+G91T+K98E+R118F+E210K, E56R+D57N+G91T+K98E+R118F+T244E, E56R+D57N+G91T+K98E+R118F+D254S, E56R+D57N+G91T+K98E+E210K+T244E, E56R+D57N+G91T+K98E+E210K+D254S, E56R+D57N+G91T+K98E+T244E+D254S, E56R+D57N+G91T+R108K+R118F+E210K, E56R+D57N+G91T+R108K+R118F+T244E, E56R+D57N+G91T+R108K+R118F+D254S, E56R+D57N+G91T+R108K+E210K+T244E, E56R+D57N+G91T+R108K+E210K+D254S, E56R+D57N+G91T+R108K+T244E+D254S, E56R+D57N+G91T+R118F+E210K+T244E, E56R+D57N+G91T+R118F+E210K+D254S, E56R+D57N+G91T+R118F+T244E+D254S, E56R+D57N+G91T+E210K+T244E+D254S, E56R+D57N+K98E+R108K+R118F+E210K, E56R+D57N+K98E+R108K+R118F+T244E, E56R+D57N+K98E+R108K+R118F+D254S, E56R+D57N+K98E+R108K+E210K+T244E, E56R+D57N+K98E+R108K+E210K+D254S, E56R+D57N+K98E+R108K+T244E+D254S, E56R+D57N+K98E+R118F+E210K+T244E, E56R+D57N+K98E+R118F+E210K+D254S, E56R+D57N+K98E+R118F+T244E+D254S, E56R+D57N+K98E+E210K+T244E+D254S, E56R+D57N+R108K+R118F+E210K+T244E, E56R+D57N+R108K+R118F+E210K+D254S, E56R+D57N+R108K+R118F+T244E+D254S, E56R+D57N+R108K+E210K+T244E+D254S, E56R+D57N+R118F+E210K+T244E+D254S, E56R+G91T+K98E+R108K+R118F+E210K, E56R+G91T+K98E+R108K+R118F+T244E, E56R+G91T+K98E+R108K+R118F+D254S, E56R+G91T+K98E+R108K+E210K+T244E, E56R+G91T+K98E+R108K+E210K+D254S, E56R+G91T+K98E+R108K+T244E+D254S, E56R+G91T+K98E+R118F+E210K+T244E, E56R+G91T+K98E+R118F+E210K+D254S, E56R+G91T+K98E+R118F+T244E+D254S, E56R+G91T+K98E+E210K+T244E+D254S, E56R+G91T+R108K+R118F+E210K+T244E, E56R+G91T+R108K+R118F+E210K+D254S, E56R+G91T+R108K+R118F+T244E+D254S, E56R+G91T+R108K+E210K+T244E+D254S, E56R+G91T+R118F+E210K+T244E+D254S, E56R+K98E+R108K+R118F+E210K+T244E, E56R+K98E+R108K+R118F+E210K+D254S, E56R+K98E+R108K+R118F+T244E+D254S, E56R+K98E+R108K+E210K+T244E+D254S, E56R+K98E+R118F+E210K+T244E+D254S, E56R+R108K+R118F+E210K+T244E+D254S, D57N+G91T+K98E+R108K+R118F+E210K, D57N+G91T+K98E+R108K+R118F+T244E, D57N+G91T+K98E+R108K+R118F+D254S, D57N+G91T+K98E+R108K+E210K+T244E, D57N+G91T+K98E+R108K+E210K+D254S, D57N+G91T+K98E+R108K+T244E+D254S, D57N+G91T+K98E+R118F+E210K+T244E, D57N+G91T+K98E+R118F+E210K+D254S, D57N+G91T+K98E+R118F+T244E+D254S, D57N+G91T+K98E+E210K+T244E+D254S, D57N+G91T+R108K+R118F+E210K+T244E, D57N+G91T+R108K+R118F+E210K+D254S, D57N+G91T+R108K+R118F+T244E+D254S, D57N+G91T+R108K+E210K+T244E+D254S, D57N+G91T+R118F+E210K+T244E+D254S, D57N+K98E+R108K+R118F+E210K+T244E, D57N+K98E+R108K+R118F+E210K+D254S, D57N+K98E+R108K+R118F+T244E+D254S, D57N+K98E+R108K+E210K+T244E+D254S, D57N+K98E+R118F+E210K+T244E+D254S, D57N+R108K+R118F+E210K+T244E+D254S, G91T+K98E+R108K+R118F+E210K+T244E, G91T+K98E+R108K+R118F+E210K+D254S, G91T+K98E+R108K+R118F+T244E+D254S, G91T+K98E+R108K+E210K+T244E+D254S, G91T+K98E+R118F+E210K+T244E+D254S, G91T+R108K+R118F+E210K+T244E+D254S, K98E+R108K+R118F+E210K+T244E+D254S,
A40E+E56R+D57N+G91T+K98E+R108K+R118F,
A40E+E56R+D57N+G91T+K98E+R108K+E210K,
A40E+E56R+D57N+G91T+K98E+R108K+T244E,
A40E+E56R+D57N+G91T+K98E+R108K+D254S,
A40E+E56R+D57N+G91T+K98E+R118F+E210K,
A40E+E56R+D57N+G91T+K98E+R118F+T244E,
A40E+E56R+D57N+G91T+K98E+R118F+D254S,
A40E+E56R+D57N+G91T+K98E+E210K+T244E,
A40E+E56R+D57N+G91T+K98E+E210K+D254S,
A40E+E56R+D57N+G91T+K98E+T244E+D254S,
A40E+E56R+D57N+G91T+R108K+R118F+E210K,
A40E+E56R+D57N+G91T+R108K+R118F+T244E,
A40E+E56R+D57N+G91T+R108K+R118F+D254S,
A40E+E56R+D57N+G91T+R108K+E210K+T244E,
A40E+E56R+D57N+G91T+R108K+E210K+D254S,
A40E+E56R+D57N+G91T+R108K+T244E+D254S,
A40E+E56R+D57N+G91T+R118F+E210K+T244E,
A40E+E56R+D57N+G91T+R118F+E210K+D254S,
A40E+E56R+D57N+G91T+R118F+T244E+D254S,
A40E+E56R+D57N+G91T+E210K+T244E+D254S,
A40E+E56R+D57N+K98E+R108K+R118F+E210K,
A40E+E56R+D57N+K98E+R108K+R118F+T244E,
A40E+E56R+D57N+K98E+R108K+R118F+D254S,
A40E+E56R+D57N+K98E+R108K+E210K+T244E,
A40E+E56R+D57N+K98E+R108K+E210K+D254S,
A40E+E56R+D57N+K98E+R108K+T244E+D254S,
A40E+E56R+D57N+K98E+R118F+E210K+T244E,
A40E+E56R+D57N+K98E+R118F+E210K+D254S,
A40E+E56R+D57N+K98E+R118F+T244E+D254S,
A40E+E56R+D57N+K98E+E210K+T244E+D254S,
A40E+E56R+D57N+R108K+R118F+E210K+T244E,
A40E+E56R+D57N+R108K+R118F+E210K+D254S,
A40E+E56R+D57N+R108K+R118F+T244E+D254S,
A40E+E56R+D57N+R108K+E210K+T244E+D254S,
A40E+E56R+D57N+R118F+E210K+T244E+D254S,
A40E+E56R+G91T+K98E+R108K+R118F+E210K,
A40E+E56R+G91T+K98E+R108K+R118F+T244E,
A40E+E56R+G91T+K98E+R108K+R118F+D254S,
A40E+E56R+G91T+K98E+R108K+E210K+T244E,
A40E+E56R+G91T+K98E+R108K+E210K+D254S,
A40E+E56R+G91T+K98E+R108K+T244E+D254S,
A40E+E56R+G91T+K98E+R118F+E210K+T244E,
A40E+E56R+G91T+K98E+R118F+E210K+D254S,
A40E+E56R+G91T+K98E+R118F+T244E+D254S,
A40E+E56R+G91T+K98E+E210K+T244E+D254S,
A40E+E56R+G91T+R108K+R118F+E210K+T244E,
A40E+E56R+G91T+R108K+R118F+E210K+D254S,
A40E+E56R+G91T+R108K+R118F+T244E+D254S,
A40E+E56R+G91T+R108K+E210K+T244E+D254S,
A40E+E56R+G91T+R118F+E210K+T244E+D254S,
A40E+E56R+K98E+R108K+R118F+E210K+T244E,
A40E+E56R+K98E+R108K+R118F+E210K+D254S,
A40E+E56R+K98E+R108K+R118F+T244E+D254S,
A40E+E56R+K98E+R108K+E210K+T244E+D254S,
A40E+E56R+K98E+R118F+E210K+T244E+D254S,
A40E+E56R+R108K+R118F+E210K+T244E+D254S,
A40E+D57N+G91T+K98E+R108K+R118F+E210K,
A40E+D57N+G91T+K98E+R108K+R118F+T244E,
A40E+D57N+G91T+K98E+R108K+R118F+D254S,
A40E+D57N+G91T+K98E+R108K+E210K+T244E,
A40E+D57N+G91T+K98E+R108K+E210K+D254S,
A40E+D57N+G91T+K98E+R108K+T244E+D254S,
A40E+D57N+G91T+K98E+R118F+E210K+T244E,
A40E+D57N+G91T+K98E+R118F+E210K+D254S,
A40E+D57N+G91T+K98E+R118F+T244E+D254S,
A40E+D57N+G91T+K98E+E210K+T244E+D254S,
A40E+D57N+G91T+R108K+R118F+E210K+T244E,
A40E+D57N+G91T+R108K+R118F+E210K+D254S,
A40E+D57N+G91T+R108K+R118F+T244E+D254S,
A40E+D57N+G91T+R108K+E210K+T244E+D254S,
A40E+D57N+G91T+R118F+E210K+T244E+D254S,
A40E+D57N+K98E+R108K+R118F+E210K+T244E,
A40E+D57N+K98E+R108K+R118F+E210K+D254S,
A40E+D57N+K98E+R108K+R118F+T244E+D254S,
A40E+D57N+K98E+R108K+E210K+T244E+D254S,
A40E+D57N+K98E+R118F+E210K+T244E+D254S,
A40E+D57N+R108K+R118F+E210K+T244E+D254S,
A40E+G91T+K98E+R108K+R118F+E210K+T244E,
A40E+G91T+K98E+R108K+R118F+E210K+D254S,
A40E+G91T+K98E+R108K+R118F+T244E+D254S,
A40E+G91T+K98E+R108K+E210K+T244E+D254S,
A40E+G91T+K98E+R118F+E210K+T244E+D254S,
A40E+G91T+R108K+R118F+E210K+T244E+D254S,
A40E+K98E+R108K+R118F+E210K+T244E+D254S,
E56R+D57N+G91T+K98E+R108K+R118F+E210K,
E56R+D57N+G91T+K98E+R108K+R118F+T244E,
E56R+D57N+G91T+K98E+R108K+R118F+D254S,
E56R+D57N+G91T+K98E+R108K+E210K+T244E,
E56R+D57N+G91T+K98E+R108K+E210K+D254S,
E56R+D57N+G91T+K98E+R108K+T244E+D254S,
E56R+D57N+G91T+K98E+R118F+E210K+T244E,
E56R+D57N+G91T+K98E+R118F+E210K+D254S,
E56R+D57N+G91T+K98E+R118F+T244E+D254S,
E56R+D57N+G91T+K98E+E210K+T244E+D254S,
E56R+D57N+G91T+R108K+R118F+E210K+T244E,
E56R+D57N+G91T+R108K+R118F+E210K+D254S,
E56R+D57N+G91T+R108K+R118F+T244E+D254S,
E56R+D57N+G91T+R108K+E210K+T244E+D254S,
E56R+D57N+G91T+R118F+E210K+T244E+D254S,
E56R+D57N+K98E+R108K+R118F+E210K+T244E,
E56R+D57N+K98E+R108K+R118F+E210K+D254S,
E56R+D57N+K98E+R108K+R118F+T244E+D254S,
E56R+D57N+K98E+R108K+E210K+T244E+D254S,
E56R+D57N+K98E+R118F+E210K+T244E+D254S,
E56R+D57N+R108K+R118F+E210K+T244E+D254S,
E56R+G91T+K98E+R108K+R118F+E210K+T244E,
E56R+G91T+K98E+R108K+R118F+E210K+D254S,
E56R+G91T+K98E+R108K+R118F+T244E+D254S,
E56R+G91T+K98E+R108K+E210K+T244E+D254S,
E56R+G91T+K98E+R118F+E210K+T244E+D254S,
E56R+G91T+R108K+R118F+E210K+T244E+D254S,
E56R+K98E+R108K+R118F+E210K+T244E+D254S,
D57N+G91T+K98E+R108K+R118F+E210K+T244E,
D57N+G91T+K98E+R108K+R118F+E210K+D254S,
D57N+G91T+K98E+R108K+R118F+T244E+D254S,
D57N+G91T+K98E+R108K+E210K+T244E+D254S,
D57N+G91T+K98E+R118F+E210K+T244E+D254S,
D57N+G91T+R108K+R118F+E210K+T244E+D254S,
D57N+K98E+R108K+R118F+E210K+T244E+D254S,
G91T+K98E+R108K+R118F+E210K+T244E+D254S,
A40E+E56R+D57N+G91T+K98E+R108K+R118F+E210K,
A40E+E56R+D57N+G91T+K98E+R108K+R118F+T244E,
A40E+E56R+D57N+G91T+K98E+R108K+R118F+D254S,
A40E+E56R+D57N+G91T+K98E+R108K+E210K+T244E,
A40E+E56R+D57N+G91T+K98E+R108K+E210K+D254S,
A40E+E56R+D57N+G91T+K98E+R108K+T244E+D254S,
A40E+E56R+D57N+G91T+K98E+R118F+E210K+T244E,
A40E+E56R+D57N+G91T+K98E+R118F+E210K+D254S,
A40E+E56R+D57N+G91T+K98E+R118F+T244E+D254S,
A40E+E56R+D57N+G91T+K98E+E210K+T244E+D254S,
A40E+E56R+D57N+G91T+R108K+R118F+E210K+T244E,
A40E+E56R+D57N+G91T+R108K+R118F+E210K+D254S,
A40E+E56R+D57N+G91T+R108K+R118F+T244E+D254S,
A40E+E56R+D57N+G91T+R108K+E210K+T244E+D254S,
A40E+E56R+D57N+G91T+R118F+E210K+T244E+D254S,
A40E+E56R+D57N+K98E+R108K+R118F+E210K+T244E,
A40E+E56R+D57N+K98E+R108K+R118F+E210K+D254S,
A40E+E56R+D57N+K98E+R108K+R118F+T244E+D254S,
A40E+E56R+D57N+K98E+R108K+E210K+T244E+D254S,
A40E+E56R+D57N+K98E+R118F+E210K+T244E+D254S,
A40E+E56R+D57N+R108K+R118F+E210K+T244E+D254S,
A40E+E56R+G91T+K98E+R108K+R118F+E210K+T244E,
A40E+E56R+G91T+K98E+R108K+R118F+E210K+D254S,
A40E+E56R+G91T+K98E+R108K+R118F+T244E+D254S,
A40E+E56R+G91T+K98E+R108K+E210K+T244E+D254S,
A40E+E56R+G91T+K98E+R118F+E210K+T244E+D254S,
A40E+E56R+G91T+R108K+R118F+E210K+T244E+D254S,
A40E+E56R+K98E+R108K+R118F+E210K+T244E+D254S,
A40E+D57N+G91T+K98E+R108K+R118F+E210K+T244E,
A40E+D57N+G91T+K98E+R108K+R118F+E210K+D254S,
A40E+D57N+G91T+K98E+R108K+R118F+T244E+D254S,
A40E+D57N+G91T+K98E+R108K+E210K+T244E+D254S,
A40E+D57N+G91T+K98E+R118F+E210K+T244E+D254S,
A40E+D57N+G91T+R108K+R118F+E210K+T244E+D254S,
A40E+D57N+K98E+R108K+R118F+E210K+T244E+D254S,
A40E+G91T+K98E+R108K+R118F+E210K+T244E+D254S,
E56R+D57N+G91T+K98E+R108K+R118F+E210K+T244E,
E56R+D57N+G91T+K98E+R108K+R118F+E210K+D254S,
E56R+D57N+G91T+K98E+R108K+R118F+T244E+D254S,
E56R+D57N+G91T+K98E+R108K+E210K+T244E+D254S,
E56R+D57N+G91T+K98E+R118F+E210K+T244E+D254S,
E56R+D57N+G91T+R108K+R118F+E210K+T244E+D254S,
E56R+D57N+K98E+R108K+R118F+E210K+T244E+D254S,
E56R+G91T+K98E+R108K+R118F+E210K+T244E+D254S,
D57N+G91T+K98E+R108K+R118F+E210K+T244E+D254S,
A40E+E56R+D57N+G91T+K98E+R108K+R118F+E210K+T244E,
A40E+E56R+D57N+G91T+K98E+R108K+R118F+E210K+D254S,
A40E+E56R+D57N+G91T+K98E+R108K+R118F+T244E+D254S,
A40E+E56R+D57N+G91T+K98E+R108K+E210K+T244E+D254S,
A40E+E56R+D57N+G91T+K98E+R118F+E210K+T244E+D254S,
A40E+E56R+D57N+G91T+R108K+R118F+E210K+T244E+D254S,
A40E+E56R+D57N+K98E+R108K+R118F+E210K+T244E+D254S,
A40E+E56R+G91T+K98E+R108K+R118F+E210K+T244E+D254S,
A40E+D57N+G91T+K98E+R108K+R118F+E210K+T244E+D254S,
E56R+D57N+G91T+K98E+R108K+R118F+E210K+T244E+D254S,
A40E+E56R+D57N+G91T+K98E+R108K+R118F+E210K+T244E+D254S.

In an embodiment, a variant of the invention has a substitution corresponding to I202H of SEQ ID NO: 8 and further comprises one of the following substitutions or set of substitutions corresponding to: G23S, D27N, A40I, F51I, E56R, V60K, R118F, T244E, P256T, G23S+D27N, G23S+A40I, G23S+F51I, G23S+E56R, G23S+V60K, G23S+R118F, G23S+T244E, G23S+P256T, D27N+A40I, D27N+F51I, D27N+E56R, D27N+V60K, D27N+R118F, D27N+T244E, D27N+P256T, A40I+F51I, A40I+E56R, A40I+V60K, A40I+R118F, A40I+T244E, A40I+P256T, F51I+E56R, F51I+V60K, F51I+R118F, F51I+T244E, F51I+P256T, E56R+V60K, E56R+R118F, E56R+T244E, E56R+P256T, V60K+R118F, V60K+T244E, V60K+P256T, R118F+T244E, R118F+P256T, T244E+P256T, G23S+D27N+A40I, G23S+D27N+F51I, G23S+D27N+E56R, G23S+D27N+V60K, G23S+D27N+R118F, G23S+D27N+T244E, G23S+D27N+P256T, G23S+A40I+F51I, G23S+A40I+E56R, G23S+A40I+V60K, G23S+A40I+R118F, G23S+A40I+T244E, G23S+A40I+P256T, G23S+F51I+E56R, G23S+F51I+V60K, G23S+F51I+R118F, G23S+F51I+T244E, G23S+F51I+P256T, G23S+E56R+V60K, G23S+E56R+R118F, G23S+E56R+T244E, G23S+E56R+P256T, G23S+V60K+R118F, G23S+V60K+T244E, G23S+V60K+P256T, G23S+R118F+T244E, G23S+R118F+P256T, G23S+T244E+P256T, D27N+A40I+F51I, D27N+A40I+E56R, D27N+A40I+V60K, D27N+A40I+R118F, D27N+A40I+T244E, D27N+A40I+P256T, D27N+F51I+E56R, D27N+F51I+V60K, D27N+F51I+R118F, D27N+F51I+T244E, D27N+F51I+P256T, D27N+E56R+V60K, D27N+E56R+R118F, D27N+E56R+T244E, D27N+E56R+P256T, D27N+V60K+R118F, D27N+V60K+T244E, D27N+V60K+P256T, D27N+R118F+T244E, D27N+R118F+P256T, D27N+T244E+P256T, A40I+F51I+E56R, A40I+F51I+V60K, A40I+F51I+R118F, A40I+F51I+T244E, A40I+F51I+P256T, A40I+E56R+V60K, A40I+E56R+R118F, A40I+E56R+T244E, A40I+E56R+P256T, A40I+V60K+R118F, A40I+V60K+T244E, A40I+V60K+P256T, A40I+R118F+T244E, A40I+R118F+P256T, A40I+T244E+P256T, F51I+E56R+V60K, F51I+E56R+R118F, F51I+E56R+T244E, F51I+E56R+P256T, F51I+V60K+R118F, F51I+V60K+T244E, F51I+V60K+P256T, F51I+R118F+T244E, F51I+R118F+P256T, F51I+T244E+P256T, E56R+V60K+R118F, E56R+V60K+T244E, E56R+V60K+P256T, E56R+R118F+T244E, E56R+R118F+P256T, E56R+T244E+P256T, V60K+R118F+T244E, V60K+R118F+P256T, V60K+T244E+P256T, R118F+T244E+P256T, G23S+D27N+A40I+F51I, G23S+D27N+A40I+E56R, G23S+D27N+A40I+V60K, G23S+D27N+A40I+R118F, G23S+D27N+A40I+T244E, G23S+D27N+A40I+P256T, G23S+D27N+F51I+E56R, G23S+D27N+F51I+V60K, G23S+D27N+F51I+R118F, G23S+D27N+F51I+T244E, G23S+D27N+F51I+P256T, G23S+D27N+E56R+V60K, G23S+D27N+E56R+R118F, G23S+D27N+E56R+T244E, G23S+D27N+E56R+P256T, G23S+D27N+V60K+R118F, G23S+D27N+V60K+T244E, G23S+D27N+V60K+P256T, G23S+D27N+R118F+T244E, G23S+D27N+R118F+P256T, G23S+D27N+T244E+P256T, G23S+A40I+F51I+E56R, G23S+A40I+F51I+V60K, G23S+A40I+F51I+R118F, G23S+A40I+F51I+T244E, G23S+A40I+F51I+P256T, G23S+A40I+E56R+V60K, G23S+A40I+E56R+R118F, G23S+A40I+E56R+T244E, G23S+A40I+E56R+P256T, G23S+A40I+V60K+R118F, G23S+A40I+V60K+T244E, G23S+A40I+V60K+P256T, G23S+A40I+R118F+T244E, G23S+A40I+R118F+P256T, G23S+A40I+T244E+P256T, G23S+F51I+E56R+V60K, G23S+F51I+E56R+R118F, G23S+F51I+E56R+T244E, G23S+F51I+E56R+P256T, G23S+F51I+V60K+R118F, G23S+F51I+V60K+T244E, G23S+F51I+V60K+P256T, G23S+F51I+R118F+T244E, G23S+F51I+R118F+P256T, G23S+F51I+T244E+P256T, G23S+E56R+V60K+R118F, G23S+E56R+V60K+T244E, G23S+E56R+V60K+P256T, G23S+E56R+R118F+T244E, G23S+E56R+R118F+P256T, G23S+E56R+T244E+P256T, G23S+V60K+R118F+T244E, G23S+V60K+R118F+P256T, G23S+V60K+T244E+P256T, G23S+R118F+T244E+P256T, D27N+A40I+F51I+E56R, D27N+A40I+F51I+V60K, D27N+A40I+F51I+R118F, D27N+A40I+F51I+T244E, D27N+A40I+F51I+P256T, D27N+A40I+E56R+V60K, D27N+A40I+E56R+R118F, D27N+A40I+E56R+T244E, D27N+A40I+E56R+P256T, D27N+A40I+V60K+R118F, D27N+A40I+V60K+T244E, D27N+A40I+V60K+P256T, D27N+A40I+R118F+T244E, D27N+A40I+R118F+P256T, D27N+A40I+T244E+P256T, D27N+F51I+E56R+V60K, D27N+F51I+E56R+R118F, D27N+F51I+E56R+T244E, D27N+F51I+E56R+P256T, D27N+F51I+V60K+R118F, D27N+F51I+V60K+T244E, D27N+F51I+V60K+P256T, D27N+F51I+R118F+T244E, D27N+F51I+R118F+P256T, D27N+F51I+T244E+P256T, D27N+E56R+V60K+R118F, D27N+E56R+V60K+T244E, D27N+E56R+V60K+P256T, D27N+E56R+R118F+T244E, D27N+E56R+R118F+P256T, D27N+E56R+T244E+P256T, D27N+V60K+R118F+T244E, D27N+V60K+R118F+P256T, D27N+V60K+T244E+P256T, D27N+R118F+T244E+P256T, A40I+F51I+E56R+V60K, A40I+F51I+E56R+R118F, A40I+F51I+E56R+T244E, A40I+F51I+E56R+P256T, A40I+F51I+V60K+R118F, A40I+F51I+V60K+T244E, A40I+F51I+V60K+P256T, A40I+F51I+R118F+T244E, A40I+F51I+R118F+P256T, A40I+F51I+T244E+P256T, A40I+E56R+V60K+R118F, A40I+E56R+V60K+T244E, A40I+E56R+V60K+P256T, A40I+E56R+R118F+T244E, A40I+E56R+R118F+P256T, A40I+E56R+T244E+P256T, A40I+V60K+R118F+T244E, A40I+V60K+R118F+P256T, A40I+V60K+T244E+P256T, A40I+R118F+T244E+P256T, F51I+E56R+V60K+R118F, F51I+E56R+V60K+T244E, F51I+E56R+V60K+P256T, F51I+E56R+R118F+T244E, F51I+E56R+R118F+P256T, F51I+E56R+T244E+P256T, F51I+V60K+R118F+T244E, F51I+V60K+R118F+P256T, F51I+V60K+T244E+P256T, F51I+R118F+T244E+P256T, E56R+V60K+R118F+T244E, E56R+V60K+R118F+P256T, E56R+V60K+T244E+P256T, E56R+R118F+T244E+P256T, V60K+R118F+T244E+P256T, G23S+D27N+A40I+F51I+E56R, G23S+D27N+A40I+F51I+V60K, G23S+D27N+A40I+F51I+R118F, G23S+D27N+A40I+F51 I+T244E, G23S+D27N+A40I+F51 I+P256T, G23S+D27N+A40I+E56R+V60K, G23S+D27N+A40I+E56R+R118F, G23S+D27N+A40I+E56R+T244E, G23S+D27N+A40I+E56R+P256T, G23S+D27N+A40I+V60K+R118F, G23S+D27N+A40I+V60K+T244E, G23S+D27N+A40I+V60K+P256T, G23S+D27N+A40I+R118F+T244E, G23S+D27N+A40I+R118F+P256T, G23S+D27N+A40I+T244E+P256T, G23S+D27N+F51I+E56R+V60K, G23S+D27N+F51I+E56R+R118F, G23S+D27N+F51I+E56R+T244E, G23S+D27N+F51 I+E56R+P256T, G23S+D27N+F51 I+V60K+R118F, G23S+D27N+F51 I+V60K+T244E, G23S+D27N+F51 I+V60K+P256T, G23S+D27N+F51 I+R118F+T244E, G23S+D27N+F51I+R118F+P256T, G23S+D27N+F51I+T244E+P256T, G23S+D27N+E56R+V60K+R118F, G23S+D27N+E56R+V60K+T244E, G23S+D27N+E56R+V60K+P256T, G23S+D27N+E56R+R118F+T244E, G23S+D27N+E56R+R118F+P256T, G23S+D27N+E56R+T244E+P256T, G23S+D27N+V60K+R118F+T244E, G23S+D27N+V60K+R118F+P256T, G23S+D27N+V60K+T244E+P256T, G23S+D27N+R118F+T244E+P256T, G23S+A40I+F51 I+E56R+V60K, G23S+A40I+F51I+E56R+R118F, G23S+A40I+F51I+E56R+T244E, G23S+A40I+F51I+E56R+P256T, G23S+A40I+F51 I+V60K+R118F, G23S+A40I+F51I+V60K+T244E, G23S+A40I+F51I+V60K+P256T, G23S+A40I+F51 I+R118F+T244E, G23S+A40I+F51I+R118F+P256T, G23S+A40I+F51I+T244E+P256T, G23S+A40I+E56R+V60K+R118F, G23S+A40I+E56R+V60K+T244E, G23S+A40I+E56R+V60K+P256T, G23S+A40I+E56R+R118F+T244E, G23S+A40I+E56R+R118F+P256T, G23S+A40I+E56R+T244E+P256T, G23S+A40I+V60K+R118F+T244E, G23S+A40I+V60K+R118F+P256T, G23S+A40I+V60K+T244E+P256T, G23S+A40I+R118F+T244E+P256T, G23S+F51I+E56R+V60K+R118F, G23S+F51I+E56R+V60K+T244E, G23S+F51I+E56R+V60K+P256T, G23S+F51I+E56R+R118F+T244E, G23S+F51I+E56R+R118F+P256T, G23S+F51I+E56R+T244E+P256T, G23S+F51I+V60K+R118F+T244E, G23S+F51 I+V60K+R118F+P256T, G23S+F51I+V60K+T244E+P256T, G23S+F51I+R118F+T244E+P256T, G23S+E56R+V60K+R118F+T244E, G23S+E56R+V60K+R118F+P256T, G23S+E56R+V60K+T244E+P256T, G23S+E56R+R118F+T244E+P256T, G23S+V60K+R118F+T244E+P256T, D27N+A40I+F51I+E56R+V60K, D27N+A40I+F51I+E56R+R118F, D27N+A40I+F51I+E56R+T244E, D27N+A40I+F51 I+E56R+P256T, D27N+A40I+F51 I+V60K+R118F, D27N+A40I+F51 I+V60K+T244E, D27N+A40I+F51I+V60K+P256T, D27N+A40I+F51I+R118F+T244E, D27N+A40I+F51I+R118F+P256T, D27N+A40I+F51I+T244E+P256T, D27N+A40I+E56R+V60K+R118F, D27N+A40I+E56R+V60K+T244E, D27N+A40I+E56R+V60K+P256T, D27N+A40I+E56R+R118F+T244E, D27N+A40I+E56R+R118F+P256T, D27N+A40I+E56R+T244E+P256T, D27N+A40I+V60K+R118F+T244E, D27N+A40I+V60K+R118F+P256T, D27N+A40I+V60K+T244E+P256T, D27N+A40I+R118F+T244E+P256T, D27N+F51I+E56R+V60K+R118F, D27N+F51I+E56R+V60K+T244E, D27N+F51 I+E56R+V60K+P256T, D27N+F51 I+E56R+R118F+T244E, D27N+F51I+E56R+R118F+P256T, D27N+F51I+E56R+T244E+P256T, D27N+F51 I+V60K+R118F+T244E, D27N+F51I+V60K+R118F+P256T, D27N+F51I+V60K+T244E+P256T, D27N+F51I+R118F+T244E+P256T, D27N+E56R+V60K+R118F+T244E, D27N+E56R+V60K+R118F+P256T, D27N+E56R+V60K+T244E+P256T, D27N+E56R+R118F+T244E+P256T, D27N+V60K+R118F+T244E+P256T, A40I+F51I+E56R+V60K+R118F, A40I+F51 I+E56R+V60K+T244E, A40I+F51 I+E56R+V60K+P256T, A40I+F51I+E56R+R118F+T244E, A40I+F51I+E56R+R118F+P256T, A40I+F51I+E56R+T244E+P256T, A40I+F51I+V60K+R118F+T244E, A40I+F51I+V60K+R118F+P256T, A40I+F51I+V60K+T244E+P256T, A40I+F51I+R118F+T244E+P256T, A40I+E56R+V60K+R118F+T244E, A40I+E56R+V60K+R118F+P256T, A40I+E56R+V60K+T244E+P256T, A40I+E56R+R118F+T244E+P256T, A40I+V60K+R118F+T244E+P256T, F51I+E56R+V60K+R118F+T244E, F51I+E56R+V60K+R118F+P256T, F51 I+E56R+V60K+T244E+P256T, F51I+E56R+R118F+T244E+P256T, F51I+V60K+R118F+T244E+P256T, E56R+V60K+R118F+T244E+P256T, G23S+D27N+A40I+F51I+E56R+V60K, G23S+D27N+A40I+F51 I+E56R+R118F, G23S+D27N+A40I+F51I+E56R+T244E, G23S+D27N+A40I+F51 I+E56R+P256T, G23S+D27N+A40I+F51I+V60K+R118F, G23S+D27N+A40I+F51 I+V60K+T244E, G23S+D27N+A40I+F51I+V60K+P256T, G23S+D27N+A40I+F51I+R118F+T244E, G23S+D27N+A40I+F51I+R118F+P256T, G23S+D27N+A40I+F51I+T244E+P256T, G23S+D27N+A40I+E56R+V60K+R118F, G23S+D27N+A40I+E56R+V60K+T244E, G23S+D27N+A40I+E56R+V60K+P256T, G23S+D27N+A40I+E56R+R118F+T244E, G23S+D27N+A40I+E56R+R118F+P256T, G23S+D27N+A40I+E56R+T244E+P256T, G23S+D27N+A40I+V60K+R118F+T244E, G23S+D27N+A40I+V60K+R118F+P256T, G23S+D27N+A40I+V60K+T244E+P256T, G23S+D27N+A40I+R118F+T244E+P256T, G23S+D27N+F51 I+E56R+V60K+R118F, G23S+D27N+F51 I+E56R+V60K+T244E, G23S+D27N+F51 I+E56R+V60K+P256T, G23S+D27N+F51I+E56R+R118F+T244E, G23S+D27N+F51I+E56R+R118F+P256T, G23S+D27N+F51 I+E56R+T244E+P256T, G23S+D27N+F51I+V60K+R118F+T244E, G23S+D27N+F51I+V60K+R118F+P256T, G23S+D27N+F51I+V60K+T244E+P256T, G23S+D27N+F51I+R118F+T244E+P256T, G23S+D27N+E56R+V60K+R118F+T244E, G23S+D27N+E56R+V60K+R118F+P256T, G23S+D27N+E56R+V60K+T244E+P256T, G23S+D27N+E56R+R118F+T244E+P256T, G23S+D27N+V60K+R118F+T244E+P256T, G23S+A40I+F51I+E56R+V60K+R118F, G23S+A40I+F51I+E56R+V60K+T244E, G23S+A40I+F51I+E56R+V60K+P256T, G23S+A40I+F51I+E56R+R118F+T244E, G23S+A40I+F51I+E56R+R118F+P256T, G23S+A40I+F51 I+E56R+T244E+P256T, G23S+A40I+F51I+V60K+R118F+T244E, G23S+A40I+F51 I+V60K+R118F+P256T, G23S+A40I+F51I+V60K+T244E+P256T, G23S+A40I+F51 I+R118F+T244E+P256T, G23S+A40I+E56R+V60K+R118F+T244E, G23S+A40I+E56R+V60K+R118F+P256T, G23S+A40I+E56R+V60K+T244E+P256T, G23S+A40I+E56R+R118F+T244E+P256T, G23S+A40I+V60K+R118F+T244E+P256T, G23S+F51I+E56R+V60K+R118F+T244E, G23S+F51I+E56R+V60K+R118F+P256T, G23S+F51I+E56R+V60K+T244E+P256T, G23S+F51I+E56R+R118F+T244E+P256T, G23S+F51I+V60K+R118F+T244E+P256T, G23S+E56R+V60K+R118F+T244E+P256T, D27N+A40I+F51 I+E56R+V60K+R118F, D27N+A40I+F51I+E56R+V60K+T244E, D27N+A40I+F51I+E56R+V60K+P256T, D27N+A40I+F51I+E56R+R118F+T244E, D27N+A40I+F51I+E56R+R118F+P256T, D27N+A40I+F51 I+E56R+T244E+P256T, D27N+A40I+F51I+V60K+R118F+T244E, D27N+A40I+F51I+V60K+R118F+P256T, D27N+A40I+F51I+V60K+T244E+P256T, D27N+A40I+F51I+R118F+T244E+P256T, D27N+A40I+E56R+V60K+R118F+T244E, D27N+A40I+E56R+V60K+R118F+P256T, D27N+A40I+E56R+V60K+T244E+P256T, D27N+A40I+E56R+R118F+T244E+P256T, D27N+A40I+V60K+R118F+T244E+P256T, D27N+F51I+E56R+V60K+R118F+T244E, D27N+F51I+E56R+V60K+R118F+P256T, D27N+F51I+E56R+V60K+T244E+P256T, D27N+F51I+E56R+R118F+T244E+P256T, D27N+F51I+V60K+R118F+T244E+P256T, D27N+E56R+V60K+R118F+T244E+P256T, A40I+F51I+E56R+V60K+R118F+T244E, A40I+F51 I+E56R+V60K+R118F+P256T, A40I+F51 I+E56R+V60K+T244E+P256T, A40I+F51I+E56R+R118F+T244E+P256T, A40I+F51I+V60K+R118F+T244E+P256T, A40I+E56R+V60K+R118F+T244E+P256T, F51I+E56R+V60K+R118F+T244E+P256T, G23S+D27N+A40I+F51I+E56R+V60K+R118F, G23S+D27N+A40I+F51I+E56R+V60K+T244E, G23S+D27N+A40I+F51I+E56R+V60K+P256T, G23S+D27N+A40I+F51I+E56R+R118F+T244E,
G23S+D27N+A40I+F51 I+E56R+R118F+P256T,
G23S+D27N+A40I+F51I+E56R+T244E+P256T,
G23S+D27N+A40I+F51I+V60K+R118F+T244E,
G23S+D27N+A40I+F51I+V60K+R118F+P256T,
G23S+D27N+A40I+F51I+V60K+T244E+P256T,
G23S+D27N+A40I+F51I+R118F+T244E+P256T,
G23S+D27N+A40I+E56R+V60K+R118F+T244E,
G23S+D27N+A40I+E56R+V60K+R118F+P256T,
G23S+D27N+A40I+E56R+V60K+T244E+P256T,
G23S+D27N+A40I+E56R+R118F+T244E+P256T,
G23S+D27N+A40I+V60K+R118F+T244E+P256T,
G23S+D27N+F51I+E56R+V60K+R118F+T244E,
G23S+D27N+F51I+E56R+V60K+R118F+P256T,
G23S+D27N+F51I+E56R+V60K+T244E+P256T,
G23S+D27N+F51I+E56R+R118F+T244E+P256T,
G23S+D27N+F51I+V60K+R118F+T244E+P256T,
G23S+D27N+E56R+V60K+R118F+T244E+P256T,
G23S+A40I+F51I+E56R+V60K+R118F+T244E, G23S+A40I+F51I+E56R+V60K+R118F+P256T,
G23S+A40I+F51I+E56R+V60K+T244E+P256T,
G23S+A40I+F51 I+E56R+R118F+T244E+P256T,
G23S+A40I+F51I+V60K+R118F+T244E+P256T,
G23S+A40I+E56R+V60K+R118F+T244E+P256T,
G23S+F51I+E56R+V60K+R118F+T244E+P256T,
D27N+A40I+F51I+E56R+V60K+R118F+T244E, D27N+A40I+F51I+E56R+V60K+R118F+P256T,
D27N+A40I+F51I+E56R+V60K+T244E+P256T,
D27N+A40I+F51I+E56R+R118F+T244E+P256T,
D27N+A40I+F51I+V60K+R118F+T244E+P256T,
D27N+A40I+E56R+V60K+R118F+T244E+P256T,
D27N+F51I+E56R+V60K+R118F+T244E+P256T,
A40I+F51I+E56R+V60K+R118F+T244E+P256T,
G23S+D27N+A40I+F51I+E56R+V60K+R118F+T244E,
G23S+D27N+A40I+F51 I+E56R+V60K+R118F+P256T,
G23S+D27N+A40I+F51I+E56R+V60K+T244E+P256T,
G23S+D27N+A40I+F51I+E56R+R118F+T244E+P256T,
G23S+D27N+A40I+F51I+V60K+R118F+T244E+P256T,
G23S+D27N+A40I+E56R+V60K+R118F+T244E+P256T,
G23S+D27N+F51I+E56R+V60K+R118F+T244E+P256T,
G23S+A40I+F51 I+E56R+V60K+R118F+T244E+P256T,
D27N+A40I+F51I+E56R+V60K+R118F+T244E+P256T,
G23S+D27N+A40I+F51I+E56R+V60K+R118F+T244E+P256T.

In an embodiment a variant of the invention has a substitution corresponding to I252H of SEQ ID NO: 8 and further comprises one of the following substitutions or set of substitutions corresponding to: G23S, D27N, A40I, F51I, E56R, V60K, R118F, T244E, P256T, G23S+D27N, G23S+A40I, G23S+F51I, G23S+E56R, G23S+V60K, G23S+R118F, G23S+T244E, G23S+P256T, D27N+A40I, D27N+F51I, D27N+E56R, D27N+V60K, D27N+R118F, D27N+T244E, D27N+P256T, A40I+F51I, A40I+E56R, A40I+V60K, A40I+R118F, A40I+T244E, A40I+P256T, F51I+E56R, F51I+V60K, F51I+R118F, F51I+T244E, F51I+P256T, E56R+V60K, E56R+R118F, E56R+T244E, E56R+P256T, V60K+R118F, V60K+T244E, V60K+P256T, R118F+T244E, R118F+P256T, T244E+P256T, G23S+D27N+A40I, G23S+D27N+F51I, G23S+D27N+E56R, G23S+D27N+V60K, G23S+D27N+R118F, G23S+D27N+T244E, G23S+D27N+P256T, G23S+A40I+F51I, G23S+A40I+E56R, G23S+A40I+V60K, G23S+A40I+R118F, G23S+A40I+T244E, G23S+A40I+P256T, G23S+F51I+E56R, G23S+F51I+V60K, G23S+F51I+R118F, G23S+F51I+T244E, G23S+F51I+P256T, G23S+E56R+V60K, G23S+E56R+R118F, G23S+E56R+T244E, G23S+E56R+P256T, G23S+V60K+R118F, G23S+V60K+T244E, G23S+V60K+P256T, G23S+R118F+T244E, G23S+R118F+P256T, G23S+T244E+P256T, D27N+A40I+F51I, D27N+A40I+E56R, D27N+A40I+V60K, D27N+A40I+R118F, D27N+A40I+T244E, D27N+A40I+P256T, D27N+F51I+E56R, D27N+F51I+V60K, D27N+F51I+R118F, D27N+F51I+T244E, D27N+F51I+P256T, D27N+E56R+V60K, D27N+E56R+R118F, D27N+E56R+T244E, D27N+E56R+P256T, D27N+V60K+R118F, D27N+V60K+T244E, D27N+V60K+P256T, D27N+R118F+T244E, D27N+R118F+P256T, D27N+T244E+P256T, A40I+F51I+E56R, A40I+F51I+V60K, A40I+F51I+R118F, A40I+F51I+T244E, A40I+F51I+P256T, A40I+E56R+V60K, A40I+E56R+R118F, A40I+E56R+T244E, A40I+E56R+P256T, A40I+V60K+R118F, A40I+V60K+T244E, A40I+V60K+P256T, A40I+R118F+T244E, A40I+R118F+P256T, A40I+T244E+P256T, F51I+E56R+V60K, F51I+E56R+R118F, F51I+E56R+T244E, F51I+E56R+P256T, F51I+V60K+R118F, F51I+V60K+T244E, F51I+V60K+P256T, F51I+R118F+T244E, F51I+R118F+P256T, F51I+T244E+P256T, E56R+V60K+R118F, E56R+V60K+T244E, E56R+V60K+P256T, E56R+R118F+T244E, E56R+R118F+P256T, E56R+T244E+P256T, V60K+R118F+T244E, V60K+R118F+P256T, V60K+T244E+P256T, R118F+T244E+P256T, G23S+D27N+A40I+F51I, G23S+D27N+A40I+E56R, G23S+D27N+A40I+V60K, G23S+D27N+A40I+R118F, G23S+D27N+A40I+T244E, G23S+D27N+A40I+P256T, G23S+D27N+F51I+E56R, G23S+D27N+F51I+V60K, G23S+D27N+F51I+R118F, G23S+D27N+F51I+T244E, G23S+D27N+F51I+P256T, G23S+D27N+E56R+V60K, G23S+D27N+E56R+R118F, G23S+D27N+E56R+T244E, G23S+D27N+E56R+P256T, G23S+D27N+V60K+R118F, G23S+D27N+V60K+T244E, G23S+D27N+V60K+P256T, G23S+D27N+R118F+T244E, G23S+D27N+R118F+P256T, G23S+D27N+T244E+P256T, G23S+A40I+F51I+E56R, G23S+A40I+F51I+V60K, G23S+A40I+F51I+R118F, G23S+A40I+F51I+T244E, G23S+A40I+F51I+P256T, G23S+A40I+E56R+V60K, G23S+A40I+E56R+R118F, G23S+A40I+E56R+T244E, G23S+A40I+E56R+P256T, G23S+A40I+V60K+R118F, G23S+A40I+V60K+T244E, G23S+A40I+V60K+P256T, G23S+A40I+R118F+T244E, G23S+A40I+R118F+P256T, G23S+A40I+T244E+P256T, G23S+F51I+E56R+V60K, G23S+F51I+E56R+R118F, G23S+F51I+E56R+T244E, G23S+F51I+E56R+P256T, G23S+F51I+V60K+R118F, G23S+F51I+V60K+T244E, G23S+F51I+V60K+P256T, G23S+F51I+R118F+T244E, G23S+F51I+R118F+P256T, G23S+F51I+T244E+P256T, G23S+E56R+V60K+R118F, G23S+E56R+V60K+T244E, G23S+E56R+V60K+P256T, G23S+E56R+R118F+T244E, G23S+E56R+R118F+P256T, G23S+E56R+T244E+P256T, G23S+V60K+R118F+T244E, G23S+V60K+R118F+P256T, G23S+V60K+T244E+P256T, G23S+R118F+T244E+P256T, D27N+A40I+F51I+E56R, D27N+A40I+F51I+V60K, D27N+A40I+F51I+R118F, D27N+A40I+F51I+T244E, D27N+A40I+F51I+P256T, D27N+A40I+E56R+V60K, D27N+A40I+E56R+R118F, D27N+A40I+E56R+T244E, D27N+A40I+E56R+P256T, D27N+A40I+V60K+R118F, D27N+A40I+V60K+T244E, D27N+A40I+V60K+P256T, D27N+A40I+R118F+T244E, D27N+A40I+R118F+P256T, D27N+A40I+T244E+P256T, D27N+F51I+E56R+V60K, D27N+F51I+E56R+R118F, D27N+F51I+E56R+T244E, D27N+F51I+E56R+P256T, D27N+F51I+V60K+R118F, D27N+F51I+V60K+T244E, D27N+F51I+V60K+P256T, D27N+F51I+R118F+T244E, D27N+F51I+R118F+P256T, D27N+F51I+T244E+P256T, D27N+E56R+V60K+R118F, D27N+E56R+V60K+T244E, D27N+E56R+V60K+P256T, D27N+E56R+R118F+T244E, D27N+E56R+R118F+P256T, D27N+E56R+T244E+P256T, D27N+V60K+R118F+T244E, D27N+V60K+R118F+P256T, D27N+V60K+T244E+P256T, D27N+R118F+T244E+P256T, A40I+F51I+E56R+V60K, A40I+F51I+E56R+R118F, A40I+F51I+E56R+T244E, A40I+F51I+E56R+P256T, A40I+F51I+V60K+R118F, A40I+F51I+V60K+T244E, A40I+F51I+V60K+P256T, A40I+F51I+R118F+T244E, A40I+F51I+R118F+P256T, A40I+F51I+T244E+P256T, A40I+E56R+V60K+R118F, A40I+E56R+V60K+T244E, A40I+E56R+V60K+P256T, A40I+E56R+R118F+T244E, A40I+E56R+R118F+P256T, A40I+E56R+T244E+P256T, A40I+V60K+R118F+T244E, A40I+V60K+R118F+P256T, A40I+V60K+T244E+P256T, A40I+R118F+T244E+P256T, F51I+E56R+V60K+R118F, F51I+E56R+V60K+T244E, F51I+E56R+V60K+P256T, F51I+E56R+R118F+T244E, F51I+E56R+R118F+P256T, F51I+E56R+T244E+P256T, F51I+V60K+R118F+T244E, F51I+V60K+R118F+P256T, F51I+V60K+T244E+P256T, F51I+R118F+T244E+P256T, E56R+V60K+R118F+T244E, E56R+V60K+R118F+P256T, E56R+V60K+T244E+P256T, E56R+R118F+T244E+P256T, V60K+R118F+T244E+P256T, G23S+D27N+A40I+F51I+E56R, G23S+D27N+A40I+F51I+V60K, G23S+D27N+A40I+F51I+R118F, G23S+D27N+A40I+F51I+T244E, G23S+D27N+A40I+F51 I+P256T, G23S+D27N+A40I+E56R+V60K, G23S+D27N+A40I+E56R+R118F, G23S+D27N+A40I+E56R+T244E, G23S+D27N+A40I+E56R+P256T, G23S+D27N+A40I+V60K+R118F, G23S+D27N+A40I+V60K+T244E, G23S+D27N+A40I+V60K+P256T, G23S+D27N+A40I+R118F+T244E, G23S+D27N+A40I+R118F+P256T, G23S+D27N+A40I+T244E+P256T, G23S+D27N+F51I+E56R+V60K, G23S+D27N+F51 I+E56R+R118F, G23S+D27N+F51I+E56R+T244E, G23S+D27N+F51 I+E56R+P256T, G23S+D27N+F51I+V60K+R118F, G23S+D27N+F51 I+V60K+T244E, G23S+D27N+F51I+V60K+P256T, G23S+D27N+F51I+R118F+T244E, G23S+D27N+F51I+R118F+P256T, G23S+D27N+F51I+T244E+P256T, G23S+D27N+E56R+V60K+R118F, G23S+D27N+E56R+V60K+T244E, G23S+D27N+E56R+V60K+P256T, G23S+D27N+E56R+R118F+T244E, G23S+D27N+E56R+R118F+P256T, G23S+D27N+E56R+T244E+P256T, G23S+D27N+V60K+R118F+T244E, G23S+D27N+V60K+R118F+P256T, G23S+D27N+V60K+T244E+P256T, G23S+D27N+R118F+T244E+P256T, G23S+A40I+F51 I+E56R+V60K, G23S+A40I+F51 I+E56R+R118F, G23S+A40I+F51I+E56R+T244E, G23S+A40I+F51I+E56R+P256T, G23S+A40I+F51 I+V60K+R118F, G23S+A40I+F51I+V60K+T244E, G23S+A40I+F51I+V60K+P256T, G23S+A40I+F51I+R118F+T244E, G23S+A40I+F51I+R118F+P256T, G23S+A40I+F51I+T244E+P256T, G23S+A40I+E56R+V60K+R118F, G23S+A40I+E56R+V60K+T244E, G23S+A40I+E56R+V60K+P256T, G23S+A40I+E56R+R118F+T244E, G23S+A40I+E56R+R118F+P256T, G23S+A40I+E56R+T244E+P256T, G23S+A40I+V60K+R118F+T244E, G23S+A40I+V60K+R118F+P256T, G23S+A40I+V60K+T244E+P256T, G23S+A40I+R118F+T244E+P256T, G23S+F51I+E56R+V60K+R118F, G23S+F51I+E56R+V60K+T244E, G23S+F51I+E56R+V60K+P256T, G23S+F51 I+E56R+R118F+T244E, G23S+F51I+E56R+R118F+P256T, G23S+F51 I+E56R+T244E+P256T, G23S+F51I+V60K+R118F+T244E, G23S+F51 I+V60K+R118F+P256T, G23S+F51 I+V60K+T244E+P256T, G23S+F51I+R118F+T244E+P256T, G23S+E56R+V60K+R118F+T244E, G23S+E56R+V60K+R118F+P256T, G23S+E56R+V60K+T244E+P256T, G23S+E56R+R118F+T244E+P256T, G23S+V60K+R118F+T244E+P256T, D27N+A40I+F51I+E56R+V60K, D27N+A40I+F51 I+E56R+R118F, D27N+A40I+F51 I+E56R+T244E, D27N+A40I+F51 I+E56R+P256T, D27N+A40I+F51I+V60K+R118F, D27N+A40I+F51 I+V60K+T244E, D27N+A40I+F51I+V60K+P256T, D27N+A40I+F51I+R118F+T244E, D27N+A40I+F51I+R118F+P256T, D27N+A40I+F51I+T244E+P256T, D27N+A40I+E56R+V60K+R118F, D27N+A40I+E56R+V60K+T244E, D27N+A40I+E56R+V60K+P256T, D27N+A40I+E56R+R118F+T244E, D27N+A40I+E56R+R118F+P256T, D27N+A40I+E56R+T244E+P256T, D27N+A40I+V60K+R118F+T244E, D27N+A40I+V60K+R118F+P256T, D27N+A40I+V60K+T244E+P256T, D27N+A40I+R118F+T244E+P256T, D27N+F51 I+E56R+V60K+R118F, D27N+F51 I+E56R+V60K+T244E, D27N+F51 I+E56R+V60K+P256T, D27N+F51 I+E56R+R118F+T244E, D27N+F51I+E56R+R118F+P256T, D27N+F51I+E56R+T244E+P256T, D27N+F51I+V60K+R118F+T244E, D27N+F51I+V60K+R118F+P256T, D27N+F51I+V60K+T244E+P256T, D27N+F51I+R118F+T244E+P256T, D27N+E56R+V60K+R118F+T244E, D27N+E56R+V60K+R118F+P256T, D27N+E56R+V60K+T244E+P256T, D27N+E56R+R118F+T244E+P256T, D27N+V60K+R118F+T244E+P256T, A40I+F51I+E56R+V60K+R118F, A40I+F51I+E56R+V60K+T244E, A40I+F51I+E56R+V60K+P256T, A40I+F51I+E56R+R118F+T244E, A40I+F51I+E56R+R118F+P256T, A40I+F51I+E56R+T244E+P256T, A40I+F51I+V60K+R118F+T244E, A40I+F51 I+V60K+R118F+P256T, A40I+F51 I+V60K+T244E+P256T, A40I+F51I+R118F+T244E+P256T, A40I+E56R+V60K+R118F+T244E, A40I+E56R+V60K+R118F+P256T, A40I+E56R+V60K+T244E+P256T, A40I+E56R+R118F+T244E+P256T, A40I+V60K+R118F+T244E+P256T, F51I+E56R+V60K+R118F+T244E, F51I+E56R+V60K+R118F+P256T, F51I+E56R+V60K+T244E+P256T, F51I+E56R+R118F+T244E+P256T, F51I+V60K+R118F+T244E+P256T, E56R+V60K+R118F+T244E+P256T, G23S+D27N+A40I+F51I+E56R+V60K, G23S+D27N+A40I+F51 I+E56R+R118F, G23S+D27N+A40I+F51I+E56R+T244E, G23S+D27N+A40I+F51 I+E56R+P256T, G23S+D27N+A40I+F51I+V60K+R118F, G23S+D27N+A40I+F51I+V60K+T244E, G23S+D27N+A40I+F51I+V60K+P256T, G23S+D27N+A40I+F51I+R118F+T244E, G23S+D27N+A40I+F51 I+R118F+P256T, G23S+D27N+A40I+F51I+T244E+P256T, G23S+D27N+A40I+E56R+V60K+R118F, G23S+D27N+A40I+E56R+V60K+T244E, G23S+D27N+A40I+E56R+V60K+P256T, G23S+D27N+A40I+E56R+R118F+T244E, G23S+D27N+A40I+E56R+R118F+P256T, G23S+D27N+A40I+E56R+T244E+P256T, G23S+D27N+A40I+V60K+R118F+T244E, G23S+D27N+A40I+V60K+R118F+P256T, G23S+D27N+A40I+V60K+T244E+P256T, G23S+D27N+A40I+R118F+T244E+P256T, G23S+D27N+F51I+E56R+V60K+R118F, G23S+D27N+F51 I+E56R+V60K+T244E, G23S+D27N+F51 I+E56R+V60K+P256T, G23S+D27N+F51I+E56R+R118F+T244E, G23S+D27N+F51I+E56R+R118F+P256T, G23S+D27N+F51I+E56R+T244E+P256T, G23S+D27N+F51I+V60K+R118F+T244E, G23S+D27N+F51I+V60K+R118F+P256T, G23S+D27N+F51I+V60K+T244E+P256T, G23S+D27N+F51I+R118F+T244E+P256T, G23S+D27N+E56R+V60K+R118F+T244E, G23S+D27N+E56R+V60K+R118F+P256T, G23S+D27N+E56R+V60K+T244E+P256T, G23S+D27N+E56R+R118F+T244E+P256T, G23S+D27N+V60K+R118F+T244E+P256T, G23S+A40I+F51I+E56R+V60K+R118F, G23S+A40I+F51I+E56R+V60K+T244E, G23S+A40I+F51I+E56R+V60K+P256T, G23S+A40I+F51I+E56R+R118F+T244E, G23S+A40I+F51I+E56R+R118F+P256T, G23S+A40I+F51 I+E56R+T244E+P256T, G23S+A40I+F51I+V60K+R118F+T244E, G23S+A40I+F51 I+V60K+R118F+P256T, G23S+A40I+F51I+V60K+T244E+P256T, G23S+A40I+F51I+R118F+T244E+P256T, G23S+A40I+E56R+V60K+R118F+T244E, G23S+A40I+E56R+V60K+R118F+P256T, G23S+A40I+E56R+V60K+T244E+P256T, G23S+A40I+E56R+R118F+T244E+P256T, G23S+A40I+V60K+R118F+T244E+P256T, G23S+F51I+E56R+V60K+R118F+T244E, G23S+F51I+E56R+V60K+R118F+P256T, G23S+F51I+E56R+V60K+T244E+P256T, G23S+F51I+E56R+R118F+T244E+P256T, G23S+F51I+V60K+R118F+T244E+P256T, G23S+E56R+V60K+R118F+T244E+P256T, D27N+A40I+F51 I+E56R+V60K+R118F, D27N+A40I+F51 I+E56R+V60K+T244E, D27N+A40I+F51 I+E56R+V60K+P256T, D27N+A40I+F51 I+E56R+R118F+T244E, D27N+A40I+F51I+E56R+R118F+P256T, D27N+A40I+F51 I+E56R+T244E+P256T, D27N+A40I+F51I+V60K+R118F+T244E, D27N+A40I+F51I+V60K+R118F+P256T, D27N+A40I+F51I+V60K+T244E+P256T, D27N+A40I+F51I+R118F+T244E+P256T, D27N+A40I+E56R+V60K+R118F+T244E, D27N+A40I+E56R+V60K+R118F+P256T, D27N+A40I+E56R+V60K+T244E+P256T, D27N+A40I+E56R+R118F+T244E+P256T, D27N+A40I+V60K+R118F+T244E+P256T, D27N+F51I+E56R+V60K+R118F+T244E, D27N+F51I+E56R+V60K+R118F+P256T, D27N+F51 I+E56R+V60K+T244E+P256T, D27N+F51I+E56R+R118F+T244E+P256T, D27N+F51I+V60K+R118F+T244E+P256T, D27N+E56R+V60K+R118F+T244E+P256T, A40I+F51I+E56R+V60K+R118F+T244E, A40I+F51I+E56R+V60K+R118F+P256T, A40I+F51I+E56R+V60K+T244E+P256T, A40I+F51I+E56R+R118F+T244E+P256T, A40I+F51 I+V60K+R118F+T244E+P256T, A40I+E56R+V60K+R118F+T244E+P256T, F51I+E56R+V60K+R118F+T244E+P256T, G23S+D27N+A40I+F51I+E56R+V60K+R118F, G23S+D27N+A40I+F51I+E56R+V60K+T244E, G23S+D27N+A40I+F51I+E56R+V60K+P256T, G23S+D27N+A40I+F51I+E56R+R118F+T244E,
G23S+D27N+A40I+F51I+E56R+R118F+P256T,
G23S+D27N+A40I+F51I+E56R+T244E+P256T,
G23S+D27N+A40I+F51I+V60K+R118F+T244E,
G23S+D27N+A40I+F51I+V60K+R118F+P256T,
G23S+D27N+A40I+F51I+V60K+T244E+P256T,
G23S+D27N+A40I+F51I+R118F+T244E+P256T,
G23S+D27N+A40I+E56R+V60K+R118F+T244E,
G23S+D27N+A40I+E56R+V60K+R118F+P256T,
G23S+D27N+A40I+E56R+V60K+T244E+P256T,
G23S+D27N+A40I+E56R+R118F+T244E+P256T,
G23S+D27N+A40I+V60K+R118F+T244E+P256T,
G23S+D27N+F51I+E56R+V60K+R118F+T244E,
G23S+D27N+F51I+E56R+V60K+R118F+P256T,
G23S+D27N+F51I+E56R+V60K+T244E+P256T,
G23S+D27N+F51I+E56R+R118F+T244E+P256T,
G23S+D27N+F51I+V60K+R118F+T244E+P256T,
G23S+D27N+E56R+V60K+R118F+T244E+P256T,
G23S+A40I+F51I+E56R+V60K+R118F+T244E, G23S+A40I+F51I+E56R+V60K+R118F+P256T,
G23S+A40I+F51I+E56R+V60K+T244E+P256T,
G23S+A40I+F51I+E56R+R118F+T244E+P256T,
G23S+A40I+F51I+V60K+R118F+T244E+P256T,
G23S+A40I+E56R+V60K+R118F+T244E+P256T,
G23S+F51I+E56R+V60K+R118F+T244E+P256T,
D27N+A40I+F51I+E56R+V60K+R118F+T244E, D27N+A40I+F51I+E56R+V60K+R118F+P256T,
D27N+A40I+F51I+E56R+V60K+T244E+P256T,
D27N+A40I+F51I+E56R+R118F+T244E+P256T,
D27N+A40I+F51I+V60K+R118F+T244E+P256T,
D27N+A40I+E56R+V60K+R118F+T244E+P256T,
D27N+F51I+E56R+V60K+R118F+T244E+P256T,
A40I+F51I+E56R+V60K+R118F+T244E+P256T,
G23S+D27N+A40I+F51I+E56R+V60K+R118F+T244E,
G23S+D27N+A40I+F51I+E56R+V60K+R118F+P256T,
G23S+D27N+A40I+F51I+E56R+V60K+T244E+P256T,
G23S+D27N+A40I+F51I+E56R+R118F+T244E+P256T,
G23S+D27N+A40I+F51I+V60K+R118F+T244E+P256T,
G23S+D27N+A40I+E56R+V60K+R118F+T244E+P256T,
G23S+D27N+F51I+E56R+V60K+R118F+T244E+P256T,
G23S+A40I+F51I+E56R+V60K+R118F+T244E+P256T,
D27N+A40I+F51 I+E56R+V60K+R118F+T244E+P256T,
G23S+D27N+A40I+F51I+E56R+V60K+R118F+T244E+P256T.

In an embodiment, a variant of the invention has a substitution corresponding to L269H of SEQ ID NO: 8 and further comprises one of the following substitutions or set of substitutions corresponding to: G23S, D27N, A40I, F51I, E56R, V60K, R118F, T244E, P256T, G23S+D27N, G23S+A40I, G23S+F51I, G23S+E56R, G23S+V60K, G23S+R118F, G23S+T244E, G23S+P256T, D27N+A40I, D27N+F51I, D27N+E56R, D27N+V60K, D27N+R118F, D27N+T244E, D27N+P256T, A40I+F51I, A40I+E56R, A40I+V60K, A40I+R118F, A40I+T244E, A40I+P256T, F51I+E56R, F51I+V60K, F51I+R118F, F51I+T244E, F51I+P256T, E56R+V60K, E56R+R118F, E56R+T244E, E56R+P256T, V60K+R118F, V60K+T244E, V60K+P256T, R118F+T244E, R118F+P256T, T244E+P256T, G23S+D27N+A40I, G23S+D27N+F51I, G23S+D27N+E56R, G23S+D27N+V60K, G23S+D27N+R118F, G23S+D27N+T244E, G23S+D27N+P256T, G23S+A40I+F51I, G23S+A40I+E56R, G23S+A40I+V60K, G23S+A40I+R118F, G23S+A40I+T244E, G23S+A40I+P256T, G23S+F51I+E56R, G23S+F51I+V60K, G23S+F51I+R118F, G23S+F51I+T244E, G23S+F51I+P256T, G23S+E56R+V60K, G23S+E56R+R118F, G23S+E56R+T244E, G23S+E56R+P256T, G23S+V60K+R118F, G23S+V60K+T244E, G23S+V60K+P256T, G23S+R118F+T244E, G23S+R118F+P256T, G23S+T244E+P256T, D27N+A40I+F51I, D27N+A40I+E56R, D27N+A40I+V60K, D27N+A40I+R118F, D27N+A40I+T244E, D27N+A40I+P256T, D27N+F51I+E56R, D27N+F51I+V60K, D27N+F51I+R118F, D27N+F51I+T244E, D27N+F51I+P256T, D27N+E56R+V60K, D27N+E56R+R118F, D27N+E56R+T244E, D27N+E56R+P256T, D27N+V60K+R118F, D27N+V60K+T244E, D27N+V60K+P256T, D27N+R118F+T244E, D27N+R118F+P256T, D27N+T244E+P256T, A40I+F51I+E56R, A40I+F51I+V60K, A40I+F51I+R118F, A40I+F51I+T244E, A40I+F51I+P256T, A40I+E56R+V60K, A40I+E56R+R118F, A40I+E56R+T244E, A40I+E56R+P256T, A40I+V60K+R118F, A40I+V60K+T244E, A40I+V60K+P256T, A40I+R118F+T244E, A40I+R118F+P256T, A40I+T244E+P256T, F51I+E56R+V60K, F51I+E56R+R118F, F51I+E56R+T244E, F51I+E56R+P256T, F51I+V60K+R118F, F51I+V60K+T244E, F51I+V60K+P256T, F51I+R118F+T244E, F51I+R118F+P256T, F51I+T244E+P256T, E56R+V60K+R118F, E56R+V60K+T244E, E56R+V60K+P256T, E56R+R118F+T244E, E56R+R118F+P256T, E56R+T244E+P256T, V60K+R118F+T244E, V60K+R118F+P256T, V60K+T244E+P256T, R118F+T244E+P256T, G23S+D27N+A40I+F51I, G23S+D27N+A40I+E56R, G23S+D27N+A40I+V60K, G23S+D27N+A40I+R118F, G23S+D27N+A40I+T244E, G23S+D27N+A40I+P256T, G23S+D27N+F51I+E56R, G23S+D27N+F51I+V60K, G23S+D27N+F51I+R118F, G23S+D27N+F51I+T244E, G23S+D27N+F51I+P256T, G23S+D27N+E56R+V60K, G23S+D27N+E56R+R118F, G23S+D27N+E56R+T244E, G23S+D27N+E56R+P256T, G23S+D27N+V60K+R118F, G23S+D27N+V60K+T244E, G23S+D27N+V60K+P256T, G23S+D27N+R118F+T244E, G23S+D27N+R118F+P256T, G23S+D27N+T244E+P256T, G23S+A40I+F51I+E56R, G23S+A40I+F51I+V60K, G23S+A40I+F51I+R118F, G23S+A40I+F51I+T244E, G23S+A40I+F51I+P256T, G23S+A40I+E56R+V60K, G23S+A40I+E56R+R118F, G23S+A40I+E56R+T244E, G23S+A40I+E56R+P256T, G23S+A40I+V60K+R118F, G23S+A40I+V60K+T244E, G23S+A40I+V60K+P256T, G23S+A40I+R118F+T244E, G23S+A40I+R118F+P256T, G23S+A40I+T244E+P256T, G23S+F51I+E56R+V60K, G23S+F51I+E56R+R118F, G23S+F51I+E56R+T244E, G23S+F51I+E56R+P256T, G23S+F51I+V60K+R118F, G23S+F51I+V60K+T244E, G23S+F51I+V60K+P256T, G23S+F51I+R118F+T244E, G23S+F51I+R118F+P256T, G23S+F51I+T244E+P256T, G23S+E56R+V60K+R118F, G23S+E56R+V60K+T244E, G23S+E56R+V60K+P256T, G23S+E56R+R118F+T244E, G23S+E56R+R118F+P256T, G23S+E56R+T244E+P256T, G23S+V60K+R118F+T244E, G23S+V60K+R118F+P256T, G23S+V60K+T244E+P256T, G23S+R118F+T244E+P256T, D27N+A40I+F51I+E56R, D27N+A40I+F51I+V60K, D27N+A40I+F51I+R118F, D27N+A40I+F51I+T244E, D27N+A40I+F51I+P256T, D27N+A40I+E56R+V60K, D27N+A40I+E56R+R118F, D27N+A40I+E56R+T244E, D27N+A40I+E56R+P256T, D27N+A40I+V60K+R118F, D27N+A40I+V60K+T244E, D27N+A40I+V60K+P256T, D27N+A40I+R118F+T244E, D27N+A40I+R118F+P256T, D27N+A40I+T244E+P256T, D27N+F51I+E56R+V60K, D27N+F51I+E56R+R118F, D27N+F51I+E56R+T244E, D27N+F51I+E56R+P256T, D27N+F51I+V60K+R118F, D27N+F51I+V60K+T244E, D27N+F51I+V60K+P256T, D27N+F51I+R118F+T244E, D27N+F51I+R118F+P256T, D27N+F51I+T244E+P256T, D27N+E56R+V60K+R118F, D27N+E56R+V60K+T244E, D27N+E56R+V60K+P256T, D27N+E56R+R118F+T244E, D27N+E56R+R118F+P256T, D27N+E56R+T244E+P256T, D27N+V60K+R118F+T244E, D27N+V60K+R118F+P256T, D27N+V60K+T244E+P256T, D27N+R118F+T244E+P256T, A40I+F51I+E56R+V60K, A40I+F51I+E56R+R118F, A40I+F51I+E56R+T244E, A40I+F51I+E56R+P256T, A40I+F51I+V60K+R118F, A40I+F51I+V60K+T244E, A40I+F51I+V60K+P256T, A40I+F51I+R118F+T244E, A40I+F51I+R118F+P256T, A40I+F51I+T244E+P256T, A40I+E56R+V60K+R118F, A40I+E56R+V60K+T244E, A40I+E56R+V60K+P256T, A40I+E56R+R118F+T244E, A40I+E56R+R118F+P256T, A40I+E56R+T244E+P256T, A40I+V60K+R118F+T244E, A40I+V60K+R118F+P256T, A40I+V60K+T244E+P256T, A40I+R118F+T244E+P256T, F51I+E56R+V60K+R118F, F51I+E56R+V60K+T244E, F51I+E56R+V60K+P256T, F51I+E56R+R118F+T244E, F51I+E56R+R118F+P256T, F51I+E56R+T244E+P256T, F51I+V60K+R118F+T244E, F51I+V60K+R118F+P256T, F51I+V60K+T244E+P256T, F51I+R118F+T244E+P256T, E56R+V60K+R118F+T244E, E56R+V60K+R118F+P256T, E56R+V60K+T244E+P256T, E56R+R118F+T244E+P256T, V60K+R118F+T244E+P256T, G23S+D27N+A40I+F51I+E56R, G23S+D27N+A40I+F51I+V60K, G23S+D27N+A40I+F51I+R118F, G23S+D27N+A40I+F51 I+T244E, G23S+D27N+A40I+F51I+P256T, G23S+D27N+A40I+E56R+V60K, G23S+D27N+A40I+E56R+R118F, G23S+D27N+A40I+E56R+T244E, G23S+D27N+A40I+E56R+P256T, G23S+D27N+A40I+V60K+R118F, G23S+D27N+A40I+V60K+T244E, G23S+D27N+A40I+V60K+P256T, G23S+D27N+A40I+R118F+T244E, G23S+D27N+A40I+R118F+P256T, G23S+D27N+A40I+T244E+P256T, G23S+D27N+F51I+E56R+V60K, G23S+D27N+F51 I+E56R+R118F, G23S+D27N+F51I+E56R+T244E, G23S+D27N+F51I+E56R+P256T, G23S+D27N+F51I+V60K+R118F, G23S+D27N+F51 I+V60K+T244E, G23S+D27N+F51 I+V60K+P256T, G23S+D27N+F51 I+R118F+T244E, G23S+D27N+F51 I+R118F+P256T, G23S+D27N+F51I+T244E+P256T, G23S+D27N+E56R+V60K+R118F, G23S+D27N+E56R+V60K+T244E, G23S+D27N+E56R+V60K+P256T, G23S+D27N+E56R+R118F+T244E, G23S+D27N+E56R+R118F+P256T, G23S+D27N+E56R+T244E+P256T, G23S+D27N+V60K+R118F+T244E, G23S+D27N+V60K+R118F+P256T, G23S+D27N+V60K+T244E+P256T, G23S+D27N+R118F+T244E+P256T, G23S+A40I+F51 I+E56R+V60K, G23S+A40I+F51 I+E56R+R118F, G23S+A40I+F51I+E56R+T244E, G23S+A40I+F51I+E56R+P256T, G23S+A40I+F51I+V60K+R118F, G23S+A40I+F51I+V60K+T244E, G23S+A40I+F51I+V60K+P256T, G23S+A40I+F51 I+R118F+T244E, G23S+A40I+F51I+R118F+P256T, G23S+A40I+F51I+T244E+P256T, G23S+A40I+E56R+V60K+R118F, G23S+A40I+E56R+V60K+T244E, G23S+A40I+E56R+V60K+P256T, G23S+A40I+E56R+R118F+T244E, G23S+A40I+E56R+R118F+P256T, G23S+A40I+E56R+T244E+P256T, G23S+A40I+V60K+R118F+T244E, G23S+A40I+V60K+R118F+P256T, G23S+A40I+V60K+T244E+P256T, G23S+A40I+R118F+T244E+P256T, G23S+F51I+E56R+V60K+R118F, G23S+F51 I+E56R+V60K+T244E, G23S+F51I+E56R+V60K+P256T, G23S+F51I+E56R+R118F+T244E, G23S+F51I+E56R+R118F+P256T, G23S+F51I+E56R+T244E+P256T, G23S+F51I+V60K+R118F+T244E, G23S+F51I+V60K+R118F+P256T, G23S+F51I+V60K+T244E+P256T, G23S+F51I+R118F+T244E+P256T, G23S+E56R+V60K+R118F+T244E, G23S+E56R+V60K+R118F+P256T, G23S+E56R+V60K+T244E+P256T, G23S+E56R+R118F+T244E+P256T, G23S+V60K+R118F+T244E+P256T, D27N+A40I+F51I+E56R+V60K, D27N+A40I+F51 I+E56R+R118F, D27N+A40I+F51I+E56R+T244E, D27N+A40I+F51I+E56R+P256T, D27N+A40I+F51I+V60K+R118F, D27N+A40I+F51 I+V60K+T244E, D27N+A40I+F51I+V60K+P256T, D27N+A40I+F51I+R118F+T244E, D27N+A40I+F51 I+R118F+P256T, D27N+A40I+F51 I+T244E+P256T, D27N+A40I+E56R+V60K+R118F, D27N+A40I+E56R+V60K+T244E, D27N+A40I+E56R+V60K+P256T, D27N+A40I+E56R+R118F+T244E, D27N+A40I+E56R+R118F+P256T, D27N+A40I+E56R+T244E+P256T, D27N+A40I+V60K+R118F+T244E, D27N+A40I+V60K+R118F+P256T, D27N+A40I+V60K+T244E+P256T, D27N+A40I+R118F+T244E+P256T, D27N+F51 I+E56R+V60K+R118F, D27N+F51 I+E56R+V60K+T244E, D27N+F51I+E56R+V60K+P256T, D27N+F51I+E56R+R118F+T244E, D27N+F51I+E56R+R118F+P256T, D27N+F51I+E56R+T244E+P256T, D27N+F51 I+V60K+R118F+T244E, D27N+F51 I+V60K+R118F+P256T, D27N+F51I+V60K+T244E+P256T, D27N+F51I+R118F+T244E+P256T, D27N+E56R+V60K+R118F+T244E, D27N+E56R+V60K+R118F+P256T, D27N+E56R+V60K+T244E+P256T, D27N+E56R+R118F+T244E+P256T, D27N+V60K+R118F+T244E+P256T, A40I+F51I+E56R+V60K+R118F, A40I+F51I+E56R+V60K+T244E, A40I+F51 I+E56R+V60K+P256T, A40I+F51I+E56R+R118F+T244E, A40I+F51I+E56R+R118F+P256T, A40I+F51I+E56R+T244E+P256T, A40I+F51I+V60K+R118F+T244E, A40I+F51I+V60K+R118F+P256T, A40I+F51I+V60K+T244E+P256T, A40I+F51I+R118F+T244E+P256T, A40I+E56R+V60K+R118F+T244E, A40I+E56R+V60K+R118F+P256T, A40I+E56R+V60K+T244E+P256T, A40I+E56R+R118F+T244E+P256T, A40I+V60K+R118F+T244E+P256T, F51I+E56R+V60K+R118F+T244E, F51I+E56R+V60K+R118F+P256T, F51I+E56R+V60K+T244E+P256T, F51I+E56R+R118F+T244E+P256T, F51I+V60K+R118F+T244E+P256T, E56R+V60K+R118F+T244E+P256T, G23S+D27N+A40I+F51I+E56R+V60K, G23S+D27N+A40I+F51 I+E56R+R118F, G23S+D27N+A40I+F51I+E56R+T244E, G23S+D27N+A40I+F51 I+E56R+P256T, G23S+D27N+A40I+F51I+V60K+R118F, G23S+D27N+A40I+F51 I+V60K+T244E, G23S+D27N+A40I+F51I+V60K+P256T, G23S+D27N+A40I+F51I+R118F+T244E, G23S+D27N+A40I+F51 I+R118F+P256T, G23S+D27N+A40I+F51I+T244E+P256T, G23S+D27N+A40I+E56R+V60K+R118F, G23S+D27N+A40I+E56R+V60K+T244E, G23S+D27N+A40I+E56R+V60K+P256T, G23S+D27N+A40I+E56R+R118F+T244E, G23S+D27N+A40I+E56R+R118F+P256T, G23S+D27N+A40I+E56R+T244E+P256T, G23S+D27N+A40I+V60K+R118F+T244E, G23S+D27N+A40I+V60K+R118F+P256T, G23S+D27N+A40I+V60K+T244E+P256T, G23S+D27N+A40I+R118F+T244E+P256T, G23S+D27N+F51I+E56R+V60K+R118F, G23S+D27N+F51I+E56R+V60K+T244E, G23S+D27N+F51 I+E56R+V60K+P256T, G23S+D27N+F51I+E56R+R118F+T244E, G23S+D27N+F51I+E56R+R118F+P256T, G23S+D27N+F51 I+E56R+T244E+P256T, G23S+D27N+F51I+V60K+R118F+T244E, G23S+D27N+F51I+V60K+R118F+P256T, G23S+D27N+F51I+V60K+T244E+P256T, G23S+D27N+F51I+R118F+T244E+P256T, G23S+D27N+E56R+V60K+R118F+T244E, G23S+D27N+E56R+V60K+R118F+P256T, G23S+D27N+E56R+V60K+T244E+P256T, G23S+D27N+E56R+R118F+T244E+P256T, G23S+D27N+V60K+R118F+T244E+P256T, G23S+A40I+F51I+E56R+V60K+R118F, G23S+A40I+F51I+E56R+V60K+T244E, G23S+A40I+F51I+E56R+V60K+P256T, G23S+A40I+F51I+E56R+R118F+T244E, G23S+A40I+F51I+E56R+R118F+P256T, G23S+A40I+F51I+E56R+T244E+P256T, G23S+A40I+F51I+V60K+R118F+T244E, G23S+A40I+F51 I+V60K+R118F+P256T, G23S+A40I+F51I+V60K+T244E+P256T, G23S+A40I+F51 I+R118F+T244E+P256T, G23S+A40I+E56R+V60K+R118F+T244E, G23S+A40I+E56R+V60K+R118F+P256T, G23S+A40I+E56R+V60K+T244E+P256T, G23S+A40I+E56R+R118F+T244E+P256T, G23S+A40I+V60K+R118F+T244E+P256T, G23S+F51I+E56R+V60K+R118F+T244E, G23S+F51I+E56R+V60K+R118F+P256T, G23S+F51I+E56R+V60K+T244E+P256T, G23S+F51I+E56R+R118F+T244E+P256T, G23S+F51I+V60K+R118F+T244E+P256T, G23S+E56R+V60K+R118F+T244E+P256T, D27N+A40I+F51 I+E56R+V60K+R118F, D27N+A40I+F51 I+E56R+V60K+T244E, D27N+A40I+F51I+E56R+V60K+P256T, D27N+A40I+F51I+E56R+R118F+T244E, D27N+A40I+F51I+E56R+R118F+P256T, D27N+A40I+F51I+E56R+T244E+P256T, D27N+A40I+F51I+V60K+R118F+T244E, D27N+A40I+F51I+V60K+R118F+P256T, D27N+A40I+F51I+V60K+T244E+P256T, D27N+A40I+F51I+R118F+T244E+P256T, D27N+A40I+E56R+V60K+R118F+T244E, D27N+A40I+E56R+V60K+R118F+P256T, D27N+A40I+E56R+V60K+T244E+P256T, D27N+A40I+E56R+R118F+T244E+P256T, D27N+A40I+V60K+R118F+T244E+P256T, D27N+F51I+E56R+V60K+R118F+T244E, D27N+F51I+E56R+V60K+R118F+P256T, D27N+F51 I+E56R+V60K+T244E+P256T, D27N+F51I+E56R+R118F+T244E+P256T, D27N+F51I+V60K+R118F+T244E+P256T, D27N+E56R+V60K+R118F+T244E+P256T, A40I+F51I+E56R+V60K+R118F+T244E, A40I+F51I+E56R+V60K+R118F+P256T, A40I+F51 I+E56R+V60K+T244E+P256T, A40I+F51I+E56R+R118F+T244E+P256T, A40I+F51 I+V60K+R118F+T244E+P256T, A40I+E56R+V60K+R118F+T244E+P256T, F51I+E56R+V60K+R118F+T244E+P256T, G23S+D27N+A40I+F51I+E56R+V60K+R118F, G23S+D27N+A40I+F51I+E56R+V60K+T244E, G23S+D27N+A40I+F51I+E56R+V60K+P256T, G23S+D27N+A40I+F51I+E56R+R118F+T244E,
G23S+D27N+A40I+F51I+E56R+R118F+P256T,
G23S+D27N+A40I+F51I+E56R+T244E+P256T,
G23S+D27N+A40I+F51 I+V60K+R118F+T244E,
G23S+D27N+A40I+F51I+V60K+R118F+P256T,
G23S+D27N+A40I+F51I+V60K+T244E+P256T,
G23S+D27N+A40I+F51I+R118F+T244E+P256T,
G23S+D27N+A40I+E56R+V60K+R118F+T244E,
G23S+D27N+A40I+E56R+V60K+R118F+P256T,
G23S+D27N+A40I+E56R+V60K+T244E+P256T,
G23S+D27N+A40I+E56R+R118F+T244E+P256T,
G23S+D27N+A40I+V60K+R118F+T244E+P256T,
G23S+D27N+F51I+E56R+V60K+R118F+T244E,
G23S+D27N+F51I+E56R+V60K+R118F+P256T,
G23S+D27N+F51I+E56R+V60K+T244E+P256T,
G23S+D27N+F51I+E56R+R118F+T244E+P256T,
G23S+D27N+F51I+V60K+R118F+T244E+P256T,
G23S+D27N+E56R+V60K+R118F+T244E+P256T,
G23S+A40I+F51I+E56R+V60K+R118F+T244E, G23S+A40I+F51I+E56R+V60K+R118F+P256T,
G23S+A40I+F51I+E56R+V60K+T244E+P256T,
G23S+A40I+F51I+E56R+R118F+T244E+P256T,
G23S+A40I+F51I+V60K+R118F+T244E+P256T,
G23S+A40I+E56R+V60K+R118F+T244E+P256T,
G23S+F51I+E56R+V60K+R118F+T244E+P256T,
D27N+A40I+F51I+E56R+V60K+R118F+T244E, D27N+A40I+F51I+E56R+V60K+R118F+P256T,
D27N+A40I+F51I+E56R+V60K+T244E+P256T,
D27N+A40I+F51I+E56R+R118F+T244E+P256T,
D27N+A40I+F51I+V60K+R118F+T244E+P256T,
D27N+A40I+E56R+V60K+R118F+T244E+P256T,
D27N+F51I+E56R+V60K+R118F+T244E+P256T,
A40I+F51I+E56R+V60K+R118F+T244E+P256T,
G23S+D27N+A40I+F51I+E56R+V60K+R118F+T244E,
G23S+D27N+A40I+F51I+E56R+V60K+R118F+P256T,
G23S+D27N+A40I+F51I+E56R+V60K+T244E+P256T,
G23S+D27N+A40I+F51 I+E56R+R118F+T244E+P256T,
G23S+D27N+A40I+F51I+V60K+R118F+T244E+P256T,
G23S+D27N+A40I+E56R+V60K+R118F+T244E+P256T,
G23S+D27N+F51I+E56R+V60K+R118F+T244E+P256T,
G23S+A40I+F51I+E56R+V60K+R118F+T244E+P256T,
D27N+A40I+F51I+E56R+V60K+R118F+T244E+P256T,
G23S+D27N+A40I+F51 I+E56R+V60K+R118F+T244E+P256T.

In a preferred embodiment, the variant of the invention comprises or consists of one of the following set of substitutions corresponding to:
A40E+E56R+D57N+G91T+K98E+R108K+R118F+E210K+T244E+D254S+I202H,
A40E+E56R+D57N+G91T+K98E+R108K+R118F+E210K+T244E+D254S+I252H,
A40E+E56R+D57N+G91T+K98E+R108K+R118F+E210K+T244E+D254S+L269H,
A40E+E56R+D57N+G91T+K98E+R108K+R118F+E210K+T244E+D254S+I202H+I252H,
A40E+E56R+D57N+G91T+K98E+R108K+R118F+E210K+T244E+D254S+I202H+L269H,
A40E+E56R+D57N+G91T+K98E+R108K+R118F+E210K+T244E+D254S+I252H+L269H,
A40E+E56R+D57N+G91T+K98E+R108K+R118F+E210K+T244E+D254S+I252H+L269H, (using SEQ ID NO: 8 for numbeing).

In a preferred embodiment, the variant pf the invention comprises or consists of one of the following set of substitutions corresponding to:
G23S+D27N+A40I+F51I+E56R+V60K+R118F+T244E+P256T+I202H,
G23S+D27N+A40I+F51I+E56R+V60K+R118F+T244E+P256T+I252H,
G23S+D27N+A40I+F51I+E56R+V60K+R118F+T244E+P256T+L269H,
G23S+D27N+A40I+F51I+E56R+V60K+R118F+T244E+P256T+I202H+I252H,
G23S+D27N+A40I+F51I+E56R+V60K+R118F+T244E+P256T+I202H+L269H,
G23S+D27N+A40I+F51I+E56R+V60K+R118F+T244E+P256T+I252H+L269H,
G23S+D27N+A40I+F51I+E56R+V60K+R118F+T244E+P256T+I202H+H252H+L269H (using SEQ ID NO: 8 for numbeing).

A variant of the invention may also, in preferred embodiments, further comprise one or more of the substitutions corresponding to F7K, F51I, F51L, F51V, F51Y, H198D, H198G, H198F, H1981, H198L, H198N, H198S, H198T, H198Y, N200Q, S224F, S224P, L227D, L227E, L227R, V228P, V230R, 1255G, 1255N, A257F, and A2571 (using SEQ ID NO: 8 for numbering).

The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for lipase activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596: 583-589.

The variant of the invention preferably consists of 269 amino acids but may also have comprise a peptide extension/addition at the N-terminal and/or C-terminal. The peptide extensions may be from 1-20 amino acids long. At the N-terminal the peptide extension/addition may preferably comprise from 1-5 of the amino acids SPIRR.

The polypeptide may be a fusion polypeptide comprising a variant of the invention. In an aspect, the variant is isolated.

The variant of the invention has an improved property relative to the parent, in that a lipase variant of the invention has reduced lipase activity and/or reduced odor generation at pHs around neutral, i.e., around pH 6-8, preferably around pH 7, and/or increased benefit risk factor (BRF) compared to the parent lipase, in particular SEQ ID NOs: 2, 4, 6 or 8, respectively.

According to the invention, the wash performance may be measured as the relative wash performance (RP (wash)) compared to the parent lipase, in particular SEQ ID NO: 2, 4, 6, or 8, respectively.

In an embodiment, the relative wash performance (RP (wash)) is greater than 1.0, preferably greater than 1.1, preferably greater than 1.2, preferably greater than 1.3, preferably greater than 1.4, preferably greater than 1.5, preferably greater than 1.6, preferably greater than 1.7, preferably greater than 1.8, preferably greater than 1.9, preferably greater than 2.0, preferably greater than 2.5, preferably greater than 3.0, preferably greater than 3.5, preferably greater than 4.0, preferably greater than 5.0, preferably greater than 6.0, preferably greater than 7.0, preferably greater than 8.0, preferably greater than 9.0, preferably greater than 10.0.

According to the invention, the odor-generation may be measured as the relative odor-generation (RP (odor)) compared to the parent lipase, in particular SEQ ID NO: 2, 4, 6, or 8, respectively.

In an embodiment, the odor-generation is less than 1.0, preferably less than 0.9, preferably less than 0.8, preferably less than 0.7, preferably less than 0.6, preferably less than 0.5, preferably less than 0.4, preferably less than 0.3, preferably less than 0.2, preferably less than 0.1.

According to the invention, the Benefit Risk factor (BRF) is the relative wash performance (Benefit) compared to the relative odor-generation (Risk) and is calculated as RP (wash)/RP (odor). If the Benefit Risk factor of a lipase variant is higher than 1.0, the lipase has better wash performance relative to the released odor compared to the reference lipase, in particular parent lipase in SEQ ID NO: 2, 4, 6, or 8, respectively.

In an embodiment the BRF is greater than 1.0, preferably greater than 1.1, preferably greater than 1.2, preferably greater than 1.3, preferably greater than 1.4, preferably greater than 1.5, preferably greater than 1.6, preferably greater than 1.7, preferably greater than 1.8, preferably greater than 1.9, preferably greater than 2.0, preferably greater than 2.5, preferably greater than 3.0, preferably greater than 3.5, preferably greater than 4.0, preferably greater than 5.0, preferably greater than 6.0, preferably greater than 7.0, preferably greater than 8.0, preferably greater than 9.0, preferably greater than 10.0.

In a preferred embodiment, both the relative wash performance (RP (wash)) and BRF are greater than 1.0, preferably 1.1, preferably greater than 1.2, preferably greater than 1.3, preferably greater than 1.4, preferably greater than 1.5, preferably greater than 1.6, preferably greater than 1.7, preferably greater than 1.8, preferably greater than 1.9, preferably greater than 2.0, preferably greater than 2.5, preferably greater than 3.0, preferably greater than 3.5, preferably greater than 4.0, preferably greater than 5.0, preferably greater than 6.0, preferably greater than 7.0, preferably greater than 8.0, preferably greater than 9.0, preferably greater than 10.0.

### Parent Lipases

A parent lipase has a sequence identity to the polypeptide of SEQ ID NOs: 2, 4, 6 or 8, respectively, of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have lipase activity.

SEQ ID NO: 2 is the mature wild-type
Thermomyces lanuginosus lipase (TLL).
EVSQDLENQF NLFAQYSAAA YCGKNNDAPA GTNITCTGNA
CPEVEKADAT FLYSFEDSGV GDVTGFLALD NTNKLIVLSF
RGSRSIENWI GNLNEDLKEI NDICSGCRGH DGFTSSWRSV
ADTLRQKVED AVREHPDYRV VETGHSLGGA LATVAGADLR
GNGYDIDVES YGAPRVGNRA FAEFLTVQTG GTLYRITHTN
DIVPRLPPRE FGYSHSSPEY WIKSGTLVPV TRNDIVKIEG
IDATGGNNQP NIPDIPAHLW YFGLIGTCL
SEQ ID NO: 4 is a variant of the lipase
in SEQ ID NO: 8.
EVSQDLENQF NLFAQYSAAA YCGKNNDAPA GTNITCTGNE
CPEVEKADAT FLYSERNSGV GDVTGFLALD NTNKLIVLSF
RGSRSIENWI INLNEDLEEI NDICSGCKGH DGETSSWFSV
ADTLRQKVED AVREHPDYRV VETGHSLGGA LATVAGADLR
GNGYDIDVES YGAPRVGNRA FAEFLTVQTG GTLYRITHTN
DIVPRLPPRK FGYSHSSPEY WIKSGTLVPV RRRDIVKIEG
IDAEGGNNQP NIPSIPAHLW YFGLIGTCL
SEQ ID NO: 6 is a variant of the lipase
shown in SEQ ID NO: 8.
EVSQDLENQF NLFAQYSAAA YCSKNNNAPA GTNITCTGNI
CPEVEKADAT ILYSFRDSGK GDVTGFLALD NTNKLIVLSF
RGSRSIENWI GNLNFDLKEI NDICSGCRGH DGETSSWESV
ADTLRQKVED AVREHPDYRV VFTGHSLGGA LATVAGADLR
GNGYDIDVES YGAPRVGNRA FAEFLTVQTG GTLYRITHTN
DIVPRLPPRE FGYSHSSPEY WIKSGTLVPV RRRDIVKIEG
IDAEGGNNQP NIPDITAHLW YFGLIGTCL
SEQ ID NO: 8 is a variant of the wild-type
Thermomyces lanuginosus lipase (TLL) shown
in SEQ ID NO: 2
EVSQDLENQF NLFAQYSAAA YCGKNNDAPA GTNITCTGNA
CPEVEKADAT FLYSFEDSGV GDVTGFLALD NTNKLIVLSF
RGSRSIENWI GNLNEDLKEI NDICSGCRGH DGETSSWRSV
ADTLRQKVED AVREHPDYRV VETGHSLGGA LATVAGADLR
GNGYDIDVES YGAPRVGNRA FAEFLTVQTG GTLYRITHTN
DIVPRLPPRE FGYSHSSPEY WIKSGTLVPV RRRDIVKIEG
IDATGGNNQP NIPDIPAHLW YFGLIGTCL

In preferred aspects, any of SEQ ID NO: 2, SEQ ID NO: 4, SEQ ID NO: 6, and SEQ ID NO: 8, respectively, can be the parent lipase.

In one aspect, the amino acid sequence of the parent differs by up to 10 amino acids, e.g., 1, 2, 3, 4, 5, 6, 7, 8, 9, or 10, from the polypeptide of SEQ ID NOs: 2, 4, 6 or 8, respectively. In another aspect, the parent comprises or consists of the amino acid sequence of SEQ ID NOs: 2, 4, 6 or 8. In another aspect, the parent is a fragment of the polypeptide of SEQ ID NO: 2, 4, 6 or 8 containing at least 200 amino acid residues, e.g., at least 250 and at least 260 amino acid residues.

The parent may be a fusion polypeptide or cleavable fusion polypeptide. A fusion polypeptide is produced by fusing a polynucleotide encoding another polypeptide to a polynucleotide of the present invention. Techniques for producing fusion polypeptides are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

The parent may be obtained from microorganisms of any genus. For purposes of the present invention, the term “obtained from” as used herein in connection with a given source shall mean that the parent encoded by a polynucleotide is produced by the source or by a strain in which the polynucleotide from the source has been inserted. In one aspect, the parent is secreted extracellularly

In a preferred embodiment, the parent is a Thermomyces lipase, in particular a wild-type Thermomyces lanuginosus lipase, especially the lipases of SEQ ID NO: 2 (which is sold under the tradename LIPOLASE™). In another preferred aspect, the parent lipase is a variant of the Thermomyces lanuginosus lipase of SEQ ID NO: 2. In a preferred embodiment the parent lipase is the one shown in SEQ ID NO: 8 (sold under the tradename LIPEX™), which is SEQ ID NO: 2 with substitutions T231R+N233R. In other specifically contemplated embodiments, the parent lipase may be the variant of SEQ ID NO: 8 shown as SEQ ID NO: 4 (which has the following substitutions compared to SEQ ID NO: 8: A40E+E56R+D57N+G91T+K98E+R108K+R118F+E210K+T244E+D254S. In another preferred embodiment, the parent lipase may be the variant of SEQ ID NO: 8, shown in SEQ ID NO: 6, which has the following mutations compared to SEQ ID NO: 8: G23S+D27N+A40I+F51I+E56R+V60K+R118F+T244E+P256T, and is also disclosed in WO 2019/063499—hereby incorporated by reference.

### Preparation of Variants

The present invention also relates to methods for obtaining variants of the invention having lipase activity, comprising:
(a) introducing into a parent lipase, one or more substitutions selected from one or more of groups (i), (ii) and (iii) comprising:
   (i) a substitution at one or more positions corresponding to positions 202, 252, and 269 of the polypeptide of SEQ ID NO: 8;
   (ii) a substitution at one or more positions corresponding to positions 40, 56, 57, 91, 98, 108, 118, 210, 244, and 254 of the polypeptide of SEQ ID NO: 8; and
   (iii) a substitution at one or more positions corresponding to positions 23, 27, 40, 51, 56, 60, 118 244 and 256 of the polypeptide of SEQ ID NO: 8;
   wherein the variant has lipase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 8, wherein the variant optionally comprises an extension of one or more amino acids at the N-terminal and/or C-terminal ends or a truncation of one or more amino acids at the N-terminal and/or C-terminal ends and wherein the variant has lipase activity; and
(b) recovering the variant of the invention.

Any variants of the invention may be produced as described above.

In a preferred embodiment, the variant produced is selected from a variant of the invention which comprises or consists of one or more substitutions, in particular all, corresponding to the positions in SEQ ID NO: 8, selected from the group consisting of:
a substitution of the amino acid residue at position 202 with H;
a substitution of the amino acid residue at position 252 with H; and
a substitution of the amino acid residue at position 269 with H.

In a preferred embodiment, the variant produced is selected from a variant of the invention comprising or consisting of one of the following set of substitutions corresponding to: 202H+252H; 202H+269H; 252H+269H; or 202H+252H+269H (using SEQ ID NO: 8 for numbering).

In another preferred embodiment, the variant produced is selected from a variant of the invention, which comprises or consists of one or more substitutions, in particular all substitutions, corresponding to the positions in SEQ ID NO: 8, selected from the group consisting of:
a substitution of the amino acid residue at position 40 with E;
a substitution of the amino acid residue at position 56 with R;
a substitution of the amino acid residue at position 57 with N;
a substitution of the amino acid residue at position 91 with T;
a substitution of the amino acid residue at position 98 with E;
a substitution of the amino acid residue at position 108 with K;
a substitution of the amino acid residue at position 118 with F;
a substitution of the amino acid residue at position 210 with K;
a substitution of the amino acid residue at position 244 with E; and
a substitution of the amino acid residue at position 254 with S.

In another embodiment, the variant produced is selected from a variant of the invention, which comprises or consists of one or more substitutions, in particular all substitutions, corresponding to the positions in SEQ ID NO: 8, selected from the group consisting of:
a substitution of the amino acid residue at position 23 with S;
a substitution of the amino acid residue at position 27 with N;
a substitution of the amino acid residue at position 40 with I;
a substitution of the amino acid residue at position 51 with I;
a substitution of the amino acid residue at position 56 with R;
a substitution of the amino acid residue at position 60 with K;
a substitution of the amino acid residue at position 118 with F;
a substitution of the amino acid residue at position 244 with E; and
a substitution of the amino acid residue at position 256 with T.

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually, the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76: 4949-4955; and Barton et al., 1990, Nucleic Acids Res. 18: 7349-4966.

Site-directed mutagenesis can also be accomplished in vivo by methods known in the art. See, e.g., US 2004/0171154; Storici et al., 2001, Nature Biotechnol. 19: 773-776; Kren et al., 1998, Nat. Med. 4: 285-290; and Calissano and Macino, 1996, Fungal Genet. Newslett. 43: 15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al., 2004, Nature 432: 1050-1054, and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204) and region-directed mutagenesis (Derbyshire et al., 1986, Gene 46: 145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Granules

The present invention also relates to enzyme granules/particles comprising a lipase variant of the invention. In an embodiment, the granule comprises a core, and optionally one or more coatings (outer layers) surrounding the core.

The core may have a diameter, measured as equivalent spherical diameter (volume based average particle size), of 20-2000 μm, particularly 50-1500 μm, 100-1500 μm or 250-1200 μm. The core diameter, measured as equivalent spherical diameter, can be determined using laser diffraction, such as using a Malvern Mastersizer and/or the method described under IS013320 (2020).

In an embodiment, the core comprises a lipase variant of the present invention.

The core may include additional materials such as fillers, fiber materials (cellulose or synthetic fibers), stabilizing agents, solubilizing agents, suspension agents, viscosity regulating agents, light spheres, plasticizers, salts, lubricants and fragrances.

The core may include a binder, such as synthetic polymer, wax, fat, or carbohydrate.

The core may include a salt of a multivalent cation, a reducing agent, an antioxidant, a peroxide decomposing catalyst and/or an acidic buffer component, typically as a homogenous blend.

The core may include an inert particle with the variant absorbed into it, or applied onto the surface, e.g., by fluid bed coating.

The core may have a diameter of 20-2000 μm, particularly 50-1500 μm, 100-1500 μm or 250-1200 μm.

The core may be surrounded by at least one coating, e.g., to improve the storage stability, to reduce dust formation during handling, or for coloring the granule. The optional coating(s) may include a salt coating, or other suitable coating materials, such as polyethylene glycol (PEG), methyl hydroxy-propyl cellulose (MHPC) and polyvinyl alcohol (PVA).

The coating may be applied in an amount of at least 0.1% by weight of the core, e.g., at least 0.5%, at least 1%, at least 5%, at least 10%, or at least 15%. The amount may be at most 100%, 70%, 50%, 40% or 30%.

The coating is preferably at least 0.1 μm thick, particularly at least 0.5 μm, at least 1 μm or at least 5 μm. In some embodiments, the thickness of the coating is below 100 μm, such as below 60 μm, or below 40 μm.

The coating should encapsulate the core unit by forming a substantially continuous layer. A substantially continuous layer is to be understood as a coating having few or no holes, so that the core unit has few or no uncoated areas. The layer or coating should, in particular, be homogeneous in thickness.

The coating can further contain other materials as known in the art, e.g., fillers, antisticking agents, pigments, dyes, plasticizers and/or binders, such as titanium dioxide, kaolin, calcium carbonate or talc.

A salt coating may comprise at least 60% by weight of a salt, e.g., at least 65%, at least 70%, at least 75%, at least 80%, at least 85%, at least 90%, at least 95% or at least 99% by weight.

To provide acceptable protection, the salt coating is preferably at least 0.1 μm thick, e.g., at least 0.5 μm, at least 1 μm, at least 2 μm, at least 4 μm, at least 5 μm, or at least 8 μm. In a particular embodiment, the thickness of the salt coating is below 100 μm, such as below 60 μm, or below 40 μm.

The salt may be added from a salt solution where the salt is completely dissolved or from a salt suspension wherein the fine particles are less than 50 μm, such as less than 10 μm or less than 5 μm.

The salt coating may comprise a single salt or a mixture of two or more salts. The salt may be water soluble, in particular, having a solubility at least 0.1 g in 100 g of water at 20° C., preferably at least 0.5 g per 100 g water, e.g., at least 1 g per 100 g water, e.g., at least 5 g per 100 g water.

The salt may be an inorganic salt, e.g., salts of sulfate, sulfite, phosphate, phosphonate, nitrate, chloride or carbonate or salts of simple organic acids (less than 10 carbon atoms, e.g., 6 or less carbon atoms) such as citrate, malonate or acetate. Examples of cations in these salts are alkali or earth alkali metal ions, the ammonium ion or metal ions of the first transition series, such as sodium, potassium, magnesium, calcium, zinc or aluminum. Examples of anions include chloride, bromide, iodide, sulfate, sulfite, bisulfite, thiosulfate, phosphate, monobasic phosphate, dibasic phosphate, hypophosphite, dihydrogen pyrophosphate, tetraborate, borate, carbonate, bicarbonate, metasilicate, citrate, malate, maleate, malonate, succinate, lactate, formate, acetate, butyrate, propionate, benzoate, tartrate, ascorbate or gluconate. In particular, alkali- or earth alkali metal salts of sulfate, sulfite, phosphate, phosphonate, nitrate, chloride or carbonate or salts of simple organic acids such as citrate, malonate or acetate may be used.

The salt in the coating may have a constant humidity at 20° C. above 60%, particularly above 70%, above 80% or above 85%, or it may be another hydrate form of such a salt (e.g., anhydrate). The salt coating may be as described in WO 00/01793 or WO 2006/034710.

Specific examples of suitable salts are NaCl (CH_{20° C.}=76%), Na₂CO₃ (CH_{20° C.}=92%), NaNO₃ (CH_{20° C.}=73%), Na₂HPO₄ (CH_{20° C.}=95%), Na₃PO₄ (CH_{25° C.}=92%), NH₄Cl (CH_{20° C.}=79.5%), (NH₄)₂HPO₄ (CH_{20° C.}=93.0%), NH₄H₂PO₄ (CH_{20° C.}=93.1%), (NH₄)₂SO₄ (CH_{20° C.}=81.1%), KCl (CH_{20° C.}=85%), K₂HPO₄ (CH_{20° C.}=92%), KH₂PO₄ (CH_{20° C.}=96.5%), KNO₃ (CH_{20° C.}=93.5%), Na₂SO₄ (CH_{20° C.}=93%), K₂SO₄ (CH_{20° C.}=98%), KHSO₄ (CH_{20° C.}=86%), MgSO₄ (CH_{20° C.}=90%), ZnSO₄ (CH_{20° C.}=90%) and sodium citrate (CH_{25° C.}=86%). Other examples include NaH₂PO₄, (NH₄)H₂PO₄, CuSO₄, Mg(NO₃)₂ and magnesium acetate.

The salt may be in anhydrous form, or it may be a hydrated salt, i.e., a crystalline salt hydrate with bound water(s) of crystallization, such as described in WO 99/32595. Specific examples include anhydrous sodium sulfate (Na₂SO₄), anhydrous magnesium sulfate (MgSO₄), magnesium sulfate heptahydrate (MgSO₄·7H₂O), zinc sulfate heptahydrate (ZnSO₄·7H₂O), sodium phosphate dibasic heptahydrate (Na₂HPO₄·7H₂O), magnesium nitrate hexahydrate (Mg(NO₃)₂(6H₂O)), sodium citrate dihydrate and magnesium acetate tetrahydrate.

Preferably the salt is applied as a solution of the salt, e.g., using a fluid bed.

The coating materials can be waxy coating materials and film-forming coating materials. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

The granule may optionally have one or more additional coatings. Examples of suitable coating materials are polyethylene glycol (PEG), methyl hydroxy-propyl cellulose (MHPC) and polyvinyl alcohol (PVA). Examples of enzyme granules with multiple coatings are described in WO 93/07263 and WO 97/23606.

The core can be prepared by granulating a blend of the ingredients, e.g., by a method comprising granulation techniques such as crystallization, precipitation, pan-coating, fluid bed coating, fluid bed agglomeration, rotary atomization, extrusion, prilling, spheronization, size reduction methods, drum granulation, and/or high shear granulation.

Methods for preparing the core can be found in the Handbook of Powder Technology; Particle size enlargement by C. E. Capes; Vol. 1; 1980; Elsevier. Preparation methods include known feed and granule formulation technologies, e.g.,
(a) Spray dried products, wherein a liquid enzyme-containing solution is atomized in a spray drying tower to form small droplets which during their way down the drying tower dry to form an enzyme-containing particulate material. Very small particles can be produced this way (Michael S. Showell (editor); Powdered detergents; Surfactant Science Series; 1998; Vol. 71; pages 140-142; Marcel Dekker).
(b) Layered products, wherein the enzyme is coated as a layer around a pre-formed inert core particle, wherein an enzyme-containing solution is atomized, typically in a fluid bed apparatus wherein the pre-formed core particles are fluidized, and the enzyme-containing solution adheres to the core particles and dries up to leave a layer of dry enzyme on the surface of the core particle. Particles of a desired size can be obtained this way if a useful core particle of the desired size can be found. This type of product is described in, e.g., WO 97/23606.
(c) Absorbed core particles, wherein rather than coating the variant as a layer around the core, the enzyme is absorbed onto and/or into the surface of the core. Such a process is described in WO 97/39116.
(d) Extrusion or pelletized products, wherein a variant-containing paste is pressed to pellets or under pressure is extruded through a small opening and cut into particles which are subsequently dried. Such particles usually have a considerable size because of the material in which the extrusion opening is made (usually a plate with bore holes) sets a limit on the allowable pressure drop over the extrusion opening. Also, very high extrusion pressures when using a small opening increase heat generation in the enzyme paste, which is harmful to the enzyme (Michael S. Showell (editor); Powdered detergents; Surfactant Science Series; 1998; Vol. 71; pages 140-142; Marcel Dekker).
(e) Prilled products, wherein a variant-containing powder is suspended in molten wax and the suspension is sprayed, e.g., through a rotating disk atomizer, into a cooling chamber where the droplets quickly solidify (Michael S. Showell (editor); Powdered detergents; Surfactant Science Series; 1998; Vol. 71; pages 140-142; Marcel Dekker). The product obtained is one wherein the variant is uniformly distributed throughout an inert material instead of being concentrated on its surface. U.S. Pat. Nos. 4,016,040 and 4,713,245 describe this technique.
(f) Mixer granulation products, wherein a variant-containing liquid is added to a dry powder composition of conventional granulating components. The liquid and the powder in a suitable proportion are mixed and as the moisture of the liquid is absorbed in the dry powder, the components of the dry powder will start to adhere and agglomerate and particles will build up, forming granulates comprising the enzyme. Such a process is described in U.S. Pat. No. 4,106,991, EP 170360, EP 304332, EP 304331, WO 90/09440 and WO 90/09428. In a particular aspect of this process, various high-shear mixers can be used as granulators. Granulates consisting of variant, fillers and binders etc. are mixed with cellulose fibers to reinforce the particles to produce a so-called T-granulate. Reinforced particles, are more robust, and release less enzymatic dust.
(g) Size reduction, wherein the cores are produced by milling or crushing of larger particles, pellets, tablets, briquettes etc. containing the enzyme. The wanted core particle fraction is obtained by sieving the milled or crushed product. Over and undersized particles can be recycled. Size reduction is described in Martin Rhodes (editor); Principles of Powder Technology; 1990; Chapter 10; John Wiley & Sons.
(h) Fluid bed granulation. Fluid bed granulation involves suspending particulates in an air stream and spraying a liquid onto the fluidized particles via nozzles. Particles hit by spray droplets get wetted and become tacky. The tacky particles collide with other particles and adhere to them to form a granule.
(i) The cores may be subjected to drying, such as in a fluid bed drier. Other known methods for drying granules in the feed or enzyme industry can be used by the skilled person. The drying preferably takes place at a product temperature of from 25 to 90° C. For some enzymes, it is important the cores comprising the variant contain a low amount of water before coating with the salt. If water sensitive enzymes are coated with a salt before excessive water is removed, the excessive water will be trapped within the core and may affect the activity of the enzyme negatively. After drying, the cores preferably contain 0.1-10% w/w water.

Non-dusting granulates may be produced, e.g., as disclosed in U.S. Pat. Nos. 4,106,991 and 4,661,452 and may optionally be coated by methods known in the art.

The granulate may further comprise one or more additional enzymes. Each enzyme will then be present in more granules securing a more uniform distribution of the enzymes, and also reduces the physical segregation of different enzymes due to different particle sizes. Methods for producing multi-enzyme co-granulates is disclosed in the ip.com disclosure IPCOM000200739D.

Another example of formulation of enzymes by the use of co-granulates is disclosed in WO 2013/188331.

The present invention also relates to protected enzymes prepared according to the method disclosed in EP 238216.

In an embodiment, the granule further comprises one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectin esterase, triacylglycerol lipase, xylanase, beta-xylosidase or any combination thereof.

### Liquid Composition

The present invention also relates to liquid compositions comprising a variant of the invention. The composition may comprise an enzyme stabilizer (examples of which include polyols such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).

In some embodiments, filler(s) or carrier material(s) are included to increase the volume of such compositions. Suitable filler or carrier materials include, but are not limited to, various salts of sulfate, carbonate and silicate as well as talc, clay and the like. Suitable filler or carrier materials for liquid compositions include, but are not limited to, water or low molecular weight primary and secondary alcohols including polyols and diols. Examples of such alcohols include, but are not limited to, methanol, ethanol, propanol and isopropanol. In some embodiments, the compositions contain from about 5% to about 90% of such materials.

In an aspect, the liquid formulation comprises 20-80% w/w of polyol. In one embodiment, the liquid formulation comprises 0.001-2% w/w preservative.

In another embodiment, the invention relates to liquid formulations comprising:
(A) 0.001-25% w/w of a variant of the present invention;
(B) 20-80% w/w of polyol;
(C) optionally 0.001-2% w/w preservative; and
(D) water.

In another embodiment, the invention relates to liquid formulations comprising:
(A) 0.001-25% w/w of a variant of the present invention;
(B) 0.001-2% w/w preservative;
(C) optionally 20-80% w/w of polyol; and
(D) water.

In another embodiment, the liquid formulation comprises one or more formulating agents, such as a formulating agent selected from the group consisting of polyol, sodium chloride, sodium benzoate, potassium sorbate, sodium sulfate, potassium sulfate, magnesium sulfate, sodium thiosulfate, calcium carbonate, sodium citrate, dextrin, glucose, sucrose, sorbitol, lactose, starch, PVA, acetate and phosphate, preferably selected from the group consisting of sodium sulfate, dextrin, cellulose, sodium thiosulfate, kaolin and calcium carbonate. In one embodiment, the polyols is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600, more preferably selected from the group consisting of glycerol, sorbitol and propylene glycol (MPG) or any combination thereof.

In another embodiment, the liquid formulation comprises 20-80% polyol (i.e., total amount of polyol), e.g., 25-75% polyol, 30-70% polyol, 35-65% polyol, or 40-60% polyol. In one embodiment, the liquid formulation comprises 20-80% polyol, e.g., 25-75% polyol, 30-70% polyol, 35-65% polyol, or 40-60% polyol, wherein the polyol is selected from the group consisting of glycerol, sorbitol, propylene glycol (MPG), ethylene glycol, diethylene glycol, triethylene glycol, 1,2-propylene glycol or 1,3-propylene glycol, dipropylene glycol, polyethylene glycol (PEG) having an average molecular weight below about 600 and polypropylene glycol (PPG) having an average molecular weight below about 600. In one embodiment, the liquid formulation comprises 20-80% polyol (i.e., total amount of polyol), e.g., 25-75% polyol, 30-70% polyol, 35-65% polyol, or 40-60% polyol, wherein the polyol is selected from the group consisting of glycerol, sorbitol and propylene glycol (MPG).

In another embodiment, the preservative is selected from the group consisting of sodium sorbate, potassium sorbate, sodium benzoate and potassium benzoate or any combination thereof. In one embodiment, the liquid formulation comprises 0.02-1.5% w/w preservative, e.g., 0.05-1% w/w preservative or 0.1-0.5% w/w preservative. In one embodiment, the liquid formulation comprises 0.001-2% w/w preservative (i.e., total amount of preservative), e.g., 0.02-1.5% w/w preservative, 0.05-1% w/w preservative, or 0.1-0.5% w/w preservative, wherein the preservative is selected from the group consisting of sodium sorbate, potassium sorbate, sodium benzoate and potassium benzoate or any combination thereof.

In another embodiment, the liquid formulation further comprises one or more additional enzymes, e.g., hydrolase, isomerase, ligase, lyase, oxidoreductase, and transferase. The one or more additional enzymes are preferably selected from the group consisting of acetylxylan esterase, acylglycerol lipase, amylase, alpha-amylase, beta-amylase, arabinofuranosidase, cellobiohydrolases, cellulase, feruloyl esterase, galactanase, alpha-galactosidase, beta-galactosidase, beta-glucanase, beta-glucosidase, lysophospholipase, lysozyme, alpha-mannosidase, beta-mannosidase (mannanase), phytase, phospholipase A1, phospholipase A2, phospholipase D, protease, pullulanase, pectin esterase, triacylglycerol lipase, xylanase, beta-xylosidase or any combination thereof.

### Compositions

The invention also concerns compositions comprising the lipase variant of the present inventions, a granule of the invention, or a liquid composition of the present invention.

A composition of the invention has reduced odor-generation and/or increased Benefit Risk factor (BRF) compared to the same composition comprising the parent lipase, in particular SEQ ID NOs: 2, 4, 6, 8, respectively.

In a preferred embodiment the composition comprising one or more surfactants.

The non-limiting list of composition components, illustrated hereinafter, are suitable for use in the compositions and methods herein may be desirably incorporated in certain embodiments of the invention, e.g., to assist or enhance cleaning performance, for treatment of the substrate to be cleaned, or to modify the aesthetics of the composition as is the case with perfumes, colorants, dyes or the like. The levels of any such components incorporated in any compositions are in addition to any materials previously recited for incorporation. The precise nature of these additional components, and levels of incorporation thereof, will depend on the physical form of the composition and the nature of the cleaning operation for which it is to be used. Although components mentioned below are categorized by general header according to a particular functionality, this is not to be construed as a limitation, as a component may comprise additional functionalities as will be appreciated by the skilled artisan.

Unless otherwise indicated the amounts in percentage is by weight of the composition (wt %). Suitable component materials include, but are not limited to, surfactants, builders, chelating agents, dye transfer inhibiting agents, dispersants, enzymes, and enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, hueing dyes, perfumes, perfume delivery systems, structure elasticizing agents, fabric softeners, carriers, hydrotropes, processing aids, solvents and/or pigments. In addition to the disclosure below, suitable examples of such other components and levels of use are found in U.S. Pat. Nos. 5,576,282, 6,306,812, and 6,326,348 hereby incorporated by reference.

Thus, in certain embodiments, the invention do not contain one or more of the following adjuncts materials: surfactants, soaps, builders, chelating agents, dye transfer inhibiting agents, dispersants, additional enzymes, enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, perfumes, perfume delivery systems, structure elasticizing agents, fabric softeners, carriers, hydrotropes, processing aids, solvents and/or pigments. However, when one or more components are present, such one or more components may be present as detailed below:

Surfactants—The compositions according to the present invention may comprise a surfactant or surfactant system wherein the surfactant can be selected from nonionic surfactants, anionic surfactants, cationic surfactants, ampholytic surfactants, zwitterionic surfactants, semi-polar nonionic surfactants and mixtures thereof. When present, surfactant is typically present at a level of from 0.1 to 60 wt %, from 0.2 to 40 wt %, from 0.5 to 30 wt %, from 1 to 50 wt %, from 1 to 40 wt %, from 1 to 30 wt %, from 1 to 20 wt %, from 3 to 10 wt %, from 3 to 5 wt %, from 5 to 40 wt %, from 5 to 30 wt %, from 5 to 15 wt %, from 3 to 20 wt %, from 3 to 10 wt %, from 8 to 12 wt %, from 10 to 12 wt %, from 20 to 25 wt % or from 25-60%.

Suitable anionic detersive surfactants include sulphate and sulphonate detersive surfactants.

Suitable sulphonate detersive surfactants include alkyl benzene sulphonate, in one aspect, C₁₀₋₁₃ alkyl benzene sulphonate. Suitable alkyl benzene sulphonate (LAS) may be obtained, by sulphonating commercially available linear alkyl benzene (LAB); suitable LAB includes low 2-phenyl LAB, such as Isochem® or Petrelab®, other suitable LAB include high 2-phenyl LAB, such as Hyblene®. A suitable anionic detersive surfactant is alkyl benzene sulphonate that is obtained by DETAL catalyzed process, although other synthesis routes, such as HF, may also be suitable. In one aspect a magnesium salt of LAS is used.

Suitable sulphate detersive surfactants include alkyl sulphate, in one aspect, C₈₋₁₈ alkyl sulphate, or predominantly C₁₂ alkyl sulphate.

Another suitable sulphate detersive surfactant is alkyl alkoxylated sulphate, in one aspect, alkyl ethoxylated sulphate, in one aspect, a C₈₋₁₈ alkyl alkoxylated sulphate, in another aspect, a C₈₋₁₈ alkyl ethoxylated sulphate, typically the alkyl alkoxylated sulphate has an average degree of alkoxylation of from 0.5 to 20, or from 0.5 to 10, typically the alkyl alkoxylated sulphate is a C₈₋₁₈ alkyl ethoxylated sulphate having an average degree of ethoxylation of from 0.5 to 10, from 0.5 to 7, from 0.5 to 5 or from 0.5 to 3.

The alkyl sulphate, alkyl alkoxylated sulphate and alkyl benzene sulphonates may be linear or branched, substituted or un-substituted.

The detersive surfactant may be a mid-chain branched detersive surfactant, in one aspect, a mid-chain branched anionic detersive surfactant, in one aspect, a mid-chain branched alkyl sulphate and/or a mid-chain branched alkyl benzene sulphonate, e.g. a mid-chain branched alkyl sulphate. In one aspect, the mid-chain branches are C₁₋₄ alkyl groups, typically methyl and/or ethyl groups.

Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or soap, and combinations thereof.

Suitable non-ionic detersive surfactants are selected from the group consisting of: C₈-C₁₈ alkyl ethoxylates, such as, NEODOL®; C₆-C₁₂ alkyl phenol alkoxylates wherein the alkoxylate units may be ethyleneoxy units, propyleneoxy units or a mixture thereof; C₁₂-C₁₈ alcohol and C₆-C₁₂ alkyl phenol condensates with ethylene oxide/propylene oxide block polymers such as Pluronic®; C₁₄-C₂₂ mid-chain branched alcohols; C₁₄-C₂₂ mid-chain branched alkyl alkoxylates, typically having an average degree of alkoxylation of from 1 to 30; alkylpolysaccharides, in one aspect, alkylpolyglycosides; polyhydroxy fatty acid amides; ether capped poly(oxyalkylated) alcohol surfactants; and mixtures thereof.

Suitable non-ionic detersive surfactants include alkyl polyglucoside and/or an alkyl alkoxylated alcohol.

In one aspect, non-ionic detersive surfactants include alkyl alkoxylated alcohols, in one aspect C₈₋₁₈ alkyl alkoxylated alcohol, e.g. a C₈₋₁₈ alkyl ethoxylated alcohol, the alkyl alkoxylated alcohol may have an average degree of alkoxylation of from 1 to 50, from 1 to 30, from 1 to 20, or from 1 to 10. In one aspect, the alkyl alkoxylated alcohol may be a C₈₋₁₈ alkyl ethoxylated alcohol having an average degree of ethoxylation of from 1 to 10, from 1 to 7, more from 1 to 5 or from 3 to 7. The alkyl alkoxylated alcohol can be linear or branched, and substituted or un-substituted. Suitable nonionic surfactants include Lutensol®.

Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN and TWEEN, and combinations thereof.

Suitable cationic detersive surfactants include alkyl pyridinium compounds, alkyl quaternary ammonium compounds, alkyl quaternary phosphonium compounds, alkyl ternary sulphonium compounds, and mixtures thereof.

Suitable cationic detersive surfactants are quaternary ammonium compounds having the general formula: (R)(R₁)(R₂)(R₃)N⁺ X⁻, wherein, R is a linear or branched, substituted or unsubstituted C₆₋₁₈ alkyl or alkenyl moiety, R₁ and R₂ are independently selected from methyl or ethyl moieties, R₃ is a hydroxyl, hydroxymethyl or a hydroxyethyl moiety, X is an anion which provides charge neutrality, suitable anions include: halides, e.g. chloride; sulphate; and sulphonate. Suitable cationic detersive surfactants are mono-C₆₋₁₈ alkyl mono-hydroxyethyl di-methyl quaternary ammonium chlorides. Highly suitable cationic detersive surfactants are mono-C₈₋₁₀ alkyl mono-hydroxyethyl di-methyl quaternary ammonium chloride, mono-C₁₀₋₁₂ alkyl mono-hydroxyethyl di-methyl quaternary ammonium chloride and mono-C₁₀ alkyl mono-hydroxyethyl di-methyl quaternary ammonium chloride.

Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

Suitable amphoteric/zwitterionic surfactants include amine oxides and betaines such as alkyldimethylbetaines, sulfobetaines, or combinations thereof. Amine-neutralized anionic surfactants—Anionic surfactants of the present invention and adjunct anionic cosurfactants, may exist in an acid form, and said acid form may be neutralized to form a surfactant salt which is desirable for use in the present detergent compositions. Typical agents for neutralization include the metal counterion base such as hydroxides, eg, NaOH or KOH. Further preferred agents for neutralizing anionic surfactants of the present invention and adjunct anionic surfactants or cosurfactants in their acid forms include ammonia, amines, or alkanolamines. Alkanolamines are preferred. Suitable non-limiting examples including monoethanolamine, diethanolamine, triethanolamine, and other linear or branched alkanolamines known in the art; e.g., highly preferred alkanolamines include 2-amino-1-propanol, 1-aminopropanol, monoisopropanolamine, or 1-amino-3-propanol. Amine neutralization may be done to a full or partial extent, e.g. part of the anionic surfactant mix may be neutralized with sodium or potassium and part of the anionic surfactant mix may be neutralized with amines or alkanolamines.

Non-limiting examples of semipolar surfactants include amine oxides (AO) such as alkyldimethylamineoxide

Surfactant systems comprising mixtures of one or more anionic and in addition one or more nonionic surfactants optionally with an additional surfactant such as a cationic surfactant, may be preferred. Preferred weight ratios of anionic to nonionic surfactant are at least 2:1, or at least 1:1 to 1:10.

In one aspect a surfactant system may comprise a mixture of isoprenoid surfactants represented by formula A and formula B:

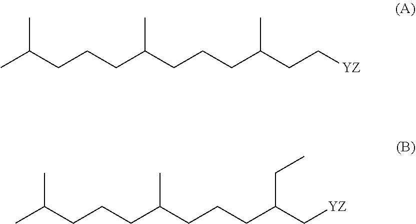

where Y is CH₂ or null, and Z may be chosen such that the resulting surfactant is selected from the following surfactants: an alkyl carboxylate surfactant, an alkyl polyalkoxy surfactant, an alkyl anionic polyalkoxy sulfate surfactant, an alkyl glycerol ester sulfonate surfactant, an alkyl dimethyl amine oxide surfactant, an alkyl polyhydroxy based surfactant, an alkyl phosphate ester surfactant, an alkyl glycerol sulfonate surfactant, an alkyl polygluconate surfactant, an alkyl polyphosphate ester surfactant, an alkyl phosphonate surfactant, an alkyl polyglycoside surfactant, an alkyl monoglycoside surfactant, an alkyl diglycoside surfactant, an alkyl sulfosuccinate surfactant, an alkyl disulfate surfactant, an alkyl disulfonate surfactant, an alkyl sulfosuccinamate surfactant, an alkyl glucamide surfactant, an alkyl taurinate surfactant, an alkyl sarcosinate surfactant, an alkyl glycinate surfactant, an alkyl isethionate surfactant, an alkyl dialkanolamide surfactant, an alkyl monoalkanolamide surfactant, an alkyl monoalkanolamide sulfate surfactant, an alkyl diglycolamide surfactant, an alkyl diglycolamide sulfate surfactant, an alkyl glycerol ester surfactant, an alkyl glycerol ester sulfate surfactant, an alkyl glycerol ether surfactant, an alkyl glycerol ether sulfate surfactant, alkyl methyl ester sulfonate surfactant, an alkyl polyglycerol ether surfactant, an alkyl polyglycerol ether sulfate surfactant, an alkyl sorbitan ester surfactant, an alkyl ammonioalkanesulfonate surfactant, an alkyl amidopropyl betaine surfactant, an alkyl allylated quat based surfactant, an alkyl monohydroxyalkyl-di-alkylated quat based surfactant, an alkyl di-hydroxyalkyl monoalkyl quat based surfactant, an alkylated quat surfactant, an alkyl trimethylammonium quat surfactant, an alkyl polyhydroxalkyl oxypropyl quat based surfactant, an alkyl glycerol ester quat surfactant, an alkyl glycol amine quat surfactant, an alkyl monomethyl dihydroxyethyl quaternary ammonium surfactant, an alkyl dimethyl monohydroxyethyl quaternary ammonium surfactant, an alkyl trimethylammonium surfactant, an alkyl imidazoline-based surfactant, an alken-2-yl-succinate surfactant, an alkyl a-sulfonated carboxylic acid surfactant, an alkyl a-sulfonated carboxylic acid alkyl ester surfactant, an alpha olefin sulfonate surfactant, an alkyl phenol ethoxylate surfactant, an alkyl benzenesulfonate surfactant, an alkyl sulfobetaine surfactant, an alkyl hydroxysulfobetaine surfactant, an alkyl ammoniocarboxylate betaine surfactant, an alkyl sucrose ester surfactant, an alkyl alkanolamide surfactant, an alkyl di(polyoxyethylene) monoalkyl ammonium surfactant, an alkyl mono(polyoxyethylene) dialkyl ammonium surfactant, an alkyl benzyl dimethylammonium surfactant, an alkyl aminopropionate surfactant, an alkyl amidopropyl dimethylamine surfactant, or a mixture thereof; and if Z is a charged moiety, Z is charge-balanced by a suitable metal or organic counter ion. Suitable counter ions include a metal counter ion, an amine, or an alkanolamine, e.g., C1-C6 alkanolammonium. More specifically, suitable counter ions include Na+, Ca+, Li+, K+, Mg+, e.g., monoethanolamine (MEA), diethanolamine (DEA), triethanolamine (TEA), 2-amino-1-propanol, 1-aminopropanol, methyldiethanolamine, dimethylethanolamine, monoisopropanolamine, triisopropanolamine, I-amino-3-propanol, or mixtures thereof. In one embodiment, the compositions contain from 5% to 97% of one or more non-isoprenoid surfactants; and one or more adjunct cleaning additives; wherein the weight ratio of surfactant of formula A to surfactant of formula B is from 50:50 to 95:5.

Soap—The compositions herein may contain soap. Without being limited by theory, it may be desirable to include soap as it acts in part as a surfactant and in part as a builder and may be useful for suppression of foam and may furthermore interact favorably with the various cationic compounds of the composition to enhance softness on textile fabrics treaded with the inventive compositions. Any soap known in the art for use in laundry detergents may be utilized. In one embodiment, the compositions contain from 0 wt % to 20 wt %, from 0.5 wt % to 20 wt %, from 4 wt % to 10 wt %, or from 4 wt % to 7 wt % of soap.

Examples of soap useful herein include oleic acid soaps, palmitic acid soaps, palm kernel fatty acid soaps, and mixtures thereof. Typical soaps are in the form of mixtures of fatty acid soaps having different chain lengths and degrees of substitution. One such mixture is topped palm kernel fatty acid.

In one embodiment, the soap is selected from free fatty acid. Suitable fatty acids are saturated and/or unsaturated and can be obtained from natural sources such a plant or animal esters (e.g., palm kernel oil, palm oil, coconut oil, babassu oil, safflower oil, tall oil, castor oil, tallow and fish oils, grease, and mixtures thereof), or synthetically prepared (e.g., via the oxidation of petroleum or by hydrogenation of carbon monoxide via the Fisher Tropsch process).

Examples of suitable saturated fatty acids for use in the compositions of this invention include captic, lauric, myristic, palmitic, stearic, arachidic and behenic acid. Suitable unsaturated fatty acid species include: palmitoleic, oleic, linoleic, linolenic and ricinoleic acid. Examples of preferred fatty acids are saturated Cn fatty acid, saturated Ci₂-Ci₄ fatty acids, and saturated or unsaturated Cn to Ci₈ fatty acids, and mixtures thereof.

When present, the weight ratio of fabric softening cationic cosurfactant to fatty acid is preferably from about 1:3 to about 3:1, more preferably from about 1:1.5 to about 1.5:1, most preferably about 1:1.

Levels of soap and of nonsoap anionic surfactants herein are percentages by weight of the detergent composition, specified on an acid form basis. However, as is commonly understood in the art, anionic surfactants and soaps are in practice neutralized using sodium, potassium or alkanolammonium bases, such as sodium hydroxide or monoethanolamine.

Hydrotropes—The compositions of the present invention may comprise one or more hydrotropes. A hydrotrope is a compound that solubilises hydrophobic compounds in aqueous solutions (or oppositely, polar substances in a non-polar environment). Typically, hydrotropes have both hydrophilic and a hydrophobic character (so-called amphiphilic properties as known from surfactants); however the molecular structure of hydrotropes generally do not favor spontaneous self-aggregation, see e.g. review by Hodgdon and Kaler (2007), Current Opinion in Colloid & Interface Science 12: 121-128. Hydrotropes do not display a critical concentration above which self-aggregation occurs as found for surfactants and lipids forming miceller, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow for example more concentrated formulations of surfactants (as in the process of compacting liquid detergents by removing water) without inducing undesired phenomena such as phase separation or high viscosity.

The detergent may contain from 0 to 10 wt %, such as from 0 to 5 wt %, 0.5 to 5 wt %, or from 3% to 5 wt %, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

Builders—The compositions of the present invention may comprise one or more builders, co-builders, builder systems or a mixture thereof. When a builder is used, the cleaning composition will typically comprise from 0 to 65 wt %, at least 1 wt %, from 2 to 60 wt % or from 5 to 10 wt % builder.

In a dish wash cleaning composition, the level of builder is typically 40 to 65 wt % or 50 to 65 wt %. The composition may be substantially free of builder; substantially free means “no deliberately added” zeolite and/or phosphate. Typical zeolite builders include zeolite A, zeolite P and zeolite MAP. A typical phosphate builder is sodium tri-polyphosphate.

The builder and/or co-builder may particularly be a chelating agent that forms water-soluble complexes with Ca and Mg. Any builder and/or co-builder known in the art for use in detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), iminodiethanol (DEA) and 2,2′,2″-nitrilotriethanol (TEA), and carboxymethylinulin (CMI), and combinations thereof.

The cleaning composition may include a co-builder alone, or in combination with a builder, e.g. a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), etheylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diylbis(phosphonic acid) (HEDP), ethylenediaminetetrakis(methylene)tetrakis(phosphonic acid) (EDTMPA), diethylenetriaminepentakis(methylene)pentakis(phosphonic acid) (DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl) aspartic acid (SMAS), N-(2-sulfoethyl) aspartic acid (SEAS), N-(2-sulfomethyl) glutamic acid (SMGL), N-(2-sulfoethyl) glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N, N-diacetic acid (SLDA), taurine-N, N-diacetic acid (TUDA) and sulfomethyl-N,N-diacetic acid (SMDA), N-(hydroxyethyl)-ethylidenediaminetriacetate (HEDTA), diethanolglycine (DEG), Diethylenetriamine Penta (Methylene Phosphonic acid) (DTPMP), aminotris(methylenephosphonic acid) (ATMP), and combinations and salts thereof. Further exemplary builders and/or co-builders are described in, e.g., WO09/102854, U.S. Pat. No. 5,977,053.

Chelating Agents and Crystal Growth Inhibitors—The compositions herein may contain a chelating agent and/or a crystal growth inhibitor. Suitable molecules include copper, iron and/or manganese chelating agents and mixtures thereof. Suitable molecules include DTPA (Diethylene triamine pentaacetic acid), HEDP (Hydroxyethane diphosphonic acid), DTPMP (Diethylene triamine penta(methylene phosphonic acid)), 1,2-Dihydroxybenzene-3,5-disulfonic acid disodium salt hydrate, ethylenediamine, diethylene triamine, ethylenediaminedisuccinic acid (EDDS), N-hydroxyethylethylenediaminetri-acetic acid (HEDTA), triethylenetetraaminehexaacetic acid (TTHA), N-hydroxyethyliminodiacetic acid (HEIDA), dihydroxyethylglycine (DHEG), ethylenediaminetetrapropionic acid (EDTP), carboxymethyl inulin and 2-Phosphonobutane 1,2,4-tricarboxylic acid (Bayhibit® AM) and derivatives thereof. Typically the composition may comprise from 0.005 to 15 wt % or from 3.0 to 10 wt % chelating agent or crystal growth inhibitor.

Bleach Component—The bleach component suitable for incorporation in the methods and compositions of the invention comprise one or a mixture of more than one bleach component.

Suitable bleach components include bleaching catalysts, photobleaches, bleach activators, hydrogen peroxide, sources of hydrogen peroxide, pre-formed peracids and mixtures thereof. In general, when a bleach component is used, the compositions of the present invention may comprise from 0 to 30 wt %, from 0.00001 to 90 wt %, 0.0001 to 50 wt %, from 0.001 to 25 wt % or from 1 to 20 wt %. Examples of suitable bleach components include:

(1) Pre-formed peracids: Suitable preformed peracids include, but are not limited to, compounds selected from the group consisting of pre-formed peroxyacids or salts thereof, typically either a peroxycarboxylic acid or salt thereof, or a peroxysulphonic acid or salt thereof.

The pre-formed peroxyacid or salt thereof is preferably a peroxycarboxylic acid or salt thereof, typically having a chemical structure corresponding to the following chemical formula:

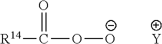

wherein: R¹⁴ is selected from alkyl, aralkyl, cycloalkyl, aryl or heterocyclic groups; the R¹⁴ group can be linear or branched, substituted or unsubstituted; and Y is any suitable counter-ion that achieves electric charge neutrality, preferably Y is selected from hydrogen, sodium or potassium. Preferably, R¹⁴ is a linear or branched, substituted or unsubstituted C₆₋₉ alkyl. Preferably, the peroxyacid or salt thereof is selected from peroxyhexanoic acid, peroxyheptanoic acid, peroxyoctanoic acid, peroxynonanoic acid, peroxydecanoic acid, any salt thereof, or any combination thereof. Particularly preferred peroxyacids are phthalimido-peroxy-alkanoic acids, in particular ε-phthahlimido peroxy hexanoic acid (PAP). Preferably, the peroxyacid or salt thereof has a melting point in the range of from 30° C. to 60° C.

The pre-formed peroxyacid or salt thereof can also be a peroxysulphonic acid or salt thereof, typically having a chemical structure corresponding to the following chemical formula:

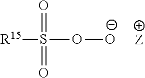

wherein: R¹⁵ is selected from alkyl, aralkyl, cycloalkyl, aryl or heterocyclic groups; the R¹⁵ group can be linear or branched, substituted or unsubstituted; and Z is any suitable counter-ion that achieves electric charge neutrality, preferably Z is selected from hydrogen, sodium or potassium. Preferably R¹⁵ is a linear or branched, substituted or unsubstituted C₆₋₉ alkyl. Preferably such bleach components may be present in the compositions of the invention in an amount from 0.01 to 50 wt % or from 0.1 to 20 wt %.

(2) Sources of hydrogen peroxide include e.g., inorganic perhydrate salts, including alkali metal salts such as sodium salts of perborate (usually mono- or tetra-hydrate), percarbonate, persulphate, perphosphate, persilicate salts and mixtures thereof. In one aspect of the invention the inorganic perhydrate salts such as those selected from the group consisting of sodium salts of perborate, percarbonate and mixtures thereof. When employed, inorganic perhydrate salts are typically present in amounts of 0.05 to 40 wt % or 1 to 30 wt % of the overall composition and are typically incorporated into such compositions as a crystalline solid that may be coated. Suitable coatings include: inorganic salts such as alkali metal silicate, carbonate or borate salts or mixtures thereof, or organic materials such as water-soluble or dispersible polymers, waxes, oils or fatty soaps. Preferably such bleach components may be present in the compositions of the invention in an amount of 0.01 to 50 wt % or 0.1 to 20 wt %.

(3) The term bleach activator is meant herein as a compound which reacts with hydrogen peroxide to form a peracid via perhydrolysis. The peracid thus formed constitutes the activated bleach. Suitable bleach activators to be used herein include those belonging to the class of esters, amides, imides or anhydrides. Suitable bleach activators are those having R—(C═O)-L wherein R is an alkyl group, optionally branched, having, when the bleach activator is hydrophobic, from 6 to 14 carbon atoms, or from 8 to 12 carbon atoms and, when the bleach activator is hydrophilic, less than 6 carbon atoms or less than 4 carbon atoms; and L is leaving group. Examples of suitable leaving groups are benzoic acid and derivatives thereof—especially benzene sulphonate. Suitable bleach activators include dodecanoyl oxybenzene sulphonate, decanoyl oxybenzene sulphonate, decanoyl oxybenzoic acid or salts thereof, 3,5,5-trimethyl hexanoyloxybenzene sulphonate, tetraacetyl ethylene diamine (TAED), sodium 4-[(3,5,5-trimethylhexanoyl)oxy]benzene-1-sulfonate (ISONOBS), 4-(dodecanoyloxy)benzene-1-sulfonate (LOBS), 4-(decanoyloxy)benzene-1-sulfonate, 4-(decanoyloxy)benzoate (DOBS or DOBA), 4-(nonanoyloxy)benzene-1-sulfonate (NOBS), and/or those disclosed in WO98/17767. A family of bleach activators is disclosed in EP624154 and particularly preferred in that family is acetyl triethyl citrate (ATC). ATC or a short chain triglyceride like triacetin has the advantage that it is environmentally friendly. Furthermore acetyl triethyl citrate and triacetin have good hydrolytical stability in the product upon storage and are efficient bleach activators. Finally ATC is multifunctional, as the citrate released in the perhydrolysis reaction may function as a builder. Alternatively, the bleaching system may comprise peroxyacids of, for example, the amide, imide, or sulfone type. The bleaching system may also comprise peracids such as 6-(phthalimido)peroxyhexanoic acid (PAP). Suitable bleach activators are also disclosed in WO98/17767. While any suitable bleach activator may be employed, in one aspect of the invention the subject cleaning composition may comprise NOBS, TAED or mixtures thereof. When present, the peracid and/or bleach activator is generally present in the composition in an amount of 0.1 to 60 wt %, 0.5 to 40 wt % or 0.6 to 10 wt % based on the fabric and home care composition. One or more hydrophobic peracids or precursors thereof may be used in combination with one or more hydrophilic peracid or precursor thereof. Preferably such bleach components may be present in the compositions of the invention in an amount of 0.01 to 50 wt %, or 0.1 to 20 wt %.

The amounts of hydrogen peroxide source and peracid or bleach activator may be selected such that the molar ratio of available oxygen (from the peroxide source) to peracid is from 1:1 to 35:1, or even 2:1 to 10:1.

(4) Diacyl peroxides—preferred diacyl peroxide bleaching species include those selected from diacyl peroxides of the general formula: R¹—C(O)—OO—(O)C—R², in which R¹ represents a C₆-C₁₈ alkyl, preferably C₆-C₁₂ alkyl group containing a linear chain of at least 5 carbon atoms and optionally containing one or more substituents (e.g. —N⁺ (CH₃)₃, —COOH or —CN) and/or one or more interrupting moieties (e.g. —CONH— or —CH═CH—) interpolated between adjacent carbon atoms of the alkyl radical, and R² represents an aliphatic group compatible with a peroxide moiety, such that R¹ and R² together contain a total of 8 to 30 carbon atoms. In one preferred aspect R¹ and R² are linear unsubstituted C₆-C₁₂ alkyl chains. Most preferably R¹ and R² are identical. Diacyl peroxides, in which both R¹ and R² are C₆-C₁₂ alkyl groups, are particularly preferred. Preferably, at least one of, most preferably only one of, the R groups (R₁ or R₂), does not contain branching or pendant rings in the alpha position, or preferably neither in the alpha nor beta positions or most preferably in none of the alpha or beta or gamma positions. In one further preferred embodiment the DAP may be asymmetric, such that preferably the hydrolysis of R1 acyl group is rapid to generate peracid, but the hydrolysis of R2 acyl group is slow.

The tetraacyl peroxide bleaching species is preferably selected from tetraacyl peroxides of the general formula: R³—C(O)—OO—C(O)—(CH₂)n-C(O)—OO—C(O)—R³, in which R³ represents a C₁-C₉ alkyl, or C₃-C₇ group and n represents an integer from 2 to 12, or 4 to 10 inclusive.

Preferably, the diacyl and/or tetraacyl peroxide bleaching species is present in an amount sufficient to provide at least 0.5 ppm, at least 10 ppm, or at least 50 ppm by weight of the wash liquor. In a preferred embodiment, the bleaching species is present in an amount sufficient to provide from 0.5 to 300 ppm, from 30 to 150 ppm by weight of the wash liquor.

Preferably the bleach component comprises a bleach catalyst (5 and 6).

(5) Preferred are organic (non-metal) bleach catalysts include bleach catalyst capable of accepting an oxygen atom from a peroxyacid and/or salt thereof, and transferring the oxygen atom to an oxidizeable substrate. Suitable bleach catalysts include, but are not limited to: iminium cations and polyions; iminium zwitterions; modified amines; modified amine oxides; N-sulphonyl imines; N-phosphonyl imines; N-acyl imines; thiadiazole dioxides; perfluoroimines; cyclic sugar ketones and mixtures thereof.

Suitable iminium cations and polyions include, but are not limited to, N-methyl-3,4-dihydroisoquinolinium tetrafluoroborate, prepared as described in Tetrahedron (1992), 49(2), 423-38 (e.g. compound 4, p. 433); N-methyl-3,4-dihydroisoquinolinium p-toluene sulphonate, prepared as described in U.S. Pat. No. 5,360,569 (e.g. Column 11, Example 1); and N-octyl-3,4-dihydroisoquinolinium p-toluene sulphonate, prepared as described in U.S. Pat. No. 5,360,568 (e.g. Column 10, Ex. 3).

Suitable iminium zwitterions include, but are not limited to, N-(3-sulfopropyl)-3,4-dihydroisoquinolinium, inner salt, prepared as described in U.S. Pat. No. 5,576,282 (e.g. Column 31, Ex. II); N-[2-(sulphooxy)dodecyl]-3,4-dihydroisoquinolinium, inner salt, prepared as described in U.S. Pat. No. 5,817,614 (e.g. Column 32, Ex. V); 2-[3-[(2-ethylhexyl)oxy]-2-(sulphooxy)propyl]-3,4-dihydroisoquinolinium, inner salt, prepared as described in WO05/047264 (e.g. p. 18, Ex. 8), and 2-[3-[(2-butyloctyl)oxy]-2-(sulphooxy)propyl]-3,4-dihydroisoquinolinium, inner salt.

Suitable modified amine oxygen transfer catalysts include, but are not limited to, 1,2,3,4-tetrahydro-2-methyl-1-isoquinolinol, which can be made according to the procedures described in Tetrahedron Letters (1987), 28(48), 6061-6064. Suitable modified amine oxide oxygen transfer catalysts include, but are not limited to, sodium 1-hydroxy-N-oxy-N-[2-(sulphooxy)decyl]-1,2,3,4-tetrahydroisoquinoline.

Suitable N-sulphonyl imine oxygen transfer catalysts include, but are not limited to, 3-methyl-1,2-benzisothiazole 1,1-dioxide, prepared according to the procedure described in the Journal of Organic Chemistry (1990), 55(4), 1254-61.

Suitable N-phosphonyl imine oxygen transfer catalysts include, but are not limited to, [R-(E)]-N-[(2-chloro-5-nitrophenyl)methylene]-P-phenyl-P-(2,4,6-trimethylphenyl)-phosphinic amide, which can be made according to the procedures described in the Journal of the Chemical Society, Chemical Communications (1994), (22), 2569-70.

Suitable N-acyl imine oxygen transfer catalysts include, but are not limited to, [N(E)]-N-(phenylmethylene)acetamide, which can be made according to the procedures described in Polish Journal of Chemistry (2003), 77(5), 577-590.

Suitable thiadiazole dioxide oxygen transfer catalysts include but are not limited to, 3-methyl-4-phenyl-1,2,5-thiadiazole 1,1-dioxide, which can be made according to the procedures described in U.S. Pat. No. 5,753,599 (Column 9, Ex. 2).

Suitable perfluoroimine oxygen transfer catalysts include, but are not limited to, (Z)-2,2,3,3,4,4,4-heptafluoro-N-(nonafluorobutyl)butanimidoyl fluoride, which can be made according to the procedures described in Tetrahedron Letters (1994), 35(34), 6329-30.

Suitable cyclic sugar ketone oxygen transfer catalysts include, but are not limited to, 1,2:4,5-di-O-isopropylidene-D-erythro-2,3-hexodiuro-2,6-pyranose as prepared in U.S. Pat. No. 6,649,085 (Column 12, Ex. 1).

Preferably, the bleach catalyst comprises an iminium and/or carbonyl functional group and is typically capable of forming an oxaziridinium and/or dioxirane functional group upon acceptance of an oxygen atom, especially upon acceptance of an oxygen atom from a peroxyacid and/or salt thereof. Preferably, the bleach catalyst comprises an oxaziridinium functional group and/or is capable of forming an oxaziridinium functional group upon acceptance of an oxygen atom, especially upon acceptance of an oxygen atom from a peroxyacid and/or salt thereof. Preferably, the bleach catalyst comprises a cyclic iminium functional group, preferably wherein the cyclic moiety has a ring size of from five to eight atoms (including the nitrogen atom), preferably six atoms. Preferably, the bleach catalyst comprises an aryliminium functional group, preferably a bi-cyclic aryliminium functional group, preferably a 3,4-dihydroisoquinolinium functional group. Typically, the imine functional group is a quaternary imine functional group and is typically capable of forming a quaternary oxaziridinium functional group upon acceptance of an oxygen atom, especially upon acceptance of an oxygen atom from a peroxyacid and/or salt thereof. In another aspect, the detergent composition comprises a bleach component having a log P_{o/w} no greater than 0, no greater than −0.5, no greater than −1.0, no greater than −1.5, no greater than −2.0, no greater than −2.5, no greater than −3.0, or no greater than −3.5. The method for determining log P_{o/w} is described in more detail below.

Typically, the bleach ingredient is capable of generating a bleaching species having a X_{SO} of from 0.01 to 0.30, from 0.05 to 0.25, or from 0.10 to 0.20. The method for determining X_{SO} is described in more detail below. For example, bleaching ingredients having an isoquinolinium structure are capable of generating a bleaching species that has an oxaziridinium structure. In this example, the X_{SO} is that of the oxaziridinium bleaching species.

Preferably, the bleach catalyst has a chemical structure corresponding to the following chemical formula:

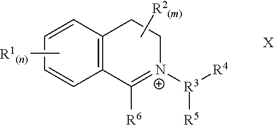

wherein: n and m are independently from 0 to 4, preferably n and m are both 0; each R¹ is independently selected from a substituted or unsubstituted radical selected from the group consisting of hydrogen, alkyl, cycloalkyl, aryl, fused aryl, heterocyclic ring, fused heterocyclic ring, nitro, halo, cyano, sulphonato, alkoxy, keto, carboxylic, and carboalkoxy radicals; and any two vicinal R¹ substituents may combine to form a fused aryl, fused carbocyclic or fused heterocyclic ring; each R² is independently selected from a substituted or unsubstituted radical independently selected from the group consisting of hydrogen, hydroxy, alkyl, cycloalkyl, alkaryl, aryl, aralkyl, alkylenes, heterocyclic ring, alkoxys, arylcarbonyl groups, carboxyalkyl groups and amide groups; any R² may be joined together with any other of R² to form part of a common ring; any geminal R² may combine to form a carbonyl; and any two R² may combine to form a substituted or unsubstituted fused unsaturated moiety; R³ is a C₁ to C₂₀ substituted or unsubstituted alkyl; R⁴ is hydrogen or the moiety Qₜ-A, wherein: Q is a branched or unbranched alkylene, t=0 or 1 and A is an anionic group selected from the group consisting of OSO₃⁻, SO₃⁻, CO2⁻, OCO₂⁻, OPO₃²⁻, OPO₃H⁻ and OPO₂⁻; R⁵ is hydrogen or the moiety—CR¹¹R¹²—Y-G_{b}-Y_{c}—[(CR⁹R¹⁰)_{y}—O]ₖ—R⁸, wherein: each Y is independently selected from the group consisting of O, S, N—H, or N—R⁸; and each R⁸ is independently selected from the group consisting of alkyl, aryl and heteroaryl, said moieties being substituted or unsubstituted, and whether substituted or unsubsituted said moieties having less than 21 carbons; each G is independently selected from the group consisting of CO, SO₂, SO, PO and PO₂; R⁹ and R¹⁰ are independently selected from the group consisting of H and C₁-C₄ alkyl; R¹¹ and R¹² are independently selected from the group consisting of H and alkyl, or when taken together may join to form a carbonyl; b=0 or 1; c can=0 or 1, but c must=0 if b=0; y is an integer from 1 to 6; k is an integer from 0 to 20; R⁶ is H, or an alkyl, aryl or heteroaryl moiety; said moieties being substituted or unsubstituted; and X, if present, is a suitable charge balancing counterion, preferably X is present when R⁴ is hydrogen, suitable X, include but are not limited to: chloride, bromide, sulphate, methosulphate, sulphonate, p-toluenesulphonate, borontetraflouride and phosphate.

In one embodiment of the present invention, the bleach catalyst has a structure corresponding to general formula below:

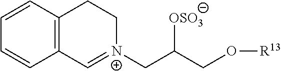

wherein R¹³ is a branched alkyl group containing from three to 24 carbon atoms (including the branching carbon atoms) or a linear alkyl group containing from one to 24 carbon atoms; preferably R¹³ is a branched alkyl group containing from eight to 18 carbon atoms or linear alkyl group containing from eight to eighteen carbon atoms; preferably R¹³ is selected from the group consisting of 2-propylheptyl, 2-butyloctyl, 2-pentylnonyl, 2-hexyldecyl, n-dodecyl, n-tetradecyl, n-hexadecyl, n-octadecyl, iso-nonyl, iso-decyl, iso-tridecyl and iso-pentadecyl; preferably R¹³ is selected from the group consisting of 2-butyloctyl, 2-pentylnonyl, 2-hexyldecyl, iso-tridecyl and iso-pentadecyl.

Preferably the bleach component comprises a source of peracid in addition to bleach catalyst, particularly organic bleach catalyst. The source of peracid may be selected from (a) pre-formed peracid; (b) percarbonate, perborate or persulfate salt (hydrogen peroxide source) preferably in combination with a bleach activator; and (c) perhydrolase enzyme and an ester for forming peracid in situ in the presence of water in a textile or hard surface treatment step.

When present, the peracid and/or bleach activator is generally present in the composition in an amount of from 0.1 to 60 wt %, from 0.5 to 40 wt % or from 0.6 to 10 wt % based on the composition. One or more hydrophobic peracids or precursors thereof may be used in combination with one or more hydrophilic peracid or precursor thereof.

The amounts of hydrogen peroxide source and peracid or bleach activator may be selected such that the molar ratio of available oxygen (from the peroxide source) to peracid is from 1:1 to 35:1, or 2:1 to 10:1.

(6) Metal-containing Bleach Catalysts—The bleach component may be provided by a catalytic metal complex. One type of metal-containing bleach catalyst is a catalyst system comprising a transition metal cation of defined bleach catalytic activity, such as copper, iron, titanium, ruthenium, tungsten, molybdenum, or manganese cations, an auxiliary metal cation having little or no bleach catalytic activity, such as zinc or aluminum cations, and a sequestrate having defined stability constants for the catalytic and auxiliary metal cations, particularly ethylenediaminetetraacetic acid, ethylenediaminetetra(methylenephosphonic acid) and water-soluble salts thereof. Such catalysts are disclosed in U.S. Pat. No. 4,430,243. Preferred catalysts are described in WO09/839406, U.S. Pat. No. 6,218,351 and WO00/012667. Particularly preferred are transition metal catalyst or ligands therefore that are cross-bridged polydentate N-donor ligands.

If desired, the compositions herein can be catalyzed by means of a manganese compound. Such compounds and levels of use are well known in the art and include, e.g., the manganese-based catalysts disclosed in U.S. Pat. No. 5,576,282.

Cobalt bleach catalysts useful herein are known, and are described, e.g., in U.S. Pat. Nos. 5,597,936; 5,595,967. Such cobalt catalysts are readily prepared by known procedures, such as taught, e.g., in U.S. Pat. Nos. 5,597,936 and 5,595,967.

Compositions herein may also suitably include a transition metal complex of ligands such as bispidones (U.S. Pat. No. 7,501,389) and/or macropolycyclic rigid ligands—abbreviated as “MRLs”. As a practical matter, and not by way of limitation, the compositions and processes herein can be adjusted to provide on the order of at least one part per hundred million of the active MRL species in the aqueous washing medium, and will typically provide from 0.005 to 25 ppm, from 0.05 to 10 ppm, or from 0.1 to 5 ppm, of the MRL in the wash liquor.

Suitable transition-metals in the instant transition-metal bleach catalyst include e.g. manganese, iron and chromium. Suitable MRLs include 5,12-diethyl-1,5,8,12-tetraazabicyclo[6.6.2]hexadecane. Suitable transition metal MRLs are readily prepared by known procedures, such as taught e.g. in U.S. Pat. No. 6,225,464 and WO00/32601.

(7) Photobleaches—suitable photobleaches include e.g. sulfonated zinc phthalocyanine sulfonated aluminium phthalocyanines, xanthene dyes and mixtures thereof. Preferred bleach components for use in the present compositions of the invention comprise a hydrogen peroxide source, bleach activator and/or organic peroxyacid, optionally generated in situ by the reaction of a hydrogen peroxide source and bleach activator, in combination with a bleach catalyst. Preferred bleach components comprise bleach catalysts, preferably organic bleach catalysts, as described above.

Particularly preferred bleach components are the bleach catalysts in particular the organic bleach catalysts.

Exemplary bleaching systems are also described, e.g. in WO2007/087258, WO2007/087244, WO2007/087259 and WO2007/087242.

Fabric Hueing Agents—The composition may comprise a fabric hueing agent. Suitable fabric hueing agents include dyes, dye-clay conjugates, and pigments. Suitable dyes include small molecule dyes and polymeric dyes. Suitable small molecule dyes include small molecule dyes selected from the group consisting of dyes falling into the Color Index (C.I.) classifications of Direct Blue, Direct Red, Direct Violet, Acid Blue, Acid Red, Acid Violet, Basic Blue, Basic Violet and Basic Red, or mixtures thereof.

In another aspect, suitable small molecule dyes include small molecule dyes selected from the group consisting of Color Index (Society of Dyers and Colorists, Bradford, UK) numbers Direct Violet 9, Direct Violet 35, Direct Violet 48, Direct Violet 51, Direct Violet 66, Direct Violet 99, Direct Blue 1, Direct Blue 71, Direct Blue 80, Direct Blue 279, Acid Red 17, Acid Red 73, Acid Red 88, Acid Red 150, Acid Violet 15, Acid Violet 17, Acid Violet 24, Acid Violet 43, Acid Red 52, Acid Violet 49, Acid Violet 50, Acid Blue 15, Acid Blue 17, Acid Blue 25, Acid Blue 29, Acid Blue 40, Acid Blue 45, Acid Blue 75, Acid Blue 80, Acid Blue 83, Acid Blue 90 and Acid Blue 113, Acid Black 1, Basic Violet 1, Basic Violet 3, Basic Violet 4, Basic Violet 10, Basic Violet 35, Basic Blue 3, Basic Blue 16, Basic Blue 22, Basic Blue 47, Basic Blue 66, Basic Blue 75, Basic Blue 159 and mixtures thereof. In another aspect, suitable small molecule dyes include small molecule dyes selected from the group consisting of Color Index (Society of Dyers and Colorists, Bradford, UK) numbers Acid Violet 17, Acid Violet 43, Acid Red 52, Acid Red 73, Acid Red 88, Acid Red 150, Acid Blue 25, Acid Blue 29, Acid Blue 45, Acid Blue 113, Acid Black 1, Direct Blue 1, Direct Blue 71, Direct Violet 51 and mixtures thereof. In another aspect, suitable small molecule dyes include small molecule dyes selected from the group consisting of Color Index (Society of Dyers and Colorists, Bradford, UK) numbers Acid Violet 17, Direct Blue 71, Direct Violet 51, Direct Blue 1, Acid Red 88, Acid Red 150, Acid Blue 29, Acid Blue 113 or mixtures thereof.

Suitable polymeric dyes include polymeric dyes selected from the group consisting of polymers containing conjugated chromogens (dye-polymer conjugates) and polymers with chromogens co-polymerized into the backbone of the polymer and mixtures thereof.

In another aspect, suitable polymeric dyes include polymeric dyes selected from the group consisting of fabric-substantive colorants sold under the name of Liquitint® (Milliken), dye-polymer conjugates formed from at least one reactive dye and a polymer selected from the group consisting of polymers comprising a moiety selected from the group consisting of a hydroxyl moiety, a primary amine moiety, a secondary amine moiety, a thiol moiety and mixtures thereof. In still another aspect, suitable polymeric dyes include polymeric dyes selected from the group consisting of Liquitint® Violet CT, carboxymethyl cellulose (CMC) conjugated with a reactive blue, reactive violet or reactive red dye such as CMC conjugated with C.I. Reactive Blue 19, sold by Megazyme, Wicklow, Ireland under the product name AZO-CM-CELLULOSE, product code S-ACMC, alkoxylated triphenyl-methane polymeric colorants, alkoxylated thiophene polymeric colorants, and mixtures thereof.

Preferred hueing dyes include the whitening agents found in WO08/87497. These whitening agents may be characterized by the following structure (1):

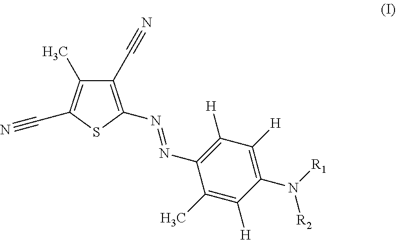

wherein R₁ and R₂ can independently be selected from:

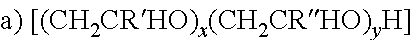

wherein R′ is selected from the group consisting of H, CH₃, CH₂O(CH₂CH₂O)_{z}H, and mixtures thereof; wherein R″ is selected from the group consisting of H, CH₂O(CH₂CH₂O)_{z}H, and mixtures thereof; wherein x+y≤5; wherein y≥1; and wherein z=0 to 5;
   b) R₁=alkyl, aryl or aryl alkyl and R₂═[(CH₂CR′HO)ₓ(CH₂CR″HO)_{y}H]
wherein R′ is selected from the group consisting of H, CH₃, CH₂O(CH₂CH₂O)_{z}H, and mixtures thereof; wherein R″ is selected from the group consisting of H, CH₂O(CH₂CH₂O)_{z}H, and mixtures thereof; wherein x+y≤10; wherein y≥1; and wherein z=0 to 5;

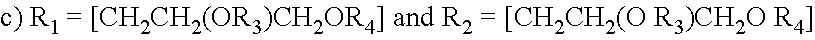

wherein R₃ is selected from the group consisting of H, (CH₂CH₂O)_{z}H, and mixtures thereof; and
wherein z=0 to 10;
wherein R₄ is selected from the group consisting of (C₁-C₁₆)alkyl, aryl groups, and mixtures thereof; and
   d) wherein R1 and R2 can independently be selected from the amino addition product of styrene oxide, glycidyl methyl ether, isobutyl glycidyl ether, isopropylglycidyl ether, t-butyl glycidyl ether, 2-ethylhexylgycidyl ether, and glycidylhexadecyl ether, followed by the addition of from 1 to 10 alkylene oxide units.

A preferred whitening agent of the present invention may be characterized by the following structure (II):

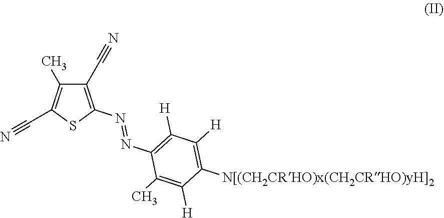

wherein R′ is selected from the group consisting of H, CH₃, CH₂O(CH₂CH₂O)_{z}H, and mixtures thereof; wherein R″ is selected from the group consisting of H, CH₂O(CH₂CH₂O)_{z}H, and mixtures thereof; wherein x+y≤5; wherein y≥1; and wherein z=0 to 5.

A further preferred whitening agent of the present invention may be characterized by the following structure (III):

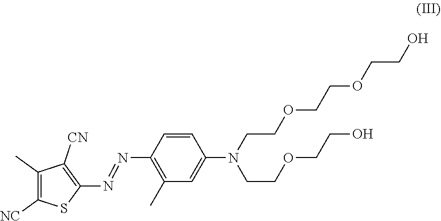 [SMILES: Cc1cc(N(CCOCCO)CCOCCOCCO)ccc1/N=N/c1sc(C#N)c(C)c1C#N]

typically comprising a mixture having a total of 5 EO groups. Suitable preferred molecules are those in Structure I having the following pendant groups in “part a” above.

TABLE 1

| | R1 | | | | R2 | | | |
|---|---|---|---|---|---|---|---|---|
| | R′ | R″ | X | y | R′ | R″ | x | y |
| A | H | H | 3 | 1 | H | H | 0 | 1 |
| B | H | H | 2 | 1 | H | H | 1 | 1 |
| c = b | H | H | 1 | 1 | H | H | 2 | 1 |
| d = a | H | H | 0 | 1 | H | H | 3 | 1 |

Further whitening agents of use include those described in US2008/34511 (Unilever). A preferred agent is “Violet 13”.

Suitable dye clay conjugates include dye clay conjugates selected from the group comprising at least one cationic/basic dye and a smectite clay, and mixtures thereof. In another aspect, suitable dye clay conjugates include dye clay conjugates selected from the group consisting of one cationic/basic dye selected from the group consisting of C.I. Basic Yellow 1 through 108, C.I. Basic Orange 1 through 69, C.I. Basic Red 1 through 118, C.I. Basic Violet 1 through 51, C.I. Basic Blue 1 through 164, C.I. Basic Green 1 through 14, C.I. Basic Brown 1 through 23, Cl Basic Black 1 through 11, and a clay selected from the group consisting of Montmorillonite clay, Hectorite clay, Saponite clay and mixtures thereof. In still another aspect, suitable dye clay conjugates include dye clay conjugates selected from the group consisting of: Montmorillonite Basic Blue B7 C.I. 42595 conjugate, Montmorillonite Basic Blue B9 C.I. 52015 conjugate, Montmorillonite Basic Violet V3 C.I. 42555 conjugate, Montmorillonite Basic Green G1 C.I. 42040 conjugate, Montmorillonite Basic Red R1 C.I. 45160 conjugate, Montmorillonite C.I. Basic Black 2 conjugate, Hectorite Basic Blue B7 C.I. 42595 conjugate, Hectorite Basic Blue B9 C.I. 52015 conjugate, Hectorite Basic Violet V3 C.I. 42555 conjugate, Hectorite Basic Green G1 C.I. 42040 conjugate, Hectorite Basic Red R1 C.I. 45160 conjugate, Hectorite C.I. Basic Black 2 conjugate, Saponite Basic Blue B7 C.I. 42595 conjugate, Saponite Basic Blue B9 C.I. 52015 conjugate, Saponite Basic Violet V3 C.I. 42555 conjugate, Saponite Basic Green G1 C.I. 42040 conjugate, Saponite Basic Red R1 C.I. 45160 conjugate, Saponite C.I. Basic Black 2 conjugate and mixtures thereof.

Suitable pigments include pigments selected from the group consisting of flavanthrone, indanthrone, chlorinated indanthrone containing from 1 to 4 chlorine atoms, pyranthrone, dichloropyranthrone, monobromodichloropyranthrone, dibromodichloropyranthrone, tetrabromopyranthrone, perylene-3,4,9,10-tetracarboxylic acid diimide, wherein the imide groups may be unsubstituted or substituted by C1-C3-alkyl or a phenyl or heterocyclic radical, and wherein the phenyl and heterocyclic radicals may additionally carry substituents which do not confer solubility in water, anthrapyrimidinecarboxylic acid amides, violanthrone, isoviolanthrone, dioxazine pigments, copper phthalocyanine which may contain up to 2 chlorine atoms per molecule, polychloro-copper phthalocyanine or polybromochloro-copper phthalocyanine containing up to 14 bromine atoms per molecule and mixtures thereof.

In another aspect, suitable pigments include pigments selected from the group consisting of Ultramarine Blue (C.I. Pigment Blue 29), Ultramarine Violet (C.I. Pigment Violet 15) and mixtures thereof.

The aforementioned fabric hueing agents can be used in combination (any mixture of fabric hueing agents can be used). Suitable hueing agents are described in more detail in U.S. Pat. No. 7,208,459. Preferred levels of dye in compositions of the invention are 0.00001 to 0.5 wt %, or 0.0001 to 0.25 wt %. The concentration of dyes preferred in water for the treatment and/or cleaning step is from 1 ppb to 5 ppm, 10 ppb to 5 ppm or 20 ppb to 5 ppm. In preferred compositions, the concentration of surfactant will be from 0.2 to 3 g/l.

Encapsulates—The composition may comprise an encapsulate. In one aspect, an encapsulate comprising a core, a shell having an inner and outer surface, said shell encapsulating said core.

In one aspect of said encapsulate, said core may comprise a material selected from the group consisting of perfumes; brighteners; dyes; insect repellants; silicones; waxes; flavors; vitamins; fabric softening agents; skin care agents in one aspect, paraffins; enzymes; anti-bacterial agents; bleaches; sensates; and mixtures thereof; and said shell may comprise a material selected from the group consisting of polyethylenes; polyamides; polyvinylalcohols, optionally containing other co-monomers; polystyrenes; polyisoprenes; polycarbonates; polyesters; polyacrylates; aminoplasts, in one aspect said aminoplast may comprise a polyureas, polyurethane, and/or polyureaurethane, in one aspect said polyurea may comprise polyoxymethyleneurea and/or melamine formaldehyde; polyolefins; polysaccharides, in one aspect said polysaccharide may comprise alginate and/or chitosan; gelatin; shellac; epoxy resins; vinyl polymers; water insoluble inorganics; silicone; and mixtures thereof.

In one aspect of said encapsulate, said core may comprise perfume.

In one aspect of said encapsulate, said shell may comprise melamine formaldehyde and/or cross-linked melamine formaldehyde.

In a one aspect, suitable encapsulates may comprise a core material and a shell, said shell at least partially surrounding said core material, is disclosed. At least 75%, 85% or 90% of said encapsulates may have a fracture strength of from 0.2 to 10 MPa, from 0.4 to 5 MPa, from 0.6 to 3.5 MPa, or from 0.7 to 3 MPa; and a benefit agent leakage of from 0 to 30%, from 0 to 20%, or from 0 to 5%.

In one aspect, at least 75%, 85% or 90% of said encapsulates may have a particle size from 1 to 80 microns, from 5 to 60 microns, from 10 to 50 microns, or from 15 to 40 microns.

In one aspect, at least 75%, 85% or 90% of said encapsulates may have a particle wall thickness from 30 to 250 nm, from 80 to 180 nm, or from 100 to 160 nm.

In one aspect, said encapsulates' core material may comprise a material selected from the group consisting of a perfume raw material and/or optionally a material selected from the group consisting of vegetable oil, including neat and/or blended vegetable oils including castor oil, coconut oil, cottonseed oil, grape oil, rapeseed, soybean oil, corn oil, palm oil, linseed oil, safflower oil, olive oil, peanut oil, coconut oil, palm kernel oil, castor oil, lemon oil and mixtures thereof; esters of vegetable oils, esters, including dibutyl adipate, dibutyl phthalate, butyl benzyl adipate, benzyl octyl adipate, tricresyl phosphate, trioctyl phosphate and mixtures thereof; straight or branched chain hydrocarbons, including those straight or branched chain hydrocarbons having a boiling point of greater than about 80° C.; partially hydrogenated terphenyls, dialkyl phthalates, alkyl biphenyls, including monoisopropylbiphenyl, alkylated naphthalene, including dipropylnaphthalene, petroleum spirits, including kerosene, mineral oil and mixtures thereof; aromatic solvents, including benzene, toluene and mixtures thereof; silicone oils; and mixtures thereof.

In one aspect, said encapsulates' wall material may comprise a suitable resin including the reaction product of an aldehyde and an amine, suitable aldehydes include formaldehyde. Suitable amines include melamine, urea, benzoguanamine, glycoluril, and mixtures thereof. Suitable melamines include methylol melamine, methylated methylol melamine, imino melamine and mixtures thereof. Suitable ureas include dimethylol urea, methylated dimethylol urea, urea-resorcinol, and mixtures thereof.

In one aspect, suitable formaldehyde scavengers may be employed with the encapsulates, e.g., in a capsule slurry and/or added to a composition before, during or after the encapsulates are added to such composition. Suitable capsules may be made by the following teaching of US2008/0305982; and/or US2009/0247449.

In a preferred aspect the composition can also comprise a deposition aid, preferably consisting of the group comprising cationic or nonionic polymers. Suitable polymers include cationic starches, cationic hydroxyethylcellulose, polyvinylformaldehyde, locust bean gum, mannans, xyloglucans, tamarind gum, polyethyleneterephthalate and polymers containing dimethylaminoethyl methacrylate, optionally with one or monomers selected from the group comprising acrylic acid and acrylamide.

Perfumes—In one aspect the composition comprises a perfume that comprises one or more perfume raw materials selected from the group consisting of 1,1′-oxybis-2-propanol; 1,4-cyclohexanedicarboxylic acid, diethyl ester; (ethoxymethoxy)cyclododecane; 1,3-nonanediol, monoacetate; (3-methylbutoxy)acetic acid, 2-propenyl ester; beta-methyl cyclododecaneethanol; 2-methyl-3-[(1,7,7-trimethylbicyclo[2.2.1]hept-2-yl)oxy]-1-propanol; oxacyclohexadecan-2-one; alpha-methyl-benzenemethanol acetate; trans-3-ethoxy-1,1,5-trimethylcyclohexane; 4-(1,1-dimethylethyl)cyclohexanol acetate; dodecahydro-3a,6,6,9a-tetramethylnaphtho[2,1-b]furan; beta-methyl benzenepropanal; beta-methyl-3-(1-methylethyl)benzenepropanal; 4-phenyl-2-butanone; 2-methylbutanoic acid, ethyl ester; benzaldehyde; 2-methylbutanoic acid, 1-methylethyl ester; dihydro-5-pentyl-2(3H)furanone; (2E)-1-(2,6,6-trimethyl-2-cyclohexen-1-yl)-2-buten-1-one; dodecanal; undecanal; 2-ethyl-alpha, alpha-dimethylbenzenepropanal; decanal; alpha, alpha-dimethylbenzeneethanol acetate; 2-(phenylmethylene)octanal; 2-[[3-[4-(1,1-dimethylethyl)phenyl]-2-methylpropylidene]amino]benzoic acid, methyl ester; 1-(2,6,6-trimethyl-3-cyclohexen-1-yl)-2-buten-1-one; 2-pentylcyclopentanone; 3-oxo-2-pentyl cyclopentaneacetic acid, methyl ester; 4-hydroxy-3-methoxybenzaldehyde; 3-ethoxy-4-hydroxybenzaldehyde; 2-heptylcyclopentanone; 1-(4-methylphenyl)ethanone; (3E)-4-(2,6,6-trimethyl-1-cyclohexen-1-yl)-3-buten-2-one; (3E)-4-(2,6,6-trimethyl-2-cyclohexen-1-yl)-3-buten-2-one; benzeneethanol; 2H-1-benzopyran-2-one; 4-methoxybenzaldehyde; 10-undecenal; propanoic acid, phenylmethyl ester; beta-methylbenzenepentanol; 1,1-diethoxy-3,7-dimethyl-2,6-octadiene; alpha, alpha-dimethylbenzeneethanol; (2E)-1-(2,6,6-trimethyl-1-cyclohexen-1-yl)-2-buten-1-one; acetic acid, phenylmethyl ester; cyclohexanepropanoic acid, 2-propenyl ester; hexanoic acid, 2-propenyl ester; 1,2-dimethoxy-4-(2-propenyl)benzene; 1,5-dimethyl-bicyclo[3.2.1]octan-8-one oxime; 4-(4-hydroxy-4-methylpentyl)-3-cyclohexene-1-carboxaldehyde; 3-buten-2-ol; 2-[[[2,4 (or 3,5)-dimethyl-3-cyclohexen-1-yl]methylene]amino]benzoic acid, methyl ester; 8-cyclohexadecen-1-one; methyl ionone; 2,6-dimethyl-7-octen-2-ol; 2-methoxy-4-(2-propenyl)phenol; (2E)-3,7-dimethyl-2,6-Octadien-1-ol; 2-hydroxy-Benzoic acid, (3Z)-3-hexenyl ester; 2-tridecenenitrile; 4-(2,2-dimethyl-6-methylenecyclohexyl)-3-methyl-3-buten-2-one; tetrahydro-4-methyl-2-(2-methyl-1-propenyl)-2H-pyran; Acetic acid, (2-methylbutoxy)-, 2-propenyl ester; Benzoic acid, 2-hydroxy-, 3-methylbutyl ester; 2-Buten-1-one, 1-(2,6,6-trimethyl-1-cyclohexen-1-yl)-, (Z)-; Cyclopentanecarboxylic acid, 2-hexyl-3-oxo-, methyl ester; Benzenepropanal, 4-ethyl-.alpha.,.alpha.-dimethyl-; 3-Cyclohexene-1-carboxaldehyde, 3-(4-hydroxy-4-methylpentyl)-; Ethanone, 1-(2,3,4,7,8,8a-hexahydro-3,6,8,8-tetramethyl-1H-3a,7-methanoazulen-5-yl)-, [3R-(3.alpha.,3a.beta.,7.beta.,8a.alpha.)]-; Undecanal, 2-methyl-2H-Pyran-2-one, 6-butyltetrahydro-; Benzenepropanal, 4-(1,1-dimethylethyl)-.alpha.-methyl-; 2(3H)-Furanone, 5-heptyldihydro-; Benzoic acid, 2-[(7-hydroxy-3,7-dimethyloctylidene)amino]-, methyl; Benzoic acid, 2-hydroxy-, phenylmethyl ester; Naphthalene, 2-methoxy-; 2-Cyclopenten-1-one, 2-hexyl-; 2(3H)-Furanone, 5-hexyldihydro-; Oxiranecarboxylic acid, 3-methyl-3-phenyl-, ethyl ester; 2-Oxabicyclo[2.2.2]octane, 1,3,3-trimethyl-; Benzenepentanol, .gamma.-methyl-; 3-Octanol, 3,7-dimethyl-; 3,7-dimethyl-2,6-octadienenitrile; 3,7-dimethyl-6-octen-1-ol; Terpineol acetate; 2-methyl-6-methylene-7-Octen-2-ol, dihydro derivative; 3a,4,5,6,7,7a-hexahydro-4,7-Methano-1H-inden-6-ol propanoate; 3-methyl-2-buten-1-ol acetate; (Z)-3-Hexen-1-ol acetate; 2-ethyl-4-(2,2,3-trimethyl-3-cyclopenten-1-yl)-2-buten-1-ol; 4-(octahydro-4,7-methano-5H-inden-5-ylidene)-butanal; 3-2,4-dimethyl-cyclohexene-1-carboxaldehyde; 1-(1,2,3,4,5,6,7,8-octahydro-2,3,8,8-tetramethyl-2-naphthalenyl)-ethanone; 2-hydroxy-benzoic acid, methyl ester; 2-hydroxy-benzoic acid, hexyl ester; 2-phenoxy-ethanol; 2-hydroxy-benzoic acid, pentyl ester; 2,3-heptanedione; 2-hexen-1-ol; 6-Octen-2-ol, 2,6-dimethyl-; damascone (alpha, beta, gamma or delta or mixtures thereof), 4,7-Methano-1H-inden-6-ol, 3a,4,5,6,7,7a-hexahydro-, acetate; 9-Undecenal; 8-Undecenal; Isocyclocitral; Ethanone, 1-(1,2,3,5,6,7,8,8a-octahydro-2,3,8,8-tetramethyl-2-naphthalenyl)-; 3-Cyclohexene-1-carboxaldehyde, 3,5-dimethyl-; 3-Cyclohexene-1-carboxaldehyde, 2,4-dimethyl-; 1,6-Octadien-3-ol, 3,7-dimethyl-; 1,6-Octadien-3-ol, 3,7-dimethyl-, acetate; Lilial (p-t-Bucinal), and Cyclopentanone, 2-[2-(4-methyl-3-cyclohexen-1-yl)propyl]- and 1-methyl-4-(1-methylethenyl)cyclohexene and mixtures thereof.

In one aspect the composition may comprise an encapsulated perfume particle comprising either a water-soluble hydroxylic compound or melamine-formaldehyde or modified polyvinyl alcohol. In one aspect the encapsulate comprises (a) an at least partially water-soluble solid matrix comprising one or more water-soluble hydroxylic compounds, preferably starch; and (b) a perfume oil encapsulated by the solid matrix.

In a further aspect the perfume may be pre-complexed with a polyamine, preferably a polyethylenimine so as to form a Schiff base.

Polymers—The composition may comprise one or more polymers. Examples are carboxymethylcellulose, poly(vinyl-pyrrolidone), poly (ethylene glycol), poly(vinyl alcohol), poly(vinylpyridine-N-oxide), poly(vinylimidazole), polycarboxylates such as polyacrylates, maleic/acrylic acid copolymers and lauryl methacrylate/acrylic acid co-polymers.

The composition may comprise one or more amphiphilic cleaning polymers such as the compound having the following general structure: bis((C₂H₅O)(C₂H₄O)n)(CH₃)—N⁺—CₓH₂ₓ—N⁺—(CH₃)-bis((C₂H₅O)(C₂H₄O)n), wherein n=from 20 to 30, and x=from 3 to 8, or sulphated or sulphonated variants thereof.

The composition may comprise amphiphilic alkoxylated grease cleaning polymers which have balanced hydrophilic and hydrophobic properties such that they remove grease particles from fabrics and surfaces. Specific embodiments of the amphiphilic alkoxylated grease cleaning polymers of the present invention comprise a core structure and a plurality of alkoxylate groups attached to that core structure. These may comprise alkoxylated polyalkylenimines, preferably having an inner polyethylene oxide block and an outer polypropylene oxide block.

Alkoxylated polycarboxylates such as those prepared from polyacrylates are useful herein to provide additional grease removal performance. Such materials are described in WO91/08281 and PCT90/01815. Chemically, these materials comprise polyacrylates having one ethoxy side-chain per every 7-8 acrylate units. The side-chains are of the formula —(CH₂CH₂O)ₘ (CH₂)ₙCH₃ wherein m is 2-3 and n is 6-12. The side-chains are ester-linked to the polyacrylate “backbone” to provide a “comb” polymer type structure. The molecular weight can vary, but is typically in the range of 2000 to 50,000. Such alkoxylated polycarboxylates can comprise from 0.05 wt % to 10 wt % of the compositions herein.

The isoprenoid-derived surfactants of the present invention, and their mixtures with other cosurfactants and other adjunct ingredients, are particularly suited to be used with an amphilic graft co-polymer, preferably the amphilic graft co-polymer comprises (i) polyethyelene glycol backbone; and (ii) and at least one pendant moiety selected from polyvinyl acetate, polyvinyl alcohol and mixtures thereof. A preferred amphilic graft co-polymer is Sokalan HP22, supplied from BASF. Suitable polymers include random graft copolymers, preferably a polyvinyl acetate grafted polyethylene oxide copolymer having a polyethylene oxide backbone and multiple polyvinyl acetate side chains. The molecular weight of the polyethylene oxide backbone is preferably 6000 and the weight ratio of the polyethylene oxide to polyvinyl acetate is 40 to 60 and no more than 1 grafting point per 50 ethylene oxide units.

Carboxylate polymer—The composition of the present invention may also include one or more carboxylate polymers such as a maleate/acrylate random copolymer or polyacrylate homopolymer. In one aspect, the carboxylate polymer is a polyacrylate homopolymer having a molecular weight of from 4,000 to 9,000 Da, or from 6,000 to 9,000 Da.

Soil release polymer—The composition of the present invention may also include one or more soil release polymers having a structure as defined by one of the following structures (I), (II) or (III):

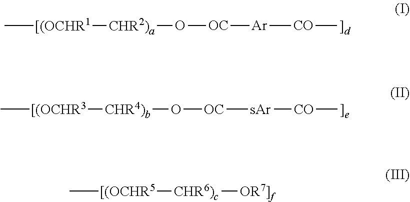

wherein:
a, b and c are from 1 to 200;
d, e and f are from 1 to 50;
Ar is a 1,4-substituted phenylene;
sAr is 1,3-substituted phenylene substituted in position 5 with SO₃Me;
Me is Li, K, Mg/2, Ca/2, Al/3, ammonium, mono-, di-, tri-, or tetraalkylammonium wherein the alkyl groups are C₁-C₁₈ alkyl or C₂-C₁₀ hydroxyalkyl, or mixtures thereof; R¹, R², R³, R⁴, R⁵ and R⁶ are independently selected from H or C₁-C₁₈ n- or iso-alkyl; and
R⁷ is a linear or branched C₁-C₁₈ alkyl, or a linear or branched C₂-C₃₀ alkenyl, or a cycloalkyl group with 5 to 9 carbon atoms, or a C₈-C₃₀ aryl group, or a C₆-C₃₀ arylalkyl group.

Suitable soil release polymers are polyester soil release polymers such as Repel-o-tex polymers, including Repel-o-tex, SF-2 and SRP6 supplied by Rhodia. Other suitable soil release polymers include Texcare polymers, including Texcare SRA100, SRA300, SRN100, SRN170, SRN240, SRN300 and SRN325 supplied by Clariant. Other suitable soil release polymers are Marloquest polymers, such as Marloquest SL supplied by Sasol.

Cellulosic polymer—The composition of the present invention may also include one or more cellulosic polymers including those selected from alkyl cellulose, alkyl alkoxyalkyl cellulose, carboxyalkyl cellulose, alkyl carboxyalkyl cellulose. In one aspect, the cellulosic polymers are selected from the group comprising carboxymethyl cellulose, methyl cellulose, methyl hydroxyethyl cellulose, methyl carboxymethyl cellulose, and mixtures thereof. In one aspect, the carboxymethyl cellulose has a degree of carboxymethyl substitution from 0.5 to 0.9 and a molecular weight from 100,000 to 300,000 Da.

Enzymes—The composition may, beside a lipase variant of the invention, comprise one or more enzymes which provide cleaning performance and/or fabric care benefits. Examples of suitable enzymes include, but are not limited to, hemicellulases, peroxidases, proteases, cellulases, xylanases, other lipolytic enzymes, phospholipases, esterases, cutinases, pectinases, mannanases, pectate lyases, keratinases, reductases, oxidases, phenoloxidases, lipoxygenases, ligninases, pullulanases, tannases, pentosanases, malanases, ß-glucanases, arabinosidases, hyaluronidase, chondroitinase, laccase, chlorophyllases, amylases, or mixtures thereof. A typical combination is an enzyme cocktail that may comprise e.g., a protease and lipase in conjunction with amylase. When present in a composition, the aforementioned additional enzymes may be present at levels from 0.00001 to 2 wt %, from 0.0001 to 1 wt % or from 0.001 to 0.5 wt % enzyme protein by weight of the composition.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

In one aspect, preferred enzymes would include a cellulase. Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO89/09259.

Especially suitable cellulases are the alkaline or neutral cellulases having colour care benefits. Examples of such cellulases are cellulases described in EP0495257, EP0531372, WO96/11262, WO96/29397, WO98/08940. Other examples are cellulase variants such as those described in WO94/07998, EP0531315, U.S. Pat. Nos. 5,457,046, 5,686,593, 5,763,254, WO95/24471, WO98/12307 and PCT/DK98/00299.

Commercially available cellulases include Celluzyme™, and Carezyme™ (Novozymes A/S), Clazinase™, and Puradax HA™ (Genencor International Inc.), and KAC-500(B)™ (Kao Corporation).

In one aspect, preferred enzymes would include a protease. Suitable proteases include those of bacterial, fungal, plant, viral or animal origin e.g. vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloproteases protease may for example be a thermolysin from e.g. family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e. the Subtilisin family, the Thermitase family, the Proteinase K family, the Lantibiotic peptidase family, the Kexin family and the Pyrolysin family.

Examples of subtilases are those derived from Bacillus such as Bacillus lentus, B. alkalophilus, B. subtilis, B. amyloliquefaciens, Bacillus pumilus and Bacillus gibsonii described in; U.S. Pat. No. 7,262,042 and WO09/021867, and subtilisin lentus, subtilisin Novo, subtilisin Carlsberg, Bacillus licheniformis, subtilisin BPN′, subtilisin 309, subtilisin 147 and subtilisin 168 described in WO89/06279 and protease PD138 described in (WO93/18140). Other useful proteases may be those described in WO92/175177, WO01/016285, WO02/026024 and WO02/016547. Examples of trypsin-like proteases are trypsin (e.g. of porcine or bovine origin) and the Fusarium protease described in WO89/06270, WO94/25583 and WO05/040372, and the chymotrypsin proteases derived from Cellumonas described in WO05/052161 and WO05/052146.

A further preferred protease is the alkaline protease from Bacillus lentus DSM 5483, as described for example in WO95/23221, and variants thereof which are described in WO92/21760, WO95/23221, EP1921147 and EP1921148.

Examples of metalloproteases are the neutral metalloprotease as described in WO07/044993 (Genencor Int.) such as those derived from Bacillus amyloliquefaciens.

Examples of useful proteases are the variants described in: WO92/19729, WO96/034946, WO98/20115, WO98/20116, WO99/011768, WO01/44452, WO03/006602, WO04/03186, WO04/041979, WO07/006305, WO11/036263, WO11/036264, especially the variants with substitutions in one or more of the following positions: 3, 4, 9, 15, 27, 36, 57, 68, 76, 87, 95, 96, 97, 98, 99, 100, 101, 102, 103, 104, 106, 118, 120, 123, 128, 129, 130, 160, 167, 170, 194, 195, 199, 205, 206, 217, 218, 222, 224, 232, 235, 236, 245, 248, 252 and 274 using the BPN′ numbering. More preferred the subtilase variants may comprise the mutations: S3T, V41, S9R, A15T, K27R, *36D, V68A, N76D, N87S,R, *97E, A98S, S99G,D,A, S99AD, S101G,M,R S103A, V1041,Y,N, S106A, G118V,R, H120D,N, N123S, S128L, P129Q, S130A, G160D, Y167A, R170S, A194P, G195E, V199M, V2051, L217D, N218D, M222S, A232V, K235L, Q236H, Q245R, N252K, T274A (using BPN′ numbering).

Suitable commercially available protease enzymes include those sold under the trade names Alcalase®, Blaze®; Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® all could be sold as Ultra® or Evity® (Novozymes A/S), those sold under the tradename Maxatase®, Maxacal®, Maxapem®, Purafect®, Purafect Prime®, Preferenz™, Purafect MA®, Purafect Ox®, Purafect OxP®, Puramax®, Properase®, Effectenz™, FN2®, FN3®, FN4®, Excellase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

In one aspect, preferred enzymes would include an amylase. Suitable amylases may be an alpha-amylase or a glucoamylase and may be of bacterial or fungal origin. Chemically modified or protein engineered mutants are included. Amylases include, for example, alpha-amylases obtained from Bacillus, e.g., a special strain of Bacillus licheniformis, described in more detail in GB1296839.

Suitable amylases include amylases having SEQ ID NO: 3 in WO95/10603 or variants having 90% sequence identity to SEQ ID NO: 3 thereof. Preferred variants are described in WO94/02597, WO94/18314, WO97/43424 and SEQ ID NO: 4 of WO99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO2006/066594 and residues 36-483 of the B. licheniformis alpha-amylase shown in SEQ ID NO: 4 of WO2006/066594 or variants having 90% sequence identity thereof. Preferred variants of this hybrid alpha-amylase are those having a substitution, a deletion or an insertion in one of more of the following positions: G48, T49, G107, H156, A181, N190, M197, 1201, A209 and Q264. Most preferred variants of the hybrid alpha-amylase comprising residues 1-33 of the alpha-amylase derived from B. amyloliquefaciens shown in SEQ ID NO: 6 of WO2006/066594 and residues 36-483 of SEQ ID NO: 4 are those having the substitutions:
M197T;
H156Y+A181T+N190F+A209V+Q264S; or
G48A+T491+G107A+H156Y+A181T+N190F+1201F+A209V+Q264S.

Further amylases which are suitable are amylases having SEQ ID NO: 6 in WO99/019467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, 1206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476. More preferred variants are those having a deletion in positions 181 and 182 or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO08/153815, SEQ ID NO: 10 in WO01/66712 or variants thereof having 90% sequence identity to SEQ ID NO: 2 of WO08/153815 or 90% sequence identity to SEQ ID NO: 10 in WO01/66712. Preferred variants of SEQ ID NO: 10 in WO01/66712 are those having a substitution, a deletion or an insertion in one of more of the following positions: 176, 177, 178, 179, 190, 201, 207, 211 and 264.

Further suitable amylases are amylases having SEQ ID NO: 2 of WO09/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E, R, Q98R, S125A, N128C, T1311, T1651, K178L, T182G, M201L, F202Y, N225E, R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:
N128C+K178L+T182G+Y305R+G475K;
N128C+K178L+T182G+F202Y+Y305R+D319T+G475K;
S125A+N128C+K178L+T182G+Y305R+G475K; or
S125A+N128C+T1311+T1651+K178L+T182G+Y305R+G475K wherein the variants are C-terminally truncated and optionally further comprises a substitution at position 243 and/or a deletion at position 180 and/or position 181.

Other suitable amylases are the alpha-amylase having SEQ ID NO: 12 in WO01/66712 or a variant having at least 90% sequence identity to SEQ ID NO: 12. Preferred amylase variants are those having a substitution, a deletion or an insertion in one of more of the following positions of SEQ ID NO: 12 in WO01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO2011/098531, WO2013/001078 and WO2013/001087.

Commercially available amylases are Duramyl™, Termamyl™, Termamyl Ultra™, Fungamyl™, Ban™, Stainzyme™, Stainzyme PIus™, Amplify®, Amplify® Prime, Supramyl™, Natalase™, Liquozyme X and BAN™ (from Novozymes A/S), KEMZYM® AT 9000 Biozym Biotech Trading GmbH Wehlistrasse 27b A-1200 Wien Austria, and Rapidase™ Purastar™/Effectenz™, Powerase, Preferenz S100, Preferenx S110, ENZYSIZE®, OPTISIZE HT PLUS®, and PURASTAR OXAM® (Danisco/DuPont) and KAM® (Kao).

Additional suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP258068 and EP305216, cutinase from Humicola, e.g. H. insolens (WO96/13580), lipase from strains of Pseudomonas (some of these now renamed to Burkholderia), e.g. P. alcaligenes or P. pseudoalcaligenes (EP218272), P. cepacia (EP331376), P. sp. strain SD705 (WO95/06720 & WO96/27002), P. wisconsinensis (WO96/12012), GDSL-type Streptomyces lipases (WO10/065455), cutinase from Magnaporthe grisea (WO10/107560), cutinase from Pseudomonas mendocina (U.S. Pat. No. 5,389,536), lipase from Thermobifida fusca (WO11/084412, WO13/033318), Geobacillus stearothermophilus lipase (WO11/084417), lipase from Bacillus subtilis (WO11/084599), and lipase from Streptomyces griseus (WO11/150157) and S. pristinaespiralis (WO12/137147).

Other examples are additional lipase variants such as those described in EP407225, WO92/05249, WO94/01541, WO94/25578, WO95/14783, WO95/30744, WO95/35381, WO95/22615, WO96/00292, WO97/04079, WO97/07202, WO00/34450, WO00/60063, WO01/92502, WO07/87508 and WO09/109500.

Preferred additional commercial lipase products include Lipolase™, Lipex™; Lipex evitry 100L, Lipex Evity 200L, Lipolex™ and Lipoclean™ (Novozymes A/S), Lumafast (originally from Genencor), Lipomax (originally from Gist-Brocades) and Preferenz L 100 (from Danisco US Inc).

Still other examples are lipases sometimes referred to as acyltransferases or perhydrolases, e.g., acyltransferases with homology to Candida antarctica lipase A (WO10/111143), acyltransferase from Mycobacterium smegmatis (WO05/56782), perhydrolases from the CE 7 family (WO09/67279), and variants of the M. smegmatis perhydrolase in particular the S54V variant used in the commercial product Gentle Power Bleach from Huntsman Textile Effects Pte Ltd (WO10/100028).

In one aspect, other preferred enzymes include microbial-derived endoglucanases exhibiting endo-beta-1,4-glucanase activity (EC3.2.1.4), including a bacterial polypeptide endogenous to a member of the genus Bacillus which has a sequence of at least 90%, 94%, 97% or 99% identity to the amino acid sequence SEQ ID NO:2 in U.S. Pat. No. 7,141,403 and mixtures thereof. Suitable endoglucanases are sold under the tradenames Celluclean® and Whitezyme® (Novozymes).

Other preferred enzymes include pectate lyases sold under the tradenames Pectawash®, Pectaway®, Xpect® and mannanases sold under the tradenames Mannaway® (Novozymes), and Purabrite® (Danisco/DuPont).

The detergent enzyme(s) may be included in a detergent composition by adding separate additives containing one or more enzymes, or by adding a combined additive comprising all of these enzymes. A detergent additive of the invention, i.e., a separate additive or a combined additive, can be formulated, for example, as granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. Nos. 4,106,991 and 4,661,452 and may optionally be coated by methods known in the art. Examples of waxy coating materials are poly(ethylene oxide) products (polyethyleneglycol, PEG) with mean molar weights of 1000 to 20000; ethoxylated nonylphenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB1483591. Liquid enzyme preparations may, for instance, be stabilized by adding a polyol such as propylene glycol, a sugar or sugar alcohol, lactic acid or boric acid according to established methods. Protected enzymes may be prepared according to the method disclosed in EP238216.

Dye Transfer Inhibiting Agents—The compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a composition, the dye transfer inhibiting agents may be present at levels from 0.0001 to 10 wt %, from 0.01 to 5 wt % or from 0.1 to 3 wt %.

Brighteners—The compositions of the present invention can also contain additional components that may tint articles being cleaned, such as fluorescent brighteners.

The composition may comprise C.I. fluorescent brightener 260 in alpha-crystalline form having the following structure:

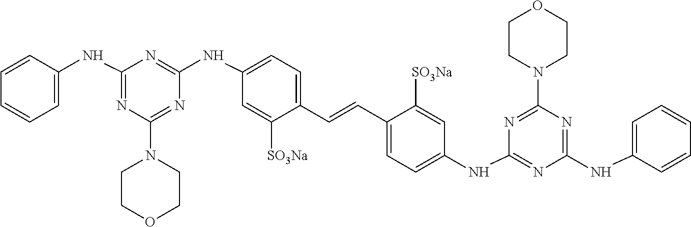

In one aspect, the brightener is a cold water soluble brightener, such as the C.I. fluorescent brightener 260 in alpha-crystalline form. In one aspect the brightener is predominantly in alpha-crystalline form, which means that typically at least 50 wt %, at least 75 wt %, at least 90 wt %, at least 99 wt %, or even substantially all, of the C.I. fluorescent brightener 260 is in alpha-crystalline form.

The brightener is typically in micronized particulate form, having a weight average primary particle size of from 3 to 30 micrometers, from 3 micrometers to 20 micrometers, or from 3 to 10 micrometers.

The composition may comprise C.I. fluorescent brightener 260 in beta-crystalline form, and the weight ratio of: (i) C.I. fluorescent brightener 260 in alpha-crystalline form, to (ii) C.I. fluorescent brightener 260 in beta-crystalline form may be at least 0.1, or at least 0.6. BE680847 relates to a process for making C.I fluorescent brightener 260 in alpha-crystalline form.

Commercial optical brighteners which may be useful in the present invention can be classified into subgroups, which include, but are not necessarily limited to, derivatives of stilbene, pyrazoline, coumarin, carboxylic acid, methinecyanines, dibenzothiophene-5,5-dioxide, azoles, 5- and 6-membered-ring heterocycles, and other miscellaneous agents. Examples of such brighteners are disclosed in “The Production and Application of Fluorescent Brightening Agents”, M. Zahradnik, Published by John Wiley & Sons, New York (1982). Specific nonlimiting examples of optical brighteners which are useful in the present compositions are those identified in U.S. Pat. Nos. 4,790,856 and 3,646,015.

A further suitable brightener has the structure below:

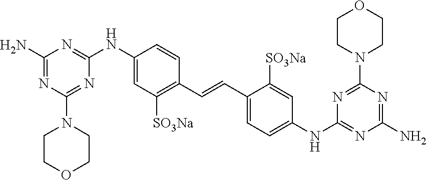

Suitable fluorescent brightener levels include lower levels of from 0.01 wt %, from 0.05 wt %, from 0.1 wt % or from 0.2 wt % to upper levels of 0.5 wt % or 0.75 wt %.

In one aspect the brightener may be loaded onto a clay to form a particle. Silicate salts—The compositions of the present invention can also contain silicate salts, such as sodium or potassium silicate. The composition may comprise of from 0 wt % to less than 10 wt % silicate salt, to 9 wt %, or to 8 wt %, or to 7 wt %, or to 6 wt %, or to 5 wt %, or to 4 wt %, or to 3 wt %, or even to 2 wt %, and from above 0 wt %, or from 0.5 wt %, or from 1 wt % silicate salt. A suitable silicate salt is sodium silicate.

Dispersants—The compositions of the present invention can also contain dispersants. Suitable water-soluble organic materials include the homo- or co-polymeric acids or their salts, in which the polycarboxylic acid comprises at least two carboxyl radicals separated from each other by not more than two carbon atoms.

Enzyme Stabilizers—Enzymes for use in compositions can be stabilized by various techniques. The enzymes employed herein can be stabilized by the presence of water-soluble sources of calcium and/or magnesium ions. Examples of conventional stabilizing agents are, e.g. a polyol such as propylene glycol or glycerol, a sugar or sugar alcohol, a peptide aldehyde, lactic acid, boric acid, or a boric acid derivative, e.g. an aromatic borate ester, or a phenyl boronic acid derivative such as 4-formylphenyl boronic acid, and the composition may be formulated as described in, for example, WO92/19709 and WO92/19708 In case of aqueous compositions comprising protease, a reversible protease inhibitor, such as a boron compound including borate, 4-formyl phenylboronic acid, phenylboronic acid and derivatives thereof, or compounds such as calcium formate, sodium formate and 1,2-propane diol can be added to further improve stability. The peptide aldehyde may be of the formula B₂—B₁—B₀—R wherein: R is hydrogen, CH₃, CX₃, CHX₂, or CH₂X, wherein X is a halogen atom; B₀ is a phenylalanine residue with an OH substituent at the p-position and/or at the m-position; B₁ is a single amino acid residue; and B₂ consists of one or more amino acid residues, optionally comprising an N-terminal protection group. Preferred peptide aldehydes include but are not limited to: Z-RAY-H, Ac-GAY-H, Z-GAY-H, Z-GAL-H, Z-GAF-H, Z-GAV-H, Z-RVY-H, Z-LVY-H, Ac-LGAY-H, Ac-FGAY-H, Ac-YGAY-H, Ac-FGVY-H orAc-WLVY-H, where Z is benzyloxycarbonyl and Ac is acetyl.

Solvents—Suitable solvents include water and other solvents such as lipophilic fluids. Examples of suitable lipophilic fluids include siloxanes, other silicones, hydrocarbons, glycol ethers, glycerine derivatives such as glycerine ethers, perfluorinated amines, perfluorinated and hydrofluoroether solvents, low-volatility nonfluorinated organic solvents, diol solvents, other environmentally-friendly solvents and mixtures thereof.

Structurant/Thickeners—Structured liquids can either be internally structured, whereby the structure is formed by primary ingredients (e.g. surfactant material) and/or externally structured by providing a three dimensional matrix structure using secondary ingredients (e.g. polymers, clay and/or silicate material). The composition may comprise a structurant, from 0.01 to 5 wt %, or from 0.1 to 2.0 wt %. The structurant is typically selected from the group consisting of diglycerides and triglycerides, ethylene glycol distearate, microcrystalline cellulose, cellulose-based materials, microfiber cellulose, hydrophobically modified alkali-swellable emulsions such as Polygel W30 (3VSigma), biopolymers, xanthan gum, gellan gum, and mixtures thereof. A suitable structurant includes hydrogenated castor oil, and non-ethoxylated derivatives thereof. A suitable structurant is disclosed in U.S. Pat. No. 6,855,680. Such structurants have a thread-like structuring system having a range of aspect ratios. Other suitable structurants and the processes for making them are described in WO10/034736.

Conditioning Agents—The composition of the present invention may include a high melting point fatty compound. The high melting point fatty compound useful herein has a melting point of 25° C. or higher, and is selected from the group consisting of fatty alcohols, fatty acids, fatty alcohol derivatives, fatty acid derivatives, and mixtures thereof. Such compounds of low melting point are not intended to be included in this section. Non-limiting examples of the high melting point compounds are found in International Cosmetic Ingredient Dictionary, Fifth Edition, 1993, and CTFA Cosmetic Ingredient Handbook, Second Edition, 1992.

The high melting point fatty compound is included in the composition at a level of from 0.1 to 40 wt %, from 1 to 30 wt %, from 1.5 to 16 wt %, from 1.5 to 8 wt % in view of providing improved conditioning benefits such as slippery feel during the application to wet hair, softness and moisturized feel on dry hair.

The compositions of the present invention may contain a cationic polymer. Concentrations of the cationic polymer in the composition typically range from 0.05 to 3 wt %, from 0.075 to 2.0 wt %, or from 0.1 to 1.0 wt %. Suitable cationic polymers will have cationic charge densities of at least 0.5 meq/gm, at least 0.9 meq/gm, at least 1.2 meq/gm, at least 1.5 meq/gm, or less than 7 meq/gm, and less than 5 meq/gm, at the pH of intended use of the composition, which pH will generally range from pH3 to pH9, or between pH4 and pH8. Herein, “cationic charge density” of a polymer refers to the ratio of the number of positive charges on the polymer to the molecular weight of the polymer. The average molecular weight of such suitable cationic polymers will generally be between 10,000 and 10 million, between 50,000 and 5 million, or between 100,000 and 3 million.

Suitable cationic polymers for use in the compositions of the present invention contain cationic nitrogen-containing moieties such as quaternary ammonium or cationic protonated amino moieties. Any anionic counterions can be used in association with the cationic polymers so long as the polymers remain soluble in water, in the composition, or in a coacervate phase of the composition, and so long as the counterions are physically and chemically compatible with the essential components of the composition or do not otherwise unduly impair composition performance, stability or aesthetics. Nonlimiting examples of such counterions include halides (e.g., chloride, fluoride, bromide, iodide), sulfate and methylsulfate.

Nonlimiting examples of such polymers are described in the CTFA Cosmetic Ingredient Dictionary, 3rd edition, edited by Estrin, Crosley, and Haynes, (The Cosmetic, Toiletry, and Fragrance Association, Inc., Washington, D.C. (1982)).

Other suitable cationic polymers for use in the composition include polysaccharide polymers, cationic guar gum derivatives, quaternary nitrogen-containing cellulose ethers, synthetic polymers, copolymers of etherified cellulose, guar and starch. When used, the cationic polymers herein are either soluble in the composition or are soluble in a complex coacervate phase in the composition formed by the cationic polymer and the anionic, amphoteric and/or zwitterionic surfactant component described hereinbefore. Complex coacervates of the cationic polymer can also be formed with other charged materials in the composition. Suitable cationic polymers are described in U.S. Pat. Nos. 3,962,418; 3,958,581; and US2007/0207109.

The composition of the present invention may include a nonionic polymer as a conditioning agent. Polyalkylene glycols having a molecular weight of more than 1000 are useful herein. Useful are those having the following general formula:

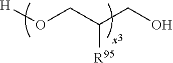

wherein R⁹⁵ is selected from the group consisting of H, methyl, and mixtures thereof. Conditioning agents, and in particular silicones, may be included in the composition. The conditioning agents useful in the compositions of the present invention typically comprise a water insoluble, water dispersible, non-volatile, liquid that forms emulsified, liquid particles. Suitable conditioning agents for use in the composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix herein. Such conditioning agents should be physically and chemically compatible with the essential components of the composition, and should not otherwise unduly impair composition stability, aesthetics or performance.

The concentration of the conditioning agent in the composition should be sufficient to provide the desired conditioning benefits. Such concentration can vary with the conditioning agent, the conditioning performance desired, the average size of the conditioning agent particles, the type and concentration of other components, and other like factors.

The concentration of the silicone conditioning agent typically ranges from 0.01 to 10 wt %. Non-limiting examples of suitable silicone conditioning agents, and optional suspending agents for the silicone, are described in U.S. Reissue Pat. No. 34,584; U.S. Pat. Nos. 5,104,646; 5,106,609; 4,152,416; 2,826,551; 3,964,500; 4,364,837; 6,607,717; 6,482,969; 5,807,956; 5,981,681; 6,207,782; 7,465,439; 7,041,767; 7,217,777; US2007/0286837A1; US2005/0048549A1; US2007/0041929A1; GB849433; DE10036533, which are all incorporated herein by reference; Chemistry and Technology of Silicones, New York: Academic Press (1968); General Electric Silicone Rubber Product Data Sheets SE 30, SE 33, SE 54 and SE 76; Silicon Compounds, Petrarch Systems, Inc. (1984); and in Encyclopedia of Polymer Science and Engineering, vol. 15, 2d ed., pp 204-308, John Wiley & Sons, Inc. (1989).

The compositions of the present invention may also comprise from 0.05 to 3 wt % of at least one organic conditioning oil as the conditioning agent, either alone or in combination with other conditioning agents, such as the silicones (described herein). Suitable conditioning oils include hydrocarbon oils, polyolefins, and fatty esters. Also suitable for use in the compositions herein are the conditioning agents described in U.S. Pat. Nos. 5,674,478 and 5,750,122 or in U.S. Pat. Nos. 4,529,586; 4,507,280; 4,663,158; 4,197,865; 4,217,914; 4,381,919; and 4,422,853.

Hygiene and malodour—The compositions of the present invention may also comprise one or more of zinc ricinoleate, thymol, quaternary ammonium salts such as Bardac®, polyethylenimines (such as Lupasol® from BASF) and zinc complexes thereof, silver and silver compounds, especially those designed to slowly release Ag⁺ or nano-silver dispersions.

Probiotics—The compositions may comprise probiotics such as those described in WO09/043709.

Suds Boosters— If high sudsing is desired, suds boosters such as the C₁₀-C₁₆ alkanolamides or C₁₀-C₁₄ alkyl sulphates can be incorporated into the compositions, typically at 1 to 10 wt % levels. The C₁₀-C₁₄ monoethanol and diethanol amides illustrate a typical class of such suds boosters. Use of such suds boosters with high sudsing adjunct surfactants such as the amine oxides, betaines and sultaines noted above is also advantageous. If desired, water-soluble magnesium and/or calcium salts such as MgCl₂, MgSO₄, CaCl₂, CaSO₄ and the like, can be added at levels of, typically, 0.1 to 2 wt %, to provide additional suds and to enhance grease removal performance.

Suds Suppressors— Compounds for reducing or suppressing the formation of suds can be incorporated into the compositions of the present invention. Suds suppression can be of particular importance in the so-called “high concentration cleaning process” as described in U.S. Pat. Nos. 4,489,455 and 4,489,574, and in front-loading-style washing machines. A wide variety of materials may be used as suds suppressors, and suds suppressors are well known to those skilled in the art. See e.g. Kirk Othmer Encyclopedia of Chemical Technology, Third Edition, Volume 7, p.430-447 (John Wiley & Sons, Inc., 1979). Examples of suds supressors include monocarboxylic fatty acid and soluble salts therein, high molecular weight hydrocarbons such as paraffin, fatty acid esters (e.g., fatty acid triglycerides), fatty acid esters of monovalent alcohols, aliphatic C₁₈-C₄₀ ketones (e.g., stearone), N-alkylated amino triazines, waxy hydrocarbons preferably having a melting point below about 100° C., silicone suds suppressors, and secondary alcohols. Suds supressors are described in U.S. Pat. Nos. 2,954,347; 4,265,779; 4,265,779; 3,455,839; 3,933,672; 4,652,392; 4,978,471; 4,983,316; 5,288,431; 4,639,489; 4,749,740; 4,798,679; 4,075,118; EP89307851.9; EP150872; and DOS 2,124,526.

For any detergent compositions to be used in automatic laundry washing machines, suds should not form to the extent that they overflow the washing machine. Suds suppressors, when utilized, are preferably present in a “suds suppressing amount. By “suds suppressing amount” is meant that the formulator of the composition can select an amount of this suds controlling agent that will sufficiently control the suds to result in a low-sudsing laundry detergent for use in automatic laundry washing machines.

The compositions herein will generally comprise from 0 to 10 wt % of suds suppressor. When utilized as suds suppressors, monocarboxylic fatty acids, and salts therein, will be present typically in amounts up to 5 wt %. Preferably, from 0.5 to 3 wt % of fatty monocarboxylate suds suppressor is utilized. Silicone suds suppressors are typically utilized in amounts up to 2.0 wt %, although higher amounts may be used. Monostearyl phosphate suds suppressors are generally utilized in amounts ranging from 0.1 to 2 wt %. Hydrocarbon suds suppressors are typically utilized in amounts ranging from 0.01 to 5.0 wt %, although higher levels can be used. The alcohol suds suppressors are typically used at 0.2 to 3 wt %.

The compositions herein may have a cleaning activity over a broad range of pH. In certain embodiments the compositions have cleaning activity from pH4 to pH 11.5. In other embodiments, the compositions are active from pH6 to pH11, from pH7 to pH11, from pH8 to pH11, from pH9 to pH11, or from pH10 to pH11.5.

The compositions herein may have cleaning activity over a wide range of temperatures, e.g., from 10° C. or lower to 90° C. Preferably the temperature will be below 50° C. or 40° C. or even 30° C. In certain embodiments, the optimum temperature range for the compositions is from 10° C. to 20° C., from 15° C. to 25° C., from 15° C. to 30° C., from 20° C. to 30° C., from 25° C. to 35° C., from 30° C. to 40° C., from 35° C. to 45° C., or from 40° C. to 50° C.

### Form of the Composition

The compositions described herein are advantageously employed for example, in laundry applications, hard surface cleaning, dishwashing applications (automatic dishwashing (ADW) and hand dishwashing (HDW), as well as cosmetic applications such as dentures, teeth, hair and skin.

The compositions of the invention are in particular solid or liquid cleaning and/or treatment compositions. In one aspect the invention relates to a composition, wherein the form of the composition is selected from the group consisting of a regular, compact or concentrated liquid; a gel; a paste; a soap bar; a regular or a compacted powder; a granulated solid; a homogenous or a multilayer tablet with two or more layers (same or different phases); a pouch having one or more compartments; a single or a multi-compartment unit dose form; or any combination thereof.

The form of the composition may separate the components physically from each other in compartments such as e.g., water dissolvable pouches or in different layers of tablets. Thereby negative storage interaction between components can be avoided. Different dissolution profiles of each of the compartments can also give rise to delayed dissolution of selected components in the wash solution.

Pouches can be configured as single or multicompartments. It can be of any form, shape and material which is suitable for hold the composition, e.g., without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water-soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water-soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water-soluble film. The compartment for liquid components can be different in composition than compartments containing solids (US2009/0011970 A1).

Water-Soluble Film— The compositions of the present invention may also be encapsulated within a water-soluble film. Preferred film materials are preferably polymeric materials. The film material can e.g. be obtained by casting, blow-moulding, extrusion or blown extrusion of the polymeric material, as known in the art. Preferred polymers, copolymers or derivatives thereof suitable for use as pouch material are selected from polyvinyl alcohols, polyvinyl pyrrolidone, polyalkylene oxides, acrylamide, acrylic acid, cellulose, cellulose ethers, cellulose esters, cellulose amides, polyvinyl acetates, polycarboxylic acids and salts, polyaminoacids or peptides, polyamides, polyacrylamide, copolymers of maleic/acrylic acids, polysaccharides including starch and gelatine, natural gums such as xanthum and carragum. More preferred polymers are selected from polyacrylates and water-soluble acrylate copolymers, methylcellulose, carboxymethylcellulose sodium, dextrin, ethylcellulose, hydroxyethyl cellulose, hydroxypropyl methylcellulose, maltodextrin, polymethacrylates, and most preferably selected from polyvinyl alcohols, polyvinyl alcohol copolymers and hydroxypropyl methyl cellulose (HPMC), and combinations thereof. Preferably, the level of polymer in the pouch material, e.g. a PVA polymer, is at least 60 wt %. The polymer can have any weight average molecular weight, preferably from about 1.000 to 1.000.000, from about 10.000 to 300.000, from about 20.000 to 150.000. Mixtures of polymers can also be used as the pouch material.

Naturally, different film material and/or films of different thickness may be employed in making the compartments of the present invention. A benefit in selecting different films is that the resulting compartments may exhibit different solubility or release characteristics.

Preferred film materials are PVA films known under the MonoSol trade reference M8630, M8900, H8779 and those described in U.S. Pat. Nos. 6,166,117 and 6,787,512 and PVA films of corresponding solubility and deformability characteristics.

The film material herein can also comprise one or more additive ingredients. For example, it can be beneficial to add plasticisers, e.g. glycerol, ethylene glycol, diethyleneglycol, propylene glycol, sorbitol and mixtures thereof. Other additives include functional detergent additives to be delivered to the wash water, e.g., organic polymeric dispersants, etc.

### Processes of Making the Compositions

The compositions of the present invention can be formulated into any suitable form and prepared by any process chosen by the formulator, non-limiting examples of which are described in Applicants' examples and in U.S. Pat. No. 4,990,280; US20030087791A1; US20030087790A1; US20050003983A1; US20040048764A1; U.S. Pat. Nos. 4,762,636; 6,291,412; US20050227891A1; EP1070115A2; U.S. Pat. Nos. 5,879,584; 5,691,297; 5,574,005; 5,569,645; 5,565,422; 5,516,448; 5,489,392; 5,486,303 all of which are incorporated herein by reference. The compositions of the invention or prepared according to the invention comprise cleaning and/or treatment composition including, but not limited to, compositions for treating fabrics, hard surfaces and any other surfaces in the area of fabric and home care, including: air care including air fresheners and scent delivery systems, car care, dishwashing, fabric conditioning (including softening and/or freshening), laundry detergency, laundry and rinse additive and/or care, hard surface cleaning and/or treatment including floor and toilet bowl cleaners, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use: car or carpet shampoos, bathroom cleaners including toilet bowl cleaners; as well as cleaning auxiliaries such as bleach additives and “stain-stick” or pre-treat types, substrate-laden compositions such as dryer added sheets. Preferred are compositions and methods for cleaning and/or treating textiles and/or hard surfaces, most preferably textiles. The compositions are preferably compositions used in a pre-treatment step or main wash step of a washing process, most preferably for use in textile washing step.

As used herein, the term “fabric and/or hard surface cleaning and/or treatment composition” is a subset of cleaning and treatment compositions that includes, unless otherwise indicated, granular or powder-form all-purpose or “heavy-duty” washing agents, especially cleaning detergents; liquid, gel or paste-form all-purpose washing agents, especially the so-called heavy-duty liquid types; liquid fine-fabric detergents; hand dishwashing agents or light duty dishwashing agents, especially those of the high-foaming type; machine dishwashing agents, including the various tablet, granular, liquid and rinse-aid types for household and institutional use; liquid cleaning and disinfecting agents, car or carpet shampoos, bathroom cleaners including toilet bowl cleaners; fabric conditioning compositions including softening and/or freshening that may be in liquid, solid and/or dryer sheet form; as well as cleaning auxiliaries such as bleach additives and “stain-stick” or pre-treat types, substrate-laden compositions such as dryer added sheets. All of such compositions which are applicable may be in standard, concentrated or even highly concentrated form even to the extent that such compositions may in certain aspect be non-aqueous.

### Method of Use

The present invention includes a method for cleaning any surface including treating a textile or a hard surface or other surfaces in the field of fabric and/or home care. It is comtemplated that cleaning as described may be both in small scale as in e.g., family household as well as in large scale as in e.g., industrial and professional settings.

In one aspect of the invention, the method comprises the step of contacting the surface to be treated in a pre-treatment step or main wash step of a washing process, most preferably for use in a textile washing step or alternatively for use in dishwashing including both manual as well as automated/mechanical dishwashing.

In one embodiment of the invention the lipase variant and other components are added sequentially into the method for cleaning and/or treating the surface. Alternatively, the lipase variant of the invention and other components are added simultaneously.

As used herein, washing includes but is not limited to, scrubbing, and mechanical agitation. Washing may be conducted with a foam composition as described in WO08/101958 and/or by applying alternating pressure (pressure/vaccum) as an addition or as an alternative to scrubbing and mechanical agitation. Drying of such surfaces or fabrics may be accomplished by any one of the common means employed either in domestic or industrial settings.

The cleaning compositions of the present invention are ideally suited for use in laundry as well as dishwashing applications (ADW or HDW). Accordingly, the present invention includes a method for cleaning an object including but not limiting to fabric, tableware, cutlery and kitchenware. The method comprises the steps of contacting the object to be cleaned with a said cleaning composition comprising at least one cleaning composition of the invention, cleaning additive or mixture thereof. The fabric may comprise any fabric capable of being laundered in normal consumer or institutional use conditions. The solution may have a pH from 8 to 10.5. The composition of the invention may be employed at concentrations from 500 to 15.000 ppm in solution. The water temperatures typically range from 5° C. to 90° C. The water to fabric ratio is typically from 1:1 to 30:1.

In one aspect, the invention relates to a method for hydrolyzing a lipase substrate comprising mixing the substrate with a lipase variant of the invention or the composition of the invention at conditions conductive for the lipase variant hydrolyzing the substrate.

In another aspect, the invention relates to a method for removing lipid stain material from a surface comprising contacting the lipid stain material with a lipase variant of the invention or the composition of the invention at conditions conductive for the lipase variant hydrolyzing the lipid stain material.

In another aspect, the invention relates to a method for lipid stain removal from a surface comprising: contacting said stain with a lipase variant of the invention, or a composition of the invention, followed by rinsing of the surface, and optionally drying, in which method the odor generation is reduced compared to the method wherein the parent lipase, in particular one of SEQ ID NOs: 2, 4, 6 or 8, is contacted with the stain.

In an aspect, the invention relates to the use of a lipase variant of the invention or composition of the invention for cleaning a surface comprising applying the lipase variant to the surface to be cleaned, rinsing the surface, and optionally drying the surface.

According to the invention, use of a lipase variant for cleaning a surface comprising: applying to said surface to be cleaned a lipase variant of the invention, or a composition of the invention, rinsing the surface, and optional drying, in which the odor generation is reduced when compared to the use wherein the parent lipase, in particular one of SEQ ID NOs: 2, 4, 6 or 8, is applied to the surface to be cleaned.

### Polynucleotides

The present invention also relates to polynucleotides encoding a lipase variant of the present invention.

The polynucleotide may be a genomic DNA, a cDNA, a synthetic DNA, a synthetic RNA, a mRNA, or a combination thereof.

In an aspect, the polynucleotide is isolated. In another aspect, the polynucleotide is purified.

### Nucleic Acid Constructs

The present invention also relates to nucleic acid constructs comprising a polynucleotide encoding a lipase variant of the present invention. In a preferred embodiment, the nucleic acid constructs comprising a polynucleotide encoding a lipase variant of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

### Promoters

The control sequence may be a promoter, a polynucleotide recognized by a host cell for expression of a polynucleotide encoding a variant of the present invention. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a filamentous fungal host cell are promoters obtained from Aspergillus, Fusarium, Rhizomucor and Trichoderma cells, such as the promoters described in Mukherjee et al., 2013, “Trichoderma: Biology and Applications”, and by Schmoll and Dattenböck, 2016, “Gene Expression Systems in Fungi: Advancements and Applications”, Fungal Biology.

### Terminators

The control sequence may also be a transcription terminator, which is recognized by a host cell to terminate transcription. The terminator is operably linked to the 3′-terminus of the polynucleotide encoding the variant. Any terminator that is functional in the host cell may be used in the present invention.

Preferred terminators for filamentous fungal host cells may be obtained from Aspergillus or Trichoderma species, such as obtained from the genes for Aspergillus niger glucoamylase, Trichoderma reesei beta-glucosidase, Trichoderma reesei cellobiohydrolase I, and Trichoderma reesei endoglucanase I, such as the terminators described in Mukherjee et al., 2013, “Trichoderma: Biology and Applications”, and by Schmoll and Dattenböck, 2016, “Gene Expression Systems in Fungi: Advancements and Applications”, Fungal Biology.

mRNA Stabilizers

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177: 3465-3471).

Examples of mRNA stabilizer regions for fungal cells are described in Geisberg et al., 2014, Cell 156(4): 812-824, and in Morozov et al., 2006, Eukaryotic Cell 5(11): 1838-1846.

### Leader Sequences

The control sequence may also be a leader, a nontranslated region of an mRNA that is important for translation by the host cell. The leader is operably linked to the 5′-terminus of the polynucleotide encoding the variant. Any leader that is functional in the host cell may be used.

Preferred leaders for filamentous fungal host cells may be obtained from the genes for Aspergillus oryzae TAKA amylase and Aspergillus nidulans triose phosphate isomerase.

### Polyadenylation Sequences

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

### Signal Peptides

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase, such as the signal peptide described by Xu et al., 2018, Biotechnology Letters 40: 949-955

### Propeptides

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active variant by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a variant and the signal peptide sequence is positioned next to the N-terminus of the propeptide sequence.

### Regulatory Sequences

It may also be desirable to add regulatory sequences that regulate expression of the variant relative to the growth of the host cell. Examples of regulatory sequences are those that cause expression of the gene to be turned on or off in response to a chemical or physical stimulus, including the presence of a regulatory compound.

In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals.

### Transcription Factors

The control sequence may also be a transcription factor, a polynucleotide encoding a polynucleotide-specific DNA-binding polypeptide that controls the rate of the transcription of genetic information from DNA to mRNA by binding to a specific polynucleotide sequence. The transcription factor may function alone and/or together with one or more other polypeptides or transcription factors in a complex by promoting or blocking the recruitment of RNA polymerase. Transcription factors are characterized by comprising at least one DNA-binding domain which often attaches to a specific DNA sequence adjacent to the genetic elements which are regulated by the transcription factor. The transcription factor may regulate the expression of a protein of interest either directly, i.e., by activating the transcription of the gene encoding the protein of interest by binding to its promoter, or indirectly, i.e., by activating the transcription of a further transcription factor which regulates the transcription of the gene encoding the protein of interest, such as by binding to the promoter of the further transcription factor. Suitable transcription factors for fungal host cells are described in WO 2017/144177. Suitable transcription factors for prokaryotic host cells are described in Seshasayee et al., 2011, Subcellular Biochemistry 52: 7-23, as well in Balleza et al., 2009, FEMS Microbiol. Rev. 33(1): 133-151.

### Expression Vectors

The present invention also relates to recombinant expression vectors comprising a polynucleotide encoding a variant of the present invention. In a preferred embodiments the recombinant expression vectors comprising a polynucleotide encoding a variant of the present invention, a promoter, and transcriptional and translational stop signals.

The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

The vector preferably contains at least one element that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous recombination, such as homology-directed repair (HDR), or non-homologous recombination, such as non-homologous end-joining (NHEJ).

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

More than one copy of a polynucleotide of the present invention may be inserted into a host cell to increase production of a polypeptide. For example, 2 or 3 or 4 or 5 or more copies are inserted into a host cell. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

### Host Cells

The present invention also relates to recombinant host cells comprising a polynucleotide of the present invention. In a preferred embodiment, the recombinant host cells comprising a polynucleotide of the present invention operably linked to one or more control sequences that direct the production of a lipase variant of the present invention.

A construct or vector comprising a polynucleotide is introduced into a host cell so that the construct or vector is maintained as a chromosomal integrant or as a self-replicating extra-chromosomal vector as described earlier. The choice of a host cell will to a large extent depend upon the gene encoding the variant and its source. The recombinant host cell may comprise a single copy, or at least two copies, e.g., three, four, five, or more copies of the polynucleotide of the present invention.

The host cell may be any cell useful in the recombinant production of a variant of the invention, e.g., a prokaryotic cell or a fungal cell.

The host cell may be any microbial cell useful in the recombinant production of a polypeptide of the present invention, e.g., a prokaryotic cell or a fungal cell.

The host cell may preferably be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

Fungal cells may be transformed by a process involving protoplast-mediated transformation, Agrobacterium-mediated transformation, electroporation, biolistic method and shock-wave-mediated transformation as reviewed by Li et al., 2017, Microbial Cell Factories 16: 168 and procedures described in EP 238023, Yelton et al., 1984, Proc. Natl. Acad. Sci. USA 81: 1470-1474, Christensen et al., 1988, Bio/Technology 6: 1419-1422, and Lubertozzi and Keasling, 2009, Biotechn. Advances 27: 53-75. However, any method known in the art for introducing DNA into a fungal host cell can be used, and the DNA can be introduced as linearized or as circular polynucleotide.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. In a preferred embodiment, the filamentous fungal host cell is an Aspergillus, Trichoderma or Fusarium cell. In a further preferred embodiment, the filamentous fungal host cell is an Aspergillus niger, Aspergillus oryzae, Trichoderma reesei, or Fusarium venenatum cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

In an aspect, the host cell is isolated. In another aspect, the host cell is purified.

### Methods of Production

The present invention also relates to methods of producing a lipase variant of the present invention, comprising
a. cultivating a recombinant host cell of the present invention under conditions conducive for production of the variant; and
b. optionally recovering the variant.

The host cell is cultivated in a nutrient medium suitable for production of the lipase variant of the invention using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the variant to be expressed and/or isolated. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be detected using methods known in the art that are specific for the variant, including, but not limited to, the use of specific antibodies, formation of an enzyme product, disappearance of an enzyme substrate, or an enzyme assay determining the relative or specific activity of the variant.

The variant may be recovered from the medium using methods known in the art, including, but not limited to, collection, centrifugation, filtration, extraction, spray-drying, evaporation, or precipitation. In one aspect, the whole fermentation broth is recovered. In another aspect, a cell-free fermentation broth comprising the polypeptide is recovered.

The variant may be purified by a variety of procedures known in the art to obtain substantially pure variants and/or fragments (see, e.g., Wingfield, 2015, Current Protocols in Protein Science; 80(1): 6.1.1-6.1.35; Labrou, 2014, Protein Downstream Processing, 1129: 3-10).

In an alternative aspect, the variant is not recovered.

In an aspect, the invention relates to a method of washing laundry, comprising the steps of
i) washing by subjecting the laundry to a lipase of the invention, a granule of the invention, a liquid composition of the invention, or a composition of the invention;
ii) rinsing the laundry; and optionally
iii) drying the laundry.

In a preferred embodiment, washing cycle in step i) is carried out at a pH around 8-11 and rising step ii) is carried out a pH around 6-8, preferably around 7.

In a preferred embodiment the wash cycle and/or rising step is caried out in an aqueous solution.

In a preferred embodiment, the washing cycle in step i) is carried out at between 10-90° C., in particular between 20° C. and 40° C. or between 50° C. and 70° C.

### Uses

In an aspect, the invention relates to the use of a lipase variant of the invention or composition of the invention for cleaning a surface comprising applying the lipase variant to the surface to be cleaned.

In an embodiment, the invention relates to the use of a lipase variant of the invention for cleaning a surface comprising: subjecting to said surface to be cleaned to a lipase variant of the invention, or a granule of the invention, a liquid composition of the invention, or a composition of the invention.

The present invention is further described by the following examples that should not be construed as limiting the scope of the invention.

The invention is described in the following numbered paragraphs:

1. A lipase variant, selected from one or more of groups (i), (ii) and (iii) comprising
(i) a substitution at one or more positions corresponding to positions 202, 252, and 269 of the polypeptide of SEQ ID NO: 8;
(ii) a substitution at one or more positions corresponding to positions 40, 56, 57, 91, 98, 108, 118, 210, 244, and 254 of the polypeptide of SEQ ID NO: 8; and
(iii) a substitution at one or more positions corresponding to positions 23, 27, 40, 51, 56, 60, 118 244 and 256 of the polypeptide of SEQ ID NO: 8;
wherein the variant has lipase activity and wherein the variant has at least 60%, e.g., at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 8, wherein the variant optionally comprises an extension of one or more amino acids at the N-terminal and/or C-terminal ends or a truncation of one or more amino acids at the N-terminal and/or C-terminal ends and wherein the variant has lipase activity.
   2. The variant of paragraph 1, which comprises or consists of one or more substitutions, in particular all substitutions, corresponding to the positions in SEQ ID NO: 8, selected from the group consisting of:
a substitution of the amino acid residue at position 202 with H;
a substitution of the amino acid residue at position 252 with H; and
a substitution of the amino acid residue at position 269 with H.
   3. The variant of paragraphs 1 or 2, wherein the variant comprises or consists of one of the following set of substitutions: 202H+252H; 202H+269H; 252H+269H; or 202H+252H+269H.
   4. The variant of any one of paragraphs 1-3, which comprises or consists of one or more substitutions, in particular all substitutions, corresponding to the positions in SEQ ID NO: 8, selected from the group consisting of:
a substitution of the amino acid residue at position 40 with E;
a substitution of the amino acid residue at position 56 with R;
a substitution of the amino acid residue at position 57 with N;
a substitution of the amino acid residue at position 91 with T;
a substitution of the amino acid residue at position 98 with E;
a substitution of the amino acid residue at position 108 with K;
a substitution of the amino acid residue at position 118 with F;
a substitution of the amino acid residue at position 210 with K;
a substitution of the amino acid residue at position 244 with E; and
a substitution of the amino acid residue at position 254 with S.

5. The variant of any of paragraphs 1-4, which comprises or consists of one or more substitutions, in particular all substitutions, corresponding to the positions in SEQ ID NO: 8, selected from the group consisting of:
a substitution of the amino acid residue at position 23 with S;
a substitution of the amino acid residue at position 27 with N;
a substitution of the amino acid residue at position 40 with I;
a substitution of the amino acid residue at position 51 with I;
a substitution of the amino acid residue at position 56 with R;
a substitution of the amino acid residue at position 60 with K;
a substitution of the amino acid residue at position 118 with F;
a substitution of the amino acid residue at position 244 with E; and
a substitution of the amino acid residue at position 256 with T.
   6. The variant of any one of paragraph 1-5, wherein the variant has a substitution corresponding to I202H of SEQ ID NO: 8 and further comprises one of the following substitutions or set of substitutions corresponding to:
A40E, E56R, D57N, G91T, K98E, R108K, R118F, E210K, T244E, D254SA40E+E56R, A40E+D57N, A40E+G91T, A40E+K98E, A40E+R108K, A40E+R118F, A40E+E210K, A40E+T244E, A40E+D254S, E56R+D57N, E56R+G91T, E56R+K98E, E56R+R108K, E56R+R118F, E56R+E210K, E56R+T244E, E56R+D254S, D57N+G91T, D57N+K98E, D57N+R108K, D57N+R118F, D57N+E210K, D57N+T244E, D57N+D254S, G91T+K98E, G91T+R108K, G91T+R118F, G91T+E210K, G91T+T244E, G91T+D254S, K98E+R108K, K98E+R118F, K98E+E210K, K98E+T244E, K98E+D254S, R108K+R118F, R108K+E210K, R108K+T244E, R108K+D254S, R118F+E210K, R118F+T244E, R118F+D254S, E210K+T244E, E210K+D254S, T244E+D254S, A40E+E56R+D57N, A40E+E56R+G91T, A40E+E56R+K98E, A40E+E56R+R108K, A40E+E56R+R118F, A40E+E56R+E210K, A40E+E56R+T244E, A40E+E56R+D254S, A40E+D57N+G91T, A40E+D57N+K98E, A40E+D57N+R108K, A40E+D57N+R118F, A40E+D57N+E210K, A40E+D57N+T244E, A40E+D57N+D254S, A40E+G91T+K98E, A40E+G91T+R108K, A40E+G91T+R118F, A40E+G91T+E210K, A40E+G91T+T244E, A40E+G91T+D254S, A40E+K98E+R108K, A40E+K98E+R118F, A40E+K98E+E210K, A40E+K98E+T244E, A40E+K98E+D254S, A40E+R108K+R118F, A40E+R108K+E210K, A40E+R108K+T244E, A40E+R108K+D254S, A40E+R118F+E210K, A40E+R118F+T244E, A40E+R118F+D254S, A40E+E210K+T244E, A40E+E210K+D254S, A40E+T244E+D254S, E56R+D57N+G91T, E56R+D57N+K98E, E56R+D57N+R108K, E56R+D57N+R118F, E56R+D57N+E210K, E56R+D57N+T244E, E56R+D57N+D254S, E56R+G91T+K98E, E56R+G91T+R108K, E56R+G91T+R118F, E56R+G91T+E210K, E56R+G91T+T244E, E56R+G91T+D254S, E56R+K98E+R108K, E56R+K98E+R118F, E56R+K98E+E210K, E56R+K98E+T244E, E56R+K98E+D254S, E56R+R108K+R118F, E56R+R108K+E210K, E56R+R108K+T244E, E56R+R108K+D254S, E56R+R118F+E210K, E56R+R118F+T244E, E56R+R118F+D254S, E56R+E210K+T244E, E56R+E210K+D254S, E56R+T244E+D254S, D57N+G91T+K98E, D57N+G91T+R108K, D57N+G91T+R118F, D57N+G91T+E210K, D57N+G91T+T244E, D57N+G91T+D254S, D57N+K98E+R108K, D57N+K98E+R118F, D57N+K98E+E210K, D57N+K98E+T244E, D57N+K98E+D254S, D57N+R108K+R118F, D57N+R108K+E210K, D57N+R108K+T244E, D57N+R108K+D254S, D57N+R118F+E210K, D57N+R118F+T244E, D57N+R118F+D254S, D57N+E210K+T244E, D57N+E210K+D254S, D57N+T244E+D254S, G91T+K98E+R108K, G91T+K98E+R118F, G91T+K98E+E210K, G91T+K98E+T244E, G91T+K98E+D254S, G91T+R108K+R118F, G91T+R108K+E210K, G91T+R108K+T244E, G91T+R108K+D254S, G91T+R118F+E210K, G91T+R118F+T244E, G91T+R118F+D254S, G91T+E210K+T244E, G91T+E210K+D254S, G91T+T244E+D254S, K98E+R108K+R118F, K98E+R108K+E210K, K98E+R108K+T244E, K98E+R108K+D254S, K98E+R118F+E210K, K98E+R118F+T244E, K98E+R118F+D254S, K98E+E210K+T244E, K98E+E210K+D254S, K98E+T244E+D254S, R108K+R118F+E210K, R108K+R118F+T244E, R108K+R118F+D254S, R108K+E210K+T244E, R108K+E210K+D254S, R108K+T244E+D254S,R118F+E210K+T244E,R118F+E210K+D254S,R118F+T244E+D254S, E210K+T244E+D254S, A40E+E56R+D57N+G91T, A40E+E56R+D57N+K98E, A40E+E56R+D57N+R108K, A40E+E56R+D57N+R118F, A40E+E56R+D57N+E210K, A40E+E56R+D57N+T244E, A40E+E56R+D57N+D254S, A40E+E56R+G91T+K98E, A40E+E56R+G91T+R108K, A40E+E56R+G91T+R118F, A40E+E56R+G91T+E210K, A40E+E56R+G91T+T244E, A40E+E56R+G91T+D254S, A40E+E56R+K98E+R108K, A40E+E56R+K98E+R118F, A40E+E56R+K98E+E210K, A40E+E56R+K98E+T244E, A40E+E56R+K98E+D254S, A40E+E56R+R108K+R118F, A40E+E56R+R108K+E210K, A40E+E56R+R108K+T244E, A40E+E56R+R108K+D254S, A40E+E56R+R118F+E210K, A40E+E56R+R118F+T244E, A40E+E56R+R118F+D254S, A40E+E56R+E210K+T244E, A40E+E56R+E210K+D254S, A40E+E56R+T244E+D254S, A40E+D57N+G91T+K98E, A40E+D57N+G91T+R108K, A40E+D57N+G91T+R118F, A40E+D57N+G91T+E210K, A40E+D57N+G91T+T244E, A40E+D57N+G91T+D254S, A40E+D57N+K98E+R108K, A40E+D57N+K98E+R118F, A40E+D57N+K98E+E210K, A40E+D57N+K98E+T244E, A40E+D57N+K98E+D254S, A40E+D57N+R108K+R118F, A40E+D57N+R108K+E210K, A40E+D57N+R108K+T244E, A40E+D57N+R108K+D254S, A40E+D57N+R118F+E210K, A40E+D57N+R118F+T244E, A40E+D57N+R118F+D254S, A40E+D57N+E210K+T244E, A40E+D57N+E210K+D254S, A40E+D57N+T244E+D254S, A40E+G91T+K98E+R108K, A40E+G91T+K98E+R118F, A40E+G91T+K98E+E210K, A40E+G91T+K98E+T244E, A40E+G91T+K98E+D254S, A40E+G91T+R108K+R118F, A40E+G91T+R108K+E210K, A40E+G91T+R108K+T244E, A40E+G91T+R108K+D254S, A40E+G91T+R118F+E210K, A40E+G91T+R118F+T244E, A40E+G91T+R118F+D254S, A40E+G91T+E210K+T244E, A40E+G91T+E210K+D254S, A40E+G91T+T244E+D254S, A40E+K98E+R108K+R118F, A40E+K98E+R108K+E210K, A40E+K98E+R108K+T244E, A40E+K98E+R108K+D254S, A40E+K98E+R118F+E210K, A40E+K98E+R118F+T244E, A40E+K98E+R118F+D254S, A40E+K98E+E210K+T244E, A40E+K98E+E210K+D254S, A40E+K98E+T244E+D254S, A40E+R108K+R118F+E210K, A40E+R108K+R118F+T244E, A40E+R108K+R118F+D254S, A40E+R108K+E210K+T244E, A40E+R108K+E210K+D254S, A40E+R108K+T244E+D254S, A40E+R118F+E210K+T244E, A40E+R118F+E210K+D254S, A40E+R118F+T244E+D254S, A40E+E210K+T244E+D254S, E56R+D57N+G91T+K98E, E56R+D57N+G91T+R108K, E56R+D57N+G91T+R118F, E56R+D57N+G91T+E210K, E56R+D57N+G91T+T244E, E56R+D57N+G91T+D254S, E56R+D57N+K98E+R108K, E56R+D57N+K98E+R118F, E56R+D57N+K98E+E210K, E56R+D57N+K98E+T244E, E56R+D57N+K98E+D254S, E56R+D57N+R108K+R118F, E56R+D57N+R108K+E210K, E56R+D57N+R108K+T244E, E56R+D57N+R108K+D254S, E56R+D57N+R118F+E210K, E56R+D57N+R118F+T244E, E56R+D57N+R118F+D254S, E56R+D57N+E210K+T244E, E56R+D57N+E210K+D254S, E56R+D57N+T244E+D254S, E56R+G91T+K98E+R108K, E56R+G91T+K98E+R118F, E56R+G91T+K98E+E210K, E56R+G91T+K98E+T244E, E56R+G91T+K98E+D254S, E56R+G91T+R108K+R118F, E56R+G91T+R108K+E210K, E56R+G91T+R108K+T244E, E56R+G91T+R108K+D254S, E56R+G91T+R118F+E210K, E56R+G91T+R118F+T244E, E56R+G91T+R118F+D254S, E56R+G91T+E210K+T244E, E56R+G91T+E210K+D254S, E56R+G91T+T244E+D254S, E56R+K98E+R108K+R118F, E56R+K98E+R108K+E210K, E56R+K98E+R108K+T244E, E56R+K98E+R108K+D254S, E56R+K98E+R118F+E210K, E56R+K98E+R118F+T244E, E56R+K98E+R118F+D254S, E56R+K98E+E210K+T244E, E56R+K98E+E210K+D254S, E56R+K98E+T244E+D254S, E56R+R108K+R118F+E210K, E56R+R108K+R118F+T244E, E56R+R108K+R118F+D254S, E56R+R108K+E210K+T244E, E56R+R108K+E210K+D254S, E56R+R108K+T244E+D254S, E56R+R118F+E210K+T244E, E56R+R118F+E210K+D254S, E56R+R118F+T244E+D254S, E56R+E210K+T244E+D254S, D57N+G91T+K98E+R108K, D57N+G91T+K98E+R118F, D57N+G91T+K98E+E210K, D57N+G91T+K98E+T244E, D57N+G91T+K98E+D254S, D57N+G91T+R108K+R118F, D57N+G91T+R108K+E210K, D57N+G91T+R108K+T244E, D57N+G91T+R108K+D254S, D57N+G91T+R118F+E210K, D57N+G91T+R118F+T244E, D57N+G91T+R118F+D254S, D57N+G91T+E210K+T244E, D57N+G91T+E210K+D254S, D57N+G91T+T244E+D254S, D57N+K98E+R108K+R118F, D57N+K98E+R108K+E210K, D57N+K98E+R108K+T244E, D57N+K98E+R108K+D254S, D57N+K98E+R118F+E210K, D57N+K98E+R118F+T244E, D57N+K98E+R118F+D254S, D57N+K98E+E210K+T244E, D57N+K98E+E210K+D254S, D57N+K98E+T244E+D254S, D57N+R108K+R118F+E210K, D57N+R108K+R118F+T244E, D57N+R108K+R118F+D254S, D57N+R108K+E210K+T244E, D57N+R108K+E210K+D254S, D57N+R108K+T244E+D254S, D57N+R118F+E210K+T244E, D57N+R118F+E210K+D254S, D57N+R118F+T244E+D254S, D57N+E210K+T244E+D254S, G91T+K98E+R108K+R118F, G91T+K98E+R108K+E210K, G91T+K98E+R108K+T244E, G91T+K98E+R108K+D254S, G91T+K98E+R118F+E210K, G91T+K98E+R118F+T244E, G91T+K98E+R118F+D254S, G91T+K98E+E210K+T244E, G91T+K98E+E210K+D254S, G91T+K98E+T244E+D254S, G91T+R108K+R118F+E210K, G91T+R108K+R118F+T244E, G91T+R108K+R118F+D254S, G91T+R108K+E210K+T244E, G91T+R108K+E210K+D254S, G91T+R108K+T244E+D254S, G91T+R118F+E210K+T244E, G91T+R118F+E210K+D254S, G91T+R118F+T244E+D254S, G91T+E210K+T244E+D254S, K98E+R108K+R118F+E210K, K98E+R108K+R118F+T244E, K98E+R108K+R118F+D254S, K98E+R108K+E210K+T244E, K98E+R108K+E210K+D254S, K98E+R108K+T244E+D254S, K98E+R118F+E210K+T244E, K98E+R118F+E210K+D254S, K98E+R118F+T244E+D254S, K98E+E210K+T244E+D254S, R108K+R118F+E210K+T244E, R108K+R118F+E210K+D254S, R108K+R118F+T244E+D254S, R108K+E210K+T244E+D254S, R118F+E210K+T244E+D254S, A40E+E56R+D57N+G91T+K98E, A40E+E56R+D57N+G91T+R108K, A40E+E56R+D57N+G91T+R118F, A40E+E56R+D57N+G91T+E210K, A40E+E56R+D57N+G91T+T244E, A40E+E56R+D57N+G91T+D254S, A40E+E56R+D57N+K98E+R108K, A40E+E56R+D57N+K98E+R118F, A40E+E56R+D57N+K98E+E210K, A40E+E56R+D57N+K98E+T244E, A40E+E56R+D57N+K98E+D254S, A40E+E56R+D57N+R108K+R118F, A40E+E56R+D57N+R108K+E210K, A40E+E56R+D57N+R108K+T244E, A40E+E56R+D57N+R108K+D254S, A40E+E56R+D57N+R118F+E210K, A40E+E56R+D57N+R118F+T244E, A40E+E56R+D57N+R118F+D254S, A40E+E56R+D57N+E210K+T244E, A40E+E56R+D57N+E210K+D254S, A40E+E56R+D57N+T244E+D254S, A40E+E56R+G91T+K98E+R108K, A40E+E56R+G91T+K98E+R118F, A40E+E56R+G91T+K98E+E210K, A40E+E56R+G91T+K98E+T244E, A40E+E56R+G91T+K98E+D254S, A40E+E56R+G91T+R108K+R118F, A40E+E56R+G91T+R108K+E210K, A40E+E56R+G91T+R108K+T244E, A40E+E56R+G91T+R108K+D254S, A40E+E56R+G91T+R118F+E210K, A40E+E56R+G91T+R118F+T244E, A40E+E56R+G91T+R118F+D254S, A40E+E56R+G91T+E210K+T244E, A40E+E56R+G91T+E210K+D254S, A40E+E56R+G91T+T244E+D254S, A40E+E56R+K98E+R108K+R118F, A40E+E56R+K98E+R108K+E210K, A40E+E56R+K98E+R108K+T244E, A40E+E56R+K98E+R108K+D254S, A40E+E56R+K98E+R118F+E210K, A40E+E56R+K98E+R118F+T244E, A40E+E56R+K98E+R118F+D254S, A40E+E56R+K98E+E210K+T244E, A40E+E56R+K98E+E210K+D254S, A40E+E56R+K98E+T244E+D254S, A40E+E56R+R108K+R118F+E210K, A40E+E56R+R108K+R118F+T244E, A40E+E56R+R108K+R118F+D254S, A40E+E56R+R108K+E210K+T244E, A40E+E56R+R108K+E210K+D254S, A40E+E56R+R108K+T244E+D254S, A40E+E56R+R118F+E210K+T244E, A40E+E56R+R118F+E210K+D254S, A40E+E56R+R118F+T244E+D254S, A40E+E56R+E210K+T244E+D254S, A40E+D57N+G91T+K98E+R108K, A40E+D57N+G91T+K98E+R118F, A40E+D57N+G91T+K98E+E210K, A40E+D57N+G91T+K98E+T244E, A40E+D57N+G91T+K98E+D254S, A40E+D57N+G91T+R108K+R118F, A40E+D57N+G91T+R108K+E210K, A40E+D57N+G91T+R108K+T244E, A40E+D57N+G91T+R108K+D254S, A40E+D57N+G91T+R118F+E210K, A40E+D57N+G91T+R118F+T244E, A40E+D57N+G91T+R118F+D254S, A40E+D57N+G91T+E210K+T244E, A40E+D57N+G91T+E210K+D254S, A40E+D57N+G91T+T244E+D254S, A40E+D57N+K98E+R108K+R118F, A40E+D57N+K98E+R108K+E210K, A40E+D57N+K98E+R108K+T244E, A40E+D57N+K98E+R108K+D254S, A40E+D57N+K98E+R118F+E210K, A40E+D57N+K98E+R118F+T244E, A40E+D57N+K98E+R118F+D254S, A40E+D57N+K98E+E210K+T244E, A40E+D57N+K98E+E210K+D254S, A40E+D57N+K98E+T244E+D254S, A40E+D57N+R108K+R118F+E210K, A40E+D57N+R108K+R118F+T244E, A40E+D57N+R108K+R118F+D254S, A40E+D57N+R108K+E210K+T244E, A40E+D57N+R108K+E210K+D254S, A40E+D57N+R108K+T244E+D254S, A40E+D57N+R118F+E210K+T244E, A40E+D57N+R118F+E210K+D254S, A40E+D57N+R118F+T244E+D254S, A40E+D57N+E210K+T244E+D254S, A40E+G91T+K98E+R108K+R118F, A40E+G91T+K98E+R108K+E210K, A40E+G91T+K98E+R108K+T244E, A40E+G91T+K98E+R108K+D254S, A40E+G91T+K98E+R118F+E210K, A40E+G91T+K98E+R118F+T244E, A40E+G91T+K98E+R118F+D254S, A40E+G91T+K98E+E210K+T244E, A40E+G91T+K98E+E210K+D254S, A40E+G91T+K98E+T244E+D254S, A40E+G91T+R108K+R118F+E210K, A40E+G91T+R108K+R118F+T244E, A40E+G91T+R108K+R118F+D254S, A40E+G91T+R108K+E210K+T244E, A40E+G91T+R108K+E210K+D254S, A40E+G91T+R108K+T244E+D254S, A40E+G91T+R118F+E210K+T244E, A40E+G91T+R118F+E210K+D254S, A40E+G91T+R118F+T244E+D254S, A40E+G91T+E210K+T244E+D254S, A40E+K98E+R108K+R118F+E210K, A40E+K98E+R108K+R118F+T244E, A40E+K98E+R108K+R118F+D254S, A40E+K98E+R108K+E210K+T244E, A40E+K98E+R108K+E210K+D254S, A40E+K98E+R108K+T244E+D254S, A40E+K98E+R118F+E210K+T244E, A40E+K98E+R118F+E210K+D254S, A40E+K98E+R118F+T244E+D254S, A40E+K98E+E210K+T244E+D254S, A40E+R108K+R118F+E210K+T244E, A40E+R108K+R118F+E210K+D254S, A40E+R108K+R118F+T244E+D254S, A40E+R108K+E210K+T244E+D254S, A40E+R118F+E210K+T244E+D254S, E56R+D57N+G91T+K98E+R108K, E56R+D57N+G91T+K98E+R118F, E56R+D57N+G91T+K98E+E210K, E56R+D57N+G91T+K98E+T244E, E56R+D57N+G91T+K98E+D254S, E56R+D57N+G91T+R108K+R118F, E56R+D57N+G91T+R108K+E210K, E56R+D57N+G91T+R108K+T244E, E56R+D57N+G91T+R108K+D254S, E56R+D57N+G91T+R118F+E210K, E56R+D57N+G91T+R118F+T244E, E56R+D57N+G91T+R118F+D254S, E56R+D57N+G91T+E210K+T244E, E56R+D57N+G91T+E210K+D254S, E56R+D57N+G91T+T244E+D254S, E56R+D57N+K98E+R108K+R118F, E56R+D57N+K98E+R108K+E210K, E56R+D57N+K98E+R108K+T244E, E56R+D57N+K98E+R108K+D254S, E56R+D57N+K98E+R118F+E210K, E56R+D57N+K98E+R118F+T244E, E56R+D57N+K98E+R118F+D254S, E56R+D57N+K98E+E210K+T244E, E56R+D57N+K98E+E210K+D254S, E56R+D57N+K98E+T244E+D254S, E56R+D57N+R108K+R118F+E210K, E56R+D57N+R108K+R118F+T244E, E56R+D57N+R108K+R118F+D254S, E56R+D57N+R108K+E210K+T244E, E56R+D57N+R108K+E210K+D254S, E56R+D57N+R108K+T244E+D254S, E56R+D57N+R118F+E210K+T244E, E56R+D57N+R118F+E210K+D254S, E56R+D57N+R118F+T244E+D254S, E56R+D57N+E210K+T244E+D254S, E56R+G91T+K98E+R108K+R118F, E56R+G91T+K98E+R108K+E210K, E56R+G91T+K98E+R108K+T244E, E56R+G91T+K98E+R108K+D254S, E56R+G91T+K98E+R118F+E210K, E56R+G91T+K98E+R118F+T244E, E56R+G91T+K98E+R118F+D254S, E56R+G91T+K98E+E210K+T244E, E56R+G91T+K98E+E210K+D254S, E56R+G91T+K98E+T244E+D254S, E56R+G91T+R108K+R118F+E210K, E56R+G91T+R108K+R118F+T244E, E56R+G91T+R108K+R118F+D254S, E56R+G91T+R108K+E210K+T244E, E56R+G91T+R108K+E210K+D254S, E56R+G91T+R108K+T244E+D254S, E56R+G91T+R118F+E210K+T244E, E56R+G91T+R118F+E210K+D254S, E56R+G91T+R118F+T244E+D254S, E56R+G91T+E210K+T244E+D254S, E56R+K98E+R108K+R118F+E210K, E56R+K98E+R108K+R118F+T244E, E56R+K98E+R108K+R118F+D254S, E56R+K98E+R108K+E210K+T244E, E56R+K98E+R108K+E210K+D254S, E56R+K98E+R108K+T244E+D254S, E56R+K98E+R118F+E210K+T244E, E56R+K98E+R118F+E210K+D254S, E56R+K98E+R118F+T244E+D254S, E56R+K98E+E210K+T244E+D254S, E56R+R108K+R118F+E210K+T244E, E56R+R108K+R118F+E210K+D254S, E56R+R108K+R118F+T244E+D254S, E56R+R108K+E210K+T244E+D254S, E56R+R118F+E210K+T244E+D254S, D57N+G91T+K98E+R108K+R118F, D57N+G91T+K98E+R108K+E210K, D57N+G91T+K98E+R108K+T244E, D57N+G91T+K98E+R108K+D254S, D57N+G91T+K98E+R118F+E210K, D57N+G91T+K98E+R118F+T244E, D57N+G91T+K98E+R118F+D254S, D57N+G91T+K98E+E210K+T244E, D57N+G91T+K98E+E210K+D254S, D57N+G91T+K98E+T244E+D254S, D57N+G91T+R108K+R118F+E210K, D57N+G91T+R108K+R118F+T244E, D57N+G91T+R108K+R118F+D254S, D57N+G91T+R108K+E210K+T244E, D57N+G91T+R108K+E210K+D254S, D57N+G91T+R108K+T244E+D254S, D57N+G91T+R118F+E210K+T244E, D57N+G91T+R118F+E210K+D254S, D57N+G91T+R118F+T244E+D254S, D57N+G91T+E210K+T244E+D254S, D57N+K98E+R108K+R118F+E210K, D57N+K98E+R108K+R118F+T244E, D57N+K98E+R108K+R118F+D254S, D57N+K98E+R108K+E210K+T244E, D57N+K98E+R108K+E210K+D254S, D57N+K98E+R108K+T244E+D254S, D57N+K98E+R118F+E210K+T244E, D57N+K98E+R118F+E210K+D254S, D57N+K98E+R118F+T244E+D254S, D57N+K98E+E210K+T244E+D254S, D57N+R108K+R118F+E210K+T244E, D57N+R108K+R118F+E210K+D254S, D57N+R108K+R118F+T244E+D254S, D57N+R108K+E210K+T244E+D254S, D57N+R118F+E210K+T244E+D254S, G91T+K98E+R108K+R118F+E210K, G91T+K98E+R108K+R118F+T244E, G91T+K98E+R108K+R118F+D254S, G91T+K98E+R108K+E210K+T244E, G91T+K98E+R108K+E210K+D254S, G91T+K98E+R108K+T244E+D254S, G91T+K98E+R118F+E210K+T244E, G91T+K98E+R118F+E210K+D254S, G91T+K98E+R118F+T244E+D254S, G91T+K98E+E210K+T244E+D254S, G91T+R108K+R118F+E210K+T244E, G91T+R108K+R118F+E210K+D254S, G91T+R108K+R118F+T244E+D254S, G91T+R108K+E210K+T244E+D254S, G91T+R118F+E210K+T244E+D254S, K98E+R108K+R118F+E210K+T244E, K98E+R108K+R118F+E210K+D254S, K98E+R108K+R118F+T244E+D254S, K98E+R108K+E210K+T244E+D254S, K98E+R118F+E210K+T244E+D254S, R108K+R118F+E210K+T244E+D254S, A40E+E56R+D57N+G91T+K98E+R108K, A40E+E56R+D57N+G91T+K98E+R118F, A40E+E56R+D57N+G91T+K98E+E210K, A40E+E56R+D57N+G91T+K98E+T244E, A40E+E56R+D57N+G91T+K98E+D254S, A40E+E56R+D57N+G91T+R108K+R118F, A40E+E56R+D57N+G91T+R108K+E210K, A40E+E56R+D57N+G91T+R108K+T244E, A40E+E56R+D57N+G91T+R108K+D254S, A40E+E56R+D57N+G91T+R118F+E210K, A40E+E56R+D57N+G91T+R118F+T244E, A40E+E56R+D57N+G91T+R118F+D254S, A40E+E56R+D57N+G91T+E210K+T244E, A40E+E56R+D57N+G91T+E210K+D254S, A40E+E56R+D57N+G91T+T244E+D254S, A40E+E56R+D57N+K98E+R108K+R118F, A40E+E56R+D57N+K98E+R108K+E210K, A40E+E56R+D57N+K98E+R108K+T244E, A40E+E56R+D57N+K98E+R108K+D254S, A40E+E56R+D57N+K98E+R118F+E210K, A40E+E56R+D57N+K98E+R118F+T244E, A40E+E56R+D57N+K98E+R118F+D254S, A40E+E56R+D57N+K98E+E210K+T244E, A40E+E56R+D57N+K98E+E210K+D254S, A40E+E56R+D57N+K98E+T244E+D254S, A40E+E56R+D57N+R108K+R118F+E210K, A40E+E56R+D57N+R108K+R118F+T244E, A40E+E56R+D57N+R108K+R118F+D254S, A40E+E56R+D57N+R108K+E210K+T244E, A40E+E56R+D57N+R108K+E210K+D254S, A40E+E56R+D57N+R108K+T244E+D254S, A40E+E56R+D57N+R118F+E210K+T244E, A40E+E56R+D57N+R118F+E210K+D254S, A40E+E56R+D57N+R118F+T244E+D254S, A40E+E56R+D57N+E210K+T244E+D254S, A40E+E56R+G91T+K98E+R108K+R118F, A40E+E56R+G91T+K98E+R108K+E210K, A40E+E56R+G91T+K98E+R108K+T244E, A40E+E56R+G91T+K98E+R108K+D254S, A40E+E56R+G91T+K98E+R118F+E210K, A40E+E56R+G91T+K98E+R118F+T244E, A40E+E56R+G91T+K98E+R118F+D254S, A40E+E56R+G91T+K98E+E210K+T244E, A40E+E56R+G91T+K98E+E210K+D254S, A40E+E56R+G91T+K98E+T244E+D254S, A40E+E56R+G91T+R108K+R118F+E210K, A40E+E56R+G91T+R108K+R118F+T244E, A40E+E56R+G91T+R108K+R118F+D254S, A40E+E56R+G91T+R108K+E210K+T244E, A40E+E56R+G91T+R108K+E210K+D254S, A40E+E56R+G91T+R108K+T244E+D254S, A40E+E56R+G91T+R118F+E210K+T244E, A40E+E56R+G91T+R118F+E210K+D254S, A40E+E56R+G91T+R118F+T244E+D254S, A40E+E56R+G91T+E210K+T244E+D254S, A40E+E56R+K98E+R108K+R118F+E210K, A40E+E56R+K98E+R108K+R118F+T244E, A40E+E56R+K98E+R108K+R118F+D254S, A40E+E56R+K98E+R108K+E210K+T244E, A40E+E56R+K98E+R108K+E210K+D254S, A40E+E56R+K98E+R108K+T244E+D254S, A40E+E56R+K98E+R118F+E210K+T244E, A40E+E56R+K98E+R118F+E210K+D254S, A40E+E56R+K98E+R118F+T244E+D254S, A40E+E56R+K98E+E210K+T244E+D254S, A40E+E56R+R108K+R118F+E210K+T244E, A40E+E56R+R108K+R118F+E210K+D254S, A40E+E56R+R108K+R118F+T244E+D254S, A40E+E56R+R108K+E210K+T244E+D254S, A40E+E56R+R118F+E210K+T244E+D254S, A40E+D57N+G91T+K98E+R108K+R118F, A40E+D57N+G91T+K98E+R108K+E210K, A40E+D57N+G91T+K98E+R108K+T244E, A40E+D57N+G91T+K98E+R108K+D254S, A40E+D57N+G91T+K98E+R118F+E210K, A40E+D57N+G91T+K98E+R118F+T244E, A40E+D57N+G91T+K98E+R118F+D254S, A40E+D57N+G91T+K98E+E210K+T244E, A40E+D57N+G91T+K98E+E210K+D254S, A40E+D57N+G91T+K98E+T244E+D254S, A40E+D57N+G91T+R108K+R118F+E210K, A40E+D57N+G91T+R108K+R118F+T244E, A40E+D57N+G91T+R108K+R118F+D254S, A40E+D57N+G91T+R108K+E210K+T244E, A40E+D57N+G91T+R108K+E210K+D254S, A40E+D57N+G91T+R108K+T244E+D254S, A40E+D57N+G91T+R118F+E210K+T244E, A40E+D57N+G91T+R118F+E210K+D254S, A40E+D57N+G91T+R118F+T244E+D254S, A40E+D57N+G91T+E210K+T244E+D254S, A40E+D57N+K98E+R108K+R118F+E210K, A40E+D57N+K98E+R108K+R118F+T244E, A40E+D57N+K98E+R108K+R118F+D254S, A40E+D57N+K98E+R108K+E210K+T244E, A40E+D57N+K98E+R108K+E210K+D254S, A40E+D57N+K98E+R108K+T244E+D254S, A40E+D57N+K98E+R118F+E210K+T244E, A40E+D57N+K98E+R118F+E210K+D254S, A40E+D57N+K98E+R118F+T244E+D254S, A40E+D57N+K98E+E210K+T244E+D254S, A40E+D57N+R108K+R118F+E210K+T244E, A40E+D57N+R108K+R118F+E210K+D254S, A40E+D57N+R108K+R118F+T244E+D254S, A40E+D57N+R108K+E210K+T244E+D254S, A40E+D57N+R118F+E210K+T244E+D254S, A40E+G91T+K98E+R108K+R118F+E210K, A40E+G91T+K98E+R108K+R118F+T244E, A40E+G91T+K98E+R108K+R118F+D254S, A40E+G91T+K98E+R108K+E210K+T244E, A40E+G91T+K98E+R108K+E210K+D254S, A40E+G91T+K98E+R108K+T244E+D254S, A40E+G91T+K98E+R118F+E210K+T244E, A40E+G91T+K98E+R118F+E210K+D254S, A40E+G91T+K98E+R118F+T244E+D254S, A40E+G91T+K98E+E210K+T244E+D254S, A40E+G91T+R108K+R118F+E210K+T244E, A40E+G91T+R108K+R118F+E210K+D254S, A40E+G91T+R108K+R118F+T244E+D254S, A40E+G91T+R108K+E210K+T244E+D254S, A40E+G91T+R118F+E210K+T244E+D254S, A40E+K98E+R108K+R118F+E210K+T244E, A40E+K98E+R108K+R118F+E210K+D254S, A40E+K98E+R108K+R118F+T244E+D254S, A40E+K98E+R108K+E210K+T244E+D254S, A40E+K98E+R118F+E210K+T244E+D254S, A40E+R108K+R118F+E210K+T244E+D254S, E56R+D57N+G91T+K98E+R108K+R118F, E56R+D57N+G91T+K98E+R108K+E210K, E56R+D57N+G91T+K98E+R108K+T244E, E56R+D57N+G91T+K98E+R108K+D254S, E56R+D57N+G91T+K98E+R118F+E210K, E56R+D57N+G91T+K98E+R118F+T244E, E56R+D57N+G91T+K98E+R118F+D254S, E56R+D57N+G91T+K98E+E210K+T244E, E56R+D57N+G91T+K98E+E210K+D254S, E56R+D57N+G91T+K98E+T244E+D254S, E56R+D57N+G91T+R108K+R118F+E210K, E56R+D57N+G91T+R108K+R118F+T244E, E56R+D57N+G91T+R108K+R118F+D254S, E56R+D57N+G91T+R108K+E210K+T244E, E56R+D57N+G91T+R108K+E210K+D254S, E56R+D57N+G91T+R108K+T244E+D254S, E56R+D57N+G91T+R118F+E210K+T244E, E56R+D57N+G91T+R118F+E210K+D254S, E56R+D57N+G91T+R118F+T244E+D254S, E56R+D57N+G91T+E210K+T244E+D254S, E56R+D57N+K98E+R108K+R118F+E210K, E56R+D57N+K98E+R108K+R118F+T244E, E56R+D57N+K98E+R108K+R118F+D254S, E56R+D57N+K98E+R108K+E210K+T244E, E56R+D57N+K98E+R108K+E210K+D254S, E56R+D57N+K98E+R108K+T244E+D254S, E56R+D57N+K98E+R118F+E210K+T244E, E56R+D57N+K98E+R118F+E210K+D254S, E56R+D57N+K98E+R118F+T244E+D254S, E56R+D57N+K98E+E210K+T244E+D254S, E56R+D57N+R108K+R118F+E210K+T244E, E56R+D57N+R108K+R118F+E210K+D254S, E56R+D57N+R108K+R118F+T244E+D254S, E56R+D57N+R108K+E210K+T244E+D254S, E56R+D57N+R118F+E210K+T244E+D254S, E56R+G91T+K98E+R108K+R118F+E210K, E56R+G91T+K98E+R108K+R118F+T244E, E56R+G91T+K98E+R108K+R118F+D254S, E56R+G91T+K98E+R108K+E210K+T244E, E56R+G91T+K98E+R108K+E210K+D254S, E56R+G91T+K98E+R108K+T244E+D254S, E56R+G91T+K98E+R118F+E210K+T244E, E56R+G91T+K98E+R118F+E210K+D254S, E56R+G91T+K98E+R118F+T244E+D254S, E56R+G91T+K98E+E210K+T244E+D254S, E56R+G91T+R108K+R118F+E210K+T244E, E56R+G91T+R108K+R118F+E210K+D254S, E56R+G91T+R108K+R118F+T244E+D254S, E56R+G91T+R108K+E210K+T244E+D254S, E56R+G91T+R118F+E210K+T244E+D254S, E56R+K98E+R108K+R118F+E210K+T244E, E56R+K98E+R108K+R118F+E210K+D254S, E56R+K98E+R108K+R118F+T244E+D254S, E56R+K98E+R108K+E210K+T244E+D254S, E56R+K98E+R118F+E210K+T244E+D254S, E56R+R108K+R118F+E210K+T244E+D254S, D57N+G91T+K98E+R108K+R118F+E210K, D57N+G91T+K98E+R108K+R118F+T244E, D57N+G91T+K98E+R108K+R118F+D254S, D57N+G91T+K98E+R108K+E210K+T244E, D57N+G91T+K98E+R108K+E210K+D254S, D57N+G91T+K98E+R108K+T244E+D254S, D57N+G91T+K98E+R118F+E210K+T244E, D57N+G91T+K98E+R118F+E210K+D254S, D57N+G91T+K98E+R118F+T244E+D254S, D57N+G91T+K98E+E210K+T244E+D254S, D57N+G91T+R108K+R118F+E210K+T244E, D57N+G91T+R108K+R118F+E210K+D254S, D57N+G91T+R108K+R118F+T244E+D254S, D57N+G91T+R108K+E210K+T244E+D254S, D57N+G91T+R118F+E210K+T244E+D254S, D57N+K98E+R108K+R118F+E210K+T244E, D57N+K98E+R108K+R118F+E210K+D254S, D57N+K98E+R108K+R118F+T244E+D254S, D57N+K98E+R108K+E210K+T244E+D254S, D57N+K98E+R118F+E210K+T244E+D254S, D57N+R108K+R118F+E210K+T244E+D254S, G91T+K98E+R108K+R118F+E210K+T244E, G91T+K98E+R108K+R118F+E210K+D254S, G91T+K98E+R108K+R118F+T244E+D254S, G91T+K98E+R108K+E210K+T244E+D254S, G91T+K98E+R118F+E210K+T244E+D254S, G91T+R108K+R118F+E210K+T244E+D254S,
K98E+R108K+R118F+E210K+T244E+D254S,
A40E+E56R+D57N+G91T+K98E+R108K+R118F,
A40E+E56R+D57N+G91T+K98E+R108K+E210K,
A40E+E56R+D57N+G91T+K98E+R108K+T244E,
A40E+E56R+D57N+G91T+K98E+R108K+D254S,
A40E+E56R+D57N+G91T+K98E+R118F+E210K,
A40E+E56R+D57N+G91T+K98E+R118F+T244E,
A40E+E56R+D57N+G91T+K98E+R118F+D254S,
A40E+E56R+D57N+G91T+K98E+E210K+T244E,
A40E+E56R+D57N+G91T+K98E+E210K+D254S,
A40E+E56R+D57N+G91T+K98E+T244E+D254S,
A40E+E56R+D57N+G91T+R108K+R118F+E210K,
A40E+E56R+D57N+G91T+R108K+R118F+T244E,
A40E+E56R+D57N+G91T+R108K+R118F+D254S,
A40E+E56R+D57N+G91T+R108K+E210K+T244E,
A40E+E56R+D57N+G91T+R108K+E210K+D254S,
A40E+E56R+D57N+G91T+R108K+T244E+D254S,
A40E+E56R+D57N+G91T+R118F+E210K+T244E,
A40E+E56R+D57N+G91T+R118F+E210K+D254S,
A40E+E56R+D57N+G91T+R118F+T244E+D254S,
A40E+E56R+D57N+G91T+E210K+T244E+D254S,
A40E+E56R+D57N+K98E+R108K+R118F+E210K,
A40E+E56R+D57N+K98E+R108K+R118F+T244E,
A40E+E56R+D57N+K98E+R108K+R118F+D254S,
A40E+E56R+D57N+K98E+R108K+E210K+T244E,
A40E+E56R+D57N+K98E+R108K+E210K+D254S,
A40E+E56R+D57N+K98E+R108K+T244E+D254S,
A40E+E56R+D57N+K98E+R118F+E210K+T244E,
A40E+E56R+D57N+K98E+R118F+E210K+D254S,
A40E+E56R+D57N+K98E+R118F+T244E+D254S,
A40E+E56R+D57N+K98E+E210K+T244E+D254S,
A40E+E56R+D57N+R108K+R118F+E210K+T244E,
A40E+E56R+D57N+R108K+R118F+E210K+D254S,
A40E+E56R+D57N+R108K+R118F+T244E+D254S,
A40E+E56R+D57N+R108K+E210K+T244E+D254S,
A40E+E56R+D57N+R118F+E210K+T244E+D254S,
A40E+E56R+G91T+K98E+R108K+R118F+E210K,
A40E+E56R+G91T+K98E+R108K+R118F+T244E,
A40E+E56R+G91T+K98E+R108K+R118F+D254S,
A40E+E56R+G91T+K98E+R108K+E210K+T244E,
A40E+E56R+G91T+K98E+R108K+E210K+D254S,
A40E+E56R+G91T+K98E+R108K+T244E+D254S,
A40E+E56R+G91T+K98E+R118F+E210K+T244E,
A40E+E56R+G91T+K98E+R118F+E210K+D254S,
A40E+E56R+G91T+K98E+R118F+T244E+D254S,
A40E+E56R+G91T+K98E+E210K+T244E+D254S,
A40E+E56R+G91T+R108K+R118F+E210K+T244E,
A40E+E56R+G91T+R108K+R118F+E210K+D254S,
A40E+E56R+G91T+R108K+R118F+T244E+D254S,
A40E+E56R+G91T+R108K+E210K+T244E+D254S,
A40E+E56R+G91T+R118F+E210K+T244E+D254S,
A40E+E56R+K98E+R108K+R118F+E210K+T244E,
A40E+E56R+K98E+R108K+R118F+E210K+D254S,
A40E+E56R+K98E+R108K+R118F+T244E+D254S,
A40E+E56R+K98E+R108K+E210K+T244E+D254S,
A40E+E56R+K98E+R118F+E210K+T244E+D254S,
A40E+E56R+R108K+R118F+E210K+T244E+D254S,
A40E+D57N+G91T+K98E+R108K+R118F+E210K,
A40E+D57N+G91T+K98E+R108K+R118F+T244E,
A40E+D57N+G91T+K98E+R108K+R118F+D254S,
A40E+D57N+G91T+K98E+R108K+E210K+T244E,
A40E+D57N+G91T+K98E+R108K+E210K+D254S,
A40E+D57N+G91T+K98E+R108K+T244E+D254S,
A40E+D57N+G91T+K98E+R118F+E210K+T244E,
A40E+D57N+G91T+K98E+R118F+E210K+D254S,
A40E+D57N+G91T+K98E+R118F+T244E+D254S,
A40E+D57N+G91T+K98E+E210K+T244E+D254S,
A40E+D57N+G91T+R108K+R118F+E210K+T244E,
A40E+D57N+G91T+R108K+R118F+E210K+D254S,
A40E+D57N+G91T+R108K+R118F+T244E+D254S,
A40E+D57N+G91T+R108K+E210K+T244E+D254S,
A40E+D57N+G91T+R118F+E210K+T244E+D254S,
A40E+D57N+K98E+R108K+R118F+E210K+T244E,
A40E+D57N+K98E+R108K+R118F+E210K+D254S,
A40E+D57N+K98E+R108K+R118F+T244E+D254S,
A40E+D57N+K98E+R108K+E210K+T244E+D254S,
A40E+D57N+K98E+R118F+E210K+T244E+D254S,
A40E+D57N+R108K+R118F+E210K+T244E+D254S,
A40E+G91T+K98E+R108K+R118F+E210K+T244E,
A40E+G91T+K98E+R108K+R118F+E210K+D254S,
A40E+G91T+K98E+R108K+R118F+T244E+D254S,
A40E+G91T+K98E+R108K+E210K+T244E+D254S,
A40E+G91T+K98E+R118F+E210K+T244E+D254S,
A40E+G91T+R108K+R118F+E210K+T244E+D254S,
A40E+K98E+R108K+R118F+E210K+T244E+D254S,
E56R+D57N+G91T+K98E+R108K+R118F+E210K,
E56R+D57N+G91T+K98E+R108K+R118F+T244E,
E56R+D57N+G91T+K98E+R108K+R118F+D254S,
E56R+D57N+G91T+K98E+R108K+E210K+T244E,
E56R+D57N+G91T+K98E+R108K+E210K+D254S,
E56R+D57N+G91T+K98E+R108K+T244E+D254S,
E56R+D57N+G91T+K98E+R118F+E210K+T244E,
E56R+D57N+G91T+K98E+R118F+E210K+D254S,
E56R+D57N+G91T+K98E+R118F+T244E+D254S,
E56R+D57N+G91T+K98E+E210K+T244E+D254S,
E56R+D57N+G91T+R108K+R118F+E210K+T244E,
E56R+D57N+G91T+R108K+R118F+E210K+D254S,
E56R+D57N+G91T+R108K+R118F+T244E+D254S,
E56R+D57N+G91T+R108K+E210K+T244E+D254S,
E56R+D57N+G91T+R118F+E210K+T244E+D254S,
E56R+D57N+K98E+R108K+R118F+E210K+T244E,
E56R+D57N+K98E+R108K+R118F+E210K+D254S,
E56R+D57N+K98E+R108K+R118F+T244E+D254S,
E56R+D57N+K98E+R108K+E210K+T244E+D254S,
E56R+D57N+K98E+R118F+E210K+T244E+D254S,
E56R+D57N+R108K+R118F+E210K+T244E+D254S,
E56R+G91T+K98E+R108K+R118F+E210K+T244E,
E56R+G91T+K98E+R108K+R118F+E210K+D254S,
E56R+G91T+K98E+R108K+R118F+T244E+D254S,
E56R+G91T+K98E+R108K+E210K+T244E+D254S,
E56R+G91T+K98E+R118F+E210K+T244E+D254S,
E56R+G91T+R108K+R118F+E210K+T244E+D254S,
E56R+K98E+R108K+R118F+E210K+T244E+D254S,
D57N+G91T+K98E+R108K+R118F+E210K+T244E,
D57N+G91T+K98E+R108K+R118F+E210K+D254S,
D57N+G91T+K98E+R108K+R118F+T244E+D254S,
D57N+G91T+K98E+R108K+E210K+T244E+D254S,
D57N+G91T+K98E+R118F+E210K+T244E+D254S,
D57N+G91T+R108K+R118F+E210K+T244E+D254S,
D57N+K98E+R108K+R118F+E210K+T244E+D254S,
G91T+K98E+R108K+R118F+E210K+T244E+D254S,
A40E+E56R+D57N+G91T+K98E+R108K+R118F+E210K,
A40E+E56R+D57N+G91T+K98E+R108K+R118F+T244E,
A40E+E56R+D57N+G91T+K98E+R108K+R118F+D254S,
A40E+E56R+D57N+G91T+K98E+R108K+E210K+T244E,
A40E+E56R+D57N+G91T+K98E+R108K+E210K+D254S,
A40E+E56R+D57N+G91T+K98E+R108K+T244E+D254S,
A40E+E56R+D57N+G91T+K98E+R118F+E210K+T244E,
A40E+E56R+D57N+G91T+K98E+R118F+E210K+D254S,
A40E+E56R+D57N+G91T+K98E+R118F+T244E+D254S,
A40E+E56R+D57N+G91T+K98E+E210K+T244E+D254S,
A40E+E56R+D57N+G91T+R108K+R118F+E210K+T244E,
A40E+E56R+D57N+G91T+R108K+R118F+E210K+D254S,
A40E+E56R+D57N+G91T+R108K+R118F+T244E+D254S,
A40E+E56R+D57N+G91T+R108K+E210K+T244E+D254S,
A40E+E56R+D57N+G91T+R118F+E210K+T244E+D254S,
A40E+E56R+D57N+K98E+R108K+R118F+E210K+T244E,
A40E+E56R+D57N+K98E+R108K+R118F+E210K+D254S,
A40E+E56R+D57N+K98E+R108K+R118F+T244E+D254S,
A40E+E56R+D57N+K98E+R108K+E210K+T244E+D254S,
A40E+E56R+D57N+K98E+R118F+E210K+T244E+D254S,
A40E+E56R+D57N+R108K+R118F+E210K+T244E+D254S,
A40E+E56R+G91T+K98E+R108K+R118F+E210K+T244E,
A40E+E56R+G91T+K98E+R108K+R118F+E210K+D254S,
A40E+E56R+G91T+K98E+R108K+R118F+T244E+D254S,
A40E+E56R+G91T+K98E+R108K+E210K+T244E+D254S,
A40E+E56R+G91T+K98E+R118F+E210K+T244E+D254S,
A40E+E56R+G91T+R108K+R118F+E210K+T244E+D254S,
A40E+E56R+K98E+R108K+R118F+E210K+T244E+D254S,
A40E+D57N+G91T+K98E+R108K+R118F+E210K+T244E,
A40E+D57N+G91T+K98E+R108K+R118F+E210K+D254S,
A40E+D57N+G91T+K98E+R108K+R118F+T244E+D254S,
A40E+D57N+G91T+K98E+R108K+E210K+T244E+D254S,
A40E+D57N+G91T+K98E+R118F+E210K+T244E+D254S,
A40E+D57N+G91T+R108K+R118F+E210K+T244E+D254S,
A40E+D57N+K98E+R108K+R118F+E210K+T244E+D254S,
A40E+G91T+K98E+R108K+R118F+E210K+T244E+D254S,
E56R+D57N+G91T+K98E+R108K+R118F+E210K+T244E,
E56R+D57N+G91T+K98E+R108K+R118F+E210K+D254S,
E56R+D57N+G91T+K98E+R108K+R118F+T244E+D254S,
E56R+D57N+G91T+K98E+R108K+E210K+T244E+D254S,
E56R+D57N+G91T+K98E+R118F+E210K+T244E+D254S,
E56R+D57N+G91T+R108K+R118F+E210K+T244E+D254S,
E56R+D57N+K98E+R108K+R118F+E210K+T244E+D254S,
E56R+G91T+K98E+R108K+R118F+E210K+T244E+D254S,
D57N+G91T+K98E+R108K+R118F+E210K+T244E+D254S,
A40E+E56R+D57N+G91T+K98E+R108K+R118F+E210K+T244E,
A40E+E56R+D57N+G91T+K98E+R108K+R118F+E210K+D254S,
A40E+E56R+D57N+G91T+K98E+R108K+R118F+T244E+D254S,
A40E+E56R+D57N+G91T+K98E+R108K+E210K+T244E+D254S,
A40E+E56R+D57N+G91T+K98E+R118F+E210K+T244E+D254S,
A40E+E56R+D57N+G91T+R108K+R118F+E210K+T244E+D254S,
A40E+E56R+D57N+K98E+R108K+R118F+E210K+T244E+D254S,
A40E+E56R+G91T+K98E+R108K+R118F+E210K+T244E+D254S,
A40E+D57N+G91T+K98E+R108K+R118F+E210K+T244E+D254S,
E56R+D57N+G91T+K98E+R108K+R118F+E210K+T244E+D254S,
A40E+E56R+D57N+G91T+K98E+R108K+R118F+E210K+T244E+D254S.
   7. The variant of any one of paragraph 1-5, wherein the variant has a substitution corresponding to I252H of SEQ ID NO: 8 and further comprises one of the following substitutions or set of substitutions corresponding to:
A40E, E56R, D57N, G91T, K98E, R108K, R118F, E210K, T244E, A40E+E56R, A40E+D57N, A40E+G91T, A40E+K98E, A40E+R108K, A40E+R118F, A40E+E210K, A40E+T244E, A40E+D254S, E56R+D57N, E56R+G91T, E56R+K98E, E56R+R108K, E56R+R118F, E56R+E210K, E56R+T244E, E56R+D254S, D57N+G91T, D57N+K98E, D57N+R108K, D57N+R118F, D57N+E210K, D57N+T244E, D57N+D254S, G91T+K98E, G91T+R108K, G91T+R118F, G91T+E210K, G91T+T244E, G91T+D254S, K98E+R108K, K98E+R118F, K98E+E210K, K98E+T244E, K98E+D254S, R108K+R118F, R108K+E210K, R108K+T244E, R108K+D254S, R118F+E210K, R118F+T244E, R118F+D254S, E210K+T244E, E210K+D254S, T244E+D254S, A40E+E56R+D57N, A40E+E56R+G91T, A40E+E56R+K98E, A40E+E56R+R108K, A40E+E56R+R118F, A40E+E56R+E210K, A40E+E56R+T244E, A40E+E56R+D254S, A40E+D57N+G91T, A40E+D57N+K98E, A40E+D57N+R108K, A40E+D57N+R118F, A40E+D57N+E210K, A40E+D57N+T244E, A40E+D57N+D254S, A40E+G91T+K98E, A40E+G91T+R108K, A40E+G91T+R118F, A40E+G91T+E210K, A40E+G91T+T244E, A40E+G91T+D254S, A40E+K98E+R108K, A40E+K98E+R118F, A40E+K98E+E210K, A40E+K98E+T244E, A40E+K98E+D254S, A40E+R108K+R118F, A40E+R108K+E210K, A40E+R108K+T244E, A40E+R108K+D254S, A40E+R118F+E210K, A40E+R118F+T244E, A40E+R118F+D254S, A40E+E210K+T244E, A40E+E210K+D254S, A40E+T244E+D254S, E56R+D57N+G91T, E56R+D57N+K98E, E56R+D57N+R108K, E56R+D57N+R118F, E56R+D57N+E210K, E56R+D57N+T244E, E56R+D57N+D254S, E56R+G91T+K98E, E56R+G91T+R108K, E56R+G91T+R118F, E56R+G91T+E210K, E56R+G91T+T244E, E56R+G91T+D254S, E56R+K98E+R108K, E56R+K98E+R118F, E56R+K98E+E210K, E56R+K98E+T244E, E56R+K98E+D254S, E56R+R108K+R118F, E56R+R108K+E210K, E56R+R108K+T244E, E56R+R108K+D254S, E56R+R118F+E210K, E56R+R118F+T244E, E56R+R118F+D254S, E56R+E210K+T244E, E56R+E210K+D254S, E56R+T244E+D254S, D57N+G91T+K98E, D57N+G91T+R108K, D57N+G91T+R118F, D57N+G91T+E210K, D57N+G91T+T244E, D57N+G91T+D254S, D57N+K98E+R108K, D57N+K98E+R118F, D57N+K98E+E210K, D57N+K98E+T244E, D57N+K98E+D254S, D57N+R108K+R118F, D57N+R108K+E210K, D57N+R108K+T244E, D57N+R108K+D254S, D57N+R118F+E210K, D57N+R118F+T244E, D57N+R118F+D254S, D57N+E210K+T244E, D57N+E210K+D254S, D57N+T244E+D254S, G91T+K98E+R108K, G91T+K98E+R118F, G91T+K98E+E210K, G91T+K98E+T244E, G91T+K98E+D254S, G91T+R108K+R118F, G91T+R108K+E210K, G91T+R108K+T244E, G91T+R108K+D254S, G91T+R118F+E210K, G91T+R118F+T244E, G91T+R118F+D254S, G91T+E210K+T244E, G91T+E210K+D254S, G91T+T244E+D254S, K98E+R108K+R118F, K98E+R108K+E210K, K98E+R108K+T244E, K98E+R108K+D254S, K98E+R118F+E210K, K98E+R118F+T244E, K98E+R118F+D254S, K98E+E210K+T244E, K98E+E210K+D254S, K98E+T244E+D254S, R108K+R118F+E210K, R108K+R118F+T244E, R108K+R118F+D254S, R108K+E210K+T244E, R108K+E210K+D254S, R108K+T244E+D254S, R118F+E210K+T244E, R118F+E210K+D254S, R118F+T244E+D254S, E210K+T244E+D254S, A40E+E56R+D57N+G91T, A40E+E56R+D57N+K98E, A40E+E56R+D57N+R108K, A40E+E56R+D57N+R118F, A40E+E56R+D57N+E210K, A40E+E56R+D57N+T244E, A40E+E56R+D57N+D254S, A40E+E56R+G91T+K98E, A40E+E56R+G91T+R108K, A40E+E56R+G91T+R118F, A40E+E56R+G91T+E210K, A40E+E56R+G91T+T244E, A40E+E56R+G91T+D254S, A40E+E56R+K98E+R108K, A40E+E56R+K98E+R118F, A40E+E56R+K98E+E210K, A40E+E56R+K98E+T244E, A40E+E56R+K98E+D254S, A40E+E56R+R108K+R118F, A40E+E56R+R108K+E210K, A40E+E56R+R108K+T244E, A40E+E56R+R108K+D254S, A40E+E56R+R118F+E210K, A40E+E56R+R118F+T244E, A40E+E56R+R118F+D254S, A40E+E56R+E210K+T244E, A40E+E56R+E210K+D254S, A40E+E56R+T244E+D254S, A40E+D57N+G91T+K98E, A40E+D57N+G91T+R108K, A40E+D57N+G91T+R118F, A40E+D57N+G91T+E210K, A40E+D57N+G91T+T244E, A40E+D57N+G91T+D254S, A40E+D57N+K98E+R108K, A40E+D57N+K98E+R118F, A40E+D57N+K98E+E210K, A40E+D57N+K98E+T244E, A40E+D57N+K98E+D254S, A40E+D57N+R108K+R118F, A40E+D57N+R108K+E210K, A40E+D57N+R108K+T244E, A40E+D57N+R108K+D254S, A40E+D57N+R118F+E210K, A40E+D57N+R118F+T244E, A40E+D57N+R118F+D254S, A40E+D57N+E210K+T244E, A40E+D57N+E210K+D254S, A40E+D57N+T244E+D254S, A40E+G91T+K98E+R108K, A40E+G91T+K98E+R118F, A40E+G91T+K98E+E210K, A40E+G91T+K98E+T244E, A40E+G91T+K98E+D254S, A40E+G91T+R108K+R118F, A40E+G91T+R108K+E210K, A40E+G91T+R108K+T244E, A40E+G91T+R108K+D254S, A40E+G91T+R118F+E210K, A40E+G91T+R118F+T244E, A40E+G91T+R118F+D254S, A40E+G91T+E210K+T244E, A40E+G91T+E210K+D254S, A40E+G91T+T244E+D254S, A40E+K98E+R108K+R118F, A40E+K98E+R108K+E210K, A40E+K98E+R108K+T244E, A40E+K98E+R108K+D254S, A40E+K98E+R118F+E210K, A40E+K98E+R118F+T244E, A40E+K98E+R118F+D254S, A40E+K98E+E210K+T244E, A40E+K98E+E210K+D254S, A40E+K98E+T244E+D254S, A40E+R108K+R118F+E210K, A40E+R108K+R118F+T244E, A40E+R108K+R118F+D254S, A40E+R108K+E210K+T244E, A40E+R108K+E210K+D254S, A40E+R108K+T244E+D254S, A40E+R118F+E210K+T244E, A40E+R118F+E210K+D254S, A40E+R118F+T244E+D254S, A40E+E210K+T244E+D254S, E56R+D57N+G91T+K98E, E56R+D57N+G91T+R108K, E56R+D57N+G91T+R118F, E56R+D57N+G91T+E210K, E56R+D57N+G91T+T244E, E56R+D57N+G91T+D254S, E56R+D57N+K98E+R108K, E56R+D57N+K98E+R118F, E56R+D57N+K98E+E210K, E56R+D57N+K98E+T244E, E56R+D57N+K98E+D254S, E56R+D57N+R108K+R118F, E56R+D57N+R108K+E210K, E56R+D57N+R108K+T244E, E56R+D57N+R108K+D254S, E56R+D57N+R118F+E210K, E56R+D57N+R118F+T244E, E56R+D57N+R118F+D254S, E56R+D57N+E210K+T244E, E56R+D57N+E210K+D254S, E56R+D57N+T244E+D254S, E56R+G91T+K98E+R108K, E56R+G91T+K98E+R118F, E56R+G91T+K98E+E210K, E56R+G91T+K98E+T244E, E56R+G91T+K98E+D254S, E56R+G91T+R108K+R118F, E56R+G91T+R108K+E210K, E56R+G91T+R108K+T244E, E56R+G91T+R108K+D254S, E56R+G91T+R118F+E210K, E56R+G91T+R118F+T244E, E56R+G91T+R118F+D254S, E56R+G91T+E210K+T244E, E56R+G91T+E210K+D254S, E56R+G91T+T244E+D254S, E56R+K98E+R108K+R118F, E56R+K98E+R108K+E210K, E56R+K98E+R108K+T244E, E56R+K98E+R108K+D254S, E56R+K98E+R118F+E210K, E56R+K98E+R118F+T244E, E56R+K98E+R118F+D254S, E56R+K98E+E210K+T244E, E56R+K98E+E210K+D254S, E56R+K98E+T244E+D254S, E56R+R108K+R118F+E210K, E56R+R108K+R118F+T244E, E56R+R108K+R118F+D254S, E56R+R108K+E210K+T244E, E56R+R108K+E210K+D254S, E56R+R108K+T244E+D254S, E56R+R118F+E210K+T244E, E56R+R118F+E210K+D254S, E56R+R118F+T244E+D254S, E56R+E210K+T244E+D254S, D57N+G91T+K98E+R108K, D57N+G91T+K98E+R118F, D57N+G91T+K98E+E210K, D57N+G91T+K98E+T244E, D57N+G91T+K98E+D254S, D57N+G91T+R108K+R118F, D57N+G91T+R108K+E210K, D57N+G91T+R108K+T244E, D57N+G91T+R108K+D254S, D57N+G91T+R118F+E210K, D57N+G91T+R118F+T244E, D57N+G91T+R118F+D254S, D57N+G91T+E210K+T244E, D57N+G91T+E210K+D254S, D57N+G91T+T244E+D254S, D57N+K98E+R108K+R118F, D57N+K98E+R108K+E210K, D57N+K98E+R108K+T244E, D57N+K98E+R108K+D254S, D57N+K98E+R118F+E210K, D57N+K98E+R118F+T244E, D57N+K98E+R118F+D254S, D57N+K98E+E210K+T244E, D57N+K98E+E210K+D254S, D57N+K98E+T244E+D254S, D57N+R108K+R118F+E210K, D57N+R108K+R118F+T244E, D57N+R108K+R118F+D254S, D57N+R108K+E210K+T244E, D57N+R108K+E210K+D254S, D57N+R108K+T244E+D254S, D57N+R118F+E210K+T244E, D57N+R118F+E210K+D254S, D57N+R118F+T244E+D254S, D57N+E210K+T244E+D254S, G91T+K98E+R108K+R118F, G91T+K98E+R108K+E210K, G91T+K98E+R108K+T244E, G91T+K98E+R108K+D254S, G91T+K98E+R118F+E210K, G91T+K98E+R118F+T244E, G91T+K98E+R118F+D254S, G91T+K98E+E210K+T244E, G91T+K98E+E210K+D254S, G91T+K98E+T244E+D254S, G91T+R108K+R118F+E210K, G91T+R108K+R118F+T244E, G91T+R108K+R118F+D254S, G91T+R108K+E210K+T244E, G91T+R108K+E210K+D254S, G91T+R108K+T244E+D254S, G91T+R118F+E210K+T244E, G91T+R118F+E210K+D254S, G91T+R118F+T244E+D254S, G91T+E210K+T244E+D254S, K98E+R108K+R118F+E210K, K98E+R108K+R118F+T244E, K98E+R108K+R118F+D254S, K98E+R108K+E210K+T244E, K98E+R108K+E210K+D254S, K98E+R108K+T244E+D254S, K98E+R118F+E210K+T244E, K98E+R118F+E210K+D254S, K98E+R118F+T244E+D254S, K98E+E210K+T244E+D254S, R108K+R118F+E210K+T244E, R108K+R118F+E210K+D254S, R108K+R118F+T244E+D254S, R108K+E210K+T244E+D254S, R118F+E210K+T244E+D254S, A40E+E56R+D57N+G91T+K98E, A40E+E56R+D57N+G91T+R108K, A40E+E56R+D57N+G91T+R118F, A40E+E56R+D57N+G91T+E210K, A40E+E56R+D57N+G91T+T244E, A40E+E56R+D57N+G91T+D254S, A40E+E56R+D57N+K98E+R108K, A40E+E56R+D57N+K98E+R118F, A40E+E56R+D57N+K98E+E210K, A40E+E56R+D57N+K98E+T244E, A40E+E56R+D57N+K98E+D254S, A40E+E56R+D57N+R108K+R118F, A40E+E56R+D57N+R108K+E210K, A40E+E56R+D57N+R108K+T244E, A40E+E56R+D57N+R108K+D254S, A40E+E56R+D57N+R118F+E210K, A40E+E56R+D57N+R118F+T244E, A40E+E56R+D57N+R118F+D254S, A40E+E56R+D57N+E210K+T244E, A40E+E56R+D57N+E210K+D254S, A40E+E56R+D57N+T244E+D254S, A40E+E56R+G91T+K98E+R108K, A40E+E56R+G91T+K98E+R118F, A40E+E56R+G91T+K98E+E210K, A40E+E56R+G91T+K98E+T244E, A40E+E56R+G91T+K98E+D254S, A40E+E56R+G91T+R108K+R118F, A40E+E56R+G91T+R108K+E210K, A40E+E56R+G91T+R108K+T244E, A40E+E56R+G91T+R108K+D254S, A40E+E56R+G91T+R118F+E210K, A40E+E56R+G91T+R118F+T244E, A40E+E56R+G91T+R118F+D254S, A40E+E56R+G91T+E210K+T244E, A40E+E56R+G91T+E210K+D254S, A40E+E56R+G91T+T244E+D254S, A40E+E56R+K98E+R108K+R118F, A40E+E56R+K98E+R108K+E210K, A40E+E56R+K98E+R108K+T244E, A40E+E56R+K98E+R108K+D254S, A40E+E56R+K98E+R118F+E210K, A40E+E56R+K98E+R118F+T244E, A40E+E56R+K98E+R118F+D254S, A40E+E56R+K98E+E210K+T244E, A40E+E56R+K98E+E210K+D254S, A40E+E56R+K98E+T244E+D254S, A40E+E56R+R108K+R118F+E210K, A40E+E56R+R108K+R118F+T244E, A40E+E56R+R108K+R118F+D254S, A40E+E56R+R108K+E210K+T244E, A40E+E56R+R108K+E210K+D254S, A40E+E56R+R108K+T244E+D254S, A40E+E56R+R118F+E210K+T244E, A40E+E56R+R118F+E210K+D254S, A40E+E56R+R118F+T244E+D254S, A40E+E56R+E210K+T244E+D254S, A40E+D57N+G91T+K98E+R108K, A40E+D57N+G91T+K98E+R118F, A40E+D57N+G91T+K98E+E210K, A40E+D57N+G91T+K98E+T244E, A40E+D57N+G91T+K98E+D254S, A40E+D57N+G91T+R108K+R118F, A40E+D57N+G91T+R108K+E210K, A40E+D57N+G91T+R108K+T244E, A40E+D57N+G91T+R108K+D254S, A40E+D57N+G91T+R118F+E210K, A40E+D57N+G91T+R118F+T244E, A40E+D57N+G91T+R118F+D254S, A40E+D57N+G91T+E210K+T244E, A40E+D57N+G91T+E210K+D254S, A40E+D57N+G91T+T244E+D254S, A40E+D57N+K98E+R108K+R118F, A40E+D57N+K98E+R108K+E210K, A40E+D57N+K98E+R108K+T244E, A40E+D57N+K98E+R108K+D254S, A40E+D57N+K98E+R118F+E210K, A40E+D57N+K98E+R118F+T244E, A40E+D57N+K98E+R118F+D254S, A40E+D57N+K98E+E210K+T244E, A40E+D57N+K98E+E210K+D254S, A40E+D57N+K98E+T244E+D254S, A40E+D57N+R108K+R118F+E210K, A40E+D57N+R108K+R118F+T244E, A40E+D57N+R108K+R118F+D254S, A40E+D57N+R108K+E210K+T244E, A40E+D57N+R108K+E210K+D254S, A40E+D57N+R108K+T244E+D254S, A40E+D57N+R118F+E210K+T244E, A40E+D57N+R118F+E210K+D254S, A40E+D57N+R118F+T244E+D254S, A40E+D57N+E210K+T244E+D254S, A40E+G91T+K98E+R108K+R118F, A40E+G91T+K98E+R108K+E210K, A40E+G91T+K98E+R108K+T244E, A40E+G91T+K98E+R108K+D254S, A40E+G91T+K98E+R118F+E210K, A40E+G91T+K98E+R118F+T244E, A40E+G91T+K98E+R118F+D254S, A40E+G91T+K98E+E210K+T244E, A40E+G91T+K98E+E210K+D254S, A40E+G91T+K98E+T244E+D254S, A40E+G91T+R108K+R118F+E210K, A40E+G91T+R108K+R118F+T244E, A40E+G91T+R108K+R118F+D254S, A40E+G91T+R108K+E210K+T244E, A40E+G91T+R108K+E210K+D254S, A40E+G91T+R108K+T244E+D254S, A40E+G91T+R118F+E210K+T244E, A40E+G91T+R118F+E210K+D254S, A40E+G91T+R118F+T244E+D254S, A40E+G91T+E210K+T244E+D254S, A40E+K98E+R108K+R118F+E210K, A40E+K98E+R108K+R118F+T244E, A40E+K98E+R108K+R118F+D254S, A40E+K98E+R108K+E210K+T244E, A40E+K98E+R108K+E210K+D254S, A40E+K98E+R108K+T244E+D254S, A40E+K98E+R118F+E210K+T244E, A40E+K98E+R118F+E210K+D254S, A40E+K98E+R118F+T244E+D254S, A40E+K98E+E210K+T244E+D254S, A40E+R108K+R118F+E210K+T244E, A40E+R108K+R118F+E210K+D254S, A40E+R108K+R118F+T244E+D254S, A40E+R108K+E210K+T244E+D254S, A40E+R118F+E210K+T244E+D254S, E56R+D57N+G91T+K98E+R108K, E56R+D57N+G91T+K98E+R118F, E56R+D57N+G91T+K98E+E210K, E56R+D57N+G91T+K98E+T244E, E56R+D57N+G91T+K98E+D254S, E56R+D57N+G91T+R108K+R118F, E56R+D57N+G91T+R108K+E210K, E56R+D57N+G91T+R108K+T244E, E56R+D57N+G91T+R108K+D254S, E56R+D57N+G91T+R118F+E210K, E56R+D57N+G91T+R118F+T244E, E56R+D57N+G91T+R118F+D254S, E56R+D57N+G91T+E210K+T244E, E56R+D57N+G91T+E210K+D254S, E56R+D57N+G91T+T244E+D254S, E56R+D57N+K98E+R108K+R118F, E56R+D57N+K98E+R108K+E210K, E56R+D57N+K98E+R108K+T244E, E56R+D57N+K98E+R108K+D254S, E56R+D57N+K98E+R118F+E210K, E56R+D57N+K98E+R118F+T244E, E56R+D57N+K98E+R118F+D254S, E56R+D57N+K98E+E210K+T244E, E56R+D57N+K98E+E210K+D254S, E56R+D57N+K98E+T244E+D254S, E56R+D57N+R108K+R118F+E210K, E56R+D57N+R108K+R118F+T244E, E56R+D57N+R108K+R118F+D254S, E56R+D57N+R108K+E210K+T244E, E56R+D57N+R108K+E210K+D254S, E56R+D57N+R108K+T244E+D254S, E56R+D57N+R118F+E210K+T244E, E56R+D57N+R118F+E210K+D254S, E56R+D57N+R118F+T244E+D254S, E56R+D57N+E210K+T244E+D254S, E56R+G91T+K98E+R108K+R118F, E56R+G91T+K98E+R108K+E210K, E56R+G91T+K98E+R108K+T244E, E56R+G91T+K98E+R108K+D254S, E56R+G91T+K98E+R118F+E210K, E56R+G91T+K98E+R118F+T244E, E56R+G91T+K98E+R118F+D254S, E56R+G91T+K98E+E210K+T244E, E56R+G91T+K98E+E210K+D254S, E56R+G91T+K98E+T244E+D254S, E56R+G91T+R108K+R118F+E210K, E56R+G91T+R108K+R118F+T244E, E56R+G91T+R108K+R118F+D254S, E56R+G91T+R108K+E210K+T244E, E56R+G91T+R108K+E210K+D254S, E56R+G91T+R108K+T244E+D254S, E56R+G91T+R118F+E210K+T244E, E56R+G91T+R118F+E210K+D254S, E56R+G91T+R118F+T244E+D254S, E56R+G91T+E210K+T244E+D254S, E56R+K98E+R108K+R118F+E210K, E56R+K98E+R108K+R118F+T244E, E56R+K98E+R108K+R118F+D254S, E56R+K98E+R108K+E210K+T244E, E56R+K98E+R108K+E210K+D254S, E56R+K98E+R108K+T244E+D254S, E56R+K98E+R118F+E210K+T244E, E56R+K98E+R118F+E210K+D254S, E56R+K98E+R118F+T244E+D254S, E56R+K98E+E210K+T244E+D254S, E56R+R108K+R118F+E210K+T244E, E56R+R108K+R118F+E210K+D254S, E56R+R108K+R118F+T244E+D254S, E56R+R108K+E210K+T244E+D254S, E56R+R118F+E210K+T244E+D254S, D57N+G91T+K98E+R108K+R118F, D57N+G91T+K98E+R108K+E210K, D57N+G91T+K98E+R108K+T244E, D57N+G91T+K98E+R108K+D254S, D57N+G91T+K98E+R118F+E210K, D57N+G91T+K98E+R118F+T244E, D57N+G91T+K98E+R118F+D254S, D57N+G91T+K98E+E210K+T244E, D57N+G91T+K98E+E210K+D254S, D57N+G91T+K98E+T244E+D254S, D57N+G91T+R108K+R118F+E210K, D57N+G91T+R108K+R118F+T244E, D57N+G91T+R108K+R118F+D254S, D57N+G91T+R108K+E210K+T244E, D57N+G91T+R108K+E210K+D254S, D57N+G91T+R108K+T244E+D254S, D57N+G91T+R118F+E210K+T244E, D57N+G91T+R118F+E210K+D254S, D57N+G91T+R118F+T244E+D254S, D57N+G91T+E210K+T244E+D254S, D57N+K98E+R108K+R118F+E210K, D57N+K98E+R108K+R118F+T244E, D57N+K98E+R108K+R118F+D254S, D57N+K98E+R108K+E210K+T244E, D57N+K98E+R108K+E210K+D254S, D57N+K98E+R108K+T244E+D254S, D57N+K98E+R118F+E210K+T244E, D57N+K98E+R118F+E210K+D254S, D57N+K98E+R118F+T244E+D254S, D57N+K98E+E210K+T244E+D254S, D57N+R108K+R118F+E210K+T244E, D57N+R108K+R118F+E210K+D254S, D57N+R108K+R118F+T244E+D254S, D57N+R108K+E210K+T244E+D254S, D57N+R118F+E210K+T244E+D254S, G91T+K98E+R108K+R118F+E210K, G91T+K98E+R108K+R118F+T244E, G91T+K98E+R108K+R118F+D254S, G91T+K98E+R108K+E210K+T244E, G91T+K98E+R108K+E210K+D254S, G91T+K98E+R108K+T244E+D254S, G91T+K98E+R118F+E210K+T244E, G91T+K98E+R118F+E210K+D254S, G91T+K98E+R118F+T244E+D254S, G91T+K98E+E210K+T244E+D254S, G91T+R108K+R118F+E210K+T244E, G91T+R108K+R118F+E210K+D254S, G91T+R108K+R118F+T244E+D254S, G91T+R108K+E210K+T244E+D254S, G91T+R118F+E210K+T244E+D254S, K98E+R108K+R118F+E210K+T244E, K98E+R108K+R118F+E210K+D254S, K98E+R108K+R118F+T244E+D254S, K98E+R108K+E210K+T244E+D254S, K98E+R118F+E210K+T244E+D254S, R108K+R118F+E210K+T244E+D254S, A40E+E56R+D57N+G91T+K98E+R108K, A40E+E56R+D57N+G91T+K98E+R118F, A40E+E56R+D57N+G91T+K98E+E210K, A40E+E56R+D57N+G91T+K98E+T244E, A40E+E56R+D57N+G91T+K98E+D254S, A40E+E56R+D57N+G91T+R108K+R118F, A40E+E56R+D57N+G91T+R108K+E210K, A40E+E56R+D57N+G91T+R108K+T244E, A40E+E56R+D57N+G91T+R108K+D254S, A40E+E56R+D57N+G91T+R118F+E210K, A40E+E56R+D57N+G91T+R118F+T244E, A40E+E56R+D57N+G91T+R118F+D254S, A40E+E56R+D57N+G91T+E210K+T244E, A40E+E56R+D57N+G91T+E210K+D254S, A40E+E56R+D57N+G91T+T244E+D254S, A40E+E56R+D57N+K98E+R108K+R118F, A40E+E56R+D57N+K98E+R108K+E210K, A40E+E56R+D57N+K98E+R108K+T244E, A40E+E56R+D57N+K98E+R108K+D254S, A40E+E56R+D57N+K98E+R118F+E210K, A40E+E56R+D57N+K98E+R118F+T244E, A40E+E56R+D57N+K98E+R118F+D254S, A40E+E56R+D57N+K98E+E210K+T244E, A40E+E56R+D57N+K98E+E210K+D254S, A40E+E56R+D57N+K98E+T244E+D254S, A40E+E56R+D57N+R108K+R118F+E210K, A40E+E56R+D57N+R108K+R118F+T244E, A40E+E56R+D57N+R108K+R118F+D254S, A40E+E56R+D57N+R108K+E210K+T244E, A40E+E56R+D57N+R108K+E210K+D254S, A40E+E56R+D57N+R108K+T244E+D254S, A40E+E56R+D57N+R118F+E210K+T244E, A40E+E56R+D57N+R118F+E210K+D254S, A40E+E56R+D57N+R118F+T244E+D254S, A40E+E56R+D57N+E210K+T244E+D254S, A40E+E56R+G91T+K98E+R108K+R118F, A40E+E56R+G91T+K98E+R108K+E210K, A40E+E56R+G91T+K98E+R108K+T244E, A40E+E56R+G91T+K98E+R108K+D254S, A40E+E56R+G91T+K98E+R118F+E210K, A40E+E56R+G91T+K98E+R118F+T244E, A40E+E56R+G91T+K98E+R118F+D254S, A40E+E56R+G91T+K98E+E210K+T244E, A40E+E56R+G91T+K98E+E210K+D254S, A40E+E56R+G91T+K98E+T244E+D254S, A40E+E56R+G91T+R108K+R118F+E210K, A40E+E56R+G91T+R108K+R118F+T244E, A40E+E56R+G91T+R108K+R118F+D254S, A40E+E56R+G91T+R108K+E210K+T244E, A40E+E56R+G91T+R108K+E210K+D254S, A40E+E56R+G91T+R108K+T244E+D254S, A40E+E56R+G91T+R118F+E210K+T244E, A40E+E56R+G91T+R118F+E210K+D254S, A40E+E56R+G91T+R118F+T244E+D254S, A40E+E56R+G91T+E210K+T244E+D254S, A40E+E56R+K98E+R108K+R118F+E210K, A40E+E56R+K98E+R108K+R118F+T244E, A40E+E56R+K98E+R108K+R118F+D254S, A40E+E56R+K98E+R108K+E210K+T244E, A40E+E56R+K98E+R108K+E210K+D254S, A40E+E56R+K98E+R108K+T244E+D254S, A40E+E56R+K98E+R118F+E210K+T244E, A40E+E56R+K98E+R118F+E210K+D254S, A40E+E56R+K98E+R118F+T244E+D254S, A40E+E56R+K98E+E210K+T244E+D254S, A40E+E56R+R108K+R118F+E210K+T244E, A40E+E56R+R108K+R118F+E210K+D254S, A40E+E56R+R108K+R118F+T244E+D254S, A40E+E56R+R108K+E210K+T244E+D254S, A40E+E56R+R118F+E210K+T244E+D254S, A40E+D57N+G91T+K98E+R108K+R118F, A40E+D57N+G91T+K98E+R108K+E210K, A40E+D57N+G91T+K98E+R108K+T244E, A40E+D57N+G91T+K98E+R108K+D254S, A40E+D57N+G91T+K98E+R118F+E210K, A40E+D57N+G91T+K98E+R118F+T244E, A40E+D57N+G91T+K98E+R118F+D254S, A40E+D57N+G91T+K98E+E210K+T244E, A40E+D57N+G91T+K98E+E210K+D254S, A40E+D57N+G91T+K98E+T244E+D254S, A40E+D57N+G91T+R108K+R118F+E210K, A40E+D57N+G91T+R108K+R118F+T244E, A40E+D57N+G91T+R108K+R118F+D254S, A40E+D57N+G91T+R108K+E210K+T244E, A40E+D57N+G91T+R108K+E210K+D254S, A40E+D57N+G91T+R108K+T244E+D254S, A40E+D57N+G91T+R118F+E210K+T244E, A40E+D57N+G91T+R118F+E210K+D254S, A40E+D57N+G91T+R118F+T244E+D254S, A40E+D57N+G91T+E210K+T244E+D254S, A40E+D57N+K98E+R108K+R118F+E210K, A40E+D57N+K98E+R108K+R118F+T244E, A40E+D57N+K98E+R108K+R118F+D254S, A40E+D57N+K98E+R108K+E210K+T244E, A40E+D57N+K98E+R108K+E210K+D254S, A40E+D57N+K98E+R108K+T244E+D254S, A40E+D57N+K98E+R118F+E210K+T244E, A40E+D57N+K98E+R118F+E210K+D254S, A40E+D57N+K98E+R118F+T244E+D254S, A40E+D57N+K98E+E210K+T244E+D254S, A40E+D57N+R108K+R118F+E210K+T244E, A40E+D57N+R108K+R118F+E210K+D254S, A40E+D57N+R108K+R118F+T244E+D254S, A40E+D57N+R108K+E210K+T244E+D254S, A40E+D57N+R118F+E210K+T244E+D254S, A40E+G91T+K98E+R108K+R118F+E210K, A40E+G91T+K98E+R108K+R118F+T244E, A40E+G91T+K98E+R108K+R118F+D254S, A40E+G91T+K98E+R108K+E210K+T244E, A40E+G91T+K98E+R108K+E210K+D254S, A40E+G91T+K98E+R108K+T244E+D254S, A40E+G91T+K98E+R118F+E210K+T244E, A40E+G91T+K98E+R118F+E210K+D254S, A40E+G91T+K98E+R118F+T244E+D254S, A40E+G91T+K98E+E210K+T244E+D254S, A40E+G91T+R108K+R118F+E210K+T244E, A40E+G91T+R108K+R118F+E210K+D254S, A40E+G91T+R108K+R118F+T244E+D254S, A40E+G91T+R108K+E210K+T244E+D254S, A40E+G91T+R118F+E210K+T244E+D254S, A40E+K98E+R108K+R118F+E210K+T244E, A40E+K98E+R108K+R118F+E210K+D254S, A40E+K98E+R108K+R118F+T244E+D254S, A40E+K98E+R108K+E210K+T244E+D254S, A40E+K98E+R118F+E210K+T244E+D254S, A40E+R108K+R118F+E210K+T244E+D254S, E56R+D57N+G91T+K98E+R108K+R118F, E56R+D57N+G91T+K98E+R108K+E210K, E56R+D57N+G91T+K98E+R108K+T244E, E56R+D57N+G91T+K98E+R108K+D254S, E56R+D57N+G91T+K98E+R118F+E210K, E56R+D57N+G91T+K98E+R118F+T244E, E56R+D57N+G91T+K98E+R118F+D254S, E56R+D57N+G91T+K98E+E210K+T244E, E56R+D57N+G91T+K98E+E210K+D254S, E56R+D57N+G91T+K98E+T244E+D254S, E56R+D57N+G91T+R108K+R118F+E210K, E56R+D57N+G91T+R108K+R118F+T244E, E56R+D57N+G91T+R108K+R118F+D254S, E56R+D57N+G91T+R108K+E210K+T244E, E56R+D57N+G91T+R108K+E210K+D254S, E56R+D57N+G91T+R108K+T244E+D254S, E56R+D57N+G91T+R118F+E210K+T244E, E56R+D57N+G91T+R118F+E210K+D254S, E56R+D57N+G91T+R118F+T244E+D254S, E56R+D57N+G91T+E210K+T244E+D254S, E56R+D57N+K98E+R108K+R118F+E210K, E56R+D57N+K98E+R108K+R118F+T244E, E56R+D57N+K98E+R108K+R118F+D254S, E56R+D57N+K98E+R108K+E210K+T244E, E56R+D57N+K98E+R108K+E210K+D254S, E56R+D57N+K98E+R108K+T244E+D254S, E56R+D57N+K98E+R118F+E210K+T244E, E56R+D57N+K98E+R118F+E210K+D254S, E56R+D57N+K98E+R118F+T244E+D254S, E56R+D57N+K98E+E210K+T244E+D254S, E56R+D57N+R108K+R118F+E210K+T244E, E56R+D57N+R108K+R118F+E210K+D254S, E56R+D57N+R108K+R118F+T244E+D254S, E56R+D57N+R108K+E210K+T244E+D254S, E56R+D57N+R118F+E210K+T244E+D254S, E56R+G91T+K98E+R108K+R118F+E210K, E56R+G91T+K98E+R108K+R118F+T244E, E56R+G91T+K98E+R108K+R118F+D254S, E56R+G91T+K98E+R108K+E210K+T244E, E56R+G91T+K98E+R108K+E210K+D254S, E56R+G91T+K98E+R108K+T244E+D254S, E56R+G91T+K98E+R118F+E210K+T244E, E56R+G91T+K98E+R118F+E210K+D254S, E56R+G91T+K98E+R118F+T244E+D254S, E56R+G91T+K98E+E210K+T244E+D254S, E56R+G91T+R108K+R118F+E210K+T244E, E56R+G91T+R108K+R118F+E210K+D254S, E56R+G91T+R108K+R118F+T244E+D254S, E56R+G91T+R108K+E210K+T244E+D254S, E56R+G91T+R118F+E210K+T244E+D254S, E56R+K98E+R108K+R118F+E210K+T244E, E56R+K98E+R108K+R118F+E210K+D254S, E56R+K98E+R108K+R118F+T244E+D254S, E56R+K98E+R108K+E210K+T244E+D254S, E56R+K98E+R118F+E210K+T244E+D254S, E56R+R108K+R118F+E210K+T244E+D254S, D57N+G91T+K98E+R108K+R118F+E210K, D57N+G91T+K98E+R108K+R118F+T244E, D57N+G91T+K98E+R108K+R118F+D254S, D57N+G91T+K98E+R108K+E210K+T244E, D57N+G91T+K98E+R108K+E210K+D254S, D57N+G91T+K98E+R108K+T244E+D254S, D57N+G91T+K98E+R118F+E210K+T244E, D57N+G91T+K98E+R118F+E210K+D254S, D57N+G91T+K98E+R118F+T244E+D254S, D57N+G91T+K98E+E210K+T244E+D254S, D57N+G91T+R108K+R118F+E210K+T244E, D57N+G91T+R108K+R118F+E210K+D254S, D57N+G91T+R108K+R118F+T244E+D254S, D57N+G91T+R108K+E210K+T244E+D254S, D57N+G91T+R118F+E210K+T244E+D254S, D57N+K98E+R108K+R118F+E210K+T244E, D57N+K98E+R108K+R118F+E210K+D254S, D57N+K98E+R108K+R118F+T244E+D254S, D57N+K98E+R108K+E210K+T244E+D254S, D57N+K98E+R118F+E210K+T244E+D254S, D57N+R108K+R118F+E210K+T244E+D254S, G91T+K98E+R108K+R118F+E210K+T244E, G91T+K98E+R108K+R118F+E210K+D254S, G91T+K98E+R108K+R118F+T244E+D254S, G91T+K98E+R108K+E210K+T244E+D254S, G91T+K98E+R118F+E210K+T244E+D254S, G91T+R108K+R118F+E210K+T244E+D254S,
K98E+R108K+R118F+E210K+T244E+D254S,
A40E+E56R+D57N+G91T+K98E+R108K+R118F,
A40E+E56R+D57N+G91T+K98E+R108K+E210K,
A40E+E56R+D57N+G91T+K98E+R108K+T244E,
A40E+E56R+D57N+G91T+K98E+R108K+D254S,
A40E+E56R+D57N+G91T+K98E+R118F+E210K,
A40E+E56R+D57N+G91T+K98E+R118F+T244E,
A40E+E56R+D57N+G91T+K98E+R118F+D254S,
A40E+E56R+D57N+G91T+K98E+E210K+T244E,
A40E+E56R+D57N+G91T+K98E+E210K+D254S,
A40E+E56R+D57N+G91T+K98E+T244E+D254S,
A40E+E56R+D57N+G91T+R108K+R118F+E210K,
A40E+E56R+D57N+G91T+R108K+R118F+T244E,
A40E+E56R+D57N+G91T+R108K+R118F+D254S,
A40E+E56R+D57N+G91T+R108K+E210K+T244E,
A40E+E56R+D57N+G91T+R108K+E210K+D254S,
A40E+E56R+D57N+G91T+R108K+T244E+D254S,
A40E+E56R+D57N+G91T+R118F+E210K+T244E,
A40E+E56R+D57N+G91T+R118F+E210K+D254S,
A40E+E56R+D57N+G91T+R118F+T244E+D254S,
A40E+E56R+D57N+G91T+E210K+T244E+D254S,
A40E+E56R+D57N+K98E+R108K+R118F+E210K,
A40E+E56R+D57N+K98E+R108K+R118F+T244E,
A40E+E56R+D57N+K98E+R108K+R118F+D254S,
A40E+E56R+D57N+K98E+R108K+E210K+T244E,
A40E+E56R+D57N+K98E+R108K+E210K+D254S,
A40E+E56R+D57N+K98E+R108K+T244E+D254S,
A40E+E56R+D57N+K98E+R118F+E210K+T244E,
A40E+E56R+D57N+K98E+R118F+E210K+D254S,
A40E+E56R+D57N+K98E+R118F+T244E+D254S,
A40E+E56R+D57N+K98E+E210K+T244E+D254S,
A40E+E56R+D57N+R108K+R118F+E210K+T244E,
A40E+E56R+D57N+R108K+R118F+E210K+D254S,
A40E+E56R+D57N+R108K+R118F+T244E+D254S,
A40E+E56R+D57N+R108K+E210K+T244E+D254S,
A40E+E56R+D57N+R118F+E210K+T244E+D254S,
A40E+E56R+G91T+K98E+R108K+R118F+E210K,
A40E+E56R+G91T+K98E+R108K+R118F+T244E,
A40E+E56R+G91T+K98E+R108K+R118F+D254S,
A40E+E56R+G91T+K98E+R108K+E210K+T244E,
A40E+E56R+G91T+K98E+R108K+E210K+D254S,
A40E+E56R+G91T+K98E+R108K+T244E+D254S,
A40E+E56R+G91T+K98E+R118F+E210K+T244E,
A40E+E56R+G91T+K98E+R118F+E210K+D254S,
A40E+E56R+G91T+K98E+R118F+T244E+D254S,
A40E+E56R+G91T+K98E+E210K+T244E+D254S,
A40E+E56R+G91T+R108K+R118F+E210K+T244E,
A40E+E56R+G91T+R108K+R118F+E210K+D254S,
A40E+E56R+G91T+R108K+R118F+T244E+D254S,
A40E+E56R+G91T+R108K+E210K+T244E+D254S,
A40E+E56R+G91T+R118F+E210K+T244E+D254S,
A40E+E56R+K98E+R108K+R118F+E210K+T244E,
A40E+E56R+K98E+R108K+R118F+E210K+D254S,
A40E+E56R+K98E+R108K+R118F+T244E+D254S,
A40E+E56R+K98E+R108K+E210K+T244E+D254S,
A40E+E56R+K98E+R118F+E210K+T244E+D254S,
A40E+E56R+R108K+R118F+E210K+T244E+D254S,
A40E+D57N+G91T+K98E+R108K+R118F+E210K,
A40E+D57N+G91T+K98E+R108K+R118F+T244E,
A40E+D57N+G91T+K98E+R108K+R118F+D254S,
A40E+D57N+G91T+K98E+R108K+E210K+T244E,
A40E+D57N+G91T+K98E+R108K+E210K+D254S,
A40E+D57N+G91T+K98E+R108K+T244E+D254S,
A40E+D57N+G91T+K98E+R118F+E210K+T244E,
A40E+D57N+G91T+K98E+R118F+E210K+D254S,
A40E+D57N+G91T+K98E+R118F+T244E+D254S,
A40E+D57N+G91T+K98E+E210K+T244E+D254S,
A40E+D57N+G91T+R108K+R118F+E210K+T244E,
A40E+D57N+G91T+R108K+R118F+E210K+D254S,
A40E+D57N+G91T+R108K+R118F+T244E+D254S,
A40E+D57N+G91T+R108K+E210K+T244E+D254S,
A40E+D57N+G91T+R118F+E210K+T244E+D254S,
A40E+D57N+K98E+R108K+R118F+E210K+T244E,
A40E+D57N+K98E+R108K+R118F+E210K+D254S,
A40E+D57N+K98E+R108K+R118F+T244E+D254S,
A40E+D57N+K98E+R108K+E210K+T244E+D254S,
A40E+D57N+K98E+R118F+E210K+T244E+D254S,
A40E+D57N+R108K+R118F+E210K+T244E+D254S,
A40E+G91T+K98E+R108K+R118F+E210K+T244E,
A40E+G91T+K98E+R108K+R118F+E210K+D254S,
A40E+G91T+K98E+R108K+R118F+T244E+D254S,
A40E+G91T+K98E+R108K+E210K+T244E+D254S,
A40E+G91T+K98E+R118F+E210K+T244E+D254S,
A40E+G91T+R108K+R118F+E210K+T244E+D254S,
A40E+K98E+R108K+R118F+E210K+T244E+D254S,
E56R+D57N+G91T+K98E+R108K+R118F+E210K,
E56R+D57N+G91T+K98E+R108K+R118F+T244E,
E56R+D57N+G91T+K98E+R108K+R118F+D254S,
E56R+D57N+G91T+K98E+R108K+E210K+T244E,
E56R+D57N+G91T+K98E+R108K+E210K+D254S,
E56R+D57N+G91T+K98E+R108K+T244E+D254S,
E56R+D57N+G91T+K98E+R118F+E210K+T244E,
E56R+D57N+G91T+K98E+R118F+E210K+D254S,
E56R+D57N+G91T+K98E+R118F+T244E+D254S,
E56R+D57N+G91T+K98E+E210K+T244E+D254S,
E56R+D57N+G91T+R108K+R118F+E210K+T244E,
E56R+D57N+G91T+R108K+R118F+E210K+D254S,
E56R+D57N+G91T+R108K+R118F+T244E+D254S,
E56R+D57N+G91T+R108K+E210K+T244E+D254S,
E56R+D57N+G91T+R118F+E210K+T244E+D254S,
E56R+D57N+K98E+R108K+R118F+E210K+T244E,
E56R+D57N+K98E+R108K+R118F+E210K+D254S,
E56R+D57N+K98E+R108K+R118F+T244E+D254S,
E56R+D57N+K98E+R108K+E210K+T244E+D254S,
E56R+D57N+K98E+R118F+E210K+T244E+D254S,
E56R+D57N+R108K+R118F+E210K+T244E+D254S,
E56R+G91T+K98E+R108K+R118F+E210K+T244E,
E56R+G91T+K98E+R108K+R118F+E210K+D254S,
E56R+G91T+K98E+R108K+R118F+T244E+D254S,
E56R+G91T+K98E+R108K+E210K+T244E+D254S,
E56R+G91T+K98E+R118F+E210K+T244E+D254S,
E56R+G91T+R108K+R118F+E210K+T244E+D254S,
E56R+K98E+R108K+R118F+E210K+T244E+D254S,
D57N+G91T+K98E+R108K+R118F+E210K+T244E,
D57N+G91T+K98E+R108K+R118F+E210K+D254S,
D57N+G91T+K98E+R108K+R118F+T244E+D254S,
D57N+G91T+K98E+R108K+E210K+T244E+D254S,
D57N+G91T+K98E+R118F+E210K+T244E+D254S,
D57N+G91T+R108K+R118F+E210K+T244E+D254S,
D57N+K98E+R108K+R118F+E210K+T244E+D254S,
G91T+K98E+R108K+R118F+E210K+T244E+D254S,
A40E+E56R+D57N+G91T+K98E+R108K+R118F+E210K,
A40E+E56R+D57N+G91T+K98E+R108K+R118F+T244E,
A40E+E56R+D57N+G91T+K98E+R108K+R118F+D254S,
A40E+E56R+D57N+G91T+K98E+R108K+E210K+T244E,
A40E+E56R+D57N+G91T+K98E+R108K+E210K+D254S,
A40E+E56R+D57N+G91T+K98E+R108K+T244E+D254S,
A40E+E56R+D57N+G91T+K98E+R118F+E210K+T244E,
A40E+E56R+D57N+G91T+K98E+R118F+E210K+D254S,
A40E+E56R+D57N+G91T+K98E+R118F+T244E+D254S,
A40E+E56R+D57N+G91T+K98E+E210K+T244E+D254S,
A40E+E56R+D57N+G91T+R108K+R118F+E210K+T244E,
A40E+E56R+D57N+G91T+R108K+R118F+E210K+D254S,
A40E+E56R+D57N+G91T+R108K+R118F+T244E+D254S,
A40E+E56R+D57N+G91T+R108K+E210K+T244E+D254S,
A40E+E56R+D57N+G91T+R118F+E210K+T244E+D254S,
A40E+E56R+D57N+K98E+R108K+R118F+E210K+T244E,
A40E+E56R+D57N+K98E+R108K+R118F+E210K+D254S,
A40E+E56R+D57N+K98E+R108K+R118F+T244E+D254S,
A40E+E56R+D57N+K98E+R108K+E210K+T244E+D254S,
A40E+E56R+D57N+K98E+R118F+E210K+T244E+D254S,
A40E+E56R+D57N+R108K+R118F+E210K+T244E+D254S,
A40E+E56R+G91T+K98E+R108K+R118F+E210K+T244E,
A40E+E56R+G91T+K98E+R108K+R118F+E210K+D254S,
A40E+E56R+G91T+K98E+R108K+R118F+T244E+D254S,
A40E+E56R+G91T+K98E+R108K+E210K+T244E+D254S,
A40E+E56R+G91T+K98E+R118F+E210K+T244E+D254S,
A40E+E56R+G91T+R108K+R118F+E210K+T244E+D254S,
A40E+E56R+K98E+R108K+R118F+E210K+T244E+D254S,
A40E+D57N+G91T+K98E+R108K+R118F+E210K+T244E,
A40E+D57N+G91T+K98E+R108K+R118F+E210K+D254S,
A40E+D57N+G91T+K98E+R108K+R118F+T244E+D254S,
A40E+D57N+G91T+K98E+R108K+E210K+T244E+D254S,
A40E+D57N+G91T+K98E+R118F+E210K+T244E+D254S,
A40E+D57N+G91T+R108K+R118F+E210K+T244E+D254S,
A40E+D57N+K98E+R108K+R118F+E210K+T244E+D254S,
A40E+G91T+K98E+R108K+R118F+E210K+T244E+D254S,
E56R+D57N+G91T+K98E+R108K+R118F+E210K+T244E,
E56R+D57N+G91T+K98E+R108K+R118F+E210K+D254S,
E56R+D57N+G91T+K98E+R108K+R118F+T244E+D254S,
E56R+D57N+G91T+K98E+R108K+E210K+T244E+D254S,
E56R+D57N+G91T+K98E+R118F+E210K+T244E+D254S,
E56R+D57N+G91T+R108K+R118F+E210K+T244E+D254S,
E56R+D57N+K98E+R108K+R118F+E210K+T244E+D254S,
E56R+G91T+K98E+R108K+R118F+E210K+T244E+D254S,
D57N+G91T+K98E+R108K+R118F+E210K+T244E+D254S,
A40E+E56R+D57N+G91T+K98E+R108K+R118F+E210K+T244E,
A40E+E56R+D57N+G91T+K98E+R108K+R118F+E210K+D254S,
A40E+E56R+D57N+G91T+K98E+R108K+R118F+T244E+D254S,
A40E+E56R+D57N+G91T+K98E+R108K+E210K+T244E+D254S,
A40E+E56R+D57N+G91T+K98E+R118F+E210K+T244E+D254S,
A40E+E56R+D57N+G91T+R108K+R118F+E210K+T244E+D254S,
A40E+E56R+D57N+K98E+R108K+R118F+E210K+T244E+D254S,
A40E+E56R+G91T+K98E+R108K+R118F+E210K+T244E+D254S,
A40E+D57N+G91T+K98E+R108K+R118F+E210K+T244E+D254S,
E56R+D57N+G91T+K98E+R108K+R118F+E210K+T244E+D254S,
A40E+E56R+D57N+G91T+K98E+R108K+R118F+E210K+T244E+D254S.
   8. The variant of any one of paragraph 1-5, wherein the variant has a substitution corresponding to L269H of SEQ ID NO: 8 and further comprises one of the following substitutions or set of substitutions corresponding to:
A40E, E56R, D57N, G91T, K98E, R108K, R118F, E210K, T244E, A40E+E56R, A40E+D57N, A40E+G91T, A40E+K98E, A40E+R108K, A40E+R118F, A40E+E210K, A40E+T244E, A40E+D254S, E56R+D57N, E56R+G91T, E56R+K98E, E56R+R108K, E56R+R118F, E56R+E210K, E56R+T244E, E56R+D254S, D57N+G91T, D57N+K98E, D57N+R108K, D57N+R118F, D57N+E210K, D57N+T244E, D57N+D254S, G91T+K98E, G91T+R108K, G91T+R118F, G91T+E210K, G91T+T244E, G91T+D254S, K98E+R108K, K98E+R118F, K98E+E210K, K98E+T244E, K98E+D254S, R108K+R118F, R108K+E210K, R108K+T244E, R108K+D254S, R118F+E210K, R118F+T244E, R118F+D254S, E210K+T244E, E210K+D254S, T244E+D254S, A40E+E56R+D57N, A40E+E56R+G91T, A40E+E56R+K98E, A40E+E56R+R108K, A40E+E56R+R118F, A40E+E56R+E210K, A40E+E56R+T244E, A40E+E56R+D254S, A40E+D57N+G91T, A40E+D57N+K98E, A40E+D57N+R108K, A40E+D57N+R118F, A40E+D57N+E210K, A40E+D57N+T244E, A40E+D57N+D254S, A40E+G91T+K98E, A40E+G91T+R108K, A40E+G91T+R118F, A40E+G91T+E210K, A40E+G91T+T244E, A40E+G91T+D254S, A40E+K98E+R108K, A40E+K98E+R118F, A40E+K98E+E210K, A40E+K98E+T244E, A40E+K98E+D254S, A40E+R108K+R118F, A40E+R108K+E210K, A40E+R108K+T244E, A40E+R108K+D254S, A40E+R118F+E210K, A40E+R118F+T244E, A40E+R118F+D254S, A40E+E210K+T244E, A40E+E210K+D254S, A40E+T244E+D254S, E56R+D57N+G91T, E56R+D57N+K98E, E56R+D57N+R108K, E56R+D57N+R118F, E56R+D57N+E210K, E56R+D57N+T244E, E56R+D57N+D254S, E56R+G91T+K98E, E56R+G91T+R108K, E56R+G91T+R118F, E56R+G91T+E210K, E56R+G91T+T244E, E56R+G91T+D254S, E56R+K98E+R108K, E56R+K98E+R118F, E56R+K98E+E210K, E56R+K98E+T244E, E56R+K98E+D254S, E56R+R108K+R118F, E56R+R108K+E210K, E56R+R108K+T244E, E56R+R108K+D254S, E56R+R118F+E210K, E56R+R118F+T244E, E56R+R118F+D254S, E56R+E210K+T244E, E56R+E210K+D254S, E56R+T244E+D254S, D57N+G91T+K98E, D57N+G91T+R108K, D57N+G91T+R118F, D57N+G91T+E210K, D57N+G91T+T244E, D57N+G91T+D254S, D57N+K98E+R108K, D57N+K98E+R118F, D57N+K98E+E210K, D57N+K98E+T244E, D57N+K98E+D254S, D57N+R108K+R118F, D57N+R108K+E210K, D57N+R108K+T244E, D57N+R108K+D254S, D57N+R118F+E210K, D57N+R118F+T244E, D57N+R118F+D254S, D57N+E210K+T244E, D57N+E210K+D254S, D57N+T244E+D254S, G91T+K98E+R108K, G91T+K98E+R118F, G91T+K98E+E210K, G91T+K98E+T244E, G91T+K98E+D254S, G91T+R108K+R118F, G91T+R108K+E210K, G91T+R108K+T244E, G91T+R108K+D254S, G91T+R118F+E210K, G91T+R118F+T244E, G91T+R118F+D254S, G91T+E210K+T244E, G91T+E210K+D254S, G91T+T244E+D254S, K98E+R108K+R118F, K98E+R108K+E210K, K98E+R108K+T244E, K98E+R108K+D254S, K98E+R118F+E210K, K98E+R118F+T244E, K98E+R118F+D254S, K98E+E210K+T244E, K98E+E210K+D254S, K98E+T244E+D254S, R108K+R118F+E210K, R108K+R118F+T244E, R108K+R118F+D254S, R108K+E210K+T244E, R108K+E210K+D254S, R108K+T244E+D254S, R118F+E210K+T244E, R118F+E210K+D254S, R118F+T244E+D254S, E210K+T244E+D254S, A40E+E56R+D57N+G91T, A40E+E56R+D57N+K98E, A40E+E56R+D57N+R108K, A40E+E56R+D57N+R118F, A40E+E56R+D57N+E210K, A40E+E56R+D57N+T244E, A40E+E56R+D57N+D254S, A40E+E56R+G91T+K98E, A40E+E56R+G91T+R108K, A40E+E56R+G91T+R118F, A40E+E56R+G91T+E210K, A40E+E56R+G91T+T244E, A40E+E56R+G91T+D254S, A40E+E56R+K98E+R108K, A40E+E56R+K98E+R118F, A40E+E56R+K98E+E210K, A40E+E56R+K98E+T244E, A40E+E56R+K98E+D254S, A40E+E56R+R108K+R118F, A40E+E56R+R108K+E210K, A40E+E56R+R108K+T244E, A40E+E56R+R108K+D254S, A40E+E56R+R118F+E210K, A40E+E56R+R118F+T244E, A40E+E56R+R118F+D254S, A40E+E56R+E210K+T244E, A40E+E56R+E210K+D254S, A40E+E56R+T244E+D254S, A40E+D57N+G91T+K98E, A40E+D57N+G91T+R108K, A40E+D57N+G91T+R118F, A40E+D57N+G91T+E210K, A40E+D57N+G91T+T244E, A40E+D57N+G91T+D254S, A40E+D57N+K98E+R108K, A40E+D57N+K98E+R118F, A40E+D57N+K98E+E210K, A40E+D57N+K98E+T244E, A40E+D57N+K98E+D254S, A40E+D57N+R108K+R118F, A40E+D57N+R108K+E210K, A40E+D57N+R108K+T244E, A40E+D57N+R108K+D254S, A40E+D57N+R118F+E210K, A40E+D57N+R118F+T244E, A40E+D57N+R118F+D254S, A40E+D57N+E210K+T244E, A40E+D57N+E210K+D254S, A40E+D57N+T244E+D254S, A40E+G91T+K98E+R108K, A40E+G91T+K98E+R118F, A40E+G91T+K98E+E210K, A40E+G91T+K98E+T244E, A40E+G91T+K98E+D254S, A40E+G91T+R108K+R118F, A40E+G91T+R108K+E210K, A40E+G91T+R108K+T244E, A40E+G91T+R108K+D254S, A40E+G91T+R118F+E210K, A40E+G91T+R118F+T244E, A40E+G91T+R118F+D254S, A40E+G91T+E210K+T244E, A40E+G91T+E210K+D254S, A40E+G91T+T244E+D254S, A40E+K98E+R108K+R118F, A40E+K98E+R108K+E210K, A40E+K98E+R108K+T244E, A40E+K98E+R108K+D254S, A40E+K98E+R118F+E210K, A40E+K98E+R118F+T244E, A40E+K98E+R118F+D254S, A40E+K98E+E210K+T244E, A40E+K98E+E210K+D254S, A40E+K98E+T244E+D254S, A40E+R108K+R118F+E210K, A40E+R108K+R118F+T244E, A40E+R108K+R118F+D254S, A40E+R108K+E210K+T244E, A40E+R108K+E210K+D254S, A40E+R108K+T244E+D254S, A40E+R118F+E210K+T244E, A40E+R118F+E210K+D254S, A40E+R118F+T244E+D254S, A40E+E210K+T244E+D254S, E56R+D57N+G91T+K98E, E56R+D57N+G91T+R108K, E56R+D57N+G91T+R118F, E56R+D57N+G91T+E210K, E56R+D57N+G91T+T244E, E56R+D57N+G91T+D254S, E56R+D57N+K98E+R108K, E56R+D57N+K98E+R118F, E56R+D57N+K98E+E210K, E56R+D57N+K98E+T244E, E56R+D57N+K98E+D254S, E56R+D57N+R108K+R118F, E56R+D57N+R108K+E210K, E56R+D57N+R108K+T244E, E56R+D57N+R108K+D254S, E56R+D57N+R118F+E210K, E56R+D57N+R118F+T244E, E56R+D57N+R118F+D254S, E56R+D57N+E210K+T244E, E56R+D57N+E210K+D254S, E56R+D57N+T244E+D254S, E56R+G91T+K98E+R108K, E56R+G91T+K98E+R118F, E56R+G91T+K98E+E210K, E56R+G91T+K98E+T244E, E56R+G91T+K98E+D254S, E56R+G91T+R108K+R118F, E56R+G91T+R108K+E210K, E56R+G91T+R108K+T244E, E56R+G91T+R108K+D254S, E56R+G91T+R118F+E210K, E56R+G91T+R118F+T244E, E56R+G91T+R118F+D254S, E56R+G91T+E210K+T244E, E56R+G91T+E210K+D254S, E56R+G91T+T244E+D254S, E56R+K98E+R108K+R118F, E56R+K98E+R108K+E210K, E56R+K98E+R108K+T244E, E56R+K98E+R108K+D254S, E56R+K98E+R118F+E210K, E56R+K98E+R118F+T244E, E56R+K98E+R118F+D254S, E56R+K98E+E210K+T244E, E56R+K98E+E210K+D254S, E56R+K98E+T244E+D254S, E56R+R108K+R118F+E210K, E56R+R108K+R118F+T244E, E56R+R108K+R118F+D254S, E56R+R108K+E210K+T244E, E56R+R108K+E210K+D254S, E56R+R108K+T244E+D254S, E56R+R118F+E210K+T244E, E56R+R118F+E210K+D254S, E56R+R118F+T244E+D254S, E56R+E210K+T244E+D254S, D57N+G91T+K98E+R108K, D57N+G91T+K98E+R118F, D57N+G91T+K98E+E210K, D57N+G91T+K98E+T244E, D57N+G91T+K98E+D254S, D57N+G91T+R108K+R118F, D57N+G91T+R108K+E210K, D57N+G91T+R108K+T244E, D57N+G91T+R108K+D254S, D57N+G91T+R118F+E210K, D57N+G91T+R118F+T244E, D57N+G91T+R118F+D254S, D57N+G91T+E210K+T244E, D57N+G91T+E210K+D254S, D57N+G91T+T244E+D254S, D57N+K98E+R108K+R118F, D57N+K98E+R108K+E210K, D57N+K98E+R108K+T244E, D57N+K98E+R108K+D254S, D57N+K98E+R118F+E210K, D57N+K98E+R118F+T244E, D57N+K98E+R118F+D254S, D57N+K98E+E210K+T244E, D57N+K98E+E210K+D254S, D57N+K98E+T244E+D254S, D57N+R108K+R118F+E210K, D57N+R108K+R118F+T244E, D57N+R108K+R118F+D254S, D57N+R108K+E210K+T244E, D57N+R108K+E210K+D254S, D57N+R108K+T244E+D254S, D57N+R118F+E210K+T244E, D57N+R118F+E210K+D254S, D57N+R118F+T244E+D254S, D57N+E210K+T244E+D254S, G91T+K98E+R108K+R118F, G91T+K98E+R108K+E210K, G91T+K98E+R108K+T244E, G91T+K98E+R108K+D254S, G91T+K98E+R118F+E210K, G91T+K98E+R118F+T244E, G91T+K98E+R118F+D254S, G91T+K98E+E210K+T244E, G91T+K98E+E210K+D254S, G91T+K98E+T244E+D254S, G91T+R108K+R118F+E210K, G91T+R108K+R118F+T244E, G91T+R108K+R118F+D254S, G91T+R108K+E210K+T244E, G91T+R108K+E210K+D254S, G91T+R108K+T244E+D254S, G91T+R118F+E210K+T244E, G91T+R118F+E210K+D254S, G91T+R118F+T244E+D254S, G91T+E210K+T244E+D254S, K98E+R108K+R118F+E210K, K98E+R108K+R118F+T244E, K98E+R108K+R118F+D254S, K98E+R108K+E210K+T244E, K98E+R108K+E210K+D254S, K98E+R108K+T244E+D254S, K98E+R118F+E210K+T244E, K98E+R118F+E210K+D254S, K98E+R118F+T244E+D254S, K98E+E210K+T244E+D254S, R108K+R118F+E210K+T244E, R108K+R118F+E210K+D254S, R108K+R118F+T244E+D254S, R108K+E210K+T244E+D254S, R118F+E210K+T244E+D254S, A40E+E56R+D57N+G91T+K98E, A40E+E56R+D57N+G91T+R108K, A40E+E56R+D57N+G91T+R118F, A40E+E56R+D57N+G91T+E210K, A40E+E56R+D57N+G91T+T244E, A40E+E56R+D57N+G91T+D254S, A40E+E56R+D57N+K98E+R108K, A40E+E56R+D57N+K98E+R118F, A40E+E56R+D57N+K98E+E210K, A40E+E56R+D57N+K98E+T244E, A40E+E56R+D57N+K98E+D254S, A40E+E56R+D57N+R108K+R118F, A40E+E56R+D57N+R108K+E210K, A40E+E56R+D57N+R108K+T244E, A40E+E56R+D57N+R108K+D254S, A40E+E56R+D57N+R118F+E210K, A40E+E56R+D57N+R118F+T244E, A40E+E56R+D57N+R118F+D254S, A40E+E56R+D57N+E210K+T244E, A40E+E56R+D57N+E210K+D254S, A40E+E56R+D57N+T244E+D254S, A40E+E56R+G91T+K98E+R108K, A40E+E56R+G91T+K98E+R118F, A40E+E56R+G91T+K98E+E210K, A40E+E56R+G91T+K98E+T244E, A40E+E56R+G91T+K98E+D254S, A40E+E56R+G91T+R108K+R118F, A40E+E56R+G91T+R108K+E210K, A40E+E56R+G91T+R108K+T244E, A40E+E56R+G91T+R108K+D254S, A40E+E56R+G91T+R118F+E210K, A40E+E56R+G91T+R118F+T244E, A40E+E56R+G91T+R118F+D254S, A40E+E56R+G91T+E210K+T244E, A40E+E56R+G91T+E210K+D254S, A40E+E56R+G91T+T244E+D254S, A40E+E56R+K98E+R108K+R118F, A40E+E56R+K98E+R108K+E210K, A40E+E56R+K98E+R108K+T244E, A40E+E56R+K98E+R108K+D254S, A40E+E56R+K98E+R118F+E210K, A40E+E56R+K98E+R118F+T244E, A40E+E56R+K98E+R118F+D254S, A40E+E56R+K98E+E210K+T244E, A40E+E56R+K98E+E210K+D254S, A40E+E56R+K98E+T244E+D254S, A40E+E56R+R108K+R118F+E210K, A40E+E56R+R108K+R118F+T244E, A40E+E56R+R108K+R118F+D254S, A40E+E56R+R108K+E210K+T244E, A40E+E56R+R108K+E210K+D254S, A40E+E56R+R108K+T244E+D254S, A40E+E56R+R118F+E210K+T244E, A40E+E56R+R118F+E210K+D254S, A40E+E56R+R118F+T244E+D254S, A40E+E56R+E210K+T244E+D254S, A40E+D57N+G91T+K98E+R108K, A40E+D57N+G91T+K98E+R118F, A40E+D57N+G91T+K98E+E210K, A40E+D57N+G91T+K98E+T244E, A40E+D57N+G91T+K98E+D254S, A40E+D57N+G91T+R108K+R118F, A40E+D57N+G91T+R108K+E210K, A40E+D57N+G91T+R108K+T244E, A40E+D57N+G91T+R108K+D254S, A40E+D57N+G91T+R118F+E210K, A40E+D57N+G91T+R118F+T244E, A40E+D57N+G91T+R118F+D254S, A40E+D57N+G91T+E210K+T244E, A40E+D57N+G91T+E210K+D254S, A40E+D57N+G91T+T244E+D254S, A40E+D57N+K98E+R108K+R118F, A40E+D57N+K98E+R108K+E210K, A40E+D57N+K98E+R108K+T244E, A40E+D57N+K98E+R108K+D254S, A40E+D57N+K98E+R118F+E210K, A40E+D57N+K98E+R118F+T244E, A40E+D57N+K98E+R118F+D254S, A40E+D57N+K98E+E210K+T244E, A40E+D57N+K98E+E210K+D254S, A40E+D57N+K98E+T244E+D254S, A40E+D57N+R108K+R118F+E210K, A40E+D57N+R108K+R118F+T244E, A40E+D57N+R108K+R118F+D254S, A40E+D57N+R108K+E210K+T244E, A40E+D57N+R108K+E210K+D254S, A40E+D57N+R108K+T244E+D254S, A40E+D57N+R118F+E210K+T244E, A40E+D57N+R118F+E210K+D254S, A40E+D57N+R118F+T244E+D254S, A40E+D57N+E210K+T244E+D254S, A40E+G91T+K98E+R108K+R118F, A40E+G91T+K98E+R108K+E210K, A40E+G91T+K98E+R108K+T244E, A40E+G91T+K98E+R108K+D254S, A40E+G91T+K98E+R118F+E210K, A40E+G91T+K98E+R118F+T244E, A40E+G91T+K98E+R118F+D254S, A40E+G91T+K98E+E210K+T244E, A40E+G91T+K98E+E210K+D254S, A40E+G91T+K98E+T244E+D254S, A40E+G91T+R108K+R118F+E210K, A40E+G91T+R108K+R118F+T244E, A40E+G91T+R108K+R118F+D254S, A40E+G91T+R108K+E210K+T244E, A40E+G91T+R108K+E210K+D254S, A40E+G91T+R108K+T244E+D254S, A40E+G91T+R118F+E210K+T244E, A40E+G91T+R118F+E210K+D254S, A40E+G91T+R118F+T244E+D254S, A40E+G91T+E210K+T244E+D254S, A40E+K98E+R108K+R118F+E210K, A40E+K98E+R108K+R118F+T244E, A40E+K98E+R108K+R118F+D254S, A40E+K98E+R108K+E210K+T244E, A40E+K98E+R108K+E210K+D254S, A40E+K98E+R108K+T244E+D254S, A40E+K98E+R118F+E210K+T244E, A40E+K98E+R118F+E210K+D254S, A40E+K98E+R118F+T244E+D254S, A40E+K98E+E210K+T244E+D254S, A40E+R108K+R118F+E210K+T244E, A40E+R108K+R118F+E210K+D254S, A40E+R108K+R118F+T244E+D254S, A40E+R108K+E210K+T244E+D254S, A40E+R118F+E210K+T244E+D254S, E56R+D57N+G91T+K98E+R108K, E56R+D57N+G91T+K98E+R118F, E56R+D57N+G91T+K98E+E210K, E56R+D57N+G91T+K98E+T244E, E56R+D57N+G91T+K98E+D254S, E56R+D57N+G91T+R108K+R118F, E56R+D57N+G91T+R108K+E210K, E56R+D57N+G91T+R108K+T244E, E56R+D57N+G91T+R108K+D254S, E56R+D57N+G91T+R118F+E210K, E56R+D57N+G91T+R118F+T244E, E56R+D57N+G91T+R118F+D254S, E56R+D57N+G91T+E210K+T244E, E56R+D57N+G91T+E210K+D254S, E56R+D57N+G91T+T244E+D254S, E56R+D57N+K98E+R108K+R118F, E56R+D57N+K98E+R108K+E210K, E56R+D57N+K98E+R108K+T244E, E56R+D57N+K98E+R108K+D254S, E56R+D57N+K98E+R118F+E210K, E56R+D57N+K98E+R118F+T244E, E56R+D57N+K98E+R118F+D254S, E56R+D57N+K98E+E210K+T244E, E56R+D57N+K98E+E210K+D254S, E56R+D57N+K98E+T244E+D254S, E56R+D57N+R108K+R118F+E210K, E56R+D57N+R108K+R118F+T244E, E56R+D57N+R108K+R118F+D254S, E56R+D57N+R108K+E210K+T244E, E56R+D57N+R108K+E210K+D254S, E56R+D57N+R108K+T244E+D254S, E56R+D57N+R118F+E210K+T244E, E56R+D57N+R118F+E210K+D254S, E56R+D57N+R118F+T244E+D254S, E56R+D57N+E210K+T244E+D254S, E56R+G91T+K98E+R108K+R118F, E56R+G91T+K98E+R108K+E210K, E56R+G91T+K98E+R108K+T244E, E56R+G91T+K98E+R108K+D254S, E56R+G91T+K98E+R118F+E210K, E56R+G91T+K98E+R118F+T244E, E56R+G91T+K98E+R118F+D254S, E56R+G91T+K98E+E210K+T244E, E56R+G91T+K98E+E210K+D254S, E56R+G91T+K98E+T244E+D254S, E56R+G91T+R108K+R118F+E210K, E56R+G91T+R108K+R118F+T244E, E56R+G91T+R108K+R118F+D254S, E56R+G91T+R108K+E210K+T244E, E56R+G91T+R108K+E210K+D254S, E56R+G91T+R108K+T244E+D254S, E56R+G91T+R118F+E210K+T244E, E56R+G91T+R118F+E210K+D254S, E56R+G91T+R118F+T244E+D254S, E56R+G91T+E210K+T244E+D254S, E56R+K98E+R108K+R118F+E210K, E56R+K98E+R108K+R118F+T244E, E56R+K98E+R108K+R118F+D254S, E56R+K98E+R108K+E210K+T244E, E56R+K98E+R108K+E210K+D254S, E56R+K98E+R108K+T244E+D254S, E56R+K98E+R118F+E210K+T244E, E56R+K98E+R118F+E210K+D254S, E56R+K98E+R118F+T244E+D254S, E56R+K98E+E210K+T244E+D254S, E56R+R108K+R118F+E210K+T244E, E56R+R108K+R118F+E210K+D254S, E56R+R108K+R118F+T244E+D254S, E56R+R108K+E210K+T244E+D254S, E56R+R118F+E210K+T244E+D254S, D57N+G91T+K98E+R108K+R118F, D57N+G91T+K98E+R108K+E210K, D57N+G91T+K98E+R108K+T244E, D57N+G91T+K98E+R108K+D254S, D57N+G91T+K98E+R118F+E210K, D57N+G91T+K98E+R118F+T244E, D57N+G91T+K98E+R118F+D254S, D57N+G91T+K98E+E210K+T244E, D57N+G91T+K98E+E210K+D254S, D57N+G91T+K98E+T244E+D254S, D57N+G91T+R108K+R118F+E210K, D57N+G91T+R108K+R118F+T244E, D57N+G91T+R108K+R118F+D254S, D57N+G91T+R108K+E210K+T244E, D57N+G91T+R108K+E210K+D254S, D57N+G91T+R108K+T244E+D254S, D57N+G91T+R118F+E210K+T244E, D57N+G91T+R118F+E210K+D254S, D57N+G91T+R118F+T244E+D254S, D57N+G91T+E210K+T244E+D254S, D57N+K98E+R108K+R118F+E210K, D57N+K98E+R108K+R118F+T244E, D57N+K98E+R108K+R118F+D254S, D57N+K98E+R108K+E210K+T244E, D57N+K98E+R108K+E210K+D254S, D57N+K98E+R108K+T244E+D254S, D57N+K98E+R118F+E210K+T244E, D57N+K98E+R118F+E210K+D254S, D57N+K98E+R118F+T244E+D254S, D57N+K98E+E210K+T244E+D254S, D57N+R108K+R118F+E210K+T244E, D57N+R108K+R118F+E210K+D254S, D57N+R108K+R118F+T244E+D254S, D57N+R108K+E210K+T244E+D254S, D57N+R118F+E210K+T244E+D254S, G91T+K98E+R108K+R118F+E210K, G91T+K98E+R108K+R118F+T244E, G91T+K98E+R108K+R118F+D254S, G91T+K98E+R108K+E210K+T244E, G91T+K98E+R108K+E210K+D254S, G91T+K98E+R108K+T244E+D254S, G91T+K98E+R118F+E210K+T244E, G91T+K98E+R118F+E210K+D254S, G91T+K98E+R118F+T244E+D254S, G91T+K98E+E210K+T244E+D254S, G91T+R108K+R118F+E210K+T244E, G91T+R108K+R118F+E210K+D254S, G91T+R108K+R118F+T244E+D254S, G91T+R108K+E210K+T244E+D254S, G91T+R118F+E210K+T244E+D254S, K98E+R108K+R118F+E210K+T244E, K98E+R108K+R118F+E210K+D254S, K98E+R108K+R118F+T244E+D254S, K98E+R108K+E210K+T244E+D254S, K98E+R118F+E210K+T244E+D254S, R108K+R118F+E210K+T244E+D254S, A40E+E56R+D57N+G91T+K98E+R108K, A40E+E56R+D57N+G91T+K98E+R118F, A40E+E56R+D57N+G91T+K98E+E210K, A40E+E56R+D57N+G91T+K98E+T244E, A40E+E56R+D57N+G91T+K98E+D254S, A40E+E56R+D57N+G91T+R108K+R118F, A40E+E56R+D57N+G91T+R108K+E210K, A40E+E56R+D57N+G91T+R108K+T244E, A40E+E56R+D57N+G91T+R108K+D254S, A40E+E56R+D57N+G91T+R118F+E210K, A40E+E56R+D57N+G91T+R118F+T244E, A40E+E56R+D57N+G91T+R118F+D254S, A40E+E56R+D57N+G91T+E210K+T244E, A40E+E56R+D57N+G91T+E210K+D254S, A40E+E56R+D57N+G91T+T244E+D254S, A40E+E56R+D57N+K98E+R108K+R118F, A40E+E56R+D57N+K98E+R108K+E210K, A40E+E56R+D57N+K98E+R108K+T244E, A40E+E56R+D57N+K98E+R108K+D254S, A40E+E56R+D57N+K98E+R118F+E210K, A40E+E56R+D57N+K98E+R118F+T244E, A40E+E56R+D57N+K98E+R118F+D254S, A40E+E56R+D57N+K98E+E210K+T244E, A40E+E56R+D57N+K98E+E210K+D254S, A40E+E56R+D57N+K98E+T244E+D254S, A40E+E56R+D57N+R108K+R118F+E210K, A40E+E56R+D57N+R108K+R118F+T244E, A40E+E56R+D57N+R108K+R118F+D254S, A40E+E56R+D57N+R108K+E210K+T244E, A40E+E56R+D57N+R108K+E210K+D254S, A40E+E56R+D57N+R108K+T244E+D254S, A40E+E56R+D57N+R118F+E210K+T244E, A40E+E56R+D57N+R118F+E210K+D254S, A40E+E56R+D57N+R118F+T244E+D254S, A40E+E56R+D57N+E210K+T244E+D254S, A40E+E56R+G91T+K98E+R108K+R118F, A40E+E56R+G91T+K98E+R108K+E210K, A40E+E56R+G91T+K98E+R108K+T244E, A40E+E56R+G91T+K98E+R108K+D254S, A40E+E56R+G91T+K98E+R118F+E210K, A40E+E56R+G91T+K98E+R118F+T244E, A40E+E56R+G91T+K98E+R118F+D254S, A40E+E56R+G91T+K98E+E210K+T244E, A40E+E56R+G91T+K98E+E210K+D254S, A40E+E56R+G91T+K98E+T244E+D254S, A40E+E56R+G91T+R108K+R118F+E210K, A40E+E56R+G91T+R108K+R118F+T244E, A40E+E56R+G91T+R108K+R118F+D254S, A40E+E56R+G91T+R108K+E210K+T244E, A40E+E56R+G91T+R108K+E210K+D254S, A40E+E56R+G91T+R108K+T244E+D254S, A40E+E56R+G91T+R118F+E210K+T244E, A40E+E56R+G91T+R118F+E210K+D254S, A40E+E56R+G91T+R118F+T244E+D254S, A40E+E56R+G91T+E210K+T244E+D254S, A40E+E56R+K98E+R108K+R118F+E210K, A40E+E56R+K98E+R108K+R118F+T244E, A40E+E56R+K98E+R108K+R118F+D254S, A40E+E56R+K98E+R108K+E210K+T244E, A40E+E56R+K98E+R108K+E210K+D254S, A40E+E56R+K98E+R108K+T244E+D254S, A40E+E56R+K98E+R118F+E210K+T244E, A40E+E56R+K98E+R118F+E210K+D254S, A40E+E56R+K98E+R118F+T244E+D254S, A40E+E56R+K98E+E210K+T244E+D254S, A40E+E56R+R108K+R118F+E210K+T244E, A40E+E56R+R108K+R118F+E210K+D254S, A40E+E56R+R108K+R118F+T244E+D254S, A40E+E56R+R108K+E210K+T244E+D254S, A40E+E56R+R118F+E210K+T244E+D254S, A40E+D57N+G91T+K98E+R108K+R118F, A40E+D57N+G91T+K98E+R108K+E210K, A40E+D57N+G91T+K98E+R108K+T244E, A40E+D57N+G91T+K98E+R108K+D254S, A40E+D57N+G91T+K98E+R118F+E210K, A40E+D57N+G91T+K98E+R118F+T244E, A40E+D57N+G91T+K98E+R118F+D254S, A40E+D57N+G91T+K98E+E210K+T244E, A40E+D57N+G91T+K98E+E210K+D254S, A40E+D57N+G91T+K98E+T244E+D254S, A40E+D57N+G91T+R108K+R118F+E210K, A40E+D57N+G91T+R108K+R118F+T244E, A40E+D57N+G91T+R108K+R118F+D254S, A40E+D57N+G91T+R108K+E210K+T244E, A40E+D57N+G91T+R108K+E210K+D254S, A40E+D57N+G91T+R108K+T244E+D254S, A40E+D57N+G91T+R118F+E210K+T244E, A40E+D57N+G91T+R118F+E210K+D254S, A40E+D57N+G91T+R118F+T244E+D254S, A40E+D57N+G91T+E210K+T244E+D254S, A40E+D57N+K98E+R108K+R118F+E210K, A40E+D57N+K98E+R108K+R118F+T244E, A40E+D57N+K98E+R108K+R118F+D254S, A40E+D57N+K98E+R108K+E210K+T244E, A40E+D57N+K98E+R108K+E210K+D254S, A40E+D57N+K98E+R108K+T244E+D254S, A40E+D57N+K98E+R118F+E210K+T244E, A40E+D57N+K98E+R118F+E210K+D254S, A40E+D57N+K98E+R118F+T244E+D254S, A40E+D57N+K98E+E210K+T244E+D254S, A40E+D57N+R108K+R118F+E210K+T244E, A40E+D57N+R108K+R118F+E210K+D254S, A40E+D57N+R108K+R118F+T244E+D254S, A40E+D57N+R108K+E210K+T244E+D254S, A40E+D57N+R118F+E210K+T244E+D254S, A40E+G91T+K98E+R108K+R118F+E210K, A40E+G91T+K98E+R108K+R118F+T244E, A40E+G91T+K98E+R108K+R118F+D254S, A40E+G91T+K98E+R108K+E210K+T244E, A40E+G91T+K98E+R108K+E210K+D254S, A40E+G91T+K98E+R108K+T244E+D254S, A40E+G91T+K98E+R118F+E210K+T244E, A40E+G91T+K98E+R118F+E210K+D254S, A40E+G91T+K98E+R118F+T244E+D254S, A40E+G91T+K98E+E210K+T244E+D254S, A40E+G91T+R108K+R118F+E210K+T244E, A40E+G91T+R108K+R118F+E210K+D254S, A40E+G91T+R108K+R118F+T244E+D254S, A40E+G91T+R108K+E210K+T244E+D254S, A40E+G91T+R118F+E210K+T244E+D254S, A40E+K98E+R108K+R118F+E210K+T244E, A40E+K98E+R108K+R118F+E210K+D254S, A40E+K98E+R108K+R118F+T244E+D254S, A40E+K98E+R108K+E210K+T244E+D254S, A40E+K98E+R118F+E210K+T244E+D254S, A40E+R108K+R118F+E210K+T244E+D254S, E56R+D57N+G91T+K98E+R108K+R118F, E56R+D57N+G91T+K98E+R108K+E210K, E56R+D57N+G91T+K98E+R108K+T244E, E56R+D57N+G91T+K98E+R108K+D254S, E56R+D57N+G91T+K98E+R118F+E210K, E56R+D57N+G91T+K98E+R118F+T244E, E56R+D57N+G91T+K98E+R118F+D254S, E56R+D57N+G91T+K98E+E210K+T244E, E56R+D57N+G91T+K98E+E210K+D254S, E56R+D57N+G91T+K98E+T244E+D254S, E56R+D57N+G91T+R108K+R118F+E210K, E56R+D57N+G91T+R108K+R118F+T244E, E56R+D57N+G91T+R108K+R118F+D254S, E56R+D57N+G91T+R108K+E210K+T244E, E56R+D57N+G91T+R108K+E210K+D254S, E56R+D57N+G91T+R108K+T244E+D254S, E56R+D57N+G91T+R118F+E210K+T244E, E56R+D57N+G91T+R118F+E210K+D254S, E56R+D57N+G91T+R118F+T244E+D254S, E56R+D57N+G91T+E210K+T244E+D254S, E56R+D57N+K98E+R108K+R118F+E210K, E56R+D57N+K98E+R108K+R118F+T244E, E56R+D57N+K98E+R108K+R118F+D254S, E56R+D57N+K98E+R108K+E210K+T244E, E56R+D57N+K98E+R108K+E210K+D254S, E56R+D57N+K98E+R108K+T244E+D254S, E56R+D57N+K98E+R118F+E210K+T244E, E56R+D57N+K98E+R118F+E210K+D254S, E56R+D57N+K98E+R118F+T244E+D254S, E56R+D57N+K98E+E210K+T244E+D254S, E56R+D57N+R108K+R118F+E210K+T244E, E56R+D57N+R108K+R118F+E210K+D254S, E56R+D57N+R108K+R118F+T244E+D254S, E56R+D57N+R108K+E210K+T244E+D254S, E56R+D57N+R118F+E210K+T244E+D254S, E56R+G91T+K98E+R108K+R118F+E210K, E56R+G91T+K98E+R108K+R118F+T244E, E56R+G91T+K98E+R108K+R118F+D254S, E56R+G91T+K98E+R108K+E210K+T244E, E56R+G91T+K98E+R108K+E210K+D254S, E56R+G91T+K98E+R108K+T244E+D254S, E56R+G91T+K98E+R118F+E210K+T244E, E56R+G91T+K98E+R118F+E210K+D254S, E56R+G91T+K98E+R118F+T244E+D254S, E56R+G91T+K98E+E210K+T244E+D254S, E56R+G91T+R108K+R118F+E210K+T244E, E56R+G91T+R108K+R118F+E210K+D254S, E56R+G91T+R108K+R118F+T244E+D254S, E56R+G91T+R108K+E210K+T244E+D254S, E56R+G91T+R118F+E210K+T244E+D254S, E56R+K98E+R108K+R118F+E210K+T244E, E56R+K98E+R108K+R118F+E210K+D254S, E56R+K98E+R108K+R118F+T244E+D254S, E56R+K98E+R108K+E210K+T244E+D254S, E56R+K98E+R118F+E210K+T244E+D254S, E56R+R108K+R118F+E210K+T244E+D254S, D57N+G91T+K98E+R108K+R118F+E210K, D57N+G91T+K98E+R108K+R118F+T244E, D57N+G91T+K98E+R108K+R118F+D254S, D57N+G91T+K98E+R108K+E210K+T244E, D57N+G91T+K98E+R108K+E210K+D254S, D57N+G91T+K98E+R108K+T244E+D254S, D57N+G91T+K98E+R118F+E210K+T244E, D57N+G91T+K98E+R118F+E210K+D254S, D57N+G91T+K98E+R118F+T244E+D254S, D57N+G91T+K98E+E210K+T244E+D254S, D57N+G91T+R108K+R118F+E210K+T244E, D57N+G91T+R108K+R118F+E210K+D254S, D57N+G91T+R108K+R118F+T244E+D254S, D57N+G91T+R108K+E210K+T244E+D254S, D57N+G91T+R118F+E210K+T244E+D254S, D57N+K98E+R108K+R118F+E210K+T244E, D57N+K98E+R108K+R118F+E210K+D254S, D57N+K98E+R108K+R118F+T244E+D254S, D57N+K98E+R108K+E210K+T244E+D254S, D57N+K98E+R118F+E210K+T244E+D254S, D57N+R108K+R118F+E210K+T244E+D254S, G91T+K98E+R108K+R118F+E210K+T244E, G91T+K98E+R108K+R118F+E210K+D254S, G91T+K98E+R108K+R118F+T244E+D254S, G91T+K98E+R108K+E210K+T244E+D254S, G91T+K98E+R118F+E210K+T244E+D254S, G91T+R108K+R118F+E210K+T244E+D254S,
K98E+R108K+R118F+E210K+T244E+D254S,
A40E+E56R+D57N+G91T+K98E+R108K+R118F,
A40E+E56R+D57N+G91T+K98E+R108K+E210K,
A40E+E56R+D57N+G91T+K98E+R108K+T244E,
A40E+E56R+D57N+G91T+K98E+R108K+D254S,
A40E+E56R+D57N+G91T+K98E+R118F+E210K,
A40E+E56R+D57N+G91T+K98E+R118F+T244E,
A40E+E56R+D57N+G91T+K98E+R118F+D254S,
A40E+E56R+D57N+G91T+K98E+E210K+T244E,
A40E+E56R+D57N+G91T+K98E+E210K+D254S,
A40E+E56R+D57N+G91T+K98E+T244E+D254S,
A40E+E56R+D57N+G91T+R108K+R118F+E210K,
A40E+E56R+D57N+G91T+R108K+R118F+T244E,
A40E+E56R+D57N+G91T+R108K+R118F+D254S,
A40E+E56R+D57N+G91T+R108K+E210K+T244E,
A40E+E56R+D57N+G91T+R108K+E210K+D254S,
A40E+E56R+D57N+G91T+R108K+T244E+D254S,
A40E+E56R+D57N+G91T+R118F+E210K+T244E,
A40E+E56R+D57N+G91T+R118F+E210K+D254S,
A40E+E56R+D57N+G91T+R118F+T244E+D254S,
A40E+E56R+D57N+G91T+E210K+T244E+D254S,
A40E+E56R+D57N+K98E+R108K+R118F+E210K,
A40E+E56R+D57N+K98E+R108K+R118F+T244E,
A40E+E56R+D57N+K98E+R108K+R118F+D254S,
A40E+E56R+D57N+K98E+R108K+E210K+T244E,
A40E+E56R+D57N+K98E+R108K+E210K+D254S,
A40E+E56R+D57N+K98E+R108K+T244E+D254S,
A40E+E56R+D57N+K98E+R118F+E210K+T244E,
A40E+E56R+D57N+K98E+R118F+E210K+D254S,
A40E+E56R+D57N+K98E+R118F+T244E+D254S,
A40E+E56R+D57N+K98E+E210K+T244E+D254S,
A40E+E56R+D57N+R108K+R118F+E210K+T244E,
A40E+E56R+D57N+R108K+R118F+E210K+D254S,
A40E+E56R+D57N+R108K+R118F+T244E+D254S,
A40E+E56R+D57N+R108K+E210K+T244E+D254S,
A40E+E56R+D57N+R118F+E210K+T244E+D254S,
A40E+E56R+G91T+K98E+R108K+R118F+E210K,
A40E+E56R+G91T+K98E+R108K+R118F+T244E,
A40E+E56R+G91T+K98E+R108K+R118F+D254S,
A40E+E56R+G91T+K98E+R108K+E210K+T244E,
A40E+E56R+G91T+K98E+R108K+E210K+D254S,
A40E+E56R+G91T+K98E+R108K+T244E+D254S,
A40E+E56R+G91T+K98E+R118F+E210K+T244E,
A40E+E56R+G91T+K98E+R118F+E210K+D254S,
A40E+E56R+G91T+K98E+R118F+T244E+D254S,
A40E+E56R+G91T+K98E+E210K+T244E+D254S,
A40E+E56R+G91T+R108K+R118F+E210K+T244E,
A40E+E56R+G91T+R108K+R118F+E210K+D254S,
A40E+E56R+G91T+R108K+R118F+T244E+D254S,
A40E+E56R+G91T+R108K+E210K+T244E+D254S,
A40E+E56R+G91T+R118F+E210K+T244E+D254S,
A40E+E56R+K98E+R108K+R118F+E210K+T244E,
A40E+E56R+K98E+R108K+R118F+E210K+D254S,
A40E+E56R+K98E+R108K+R118F+T244E+D254S,
A40E+E56R+K98E+R108K+E210K+T244E+D254S,
A40E+E56R+K98E+R118F+E210K+T244E+D254S,
A40E+E56R+R108K+R118F+E210K+T244E+D254S,
A40E+D57N+G91T+K98E+R108K+R118F+E210K,
A40E+D57N+G91T+K98E+R108K+R118F+T244E,
A40E+D57N+G91T+K98E+R108K+R118F+D254S,
A40E+D57N+G91T+K98E+R108K+E210K+T244E,
A40E+D57N+G91T+K98E+R108K+E210K+D254S,
A40E+D57N+G91T+K98E+R108K+T244E+D254S,
A40E+D57N+G91T+K98E+R118F+E210K+T244E,
A40E+D57N+G91T+K98E+R118F+E210K+D254S,
A40E+D57N+G91T+K98E+R118F+T244E+D254S,
A40E+D57N+G91T+K98E+E210K+T244E+D254S,
A40E+D57N+G91T+R108K+R118F+E210K+T244E,
A40E+D57N+G91T+R108K+R118F+E210K+D254S,
A40E+D57N+G91T+R108K+R118F+T244E+D254S,
A40E+D57N+G91T+R108K+E210K+T244E+D254S,
A40E+D57N+G91T+R118F+E210K+T244E+D254S,
A40E+D57N+K98E+R108K+R118F+E210K+T244E,
A40E+D57N+K98E+R108K+R118F+E210K+D254S,
A40E+D57N+K98E+R108K+R118F+T244E+D254S,
A40E+D57N+K98E+R108K+E210K+T244E+D254S,
A40E+D57N+K98E+R118F+E210K+T244E+D254S,
A40E+D57N+R108K+R118F+E210K+T244E+D254S,
A40E+G91T+K98E+R108K+R118F+E210K+T244E,
A40E+G91T+K98E+R108K+R118F+E210K+D254S,
A40E+G91T+K98E+R108K+R118F+T244E+D254S,
A40E+G91T+K98E+R108K+E210K+T244E+D254S,
A40E+G91T+K98E+R118F+E210K+T244E+D254S,
A40E+G91T+R108K+R118F+E210K+T244E+D254S,
A40E+K98E+R108K+R118F+E210K+T244E+D254S,
E56R+D57N+G91T+K98E+R108K+R118F+E210K,
E56R+D57N+G91T+K98E+R108K+R118F+T244E,
E56R+D57N+G91T+K98E+R108K+R118F+D254S,
E56R+D57N+G91T+K98E+R108K+E210K+T244E,
E56R+D57N+G91T+K98E+R108K+E210K+D254S,
E56R+D57N+G91T+K98E+R108K+T244E+D254S,
E56R+D57N+G91T+K98E+R118F+E210K+T244E,
E56R+D57N+G91T+K98E+R118F+E210K+D254S,
E56R+D57N+G91T+K98E+R118F+T244E+D254S,
E56R+D57N+G91T+K98E+E210K+T244E+D254S,
E56R+D57N+G91T+R108K+R118F+E210K+T244E,
E56R+D57N+G91T+R108K+R118F+E210K+D254S,
E56R+D57N+G91T+R108K+R118F+T244E+D254S,
E56R+D57N+G91T+R108K+E210K+T244E+D254S,
E56R+D57N+G91T+R118F+E210K+T244E+D254S,
E56R+D57N+K98E+R108K+R118F+E210K+T244E,
E56R+D57N+K98E+R108K+R118F+E210K+D254S,
E56R+D57N+K98E+R108K+R118F+T244E+D254S,
E56R+D57N+K98E+R108K+E210K+T244E+D254S,
E56R+D57N+K98E+R118F+E210K+T244E+D254S,
E56R+D57N+R108K+R118F+E210K+T244E+D254S,
E56R+G91T+K98E+R108K+R118F+E210K+T244E,
E56R+G91T+K98E+R108K+R118F+E210K+D254S,
E56R+G91T+K98E+R108K+R118F+T244E+D254S,
E56R+G91T+K98E+R108K+E210K+T244E+D254S,
E56R+G91T+K98E+R118F+E210K+T244E+D254S,
E56R+G91T+R108K+R118F+E210K+T244E+D254S,
E56R+K98E+R108K+R118F+E210K+T244E+D254S,
D57N+G91T+K98E+R108K+R118F+E210K+T244E,
D57N+G91T+K98E+R108K+R118F+E210K+D254S,
D57N+G91T+K98E+R108K+R118F+T244E+D254S,
D57N+G91T+K98E+R108K+E210K+T244E+D254S,
D57N+G91T+K98E+R118F+E210K+T244E+D254S,
D57N+G91T+R108K+R118F+E210K+T244E+D254S,
D57N+K98E+R108K+R118F+E210K+T244E+D254S,
G91T+K98E+R108K+R118F+E210K+T244E+D254S,
A40E+E56R+D57N+G91T+K98E+R108K+R118F+E210K,
A40E+E56R+D57N+G91T+K98E+R108K+R118F+T244E,
A40E+E56R+D57N+G91T+K98E+R108K+R118F+D254S,
A40E+E56R+D57N+G91T+K98E+R108K+E210K+T244E,
A40E+E56R+D57N+G91T+K98E+R108K+E210K+D254S,
A40E+E56R+D57N+G91T+K98E+R108K+T244E+D254S,
A40E+E56R+D57N+G91T+K98E+R118F+E210K+T244E,
A40E+E56R+D57N+G91T+K98E+R118F+E210K+D254S,
A40E+E56R+D57N+G91T+K98E+R118F+T244E+D254S,
A40E+E56R+D57N+G91T+K98E+E210K+T244E+D254S,
A40E+E56R+D57N+G91T+R108K+R118F+E210K+T244E,
A40E+E56R+D57N+G91T+R108K+R118F+E210K+D254S,
A40E+E56R+D57N+G91T+R108K+R118F+T244E+D254S,
A40E+E56R+D57N+G91T+R108K+E210K+T244E+D254S,
A40E+E56R+D57N+G91T+R118F+E210K+T244E+D254S,
A40E+E56R+D57N+K98E+R108K+R118F+E210K+T244E,
A40E+E56R+D57N+K98E+R108K+R118F+E210K+D254S,
A40E+E56R+D57N+K98E+R108K+R118F+T244E+D254S,
A40E+E56R+D57N+K98E+R108K+E210K+T244E+D254S,
A40E+E56R+D57N+K98E+R118F+E210K+T244E+D254S,
A40E+E56R+D57N+R108K+R118F+E210K+T244E+D254S,
A40E+E56R+G91T+K98E+R108K+R118F+E210K+T244E,
A40E+E56R+G91T+K98E+R108K+R118F+E210K+D254S,
A40E+E56R+G91T+K98E+R108K+R118F+T244E+D254S,
A40E+E56R+G91T+K98E+R108K+E210K+T244E+D254S,
A40E+E56R+G91T+K98E+R118F+E210K+T244E+D254S,
A40E+E56R+G91T+R108K+R118F+E210K+T244E+D254S,
A40E+E56R+K98E+R108K+R118F+E210K+T244E+D254S,
A40E+D57N+G91T+K98E+R108K+R118F+E210K+T244E,
A40E+D57N+G91T+K98E+R108K+R118F+E210K+D254S,
A40E+D57N+G91T+K98E+R108K+R118F+T244E+D254S,
A40E+D57N+G91T+K98E+R108K+E210K+T244E+D254S,
A40E+D57N+G91T+K98E+R118F+E210K+T244E+D254S,
A40E+D57N+G91T+R108K+R118F+E210K+T244E+D254S,
A40E+D57N+K98E+R108K+R118F+E210K+T244E+D254S,
A40E+G91T+K98E+R108K+R118F+E210K+T244E+D254S,
E56R+D57N+G91T+K98E+R108K+R118F+E210K+T244E,
E56R+D57N+G91T+K98E+R108K+R118F+E210K+D254S,
E56R+D57N+G91T+K98E+R108K+R118F+T244E+D254S,
E56R+D57N+G91T+K98E+R108K+E210K+T244E+D254S,
E56R+D57N+G91T+K98E+R118F+E210K+T244E+D254S,
E56R+D57N+G91T+R108K+R118F+E210K+T244E+D254S,
E56R+D57N+K98E+R108K+R118F+E210K+T244E+D254S,
E56R+G91T+K98E+R108K+R118F+E210K+T244E+D254S,
D57N+G91T+K98E+R108K+R118F+E210K+T244E+D254S,
A40E+E56R+D57N+G91T+K98E+R108K+R118F+E210K+T244E,
A40E+E56R+D57N+G91T+K98E+R108K+R118F+E210K+D254S,
A40E+E56R+D57N+G91T+K98E+R108K+R118F+T244E+D254S,
A40E+E56R+D57N+G91T+K98E+R108K+E210K+T244E+D254S,
A40E+E56R+D57N+G91T+K98E+R118F+E210K+T244E+D254S,
A40E+E56R+D57N+G91T+R108K+R118F+E210K+T244E+D254S,
A40E+E56R+D57N+K98E+R108K+R118F+E210K+T244E+D254S,
A40E+E56R+G91T+K98E+R108K+R118F+E210K+T244E+D254S,
A40E+D57N+G91T+K98E+R108K+R118F+E210K+T244E+D254S,
E56R+D57N+G91T+K98E+R108K+R118F+E210K+T244E+D254S,
A40E+E56R+D57N+G91T+K98E+R108K+R118F+E210K+T244E+D254S.
   9. The variant of any one of paragraph 1-5, wherein the variant has a substitution corresponding to I202H of SEQ ID NO: 8 and further comprises one of the following substitutions or set of substitutions corresponding to:
G23S, D27N, A40I, F51I, E56R, V60K, R118F, T244E, P256T, G23S+D27N, G23S+A40I, G23S+F51I, G23S+E56R, G23S+V60K, G23S+R118F, G23S+T244E, G23S+P256T, D27N+A40I, D27N+F51I, D27N+E56R, D27N+V60K, D27N+R118F, D27N+T244E, D27N+P256T, A40I+F51I, A40I+E56R, A40I+V60K, A40I+R118F, A40I+T244E, A40I+P256T, F51I+E56R, F51I+V60K, F51I+R118F, F51I+T244E, F51I+P256T, E56R+V60K, E56R+R118F, E56R+T244E, E56R+P256T, V60K+R118F, V60K+T244E, V60K+P256T, R118F+T244E, R118F+P256T, T244E+P256T, G23S+D27N+A40I, G23S+D27N+F51I, G23S+D27N+E56R, G23S+D27N+V60K, G23S+D27N+R118F, G23S+D27N+T244E, G23S+D27N+P256T, G23S+A40I+F51I, G23S+A40I+E56R, G23S+A40I+V60K, G23S+A40I+R118F, G23S+A40I+T244E, G23S+A40I+P256T, G23S+F51I+E56R, G23S+F51I+V60K, G23S+F51I+R118F, G23S+F51I+T244E, G23S+F51I+P256T, G23S+E56R+V60K, G23S+E56R+R118F, G23S+E56R+T244E, G23S+E56R+P256T, G23S+V60K+R118F, G23S+V60K+T244E, G23S+V60K+P256T, G23S+R118F+T244E, G23S+R118F+P256T, G23S+T244E+P256T, D27N+A40I+F51I, D27N+A40I+E56R, D27N+A40I+V60K, D27N+A40I+R118F, D27N+A40I+T244E, D27N+A40I+P256T, D27N+F51I+E56R, D27N+F51I+V60K, D27N+F51I+R118F, D27N+F51I+T244E, D27N+F51I+P256T, D27N+E56R+V60K, D27N+E56R+R118F, D27N+E56R+T244E, D27N+E56R+P256T, D27N+V60K+R118F, D27N+V60K+T244E, D27N+V60K+P256T, D27N+R118F+T244E, D27N+R118F+P256T, D27N+T244E+P256T, A40I+F51I+E56R, A40I+F51I+V60K, A40I+F51I+R118F, A40I+F51I+T244E, A40I+F51I+P256T, A40I+E56R+V60K, A40I+E56R+R118F, A40I+E56R+T244E, A40I+E56R+P256T, A40I+V60K+R118F, A40I+V60K+T244E, A40I+V60K+P256T, A40I+R118F+T244E, A40I+R118F+P256T, A40I+T244E+P256T, F51I+E56R+V60K, F51I+E56R+R118F, F51I+E56R+T244E, F51I+E56R+P256T, F51I+V60K+R118F, F51I+V60K+T244E, F51I+V60K+P256T, F51I+R118F+T244E, F51I+R118F+P256T, F51I+T244E+P256T, E56R+V60K+R118F, E56R+V60K+T244E, E56R+V60K+P256T, E56R+R118F+T244E, E56R+R118F+P256T, E56R+T244E+P256T, V60K+R118F+T244E, V60K+R118F+P256T, V60K+T244E+P256T, R118F+T244E+P256T, G23S+D27N+A40I+F51I, G23S+D27N+A40I+E56R, G23S+D27N+A40I+V60K, G23S+D27N+A40I+R118F, G23S+D27N+A40I+T244E, G23S+D27N+A40I+P256T, G23S+D27N+F51I+E56R, G23S+D27N+F51I+V60K, G23S+D27N+F51I+R118F, G23S+D27N+F51I+T244E, G23S+D27N+F51I+P256T, G23S+D27N+E56R+V60K, G23S+D27N+E56R+R118F, G23S+D27N+E56R+T244E, G23S+D27N+E56R+P256T, G23S+D27N+V60K+R118F, G23S+D27N+V60K+T244E, G23S+D27N+V60K+P256T, G23S+D27N+R118F+T244E, G23S+D27N+R118F+P256T, G23S+D27N+T244E+P256T, G23S+A40I+F51I+E56R, G23S+A40I+F51I+V60K, G23S+A40I+F51I+R118F, G23S+A40I+F51I+T244E, G23S+A40I+F51I+P256T, G23S+A40I+E56R+V60K, G23S+A40I+E56R+R118F, G23S+A40I+E56R+T244E, G23S+A40I+E56R+P256T, G23S+A40I+V60K+R118F, G23S+A40I+V60K+T244E, G23S+A40I+V60K+P256T, G23S+A40I+R118F+T244E, G23S+A40I+R118F+P256T, G23S+A40I+T244E+P256T, G23S+F51I+E56R+V60K, G23S+F51I+E56R+R118F, G23S+F51I+E56R+T244E, G23S+F51I+E56R+P256T, G23S+F51I+V60K+R118F, G23S+F51I+V60K+T244E, G23S+F51I+V60K+P256T, G23S+F51I+R118F+T244E, G23S+F51I+R118F+P256T, G23S+F51I+T244E+P256T, G23S+E56R+V60K+R118F, G23S+E56R+V60K+T244E, G23S+E56R+V60K+P256T, G23S+E56R+R118F+T244E, G23S+E56R+R118F+P256T, G23S+E56R+T244E+P256T, G23S+V60K+R118F+T244E, G23S+V60K+R118F+P256T, G23S+V60K+T244E+P256T, G23S+R118F+T244E+P256T, D27N+A40I+F51I+E56R, D27N+A40I+F51I+V60K, D27N+A40I+F51I+R118F, D27N+A40I+F51I+T244E, D27N+A40I+F51I+P256T, D27N+A40I+E56R+V60K, D27N+A40I+E56R+R118F, D27N+A40I+E56R+T244E, D27N+A40I+E56R+P256T, D27N+A40I+V60K+R118F, D27N+A40I+V60K+T244E, D27N+A40I+V60K+P256T, D27N+A40I+R118F+T244E, D27N+A40I+R118F+P256T, D27N+A40I+T244E+P256T, D27N+F51I+E56R+V60K, D27N+F51I+E56R+R118F, D27N+F51I+E56R+T244E, D27N+F51I+E56R+P256T, D27N+F51I+V60K+R118F, D27N+F51I+V60K+T244E, D27N+F51I+V60K+P256T, D27N+F51I+R118F+T244E, D27N+F51I+R118F+P256T, D27N+F51I+T244E+P256T, D27N+E56R+V60K+R118F, D27N+E56R+V60K+T244E, D27N+E56R+V60K+P256T, D27N+E56R+R118F+T244E, D27N+E56R+R118F+P256T, D27N+E56R+T244E+P256T, D27N+V60K+R118F+T244E, D27N+V60K+R118F+P256T, D27N+V60K+T244E+P256T, D27N+R118F+T244E+P256T, A40I+F51I+E56R+V60K, A40I+F51I+E56R+R118F, A40I+F51I+E56R+T244E, A40I+F51I+E56R+P256T, A40I+F51I+V60K+R118F, A40I+F51I+V60K+T244E, A40I+F51I+V60K+P256T, A40I+F51I+R118F+T244E, A40I+F51I+R118F+P256T, A40I+F51I+T244E+P256T, A40I+E56R+V60K+R118F, A40I+E56R+V60K+T244E, A40I+E56R+V60K+P256T, A40I+E56R+R118F+T244E, A40I+E56R+R118F+P256T, A40I+E56R+T244E+P256T, A40I+V60K+R118F+T244E, A40I+V60K+R118F+P256T, A40I+V60K+T244E+P256T, A40I+R118F+T244E+P256T, F51I+E56R+V60K+R118F, F51I+E56R+V60K+T244E, F51I+E56R+V60K+P256T, F51I+E56R+R118F+T244E, F51I+E56R+R118F+P256T, F51I+E56R+T244E+P256T, F51I+V60K+R118F+T244E, F51I+V60K+R118F+P256T, F51I+V60K+T244E+P256T, F51I+R118F+T244E+P256T, E56R+V60K+R118F+T244E, E56R+V60K+R118F+P256T, E56R+V60K+T244E+P256T, E56R+R118F+T244E+P256T, V60K+R118F+T244E+P256T, G23S+D27N+A40I+F51I+E56R, G23S+D27N+A40I+F51I+V60K, G23S+D27N+A40I+F51I+R118F, G23S+D27N+A40I+F51 I+T244E, G23S+D27N+A40I+F51 I+P256T, G23S+D27N+A40I+E56R+V60K, G23S+D27N+A40I+E56R+R118F, G23S+D27N+A40I+E56R+T244E, G23S+D27N+A40I+E56R+P256T, G23S+D27N+A40I+V60K+R118F, G23S+D27N+A40I+V60K+T244E, G23S+D27N+A40I+V60K+P256T, G23S+D27N+A40I+R118F+T244E, G23S+D27N+A40I+R118F+P256T, G23S+D27N+A40I+T244E+P256T, G23S+D27N+F51I+E56R+V60K, G23S+D27N+F51 I+E56R+R118F, G23S+D27N+F51I+E56R+T244E, G23S+D27N+F51 I+E56R+P256T, G23S+D27N+F51I+V60K+R118F, G23S+D27N+F51I+V60K+T244E, G23S+D27N+F51I+V60K+P256T, G23S+D27N+F51 I+R118F+T244E, G23S+D27N+F51I+R118F+P256T, G23S+D27N+F51I+T244E+P256T, G23S+D27N+E56R+V60K+R118F, G23S+D27N+E56R+V60K+T244E, G23S+D27N+E56R+V60K+P256T, G23S+D27N+E56R+R118F+T244E, G23S+D27N+E56R+R118F+P256T, G23S+D27N+E56R+T244E+P256T, G23S+D27N+V60K+R118F+T244E, G23S+D27N+V60K+R118F+P256T, G23S+D27N+V60K+T244E+P256T, G23S+D27N+R118F+T244E+P256T, G23S+A40I+F51 I+E56R+V60K, G23S+A40I+F51 I+E56R+R118F, G23S+A40I+F51I+E56R+T244E, G23S+A40I+F51I+E56R+P256T, G23S+A40I+F51 I+V60K+R118F, G23S+A40I+F51I+V60K+T244E, G23S+A40I+F51I+V60K+P256T, G23S+A40I+F51 I+R118F+T244E, G23S+A40I+F51I+R118F+P256T, G23S+A40I+F51I+T244E+P256T, G23S+A40I+E56R+V60K+R118F, G23S+A40I+E56R+V60K+T244E, G23S+A40I+E56R+V60K+P256T, G23S+A40I+E56R+R118F+T244E, G23S+A40I+E56R+R118F+P256T, G23S+A40I+E56R+T244E+P256T, G23S+A40I+V60K+R118F+T244E, G23S+A40I+V60K+R118F+P256T, G23S+A40I+V60K+T244E+P256T, G23S+A40I+R118F+T244E+P256T, G23S+F51I+E56R+V60K+R118F, G23S+F51 I+E56R+V60K+T244E, G23S+F51 I+E56R+V60K+P256T, G23S+F51I+E56R+R118F+T244E, G23S+F51I+E56R+R118F+P256T, G23S+F51I+E56R+T244E+P256T, G23S+F51I+V60K+R118F+T244E, G23S+F51 I+V60K+R118F+P256T, G23S+F51I+V60K+T244E+P256T, G23S+F51I+R118F+T244E+P256T, G23S+E56R+V60K+R118F+T244E, G23S+E56R+V60K+R118F+P256T, G23S+E56R+V60K+T244E+P256T, G23S+E56R+R118F+T244E+P256T, G23S+V60K+R118F+T244E+P256T, D27N+A40I+F51I+E56R+V60K, D27N+A40I+F51 I+E56R+R118F, D27N+A40I+F51 I+E56R+T244E, D27N+A40I+F51 I+E56R+P256T, D27N+A40I+F51I+V60K+R118F, D27N+A40I+F51I+V60K+T244E, D27N+A40I+F51 I+V60K+P256T, D27N+A40I+F51 I+R118F+T244E, D27N+A40I+F51I+R118F+P256T, D27N+A40I+F51I+T244E+P256T, D27N+A40I+E56R+V60K+R118F, D27N+A40I+E56R+V60K+T244E, D27N+A40I+E56R+V60K+P256T, D27N+A40I+E56R+R118F+T244E, D27N+A40I+E56R+R118F+P256T, D27N+A40I+E56R+T244E+P256T, D27N+A40I+V60K+R118F+T244E, D27N+A40I+V60K+R118F+P256T, D27N+A40I+V60K+T244E+P256T, D27N+A40I+R118F+T244E+P256T, D27N+F51 I+E56R+V60K+R118F, D27N+F51 I+E56R+V60K+T244E, D27N+F51 I+E56R+V60K+P256T, D27N+F51 I+E56R+R118F+T244E, D27N+F51I+E56R+R118F+P256T, D27N+F51I+E56R+T244E+P256T, D27N+F51 I+V60K+R118F+T244E, D27N+F51I+V60K+R118F+P256T, D27N+F51I+V60K+T244E+P256T, D27N+F51I+R118F+T244E+P256T, D27N+E56R+V60K+R118F+T244E, D27N+E56R+V60K+R118F+P256T, D27N+E56R+V60K+T244E+P256T, D27N+E56R+R118F+T244E+P256T, D27N+V60K+R118F+T244E+P256T, A40I+F51I+E56R+V60K+R118F, A40I+F51 I+E56R+V60K+T244E, A40I+F51 I+E56R+V60K+P256T, A40I+F51I+E56R+R118F+T244E, A40I+F51I+E56R+R118F+P256T, A40I+F51I+E56R+T244E+P256T, A40I+F51I+V60K+R118F+T244E, A40I+F51I+V60K+R118F+P256T, A40I+F51I+V60K+T244E+P256T, A40I+F51I+R118F+T244E+P256T, A40I+E56R+V60K+R118F+T244E, A40I+E56R+V60K+R118F+P256T, A40I+E56R+V60K+T244E+P256T, A40I+E56R+R118F+T244E+P256T, A40I+V60K+R118F+T244E+P256T, F51I+E56R+V60K+R118F+T244E, F51I+E56R+V60K+R118F+P256T, F51I+E56R+V60K+T244E+P256T, F51I+E56R+R118F+T244E+P256T, F51I+V60K+R118F+T244E+P256T, E56R+V60K+R118F+T244E+P256T, G23S+D27N+A40I+F51I+E56R+V60K, G23S+D27N+A40I+F51 I+E56R+R118F, G23S+D27N+A40I+F51 I+E56R+T244E, G23S+D27N+A40I+F51 I+E56R+P256T, G23S+D27N+A40I+F51I+V60K+R118F, G23S+D27N+A40I+F51I+V60K+T244E, G23S+D27N+A40I+F51I+V60K+P256T, G23S+D27N+A40I+F51I+R118F+T244E, G23S+D27N+A40I+F51I+R118F+P256T, G23S+D27N+A40I+F51I+T244E+P256T, G23S+D27N+A40I+E56R+V60K+R118F, G23S+D27N+A40I+E56R+V60K+T244E, G23S+D27N+A40I+E56R+V60K+P256T, G23S+D27N+A40I+E56R+R118F+T244E, G23S+D27N+A40I+E56R+R118F+P256T, G23S+D27N+A40I+E56R+T244E+P256T, G23S+D27N+A40I+V60K+R118F+T244E, G23S+D27N+A40I+V60K+R118F+P256T, G23S+D27N+A40I+V60K+T244E+P256T, G23S+D27N+A40I+R118F+T244E+P256T, G23S+D27N+F51 I+E56R+V60K+R118F, G23S+D27N+F51 I+E56R+V60K+T244E, G23S+D27N+F51I+E56R+V60K+P256T, G23S+D27N+F51I+E56R+R118F+T244E, G23S+D27N+F51I+E56R+R118F+P256T, G23S+D27N+F51 I+E56R+T244E+P256T, G23S+D27N+F51I+V60K+R118F+T244E, G23S+D27N+F51I+V60K+R118F+P256T, G23S+D27N+F51I+V60K+T244E+P256T, G23S+D27N+F51I+R118F+T244E+P256T, G23S+D27N+E56R+V60K+R118F+T244E, G23S+D27N+E56R+V60K+R118F+P256T, G23S+D27N+E56R+V60K+T244E+P256T, G23S+D27N+E56R+R118F+T244E+P256T, G23S+D27N+V60K+R118F+T244E+P256T, G23S+A40I+F51I+E56R+V60K+R118F, G23S+A40I+F51I+E56R+V60K+T244E, G23S+A40I+F51I+E56R+V60K+P256T, G23S+A40I+F51I+E56R+R118F+T244E, G23S+A40I+F51I+E56R+R118F+P256T, G23S+A40I+F51 I+E56R+T244E+P256T, G23S+A40I+F51I+V60K+R118F+T244E, G23S+A40I+F51I+V60K+R118F+P256T, G23S+A40I+F51I+V60K+T244E+P256T, G23S+A40I+F51 I+R118F+T244E+P256T, G23S+A40I+E56R+V60K+R118F+T244E, G23S+A40I+E56R+V60K+R118F+P256T, G23S+A40I+E56R+V60K+T244E+P256T, G23S+A40I+E56R+R118F+T244E+P256T, G23S+A40I+V60K+R118F+T244E+P256T, G23S+F51I+E56R+V60K+R118F+T244E, G23S+F51I+E56R+V60K+R118F+P256T, G23S+F51I+E56R+V60K+T244E+P256T, G23S+F51I+E56R+R118F+T244E+P256T, G23S+F51I+V60K+R118F+T244E+P256T, G23S+E56R+V60K+R118F+T244E+P256T, D27N+A40I+F51 I+E56R+V60K+R118F, D27N+A40I+F51 I+E56R+V60K+T244E, D27N+A40I+F51 I+E56R+V60K+P256T, D27N+A40I+F51I+E56R+R118F+T244E, D27N+A40I+F51I+E56R+R118F+P256T, D27N+A40I+F51 I+E56R+T244E+P256T, D27N+A40I+F51I+V60K+R118F+T244E, D27N+A40I+F51I+V60K+R118F+P256T, D27N+A40I+F51I+V60K+T244E+P256T, D27N+A40I+F51I+R118F+T244E+P256T, D27N+A40I+E56R+V60K+R118F+T244E, D27N+A40I+E56R+V60K+R118F+P256T, D27N+A40I+E56R+V60K+T244E+P256T, D27N+A40I+E56R+R118F+T244E+P256T, D27N+A40I+V60K+R118F+T244E+P256T, D27N+F51I+E56R+V60K+R118F+T244E, D27N+F51I+E56R+V60K+R118F+P256T, D27N+F51I+E56R+V60K+T244E+P256T, D27N+F51I+E56R+R118F+T244E+P256T, D27N+F51I+V60K+R118F+T244E+P256T, D27N+E56R+V60K+R118F+T244E+P256T, A40I+F51I+E56R+V60K+R118F+T244E, A40I+F51 I+E56R+V60K+R118F+P256T, A40I+F51 I+E56R+V60K+T244E+P256T, A40I+F51I+E56R+R118F+T244E+P256T, A40I+F51I+V60K+R118F+T244E+P256T, A40I+E56R+V60K+R118F+T244E+P256T, F51I+E56R+V60K+R118F+T244E+P256T, G23S+D27N+A40I+F51I+E56R+V60K+R118F, G23S+D27N+A40I+F51I+E56R+V60K+T244E, G23S+D27N+A40I+F51I+E56R+V60K+P256T, G23S+D27N+A40I+F51I+E56R+R118F+T244E,
G23S+D27N+A40I+F51I+E56R+R118F+P256T,
G23S+D27N+A40I+F51I+E56R+T244E+P256T,
G23S+D27N+A40I+F51I+V60K+R118F+T244E,
G23S+D27N+A40I+F51I+V60K+R118F+P256T,
G23S+D27N+A40I+F51I+V60K+T244E+P256T,
G23S+D27N+A40I+F51I+R118F+T244E+P256T,
G23S+D27N+A40I+E56R+V60K+R118F+T244E,
G23S+D27N+A40I+E56R+V60K+R118F+P256T,
G23S+D27N+A40I+E56R+V60K+T244E+P256T,
G23S+D27N+A40I+E56R+R118F+T244E+P256T,
G23S+D27N+A40I+V60K+R118F+T244E+P256T,
G23S+D27N+F51I+E56R+V60K+R118F+T244E,
G23S+D27N+F51I+E56R+V60K+R118F+P256T,
G23S+D27N+F51I+E56R+V60K+T244E+P256T,
G23S+D27N+F51I+E56R+R118F+T244E+P256T,
G23S+D27N+F51I+V60K+R118F+T244E+P256T,
G23S+D27N+E56R+V60K+R118F+T244E+P256T,
G23S+A40I+F51I+E56R+V60K+R118F+T244E, G23S+A40I+F51I+E56R+V60K+R118F+P256T,
G23S+A40I+F51I+E56R+V60K+T244E+P256T,
G23S+A40I+F51I+E56R+R118F+T244E+P256T,
G23S+A40I+F51I+V60K+R118F+T244E+P256T,
G23S+A40I+E56R+V60K+R118F+T244E+P256T,
G23S+F51I+E56R+V60K+R118F+T244E+P256T,
D27N+A40I+F51I+E56R+V60K+R118F+T244E, D27N+A40I+F51I+E56R+V60K+R118F+P256T,
D27N+A40I+F51I+E56R+V60K+T244E+P256T,
D27N+A40I+F51I+E56R+R118F+T244E+P256T,
D27N+A40I+F51I+V60K+R118F+T244E+P256T,
D27N+A40I+E56R+V60K+R118F+T244E+P256T,
D27N+F51I+E56R+V60K+R118F+T244E+P256T,
A40I+F51I+E56R+V60K+R118F+T244E+P256T,
G23S+D27N+A40I+F51I+E56R+V60K+R118F+T244E,
G23S+D27N+A40I+F51I+E56R+V60K+R118F+P256T,
G23S+D27N+A40I+F51I+E56R+V60K+T244E+P256T,
G23S+D27N+A40I+F51I+E56R+R118F+T244E+P256T,
G23S+D27N+A40I+F51I+V60K+R118F+T244E+P256T,
G23S+D27N+A40I+E56R+V60K+R118F+T244E+P256T,
G23S+D27N+F51I+E56R+V60K+R118F+T244E+P256T,
G23S+A40I+F51I+E56R+V60K+R118F+T244E+P256T,
D27N+A40I+F51I+E56R+V60K+R118F+T244E+P256T,
G23S+D27N+A40I+F51I+E56R+V60K+R118F+T244E+P256T.
   10. The variant of any one of paragraph 1-5, wherein the variant has a substitution corresponding to I252H of SEQ ID NO: 8 and further comprises one of the following substitutions or set of substitutions corresponding to:
G23S, D27N, A40I, F51I, E56R, V60K, R118F, T244E, P256T, G23S+D27N, G23S+A40I, G23S+F51I, G23S+E56R, G23S+V60K, G23S+R118F, G23S+T244E, G23S+P256T, D27N+A40I, D27N+F51I, D27N+E56R, D27N+V60K, D27N+R118F, D27N+T244E, D27N+P256T, A40I+F51I, A40I+E56R, A40I+V60K, A40I+R118F, A40I+T244E, A40I+P256T, F51I+E56R, F51I+V60K, F51I+R118F, F51I+T244E, F51I+P256T, E56R+V60K, E56R+R118F, E56R+T244E, E56R+P256T, V60K+R118F, V60K+T244E, V60K+P256T, R118F+T244E, R118F+P256T, T244E+P256T, G23S+D27N+A40I, G23S+D27N+F51I, G23S+D27N+E56R, G23S+D27N+V60K, G23S+D27N+R118F, G23S+D27N+T244E, G23S+D27N+P256T, G23S+A40I+F51I, G23S+A40I+E56R, G23S+A40I+V60K, G23S+A40I+R118F, G23S+A40I+T244E, G23S+A40I+P256T, G23S+F51I+E56R, G23S+F51I+V60K, G23S+F51I+R118F, G23S+F51I+T244E, G23S+F51I+P256T, G23S+E56R+V60K, G23S+E56R+R118F, G23S+E56R+T244E, G23S+E56R+P256T, G23S+V60K+R118F, G23S+V60K+T244E, G23S+V60K+P256T, G23S+R118F+T244E, G23S+R118F+P256T, G23S+T244E+P256T, D27N+A40I+F51I, D27N+A40I+E56R, D27N+A40I+V60K, D27N+A40I+R118F, D27N+A40I+T244E, D27N+A40I+P256T, D27N+F51I+E56R, D27N+F51I+V60K, D27N+F51I+R118F, D27N+F51I+T244E, D27N+F51I+P256T, D27N+E56R+V60K, D27N+E56R+R118F, D27N+E56R+T244E, D27N+E56R+P256T, D27N+V60K+R118F, D27N+V60K+T244E, D27N+V60K+P256T, D27N+R118F+T244E, D27N+R118F+P256T, D27N+T244E+P256T, A40I+F51I+E56R, A40I+F51I+V60K, A40I+F51I+R118F, A40I+F51I+T244E, A40I+F51I+P256T, A40I+E56R+V60K, A40I+E56R+R118F, A40I+E56R+T244E, A40I+E56R+P256T, A40I+V60K+R118F, A40I+V60K+T244E, A40I+V60K+P256T, A40I+R118F+T244E, A40I+R118F+P256T, A40I+T244E+P256T, F51I+E56R+V60K, F51I+E56R+R118F, F51I+E56R+T244E, F51I+E56R+P256T, F51I+V60K+R118F, F51I+V60K+T244E, F51I+V60K+P256T, F51I+R118F+T244E, F51I+R118F+P256T, F51I+T244E+P256T, E56R+V60K+R118F, E56R+V60K+T244E, E56R+V60K+P256T, E56R+R118F+T244E, E56R+R118F+P256T, E56R+T244E+P256T, V60K+R118F+T244E, V60K+R118F+P256T, V60K+T244E+P256T, R118F+T244E+P256T, G23S+D27N+A40I+F51I, G23S+D27N+A40I+E56R, G23S+D27N+A40I+V60K, G23S+D27N+A40I+R118F, G23S+D27N+A40I+T244E, G23S+D27N+A40I+P256T, G23S+D27N+F51I+E56R, G23S+D27N+F51I+V60K, G23S+D27N+F51I+R118F, G23S+D27N+F51I+T244E, G23S+D27N+F51I+P256T, G23S+D27N+E56R+V60K, G23S+D27N+E56R+R118F, G23S+D27N+E56R+T244E, G23S+D27N+E56R+P256T, G23S+D27N+V60K+R118F, G23S+D27N+V60K+T244E, G23S+D27N+V60K+P256T, G23S+D27N+R118F+T244E, G23S+D27N+R118F+P256T, G23S+D27N+T244E+P256T, G23S+A40I+F51I+E56R, G23S+A40I+F51I+V60K, G23S+A40I+F51I+R118F, G23S+A40I+F51I+T244E, G23S+A40I+F51I+P256T, G23S+A40I+E56R+V60K, G23S+A40I+E56R+R118F, G23S+A40I+E56R+T244E, G23S+A40I+E56R+P256T, G23S+A40I+V60K+R118F, G23S+A40I+V60K+T244E, G23S+A40I+V60K+P256T, G23S+A40I+R118F+T244E, G23S+A40I+R118F+P256T, G23S+A40I+T244E+P256T, G23S+F51I+E56R+V60K, G23S+F51I+E56R+R118F, G23S+F51I+E56R+T244E, G23S+F51I+E56R+P256T, G23S+F51I+V60K+R118F, G23S+F51I+V60K+T244E, G23S+F51I+V60K+P256T, G23S+F51I+R118F+T244E, G23S+F51I+R118F+P256T, G23S+F51I+T244E+P256T, G23S+E56R+V60K+R118F, G23S+E56R+V60K+T244E, G23S+E56R+V60K+P256T, G23S+E56R+R118F+T244E, G23S+E56R+R118F+P256T, G23S+E56R+T244E+P256T, G23S+V60K+R118F+T244E, G23S+V60K+R118F+P256T, G23S+V60K+T244E+P256T, G23S+R118F+T244E+P256T, D27N+A40I+F51I+E56R, D27N+A40I+F51I+V60K, D27N+A40I+F51I+R118F, D27N+A40I+F51I+T244E, D27N+A40I+F51I+P256T, D27N+A40I+E56R+V60K, D27N+A40I+E56R+R118F, D27N+A40I+E56R+T244E, D27N+A40I+E56R+P256T, D27N+A40I+V60K+R118F, D27N+A40I+V60K+T244E, D27N+A40I+V60K+P256T, D27N+A40I+R118F+T244E, D27N+A40I+R118F+P256T, D27N+A40I+T244E+P256T, D27N+F51I+E56R+V60K, D27N+F51I+E56R+R118F, D27N+F51I+E56R+T244E, D27N+F51I+E56R+P256T, D27N+F51I+V60K+R118F, D27N+F51I+V60K+T244E, D27N+F51I+V60K+P256T, D27N+F51I+R118F+T244E, D27N+F51I+R118F+P256T, D27N+F51I+T244E+P256T, D27N+E56R+V60K+R118F, D27N+E56R+V60K+T244E, D27N+E56R+V60K+P256T, D27N+E56R+R118F+T244E, D27N+E56R+R118F+P256T, D27N+E56R+T244E+P256T, D27N+V60K+R118F+T244E, D27N+V60K+R118F+P256T, D27N+V60K+T244E+P256T, D27N+R118F+T244E+P256T, A40I+F51I+E56R+V60K, A40I+F51I+E56R+R118F, A40I+F51I+E56R+T244E, A40I+F51I+E56R+P256T, A40I+F51I+V60K+R118F, A40I+F51I+V60K+T244E, A40I+F51I+V60K+P256T, A40I+F51I+R118F+T244E, A40I+F51I+R118F+P256T, A40I+F51I+T244E+P256T, A40I+E56R+V60K+R118F, A40I+E56R+V60K+T244E, A40I+E56R+V60K+P256T, A40I+E56R+R118F+T244E, A40I+E56R+R118F+P256T, A40I+E56R+T244E+P256T, A40I+V60K+R118F+T244E, A40I+V60K+R118F+P256T, A40I+V60K+T244E+P256T, A40I+R118F+T244E+P256T, F51I+E56R+V60K+R118F, F51I+E56R+V60K+T244E, F51I+E56R+V60K+P256T, F51I+E56R+R118F+T244E, F51I+E56R+R118F+P256T, F51I+E56R+T244E+P256T, F51I+V60K+R118F+T244E, F51I+V60K+R118F+P256T, F51I+V60K+T244E+P256T, F51I+R118F+T244E+P256T, E56R+V60K+R118F+T244E, E56R+V60K+R118F+P256T, E56R+V60K+T244E+P256T, E56R+R118F+T244E+P256T, V60K+R118F+T244E+P256T, G23S+D27N+A40I+F51I+E56R, G23S+D27N+A40I+F51I+V60K, G23S+D27N+A40I+F51I+R118F, G23S+D27N+A40I+F51I+T244E, G23S+D27N+A40I+F51 I+P256T, G23S+D27N+A40I+E56R+V60K, G23S+D27N+A40I+E56R+R118F, G23S+D27N+A40I+E56R+T244E, G23S+D27N+A40I+E56R+P256T, G23S+D27N+A40I+V60K+R118F, G23S+D27N+A40I+V60K+T244E, G23S+D27N+A40I+V60K+P256T, G23S+D27N+A40I+R118F+T244E, G23S+D27N+A40I+R118F+P256T, G23S+D27N+A40I+T244E+P256T, G23S+D27N+F51I+E56R+V60K, G23S+D27N+F51I+E56R+R118F, G23S+D27N+F51I+E56R+T244E, G23S+D27N+F51 I+E56R+P256T, G23S+D27N+F51I+V60K+R118F, G23S+D27N+F51 I+V60K+T244E, G23S+D27N+F51I+V60K+P256T, G23S+D27N+F51 I+R118F+T244E, G23S+D27N+F51I+R118F+P256T, G23S+D27N+F51I+T244E+P256T, G23S+D27N+E56R+V60K+R118F, G23S+D27N+E56R+V60K+T244E, G23S+D27N+E56R+V60K+P256T, G23S+D27N+E56R+R118F+T244E, G23S+D27N+E56R+R118F+P256T, G23S+D27N+E56R+T244E+P256T, G23S+D27N+V60K+R118F+T244E, G23S+D27N+V60K+R118F+P256T, G23S+D27N+V60K+T244E+P256T, G23S+D27N+R118F+T244E+P256T, G23S+A40I+F51 I+E56R+V60K, G23S+A40I+F51I+E56R+R118F, G23S+A40I+F51I+E56R+T244E, G23S+A40I+F51I+E56R+P256T, G23S+A40I+F51 I+V60K+R118F, G23S+A40I+F51I+V60K+T244E, G23S+A40I+F51I+V60K+P256T, G23S+A40I+F51 I+R118F+T244E, G23S+A40I+F51I+R118F+P256T, G23S+A40I+F51I+T244E+P256T, G23S+A40I+E56R+V60K+R118F, G23S+A40I+E56R+V60K+T244E, G23S+A40I+E56R+V60K+P256T, G23S+A40I+E56R+R118F+T244E, G23S+A40I+E56R+R118F+P256T, G23S+A40I+E56R+T244E+P256T, G23S+A40I+V60K+R118F+T244E, G23S+A40I+V60K+R118F+P256T, G23S+A40I+V60K+T244E+P256T, G23S+A40I+R118F+T244E+P256T, G23S+F51I+E56R+V60K+R118F, G23S+F51I+E56R+V60K+T244E, G23S+F51I+E56R+V60K+P256T, G23S+F51I+E56R+R118F+T244E, G23S+F51 I+E56R+R118F+P256T, G23S+F51 I+E56R+T244E+P256T, G23S+F51I+V60K+R118F+T244E, G23S+F51 I+V60K+R118F+P256T, G23S+F51I+V60K+T244E+P256T, G23S+F51I+R118F+T244E+P256T, G23S+E56R+V60K+R118F+T244E, G23S+E56R+V60K+R118F+P256T, G23S+E56R+V60K+T244E+P256T, G23S+E56R+R118F+T244E+P256T, G23S+V60K+R118F+T244E+P256T, D27N+A40I+F51I+E56R+V60K, D27N+A40I+F51 I+E56R+R118F, D27N+A40I+F51 I+E56R+T244E, D27N+A40I+F51 I+E56R+P256T, D27N+A40I+F51I+V60K+R118F, D27N+A40I+F51 I+V60K+T244E, D27N+A40I+F51 I+V60K+P256T, D27N+A40I+F51 I+R118F+T244E, D27N+A40I+F51I+R118F+P256T, D27N+A40I+F51I+T244E+P256T, D27N+A40I+E56R+V60K+R118F, D27N+A40I+E56R+V60K+T244E, D27N+A40I+E56R+V60K+P256T, D27N+A40I+E56R+R118F+T244E, D27N+A40I+E56R+R118F+P256T, D27N+A40I+E56R+T244E+P256T, D27N+A40I+V60K+R118F+T244E, D27N+A40I+V60K+R118F+P256T, D27N+A40I+V60K+T244E+P256T, D27N+A40I+R118F+T244E+P256T, D27N+F51I+E56R+V60K+R118F, D27N+F51I+E56R+V60K+T244E, D27N+F51 I+E56R+V60K+P256T, D27N+F51 I+E56R+R118F+T244E, D27N+F51I+E56R+R118F+P256T, D27N+F51I+E56R+T244E+P256T, D27N+F51 I+V60K+R118F+T244E, D27N+F51I+V60K+R118F+P256T, D27N+F51I+V60K+T244E+P256T, D27N+F51I+R118F+T244E+P256T, D27N+E56R+V60K+R118F+T244E, D27N+E56R+V60K+R118F+P256T, D27N+E56R+V60K+T244E+P256T, D27N+E56R+R118F+T244E+P256T, D27N+V60K+R118F+T244E+P256T, A40I+F51I+E56R+V60K+R118F, A40I+F51 I+E56R+V60K+T244E, A40I+F51 I+E56R+V60K+P256T, A40I+F51I+E56R+R118F+T244E, A40I+F51I+E56R+R118F+P256T, A40I+F51I+E56R+T244E+P256T, A40I+F51I+V60K+R118F+T244E, A40I+F51I+V60K+R118F+P256T, A40I+F51I+V60K+T244E+P256T, A40I+F51I+R118F+T244E+P256T, A40I+E56R+V60K+R118F+T244E, A40I+E56R+V60K+R118F+P256T, A40I+E56R+V60K+T244E+P256T, A40I+E56R+R118F+T244E+P256T, A40I+V60K+R118F+T244E+P256T, F51I+E56R+V60K+R118F+T244E, F51I+E56R+V60K+R118F+P256T, F51 I+E56R+V60K+T244E+P256T, F51 I+E56R+R118F+T244E+P256T, F51I+V60K+R118F+T244E+P256T, E56R+V60K+R118F+T244E+P256T, G23S+D27N+A40I+F51I+E56R+V60K, G23S+D27N+A40I+F51 I+E56R+R118F, G23S+D27N+A40I+F51 I+E56R+T244E, G23S+D27N+A40I+F51 I+E56R+P256T, G23S+D27N+A40I+F51I+V60K+R118F, G23S+D27N+A40I+F51I+V60K+T244E, G23S+D27N+A40I+F51I+V60K+P256T, G23S+D27N+A40I+F51I+R118F+T244E, G23S+D27N+A40I+F51 I+R118F+P256T, G23S+D27N+A40I+F51 I+T244E+P256T, G23S+D27N+A40I+E56R+V60K+R118F, G23S+D27N+A40I+E56R+V60K+T244E, G23S+D27N+A40I+E56R+V60K+P256T, G23S+D27N+A40I+E56R+R118F+T244E, G23S+D27N+A40I+E56R+R118F+P256T, G23S+D27N+A40I+E56R+T244E+P256T, G23S+D27N+A40I+V60K+R118F+T244E, G23S+D27N+A40I+V60K+R118F+P256T, G23S+D27N+A40I+V60K+T244E+P256T, G23S+D27N+A40I+R118F+T244E+P256T, G23S+D27N+F51 I+E56R+V60K+R118F, G23S+D27N+F51 I+E56R+V60K+T244E, G23S+D27N+F51 I+E56R+V60K+P256T, G23S+D27N+F51I+E56R+R118F+T244E, G23S+D27N+F51I+E56R+R118F+P256T, G23S+D27N+F51 I+E56R+T244E+P256T, G23S+D27N+F51I+V60K+R118F+T244E, G23S+D27N+F51I+V60K+R118F+P256T, G23S+D27N+F51I+V60K+T244E+P256T, G23S+D27N+F51I+R118F+T244E+P256T, G23S+D27N+E56R+V60K+R118F+T244E, G23S+D27N+E56R+V60K+R118F+P256T, G23S+D27N+E56R+V60K+T244E+P256T, G23S+D27N+E56R+R118F+T244E+P256T, G23S+D27N+V60K+R118F+T244E+P256T, G23S+A40I+F51I+E56R+V60K+R118F, G23S+A40I+F51I+E56R+V60K+T244E, G23S+A40I+F51I+E56R+V60K+P256T, G23S+A40I+F51I+E56R+R118F+T244E, G23S+A40I+F51I+E56R+R118F+P256T, G23S+A40I+F51 I+E56R+T244E+P256T, G23S+A40I+F51I+V60K+R118F+T244E, G23S+A40I+F51 I+V60K+R118F+P256T, G23S+A40I+F51I+V60K+T244E+P256T, G23S+A40I+F51I+R118F+T244E+P256T, G23S+A40I+E56R+V60K+R118F+T244E, G23S+A40I+E56R+V60K+R118F+P256T, G23S+A40I+E56R+V60K+T244E+P256T, G23S+A40I+E56R+R118F+T244E+P256T, G23S+A40I+V60K+R118F+T244E+P256T, G23S+F51I+E56R+V60K+R118F+T244E, G23S+F51I+E56R+V60K+R118F+P256T, G23S+F51I+E56R+V60K+T244E+P256T, G23S+F51I+E56R+R118F+T244E+P256T, G23S+F51I+V60K+R118F+T244E+P256T, G23S+E56R+V60K+R118F+T244E+P256T, D27N+A40I+F51 I+E56R+V60K+R118F, D27N+A40I+F51 I+E56R+V60K+T244E, D27N+A40I+F51I+E56R+V60K+P256T, D27N+A40I+F51I+E56R+R118F+T244E, D27N+A40I+F51 I+E56R+R118F+P256T, D27N+A40I+F51 I+E56R+T244E+P256T, D27N+A40I+F51I+V60K+R118F+T244E, D27N+A40I+F51I+V60K+R118F+P256T, D27N+A40I+F51I+V60K+T244E+P256T, D27N+A40I+F51I+R118F+T244E+P256T, D27N+A40I+E56R+V60K+R118F+T244E, D27N+A40I+E56R+V60K+R118F+P256T, D27N+A40I+E56R+V60K+T244E+P256T, D27N+A40I+E56R+R118F+T244E+P256T, D27N+A40I+V60K+R118F+T244E+P256T, D27N+F51I+E56R+V60K+R118F+T244E, D27N+F51I+E56R+V60K+R118F+P256T, D27N+F51 I+E56R+V60K+T244E+P256T, D27N+F51I+E56R+R118F+T244E+P256T, D27N+F51I+V60K+R118F+T244E+P256T, D27N+E56R+V60K+R118F+T244E+P256T, A40I+F51I+E56R+V60K+R118F+T244E, A40I+F51I+E56R+V60K+R118F+P256T, A40I+F51I+E56R+V60K+T244E+P256T, A40I+F51I+E56R+R118F+T244E+P256T, A40I+F51 I+V60K+R118F+T244E+P256T, A40I+E56R+V60K+R118F+T244E+P256T, F51I+E56R+V60K+R118F+T244E+P256T, G23S+D27N+A40I+F51I+E56R+V60K+R118F, G23S+D27N+A40I+F51I+E56R+V60K+T244E, G23S+D27N+A40I+F51I+E56R+V60K+P256T, G23S+D27N+A40I+F51I+E56R+R118F+T244E,
G23S+D27N+A40I+F51I+E56R+R118F+P256T,
G23S+D27N+A40I+F51I+E56R+T244E+P256T,
G23S+D27N+A40I+F51I+V60K+R118F+T244E,
G23S+D27N+A40I+F51I+V60K+R118F+P256T,
G23S+D27N+A40I+F51I+V60K+T244E+P256T,
G23S+D27N+A40I+F51I+R118F+T244E+P256T,
G23S+D27N+A40I+E56R+V60K+R118F+T244E,
G23S+D27N+A40I+E56R+V60K+R118F+P256T,
G23S+D27N+A40I+E56R+V60K+T244E+P256T,
G23S+D27N+A40I+E56R+R118F+T244E+P256T,
G23S+D27N+A40I+V60K+R118F+T244E+P256T,
G23S+D27N+F51I+E56R+V60K+R118F+T244E,
G23S+D27N+F51I+E56R+V60K+R118F+P256T,
G23S+D27N+F51I+E56R+V60K+T244E+P256T,
G23S+D27N+F51I+E56R+R118F+T244E+P256T,
G23S+D27N+F51I+V60K+R118F+T244E+P256T,
G23S+D27N+E56R+V60K+R118F+T244E+P256T,
G23S+A40I+F51I+E56R+V60K+R118F+T244E, G23S+A40I+F51I+E56R+V60K+R118F+P256T,
G23S+A40I+F51I+E56R+V60K+T244E+P256T,
G23S+A40I+F51I+E56R+R118F+T244E+P256T,
G23S+A40I+F51I+V60K+R118F+T244E+P256T,
G23S+A40I+E56R+V60K+R118F+T244E+P256T,
G23S+F51I+E56R+V60K+R118F+T244E+P256T,
D27N+A40I+F51I+E56R+V60K+R118F+T244E, D27N+A40I+F51I+E56R+V60K+R118F+P256T,
D27N+A40I+F51I+E56R+V60K+T244E+P256T,
D27N+A40I+F51I+E56R+R118F+T244E+P256T,
D27N+A40I+F51I+V60K+R118F+T244E+P256T,
D27N+A40I+E56R+V60K+R118F+T244E+P256T,
D27N+F51I+E56R+V60K+R118F+T244E+P256T,
A40I+F51I+E56R+V60K+R118F+T244E+P256T,
G23S+D27N+A40I+F51I+E56R+V60K+R118F+T244E,
G23S+D27N+A40I+F51I+E56R+V60K+R118F+P256T,
G23S+D27N+A40I+F51I+E56R+V60K+T244E+P256T,
G23S+D27N+A40I+F51I+E56R+R118F+T244E+P256T,
G23S+D27N+A40I+F51I+V60K+R118F+T244E+P256T,
G23S+D27N+A40I+E56R+V60K+R118F+T244E+P256T,
G23S+D27N+F51I+E56R+V60K+R118F+T244E+P256T,
G23S+A40I+F51I+E56R+V60K+R118F+T244E+P256T,
D27N+A40I+F51I+E56R+V60K+R118F+T244E+P256T,
G23S+D27N+A40I+F51I+E56R+V60K+R118F+T244E+P256T.
   11. The variant of any one of paragraph 1-5, wherein the variant has a substitution corresponding to L269H of SEQ ID NO: 8 and further comprises one of the following substitutions or set of substitutions corresponding to:
G23S, D27N, A40I, F51I, E56R, V60K, R118F, T244E, P256T, G23S+D27N, G23S+A40I, G23S+F51I, G23S+E56R, G23S+V60K, G23S+R118F, G23S+T244E, G23S+P256T, D27N+A40I, D27N+F51I, D27N+E56R, D27N+V60K, D27N+R118F, D27N+T244E, D27N+P256T, A40I+F51I, A40I+E56R, A40I+V60K, A40I+R118F, A40I+T244E, A40I+P256T, F51I+E56R, F51I+V60K, F51I+R118F, F51I+T244E, F51I+P256T, E56R+V60K, E56R+R118F, E56R+T244E, E56R+P256T, V60K+R118F, V60K+T244E, V60K+P256T, R118F+T244E, R118F+P256T, T244E+P256T, G23S+D27N+A40I, G23S+D27N+F51I, G23S+D27N+E56R, G23S+D27N+V60K, G23S+D27N+R118F, G23S+D27N+T244E, G23S+D27N+P256T, G23S+A40I+F51I, G23S+A40I+E56R, G23S+A40I+V60K, G23S+A40I+R118F, G23S+A40I+T244E, G23S+A40I+P256T, G23S+F51i+E56R, G23S+F51 I+V60K, G23S+F51I+R118F, G23S+F51 I+T244E, G23S+F51I+P256T, G23S+E56R+V60K, G23S+E56R+R118F, G23S+E56R+T244E, G23S+E56R+P256T, G23S+V60K+R118F, G23S+V60K+T244E, G23S+V60K+P256T, G23S+R118F+T244E, G23S+R118F+P256T, G23S+T244E+P256T, D27N+A40I+F51I, D27N+A40I+E56R, D27N+A40I+V60K, D27N+A40I+R118F, D27N+A40I+T244E, D27N+A40I+P256T, D27N+F51I+E56R, D27N+F51I+V60K, D27N+F51 l+R118F, D27N+F51I+T244E, D27N+F51I+P256T, D27N+E56R+V60K, D27N+E56R+R118F, D27N+E56R+T244E, D27N+E56R+P256T, D27N+V60K+R118F, D27N+V60K+T244E, D27N+V60K+P256T, D27N+R118F+T244E, D27N+R118F+P256T, D27N+T244E+P256T, A40I+F51I+E56R, A40I+F51 I+V60K, A40I+F51l+R118F, A40I+F51I+T244E, A40I+F51 I+P256T, A40I+E56R+V60K, A40I+E56R+R118F, A40I+E56R+T244E, A40I+E56R+P256T, A40I+V60K+R118F, A40I+V60K+T244E, A40I+V60K+P256T, A40I+R118F+T244E, A40I+R118F+P256T, A40I+T244E+P256T, F51I+E56R+V60K, F51I+E56R+R118F, F51I+E56R+T244E, F51I+E56R+P256T, F51I+V60K+R118F, F51I+V60K+T244E, F51I+V60K+P256T, F51I+R118F+T244E, F51I+R118F+P256T, F51I+T244E+P256T, E56R+V60K+R118F, E56R+V60K+T244E, E56R+V60K+P256T, E56R+R118F+T244E, E56R+R118F+P256T, E56R+T244E+P256T, V60K+R118F+T244E, V60K+R118F+P256T, V60K+T244E+P256T, R118F+T244E+P256T, G23S+D27N+A40I+F51I, G23S+D27N+A40I+E56R, G23S+D27N+A40I+V60K, G23S+D27N+A40I+R118F, G23S+D27N+A40I+T244E, G23S+D27N+A40I+P256T, G23S+D27N+F51I+E56R, G23S+D27N+F51I+V60K, G23S+D27N+F51 l+R118F, G23S+D27N+F51I+T244E, G23S+D27N+F51I+P256T, G23S+D27N+E56R+V60K, G23S+D27N+E56R+R118F, G23S+D27N+E56R+T244E, G23S+D27N+E56R+P256T, G23S+D27N+V60K+R118F, G23S+D27N+V60K+T244E, G23S+D27N+V60K+P256T, G23S+D27N+R118F+T244E, G23S+D27N+R118F+P256T, G23S+D27N+T244E+P256T, G23S+A40I+F51I+E56R, G23S+A40I+F51I+V60K, G23S+A40I+F51I+R118F, G23S+A40I+F51I+T244E, G23S+A40I+F51I+P256T, G23S+A40I+E56R+V60K, G23S+A40I+E56R+R118F, G23S+A40I+E56R+T244E, G23S+A40I+E56R+P256T, G23S+A40I+V60K+R118F, G23S+A40I+V60K+T244E, G23S+A40I+V60K+P256T, G23S+A40I+R118F+T244E, G23S+A40I+R118F+P256T, G23S+A40I+T244E+P256T, G23S+F51I+E56R+V60K, G23S+F51I+E56R+R118F, G23S+F51I+E56R+T244E, G23S+F51I+E56R+P256T, G23S+F51I+V60K+R118F, G23S+F51I+V60K+T244E, G23S+F51I+V60K+P256T, G23S+F51I+R118F+T244E, G23S+F51I+R118F+P256T, G23S+F51I+T244E+P256T, G23S+E56R+V60K+R118F, G23S+E56R+V60K+T244E, G23S+E56R+V60K+P256T, G23S+E56R+R118F+T244E, G23S+E56R+R118F+P256T, G23S+E56R+T244E+P256T, G23S+V60K+R118F+T244E, G23S+V60K+R118F+P256T, G23S+V60K+T244E+P256T, G23S+R118F+T244E+P256T, D27N+A40I+F51I+E56R, D27N+A40I+F51I+V60K, D27N+A40I+F51I+R118F, D27N+A40I+F51I+T244E, D27N+A40I+F51I+P256T, D27N+A40I+E56R+V60K, D27N+A40I+E56R+R118F, D27N+A40I+E56R+T244E, D27N+A40I+E56R+P256T, D27N+A40I+V60K+R118F, D27N+A40I+V60K+T244E, D27N+A40I+V60K+P256T, D27N+A40I+R118F+T244E, D27N+A40I+R118F+P256T, D27N+A40I+T244E+P256T, D27N+F51I+E56R+V60K, D27N+F51I+E56R+R118F, D27N+F51I+E56R+T244E, D27N+F51I+E56R+P256T, D27N+F51I+V60K+R118F, D27N+F51I+V60K+T244E, D27N+F51I+V60K+P256T, D27N+F51I+R118F+T244E, D27N+F51I+R118F+P256T, D27N+F51I+T244E+P256T, D27N+E56R+V60K+R118F, D27N+E56R+V60K+T244E, D27N+E56R+V60K+P256T, D27N+E56R+R118F+T244E, D27N+E56R+R118F+P256T, D27N+E56R+T244E+P256T, D27N+V60K+R118F+T244E, D27N+V60K+R118F+P256T, D27N+V60K+T244E+P256T, D27N+R118F+T244E+P256T, A40I+F51I+E56R+V60K, A40I+F51I+E56R+R118F, A40I+F51I+E56R+T244E, A40I+F51I+E56R+P256T, A40I+F51I+V60K+R118F, A40I+F51I+V60K+T244E, A40I+F51I+V60K+P256T, A40I+F51I+R118F+T244E, A40I+F51I+R118F+P256T, A40I+F51I+T244E+P256T, A40I+E56R+V60K+R118F, A40I+E56R+V60K+T244E, A40I+E56R+V60K+P256T, A40I+E56R+R118F+T244E, A40I+E56R+R118F+P256T, A40I+E56R+T244E+P256T, A40I+V60K+R118F+T244E, A40I+V60K+R118F+P256T, A40I+V60K+T244E+P256T, A40I+R118F+T244E+P256T, F51I+E56R+V60K+R118F, F51I+E56R+V60K+T244E, F51I+E56R+V60K+P256T, F51I+E56R+R118F+T244E, F51I+E56R+R118F+P256T, F51I+E56R+T244E+P256T, F51I+V60K+R118F+T244E, F51I+V60K+R118F+P256T, F51I+V60K+T244E+P256T, F51I+R118F+T244E+P256T, E56R+V60K+R118F+T244E, E56R+V60K+R118F+P256T, E56R+V60K+T244E+P256T, E56R+R118F+T244E+P256T, V60K+R118F+T244E+P256T, G23S+D27N+A40I+F51I+E56R, G23S+D27N+A40I+F51I+V60K, G23S+D27N+A40I+F51I+R118F, G23S+D27N+A40I+F51 I+T244E, G23S+D27N+A40I+F51I+P256T, G23S+D27N+A40I+E56R+V60K, G23S+D27N+A40I+E56R+R118F, G23S+D27N+A40I+E56R+T244E, G23S+D27N+A40I+E56R+P256T, G23S+D27N+A40I+V60K+R118F, G23S+D27N+A40I+V60K+T244E, G23S+D27N+A40I+V60K+P256T, G23S+D27N+A40I+R118F+T244E, G23S+D27N+A40I+R118F+P256T, G23S+D27N+A40I+T244E+P256T, G23S+D27N+F51I+E56R+V60K, G23S+D27N+F51 I+E56R+R118F, G23S+D27N+F51I+E56R+T244E, G23S+D27N+F51 I+E56R+P256T, G23S+D27N+F51I+V60K+R118F, G23S+D27N+F51 I+V60K+T244E, G23S+D27N+F51 I+V60K+P256T, G23S+D27N+F51I+R118F+T244E, G23S+D27N+F51I+R118F+P256T, G23S+D27N+F51I+T244E+P256T, G23S+D27N+E56R+V60K+R118F, G23S+D27N+E56R+V60K+T244E, G23S+D27N+E56R+V60K+P256T, G23S+D27N+E56R+R118F+T244E, G23S+D27N+E56R+R118F+P256T, G23S+D27N+E56R+T244E+P256T, G23S+D27N+V60K+R118F+T244E, G23S+D27N+V60K+R118F+P256T, G23S+D27N+V60K+T244E+P256T, G23S+D27N+R118F+T244E+P256T, G23S+A40I+F51 I+E56R+V60K, G23S+A40I+F51 I+E56R+R118F, G23S+A40I+F51I+E56R+T244E, G23S+A40I+F51I+E56R+P256T, G23S+A40I+F51 I+V60K+R118F, G23S+A40I+F51I+V60K+T244E, G23S+A40I+F51I+V60K+P256T, G23S+A40I+F51I+R118F+T244E, G23S+A40I+F51I+R118F+P256T, G23S+A40I+F51I+T244E+P256T, G23S+A40I+E56R+V60K+R118F, G23S+A40I+E56R+V60K+T244E, G23S+A40I+E56R+V60K+P256T, G23S+A40I+E56R+R118F+T244E, G23S+A40I+E56R+R118F+P256T, G23S+A40I+E56R+T244E+P256T, G23S+A40I+V60K+R118F+T244E, G23S+A40I+V60K+R118F+P256T, G23S+A40I+V60K+T244E+P256T, G23S+A40I+R118F+T244E+P256T, G23S+F51I+E56R+V60K+R118F, G23S+F51 I+E56R+V60K+T244E, G23S+F51I+E56R+V60K+P256T, G23S+F51I+E56R+R118F+T244E, G23S+F51I+E56R+R118F+P256T, G23S+F51 I+E56R+T244E+P256T, G23S+F51I+V60K+R118F+T244E, G23S+F51I+V60K+R118F+P256T, G23S+F51I+V60K+T244E+P256T, G23S+F51I+R118F+T244E+P256T, G23S+E56R+V60K+R118F+T244E, G23S+E56R+V60K+R118F+P256T, G23S+E56R+V60K+T244E+P256T, G23S+E56R+R118F+T244E+P256T, G23S+V60K+R118F+T244E+P256T, D27N+A40I+F51I+E56R+V60K, D27N+A40I+F51 I+E56R+R118F, D27N+A40I+F51I+E56R+T244E, D27N+A40I+F51I+E56R+P256T, D27N+A40I+F51I+V60K+R118F, D27N+A40I+F51 I+V60K+T244E, D27N+A40I+F51I+V60K+P256T, D27N+A40I+F51I+R118F+T244E, D27N+A40I+F51 I+R118F+P256T, D27N+A40I+F51I+T244E+P256T, D27N+A40I+E56R+V60K+R118F, D27N+A40I+E56R+V60K+T244E, D27N+A40I+E56R+V60K+P256T, D27N+A40I+E56R+R118F+T244E, D27N+A40I+E56R+R118F+P256T, D27N+A40I+E56R+T244E+P256T, D27N+A40I+V60K+R118F+T244E, D27N+A40I+V60K+R118F+P256T, D27N+A40I+V60K+T244E+P256T, D27N+A40I+R118F+T244E+P256T, D27N+F51 I+E56R+V60K+R118F, D27N+F51 I+E56R+V60K+T244E, D27N+F51 I+E56R+V60K+P256T, D27N+F51 I+E56R+R118F+T244E, D27N+F51I+E56R+R118F+P256T, D27N+F51I+E56R+T244E+P256T, D27N+F51I+V60K+R118F+T244E, D27N+F51I+V60K+R118F+P256T, D27N+F51I+V60K+T244E+P256T, D27N+F51I+R118F+T244E+P256T, D27N+E56R+V60K+R118F+T244E, D27N+E56R+V60K+R118F+P256T, D27N+E56R+V60K+T244E+P256T, D27N+E56R+R118F+T244E+P256T, D27N+V60K+R118F+T244E+P256T, A40I+F51I+E56R+V60K+R118F, A40I+F51I+E56R+V60K+T244E, A40I+F51I+E56R+V60K+P256T, A40I+F51I+E56R+R118F+T244E, A40I+F51I+E56R+R118F+P256T, A40I+F51I+E56R+T244E+P256T, A40I+F51I+V60K+R118F+T244E, A40I+F51I+V60K+R118F+P256T, A40I+F51I+V60K+T244E+P256T, A40I+F51 I+R118F+T244E+P256T, A40I+E56R+V60K+R118F+T244E, A40I+E56R+V60K+R118F+P256T, A40I+E56R+V60K+T244E+P256T, A40I+E56R+R118F+T244E+P256T, A40I+V60K+R118F+T244E+P256T, F51I+E56R+V60K+R118F+T244E, F51I+E56R+V60K+R118F+P256T, F51 I+E56R+V60K+T244E+P256T, F51 I+E56R+R118F+T244E+P256T, F51I+V60K+R118F+T244E+P256T, E56R+V60K+R118F+T244E+P256T, G23S+D27N+A40I+F51I+E56R+V60K, G23S+D27N+A40I+F51I+E56R+R118F, G23S+D27N+A40I+F51I+E56R+T244E, G23S+D27N+A40I+F51 I+E56R+P256T, G23S+D27N+A40I+F51I+V60K+R118F, G23S+D27N+A40I+F51 I+V60K+T244E, G23S+D27N+A40I+F51I+V60K+P256T, G23S+D27N+A40I+F51I+R118F+T244E, G23S+D27N+A40I+F51 I+R118F+P256T, G23S+D27N+A40I+F51I+T244E+P256T, G23S+D27N+A40I+E56R+V60K+R118F, G23S+D27N+A40I+E56R+V60K+T244E, G23S+D27N+A40I+E56R+V60K+P256T, G23S+D27N+A40I+E56R+R118F+T244E, G23S+D27N+A40I+E56R+R118F+P256T, G23S+D27N+A40I+E56R+T244E+P256T, G23S+D27N+A40I+V60K+R118F+T244E, G23S+D27N+A40I+V60K+R118F+P256T, G23S+D27N+A40I+V60K+T244E+P256T, G23S+D27N+A40I+R118F+T244E+P256T, G23S+D27N+F51I+E56R+V60K+R118F, G23S+D27N+F51I+E56R+V60K+T244E, G23S+D27N+F51 I+E56R+V60K+P256T, G23S+D27N+F51I+E56R+R118F+T244E, G23S+D27N+F51I+E56R+R118F+P256T, G23S+D27N+F51 I+E56R+T244E+P256T, G23S+D27N+F51I+V60K+R118F+T244E, G23S+D27N+F51I+V60K+R118F+P256T, G23S+D27N+F51I+V60K+T244E+P256T, G23S+D27N+F51I+R118F+T244E+P256T, G23S+D27N+E56R+V60K+R118F+T244E, G23S+D27N+E56R+V60K+R118F+P256T, G23S+D27N+E56R+V60K+T244E+P256T, G23S+D27N+E56R+R118F+T244E+P256T, G23S+D27N+V60K+R118F+T244E+P256T, G23S+A40I+F51I+E56R+V60K+R118F, G23S+A40I+F51I+E56R+V60K+T244E, G23S+A40I+F51 I+E56R+V60K+P256T, G23S+A40I+F51I+E56R+R118F+T244E, G23S+A40I+F51I+E56R+R118F+P256T, G23S+A40I+F51 I+E56R+T244E+P256T, G23S+A40I+F51I+V60K+R118F+T244E, G23S+A40I+F51 I+V60K+R118F+P256T, G23S+A40I+F51I+V60K+T244E+P256T, G23S+A40I+F51I+R118F+T244E+P256T, G23S+A40I+E56R+V60K+R118F+T244E, G23S+A40I+E56R+V60K+R118F+P256T, G23S+A40I+E56R+V60K+T244E+P256T, G23S+A40I+E56R+R118F+T244E+P256T, G23S+A40I+V60K+R118F+T244E+P256T, G23S+F51I+E56R+V60K+R118F+T244E, G23S+F51I+E56R+V60K+R118F+P256T, G23S+F51I+E56R+V60K+T244E+P256T, G23S+F51I+E56R+R118F+T244E+P256T, G23S+F51I+V60K+R118F+T244E+P256T, G23S+E56R+V60K+R118F+T244E+P256T, D27N+A40I+F51 I+E56R+V60K+R118F, D27N+A40I+F51 I+E56R+V60K+T244E, D27N+A40I+F51I+E56R+V60K+P256T, D27N+A40I+F51I+E56R+R118F+T244E, D27N+A40I+F51I+E56R+R118F+P256T, D27N+A40I+F51 I+E56R+T244E+P256T, D27N+A40I+F51I+V60K+R118F+T244E, D27N+A40I+F51I+V60K+R118F+P256T, D27N+A40I+F51I+V60K+T244E+P256T, D27N+A40I+F51I+R118F+T244E+P256T, D27N+A40I+E56R+V60K+R118F+T244E, D27N+A40I+E56R+V60K+R118F+P256T, D27N+A40I+E56R+V60K+T244E+P256T, D27N+A40I+E56R+R118F+T244E+P256T, D27N+A40I+V60K+R118F+T244E+P256T, D27N+F51I+E56R+V60K+R118F+T244E, D27N+F51I+E56R+V60K+R118F+P256T, D27N+F51 I+E56R+V60K+T244E+P256T, D27N+F51I+E56R+R118F+T244E+P256T, D27N+F51I+V60K+R118F+T244E+P256T, D27N+E56R+V60K+R118F+T244E+P256T, A40I+F51I+E56R+V60K+R118F+T244E, A40I+F51I+E56R+V60K+R118F+P256T, A40I+F51 I+E56R+V60K+T244E+P256T, A40I+F51I+E56R+R118F+T244E+P256T, A40I+F51 I+V60K+R118F+T244E+P256T, A40I+E56R+V60K+R118F+T244E+P256T, F51I+E56R+V60K+R118F+T244E+P256T, G23S+D27N+A40I+F51I+E56R+V60K+R118F, G23S+D27N+A40I+F51I+E56R+V60K+T244E, G23S+D27N+A40I+F51I+E56R+V60K+P256T, G23S+D27N+A40I+F51I+E56R+R118F+T244E,
G23S+D27N+A40I+F51I+E56R+R118F+P256T,
G23S+D27N+A40I+F51I+E56R+T244E+P256T,
G23S+D27N+A40I+F51 I+V60K+R118F+T244E,
G23S+D27N+A40I+F51I+V60K+R118F+P256T,
G23S+D27N+A40I+F51I+V60K+T244E+P256T,
G23S+D27N+A40I+F51I+R118F+T244E+P256T,
G23S+D27N+A40I+E56R+V60K+R118F+T244E,
G23S+D27N+A40I+E56R+V60K+R118F+P256T,
G23S+D27N+A40I+E56R+V60K+T244E+P256T,
G23S+D27N+A40I+E56R+R118F+T244E+P256T,
G23S+D27N+A40I+V60K+R118F+T244E+P256T,
G23S+D27N+F51I+E56R+V60K+R118F+T244E,
G23S+D27N+F51I+E56R+V60K+R118F+P256T,
G23S+D27N+F51I+E56R+V60K+T244E+P256T,
G23S+D27N+F51I+E56R+R118F+T244E+P256T,
G23S+D27N+F51I+V60K+R118F+T244E+P256T,
G23S+D27N+E56R+V60K+R118F+T244E+P256T,
G23S+A40I+F51I+E56R+V60K+R118F+T244E, G23S+A40I+F51I+E56R+V60K+R118F+P256T,
G23S+A40I+F51I+E56R+V60K+T244E+P256T,
G23S+A40I+F51I+E56R+R118F+T244E+P256T,
G23S+A40I+F51I+V60K+R118F+T244E+P256T,
G23S+A40I+E56R+V60K+R118F+T244E+P256T,
G23S+F51I+E56R+V60K+R118F+T244E+P256T,
D27N+A40I+F51I+E56R+V60K+R118F+T244E, D27N+A40I+F51I+E56R+V60K+R118F+P256T,
D27N+A40I+F51I+E56R+V60K+T244E+P256T,
D27N+A40I+F51I+E56R+R118F+T244E+P256T,
D27N+A40I+F51I+V60K+R118F+T244E+P256T,
D27N+A40I+E56R+V60K+R118F+T244E+P256T,
D27N+F51I+E56R+V60K+R118F+T244E+P256T,
A40I+F51I+E56R+V60K+R118F+T244E+P256T,
G23S+D27N+A40I+F51I+E56R+V60K+R118F+T244E,
G23S+D27N+A40I+F51I+E56R+V60K+R118F+P256T,
G23S+D27N+A40I+F51I+E56R+V60K+T244E+P256T,
G23S+D27N+A40I+F51 I+E56R+R118F+T244E+P256T,
G23S+D27N+A40I+F51I+V60K+R118F+T244E+P256T,
G23S+D27N+A40I+E56R+V60K+R118F+T244E+P256T,
G23S+D27N+F51I+E56R+V60K+R118F+T244E+P256T,
G23S+A40I+F51I+E56R+V60K+R118F+T244E+P256T,
D27N+A40I+F51I+E56R+V60K+R118F+T244E+P256T,
G23S+D27N+A40I+F51 I+E56R+V60K+R118F+T244E+P256T.
   12. The variant of any one of any of paragraphs 1-11, wherein the variant comprises or consists of one of the following set of substitutions corresponding to:
A40E+E56R+D57N+G91T+K98E+R108K+R118F+E210K+T244E+D254S+I202H,
A40E+E56R+D57N+G91T+K98E+R108K+R118F+E210K+T244E+D254S+I252H,
A40E+E56R+D57N+G91T+K98E+R108K+R118F+E210K+T244E+D254S+L269H,
A40E+E56R+D57N+G91T+K98E+R108K+R118F+E210K+T244E+D254S+I202H+I252H,
A40E+E56R+D57N+G91T+K98E+R108K+R118F+E210K+T244E+D254S+I202H+L269H,
A40E+E56R+D57N+G91T+K98E+R108K+R118F+E210K+T244E+D254S+I252H+L269H,
A40E+E56R+D57N+G91T+K98E+R108K+R118F+E210K+T244E+D254S+I252H+L269H (using
SEQ ID NO: 8 for numberig).
   13. The variant of any one of any of paragraphs 1-12, wherein the variant comprises or consists of one of the following set of substitutions corresponding to:
G23S+D27N+A40I+F51I+E56R+V60K+R118F+T244E+P256T+I202H,
G23S+D27N+A40I+F51I+E56R+V60K+R118F+T244E+P256T+I252H,
G23S+D27N+A40I+F51I+E56R+V60K+R118F+T244E+P256T+L269H,
G23S+D27N+A40I+F51 I+E56R+V60K+R118F+T244E+P256T+I202H+I252H,
G23S+D27N+A40I+F51I+E56R+V60K+R118F+T244E+P256T+I202H+L269H,
G23S+D27N+A40I+F51I+E56R+V60K+R118F+T244E+P256T+I252H+L269H,
G23S+D27N+A40I+F51I+E56R+V60K+R118F+T244E+P256T+I202H+H252H+L269H (using
SEQ ID NO: 8 for numbeirng).
   14. The variant of any one of paragraphs 1-13, which variant further comprises one or more substitutions corresponding to F7K, F51I, F51L, F51V, F51Y, H198D, H198G, H198F, H1981, H198L, H198N, H198S, H198T, H198Y, N200Q, S224F, S224P, L227D, L227E, L227R, V228P, V230R, 1255G, 1255N, A257F, and A2571 (using SEQ ID NO: 8 for numbering).
   15. The variant of any one of paragraphs 1-14, wherein the lipase variant has reduced lipase activity and/or reduced odor generation at pHs around neutral, i.e., around pH 6-8, preferably around pH 7 and/or increased benefit risk factor (BRF) compared to the parent lipase, in particular SEQ ID NOs: 2, 4, 6 or 8, respectively.
   16. A granule, which comprises:
(a) a core comprising the variant of any one of paragraphs 1-15, and, optionally
(b) a coating consisting of one or more layer(s) surrounding the core.
   17. A granule, which comprises:
(a) a core, and
(b) a coating consisting of one or more layer(s) surrounding the core, wherein the coating comprises the variant of any one of paragraphs 1-15.
   18. A liquid composition comprising the variant of any one of paragraphs 1-15 and an enzyme stabilizer, e.g., a polyol such as propylene glycol or glycerol, sugar or sugar alcohol, lactic acid, reversible protease inhibitor, boric acid, or a boric acid derivative, e.g., an aromatic borate ester, or a phenyl boronic acid derivative such as 4-formylphenyl boronic acid).
   19. The liquid composition of paragraph 18, further comprising a filler or carrier material.
   20. The liquid composition of paragraph 18 or 19, further comprising a preservative.
   21. A composition comprising the variant of any one of paragraphs 1-15, the granule of paragraph 16 or 17, or the liquid compositions of any one of paragraphs 18-20.
   22. The composition of paragraph 21, wherein the composition comprises one or more surfactants.
   23. The composition of any paragraph 21 or 22, wherein the surfactant(s) is(are) present at a level of from 0.1 to 60 wt %, from 0.2 to 40 wt %, from 0.5 to 30 wt %, from 1 to 50 wt %, from 1 to 40 wt %, from 1 to 30 wt %, from 1 to 20 wt %, from 3 to 10 wt %, from 3 to 5 wt %, from 5 to 40 wt %, from 5 to 30 wt %, from 5 to 15 wt %, from 3 to 20 wt %, from 3 to 10 wt %, from 8 to 12 wt %, from 10 to 12 wt %, from 20 to 25 wt % or from 25-60 wt %.
   24. The composition of any one of paragraphs 21-23, wherein the composition comprising one or more surfactants wherein the one or more surfactants are selected from nonionic surfactants, anionic surfactants, cationic surfactants, ampholytic surfactants, zwitterionic surfactants, semi-polar nonionic surfactants and mixtures thereof.
   25. The composition of any one of paragraphs 21-24, wherein the composition comprises one or more anionic surfactant and/or one or more nonionic surfactant.
   26. The composition of any one of paragraphs 21-25, wherein the composition comprises one or more anionic surfactants, preferably linear alkylbenzenesulfonic acid (LAS), alcohol ethersulfate (AEOS) and/or alkyl sulfate (AS), in particular sodium lauryl sulfate (SLS).
   27. The composition of any one of paragraphs 21-26, wherein the composition comprises one or more non-ionic surfactants, preferably alcohol ethoxylate (AEO), in particular linear alcohol (C12-15) ethoxylate.
   28. The composition of any one of paragraphs 21-27, wherein the composition comprises one or more anionic surfactants and one or more nonionic surfactants.
   29. The composition of any one of paragraphs 21-28, wherein the composition comprises the anionic surfactants linear alkylbenzenesulfonic acid (LAS) and a nonionic surfactant alcohol ethoxylate (AEO).
   30. The composition of any one of paragraphs 21-29, wherein the composition further comprises one or more components selected from the group of builders, chelating agents, dye transfer inhibiting agents, dispersants, enzymes, and enzyme stabilizers, catalytic materials, bleach activators, hydrogen peroxide, sources of hydrogen peroxide, preformed peracids, polymeric dispersing agents, clay soil removal/anti-redeposition agents, brighteners, suds suppressors, dyes, hueing dyes, perfumes, perfume delivery systems, structure elasticizing agents, fabric softeners, carriers, hydrotropes, processing aids, solvents and/or pigments.
   31. The composition of any one of paragraphs 21-30, wherein the composition is formulated as a regular, compact or concentrated liquid; a gel; a paste; a soap bar; a regular or a compacted powder; a granulated solid; a homogenous or a multilayer tablet with two or more layers (same or different phases); a pouch having one or more compartments; a single or a multi-compartment unit dose form; or any combination thereof.
   32. The composition of any one of paragraphs 21-31, wherein the composition has reduced lipase activity and/or reduced odor generation at pHs around neutral, i.e., around pH 6-8, preferably around pH 7 and/or increased benefit risk factor (BRF) compared to the parent lipase, in particular SEQ ID NOs: 2, 4, 6 or 8 respectively.
   33. A polynucleotide encoding the variant of any one of paragraphs 1-15.
   34. A nucleic acid construct or expression vector comprising the polynucleotide of paragraph 33.
   35. A recombinant host cell transformed with the polynucleotide of paragraph 33.
   36. A method of producing a lipase variant, comprising:
a. cultivating the recombinant host cell of paragraph 35 under conditions suitable for expression of the variant; and
b. optionally recovering the variant.
   37. A method for hydrolyzing a lipase substrate comprising mixing the substrate with a lipase variant according to any one of paragraphs 1 to 15, or the composition of any one of paragraphs 21 to 32, at conditions conductive for the lipase variant hydrolyzing the substrate.
   38. A method for removing lipid stain material from a surface comprising contacting the lipid stain material with a lipase variant according to any one of paragraphs 1 to 15, or the composition of any one of paragraphs 21 to 32, at conditions conductive for the lipase variant hydrolyzing the lipid stain material.
   39. A method for lipid stain removal from a surface comprising: contacting said stain with a lipase variant of any of paragraphs 1 to 15, or a composition of any one of paragraphs 21 to 32, followed by rinsing of the surface, and optionally drying.
   40. A method for lipid stain removal from a surface comprising: contacting said stain with a lipase variant of any of paragraphs 1 to 15, or a composition of any of paragraphs 21 to 32, followed by rinsing of the surface, and optionally drying, in which method, the odor generation is reduced when compared to the method wherein the parent lipase, in particular one of SEQ ID NOs: 2, 4, 6, or 8, respectively, is contacted with the stain.
   41. A method for washing laundry, comprising the steps of
i) washing by subjecting said laundry to a lipase of any one of paragraphs 1-15, a granule of any one of paragraphs 16 or 17, a liquid composition of any one of paragraphs 18-20, or a composition of any of paragraphs 21-32;
ii) rinsing the laundry; and
iii) optionally drying the laundry.
   42. The method of paragraph 41, wherein washing cycle in step i) is carried out at a pH around 8-11 and rising step ii) is carried out a pH around 6-8, preferably around 7.
   43. The method of paragraphs 41 or 42, wherein the wash cycle and/or rising step is caried out in an aqueous solution.
   44. The method of any one of paragraphs 41-43, wherein the washing cycle in step i) is carried out at between 10-90° C., in particular between 20° C. and 40° C. or between 50° C. and 70° C.
   45. Use of a lipase variant according to any one of paragraphs 1 to 15, a granule of any one of paragraphs 16 or 17, a liquid composition of any one of paragraphs 18-20, or a composition of any one of paragraphs 21 to 32 for cleaning a surface comprising applying the lipase variant to the surface to be cleaned.
   46. Use of a lipase variant for cleaning a surface comprising: subjecting to said surface to be cleaned to a lipase variant of any one of paragraphs 1-15, a granule of any one of paragraphs 16 or 17, a liquid composition of any one of paragraphs 18-20, or a composition of any one of paragraphs 21-32.

### Materials & Methods

### Model Detergent A2 (Liquid)

| Composition | Active |
|---|---|
| Detergent | content |
| Sodium linear alkylbenzene sulfonate (Na-LAS) | 12.0% |
| Sodium lauryl ether sulfate (SLES) | 4.1% |
| Alcohol ethoxylate with 7 mol EO (AEO) | 12.0% |
| Topped Palm Kernel Fatty Acid | 3.0% |
| Sodium hydroxide | 0.5% |
| Ethanol | 6.0% |
| Propylene glycol (MPG) | 2.0% |
| Triethanolamine (TEA) | 2.0% |
| Trisodium citrate dihydrate | 3.9% |
| Diethylenetriaminepentakis(methylene)pentakis(phosphonic | 1.6% |
| acid), heptasodium salt (DTPMP Na7) | |
| 2-Phenoxyethanol | 0.5% |
| Water (demineralized) | Ad 100% |
| pH target 8.2-8.6 | 8.4 |

### Terg-O-Tometer (TOM)

The Terg-O-tometer (TOM) is a medium scale model wash system that can be applied to test 12 different wash conditions simultaneously. A TOM is basically a large temperature-controlled water bath with up to 12 open metal beakers submerged into it. Each beaker constitutes one small top loader style washing machine and during an experiment, each of them will contain a solution of a specific detergent/enzyme system and the soiled and unsoiled fabrics its performance is tested on. Mechanical stress is achieved by a rotating stirring arm, which stirs the liquid within each beaker. Because the TOM beakers have no lid, it is possible to withdraw samples during a TOM experiment and assay for information on-line during wash.

### EXAMPLES

### Example 1

Growth of Lipase His-Variants Lipase p-Nitrophenyl (pNP) Activity Assay.

Aspergillus oryzae strains expressing lipase variants were inoculated in 200 μl MDU2 media with 0.5% urea in a 96 well MTP. The plate was incubated at 30° C. for 4 days in a box with wet cloth (to avoid drying out) without any shaking.

After incubation, the media containing the expressed lipase variants, was taken from each of the outgrown wells.

10 μl of media was diluted in 90 ml buffer (either 50 mM Tris pH7.0, 50 mM Tris pH9.0 or 50 mM glycine-buffer pH10) from each well.

Lipase activity in the diluted media was determined as follows: 200 μl of a 1 mM pNP-palmitate solution (in either 50 mM Tris pH7.0, 50 mM Tris pH9.0 or 50 mM glycine-buffer pH10 plus 0.4% TritonX-100 and 10 mM CaCl₂) was added to 10 μl of diluted media in a 96 well MTP. The activity measurement was also performed with a 1 mM pNP-palmitate solution in 3.3 g/L Model detergent A2 in 15° dH water hardness. The activity was measured by measuring increase in absorbance at 405 nm (due to the release of pNP (para-nitrophenol)). The slope of the initial activity in the linear range was determined, as an indication of the given specific activity at the given pH. The steeper the slope, the more active the lipase variant is.

### Example 2

Lipase activity for His-Variants at pH 7 and pH 9

The purpose of this experiment is to show that the lipase activity around neutral pHs is reduced compared to at higher pHs.

The tests were repeated in triplicate with the following outcome (Vmax-data).

| | | | Activity in |
|---|---|---|---|
| | Activity at | Activity at | Model A2 |
| Mutations | pH 7 (buffer) | pH 9 (buffer) | detergent |
| SEQ ID NO: 8 (parent) | 100 | 100 | 100 |
| SEQ ID NO: 8 + I202H | 3 | 22 | 79 |
| SEQ ID NO: 8 + I252H | 68 | 98 | 93 |
| SEQ ID NO: 8 + L269H | 65 | 109 | 100 |

The data shows that I202H, I252H and L269H variants have reduced lipase activity at pH 7 compared to pH 9. This shows that the lipase activity (and odor generation) during the wash cycle is higher than during the rinse cycle

### Example 3

Wash Performance, Odor Generation and BRF of his-Variants

Lard was melted and 100 μL applied to cotton fabric swatches 5×5 cm (WFK80A, 100% Cotton, Blue) and incubated at 100° C. for 20 minutes. After cooling the swatches were weighed on an analytical balance.

Four swatches were washed in a Terg-O-tometer washing machine using 3.3 g Model detergent A2 and 0.05 mg of lipase per L wash solution. The water hardness was 15° dH Ca⁺⁺/Mg⁺⁺/HCO³⁻ (ratio 4:1:7.5). The swatches were washed at 20° C., 120 rpm, pH 8.2 (before and after wash) for 20 minutes and then rinsed in 5 L beakers (pH 7.4 after rinse) under running tap water.

After washing and rinsing the swatches were incubated at 100° C. for 20 minutes and after cooling the swatches were weighed on an analytical balance.

The wash test consisted of two replicates for each treatment.

Fat removal is calculated as:

% Fat removal=((weight of lard soiled swatches before wash)−(weight of lard soiled swatches after wash))/((weight of lard soiled swatches before wash)−(weight of swatches before soiling))

Wash performance and odor generation was measured, and the Benefit Risk Factor (BRF) relationship was calculated. The better performance compared to odor, the better the relationship. The template (SEQ ID NO: 8) was set to 1.0.

| | Wash | | |
|---|---|---|---|
| Mutation | Performance | Odor-generation | BRF |
| SEQ ID NO: 8 (parent) | 1.0 | 1.0 | 1.0 |
| SEQ ID NO: 8 + I202H | 0.70 | 0.30 | 2.3 |
| SEQ ID NO: 8 + I252H | 0.85 | 0.61 | 1.4 |
| SEQ ID NO: 8 + L269H | 0.93 | 0.56 | 1.7 |

### Example 4

Growth of Lipase His-Variants and pNP-Assay on His-Variants at pH7 and pH10

Aspergillus oryzae strains expressing the lipase variants were inoculated in 200 μl MDU2 media with 0.5% urea in a 96 well MTP. The plate was incubated at 30° C. for 4 days in a box with wet cloth (to avoid drying out) without any shaking.

After incubation, the media containing the expressed lipase variants was taken from each of the outgrown wells. 10 μl of media was diluted in 90 μl buffer (either 50 mM Tris pH7.0 or 50 mM glycine-buffer pH10) from each well.

Activity in the diluted media was determined as follows: 200 μl of a 1 mM pNP-palmitate solution (either 50 mM Tris pH7.0 or 50 mM glycine-buffer pH10 plus 0.4% TritonX-100 and 10 mM CaCl₂) was added to 10 μl of diluted media in a 96 well MTP. The activity was measured by measuring increase in absorbance at 405 nm (due to the release of pNP (para-nitrophenol)). The slope of the initial activity in the linear range was determined, as an indication of the given specific activity at the given pH. The steeper the slope, the more active the lipase variant is.

| | Activity at pH 7 | Activity at pH 10 |
|---|---|---|
| Mutations | (buffer) | (buffer) |
| SEQ ID NO: 8 (parent) | 100 | 100 |
| SEQ ID NO: 8 + L206H + | 46 | 100 |
| I252H + L269H | | |
| SEQ ID NO: 6 (parent) | 100 | 100 |
| SEQ ID NO: 6 + I202H + | 49 | 100 |
| I252H + L269H | | |
| SEQ ID NO: 6 + L269H | 65 | 100 |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A lipase variant, selected from one or more of groups (i), (ii) and (iii) comprising
(i) a substitution at one or more positions corresponding to positions 252, 202, and 269 of the polypeptide of SEQ ID NO: 8;
(ii) a substitution at one or more positions corresponding to positions 40, 56, 57, 91, 98, 108, 118, 210, 244, and 254 of the polypeptide of SEQ ID NO: 8; and
(iii) a substitution at one or more positions corresponding to positions 23, 27, 40, 51, 56, 60, 118 244 and 256 of the polypeptide of SEQ ID NO: 8;
wherein the variant has lipase activity and wherein the variant has at least 60%, at least 65%, at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 8.

2. The variant of claim 1, which comprises or consists of one or more substitutions, in particular all substitutions, corresponding to the positions in SEQ ID NO: 8, selected from the group consisting of:
a substitution of the amino acid residue at position 202 with H;
a substitution of the amino acid residue at position 252 with H; and
a substitution of the amino acid residue at position 269 with H; or
wherein the variant comprises or consists of one of the following sets of substitutions:
202H+252H; 202H+269H; 252H+269H; or 202H+252H+269H.

3. The variant of claim 1, which comprises or consists of one or more substitutions, in particular all substitutions, corresponding to the positions in SEQ ID NO: 8, selected from the group consisting of:
a substitution of the amino acid residue at position 40 with E;
a substitution of the amino acid residue at position 56 with R;
a substitution of the amino acid residue at position 57 with N;
a substitution of the amino acid residue at position 91 with T;
a substitution of the amino acid residue at position 98 with E;
a substitution of the amino acid residue at position 108 with K;
a substitution of the amino acid residue at position 118 with F;
a substitution of the amino acid residue at position 210 with K;
a substitution of the amino acid residue at position 244 with E; and
a substitution of the amino acid residue at position 254 with S.

4. The variant of claim 1, which comprises or consists of one or more substitutions, in particular all substitutions, corresponding to the positions in SEQ ID NO: 8, selected from the group consisting of:
a substitution of the amino acid residue at position 23 with S;
a substitution of the amino acid residue at position 27 with N;
a substitution of the amino acid residue at position 40 with I;
a substitution of the amino acid residue at position 51 with I;
a substitution of the amino acid residue at position 56 with R;
a substitution of the amino acid residue at position 60 with K;
a substitution of the amino acid residue at position 118 with F;
a substitution of the amino acid residue at position 244 with E; and
a substitution of the amino acid residue at position 256 with T.

5. The variant of claim 1, wherein the lipase variant has reduced lipase activity and/or reduced odor generation at pH 6-8 and/or increased benefit risk factor (BRF) compared to the parent lipase, wherein the parent lipase comprises the amino acid sequence of SEQ ID NOs: 2, 4, 6 or 8.

6. A granule, which comprises:
(a) a core and
(b) a coating consisting of one or more layer(s) surrounding the core,
wherein the coating or the core comprises the variant of claim 1.

7. A liquid composition comprising the variant of claim 1 and an enzyme stabilizer.

8. A composition comprising the variant of claim 1.

9. A polynucleotide encoding the variant of claim 1.

10. A nucleic acid construct or expression vector comprising the polynucleotide of claim 9.

11. A recombinant host cell transformed with the polynucleotide of claim 9.

12. A method of producing a lipase variant, comprising:
a. cultivating the recombinant host cell of claim 11 under conditions suitable for expression of the variant; and
b. recovering the variant.

13. A method for hydrolyzing a lipase substrate comprising mixing the substrate with a lipase variant according to claim 1, at conditions conductive for the lipase variant hydrolyzing the substrate.

14. A method for lipid stain removal from a surface comprising: contacting said stain with the composition of claim 8, followed by rinsing of the surface.

15. A method for washing laundry, comprising the steps of
i) washing by subjecting said laundry to the composition of claim 8; and
ii) rinsing the laundry.

16. The variant of claim 1, wherein the variant has lipase activity and comprises an extension of one or more amino acids at the N-terminal and/or C-terminal ends or a truncation of one or more amino acids at the N-terminal and/or C-terminal ends.
